(19) United States
(12) Patent Application Publication
(10) Pub. No.: US 2025/0152569 A1
(43) Pub. Date: May 15, 2025

(54) TREATMENT OF CELL PROLIFERATION-ASSOCIATED CONDITIONS USING A COMBINATION OF A CLB-B INHIBITOR AND AN ADDITIONAL THERAPEUTIC AGENT

(71) Applicant: HOTSPOT THERAPEUTICS, INC., Boston, MA (US)

(72) Inventors: Fang WANG, Belmont, MA (US); Yilin QI, Belmont, MA (US); Timothy P. REILLY, New Hope, PA (US)

(21) Appl. No.: 19/016,716

(22) Filed: Jan. 10, 2025

Related U.S. Application Data
(63) Continuation of application No. PCT/US2023/070042, filed on Jul. 12, 2023.
(60) Provisional application No. 63/388,518, filed on Jul. 12, 2022.

(51) Int. Cl. A61K 31/4439; A61K 45/06; A61P 35/00

(52) U.S. Cl. CPC A61K 31/4439; A61K 45/06; A61P 35/00

(57) ABSTRACT

The present disclosure relates to treatment of a subject in need thereof of comprising administering a therapeutically effective amount of a CBL-B inhibitor and a therapeutically effective amount of an additional therapeutic agent.

### CROSS-REFERENCE TO RELATED APPLICATIONS

This application is a continuation of International (PCT) Patent Application No. PCT/US2023/070042, filed on Jul. 12, 2023, which claims the benefit of, and priority to, U.S. Patent Application No. 63/388,518, filed on Jul. 12, 2022, the content of each of which is hereby incorporated by reference in its entirety.

### BACKGROUND

E3 ligase Casitas B-Lineage Lymphoma Proto-Oncogene B (CBL-B) is a key negative modulator of T-cell receptor and co-stimulatory regulation. CBL-B inhibition lowers the threshold of antigen-specific T cell activation, even in absence of co-stimulatory signaling or in the presence of an immune suppressive environment. Genetic ablation of CBL-B or functional inactivation of its E3 ligase activity in mice or primary human T cells enhances immune-mediated tumor growth control. Therefore, CBL-B inhibition may address the suboptimal response to current immunotherapies due to low inflammation, no/low co-stimulation signal or a high immune suppressive environment.

### SUMMARY

The present disclosure includes a method of treating a disease or condition associated with cell proliferation comprising administering a therapeutically effective amount of a CBL-B inhibitor in combination with an additional therapeutic agent.

The present disclosure is further defined in the appended claims.

### BRIEF DESCRIPTION OF THE DRAWINGS

FIG. 1 is a graph illustrating mean tumor volume curve of vehicle and treatment group with SEM as described in Example 4.

FIG. 2A and FIG. 2B are graphs of Compound 29 and Doxrubicin in combination that shows improved effect on tumor inhibition as compared to each of the agents as monotherapies

FIG. 3A and FIG. 3B are graphs of Compound 29 and Gemcitabine in combination that shows improved effect on tumor inhibition as compared to each of the agents as monotherapies.

FIG. 4A and FIG. 4B are graphs of Compound 29 and Paclitaxel in combination that shows improved effect on tumor inhibition as compared to each of the agents as monotherapies

FIG. 5A and FIG. 5B are graphs of Compound 29 and ALT-803 in combination that shows improved effect on tumor inhibition as compared to each of the agents as monotherapies.

### DETAILED DESCRIPTION

### CBL-B Inhibitors

An “CBL-B Inhibitor” refers to a compound that, upon administration to a subject, results in inhibition or down-regulation of a biological activity associated with activation of CBL-B in the patient, including any of the downstream biological effects otherwise resulting from the binding to CBL-B of its natural ligand. Such CBL-B Inhibitors include any agent that can block activation of CBL-B or any of the downstream biological effects of CBL-B activation.

In some embodiments, a CBL-B inhibitor is a compound of formula (A):

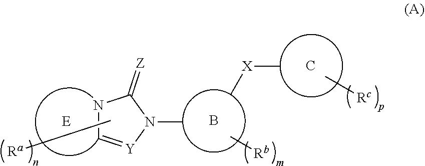

or pharmaceutically acceptable salts thereof,
   wherein
   Y is selected from the group ═C(H)—, ═C(R^{a})— or ═N—;

### Z is ═O or ═S;

E is optionally substituted 5-6 membered heterocyclyl;
B is optionally substituted phenyl, optionally substituted 8-10 membered bicyclyl, or optionally substituted 5-6 membered heteroaryl;
C is optionally substituted 5-6 membered heterocyclyl;
X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl, wherein X is optionally substituted with an optionally substituted group selected from a group consisting of halogen, C₁-C₃ aliphatic, phenyl, 3-6-membered heteroaryl, 3-6-membered heterocylyl, and —(CH₂)(3-6-membered carbocyclyl);
each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
   each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
   n is 0, 1, 2, 3, 4, or 5;
   m is 0, 1, 2, 3, or 4; and
   p is 0, 1, 2, 3, or 4.

In some embodiments, the present disclosure includes a compound of Formula (B):

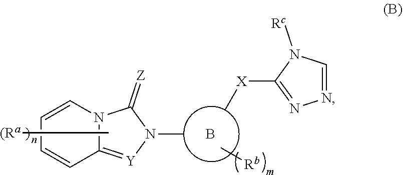

or pharmaceutically acceptable salts thereof,
   wherein
   Y is selected from the group ═C(H)—, ═C(R^{a})— or ═N—;

### Z is ═O or ═S;

B is optionally substituted phenyl, substituted 5-6 membered heteroaryl or optionally substituted 8-10 membered bicyclyl;
X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl,
each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, —OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
   each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
   n is 0, 1, 2, 3, 4, or 5; and
   m is 0, 1, 2, 3, or 4.

In some embodiments, the present disclosure includes a compound of formula (I):

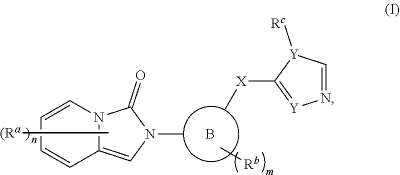

or pharmaceutically acceptable salts thereof,
   wherein
   X is an optionally substituted C₁-C₃ alkylene chain, wherein one or more methylene units is optionally replaced by —N(H)—, —N(R¹)—, —O—, —S—, —SO—, —SO₂—,

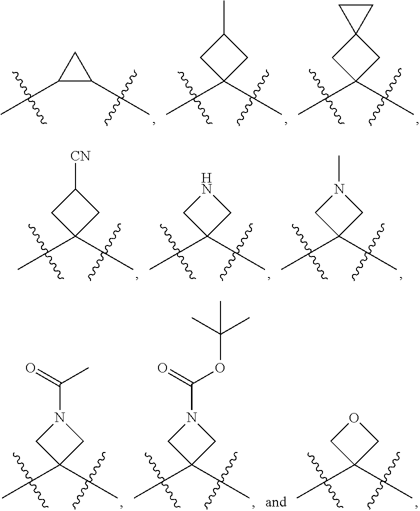

and wherein each methylene unit may be substituted with 1-2 substituents independently selected from the group consisting of halogen, optionally substituted C₁-C₃ aliphatic, optionally substituted 5-membered heteroaryl, optionally substituted phenyl, optionally substituted C₃-C₄ carbocylyl, and optionally substituted C₃-C₄ heterocyclyl;
each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
each Y is independently selected from the group consisting of —C═, —O—, —N═, and —S—;
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
B is optionally substituted phenyl, substituted 5-6 membered heteroaryl, or optionally substituted 8-10 membered bicyclyl;
each R^{b} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, wherein the—optionally deuterated optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
   each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
   n is 0, 1, 2, 3, 4, or 5; and
   m is 0, 1, 2, 3, or 4.

In some embodiments, present disclosure includes a compound is of formula (Ia) or (IIa):

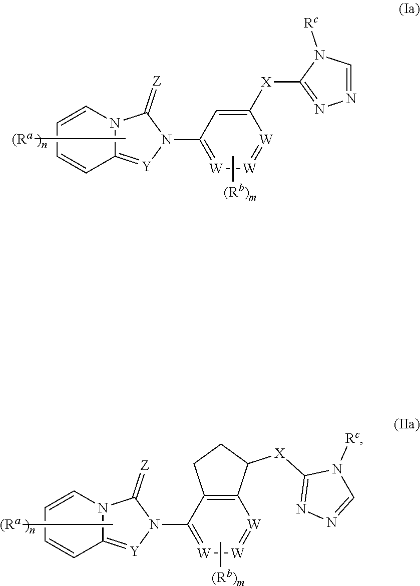

or pharmaceutically acceptable salts thereof,
wherein each W is independently selected from N or C; and
X, Y, Z, R^{a}, R^{b}, R^{c}, n, and m are defined above and described in classes and subclasses herein.

In some embodiments, present disclosure includes a compound is of formula (Ia1) or (IIa1):

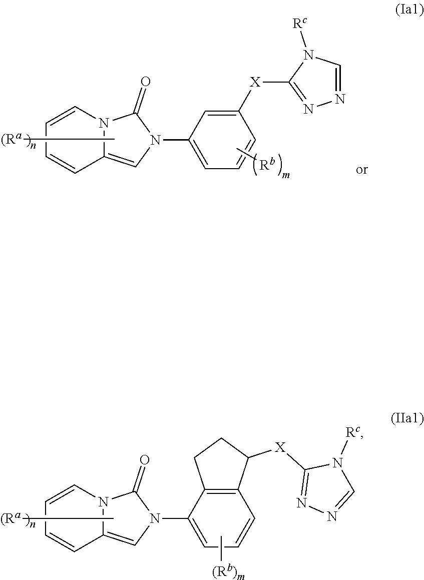

or a pharmaceutically acceptable salt thereof, wherein X, Y, Z, R^{a}, R^{b}, R^{c}, n, and m are defined above and described in classes and subclasses herein.

In some embodiments, present disclosure includes a compound is of formula (Ia2), (Ia3), or (Ia4):

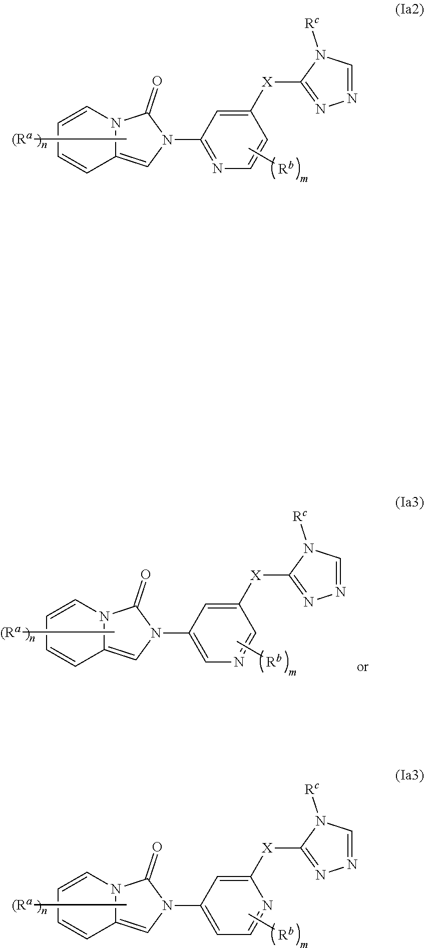

or pharmaceutically acceptable salts thereof, wherein X, Y, Z, R^{a}, R^{b}, R^{c}, n, and m are defined above and described in classes and subclasses herein.

In some embodiments, present disclosure includes a compound is of formula (Ia1) or (IIa1):

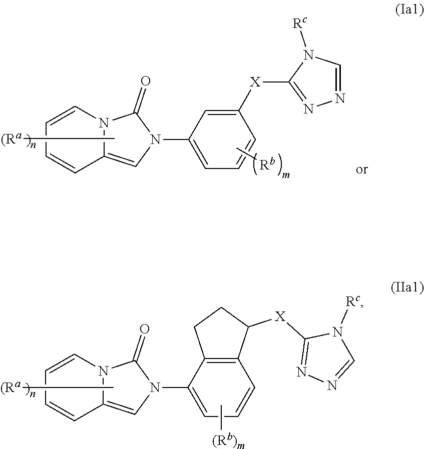

or pharmaceutically acceptable salts thereof,
   wherein
   X is an optionally substituted C₁-C₃ alkylene chain, wherein one or more methylene units is optionally replaced by —N(H)—, —N(R¹)—, —O—, —S—, —SO—, —SO₂—,

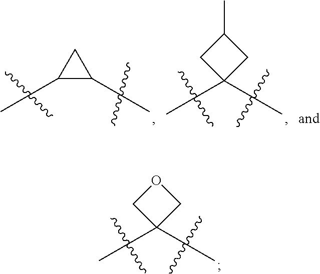

each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
   each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
   n is 0, 1, 2, 3, 4, or 5; and
   m is 0, 1, 2, 3, or 4.

In some embodiments, present disclosure includes a compound is of formula (Ib) or (IIb):

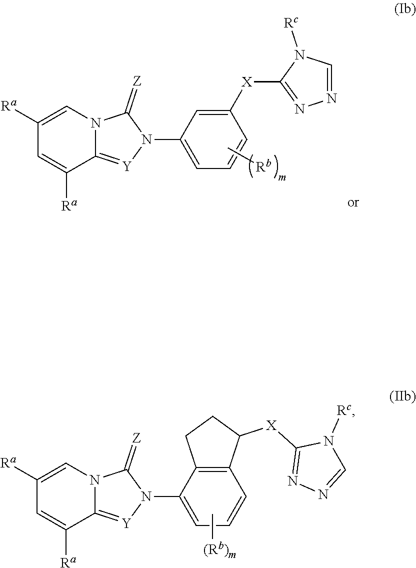

or pharmaceutically acceptable salts thereof, wherein X, Y, Z, R^{a}, R^{b}, R^{c}, and m are defined above and described in classes and subclasses herein.

In some embodiments, present disclosure includes a compound of formula (Ib1) or (IIb1):

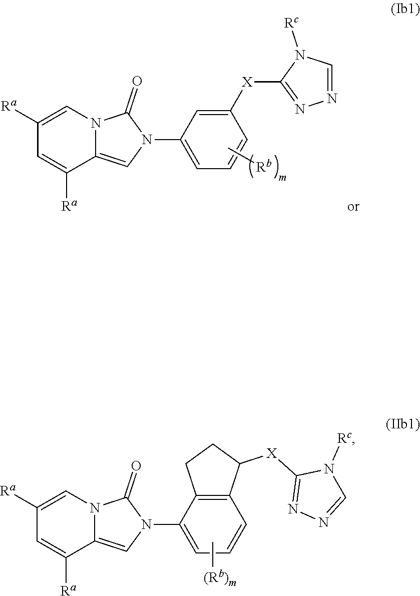

or a pharmaceutically acceptable salt thereof, wherein X, R^{a}, R^{b}, R^{c} and m are defined above and described in classes and subclasses herein.

In some embodiments, present disclosure includes a compound of formula (Ib2), (Ib3), or (Ib4):

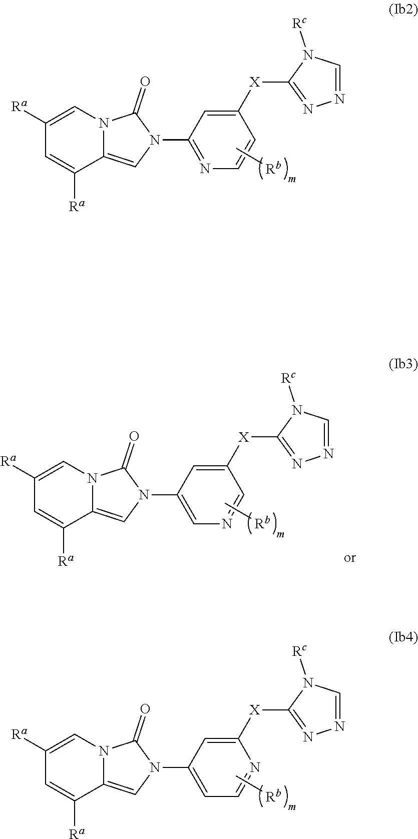

or a pharmaceutically acceptable salt thereof, wherein X, R^{a}, R^{b}, R^{c} and m are defined above and described in classes and subclasses herein.

In some embodiments, present disclosure includes a compound of formula (Ic) or (IIc):

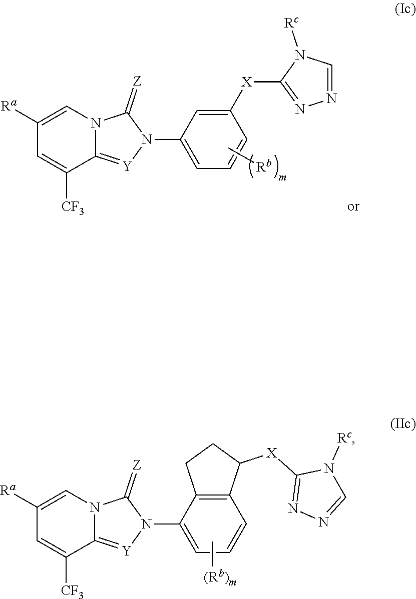

or a pharmaceutically acceptable salt thereof, wherein X, Y, Z, R^{a}, R^{b}, R^{c} and m are defined above and described in classes and subclasses herein.

In some embodiments, present disclosure includes a compound of formula (Ic1) or (IIc1):

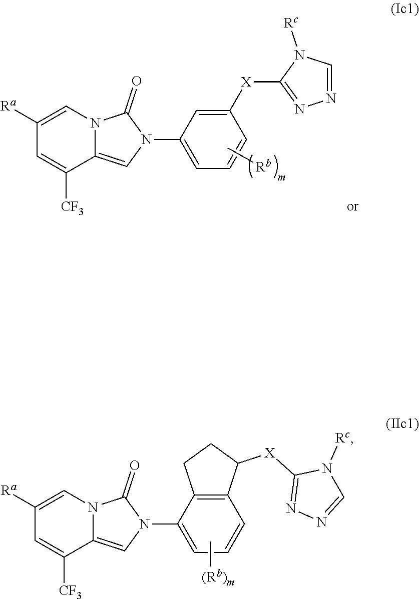

or a pharmaceutically acceptable salt thereof, wherein X, R^{a}, R^{b}, R^{c} and m are defined above and described in classes and subclasses herein.

In some embodiments, present disclosure includes a compound of formula (Id) or (IId):

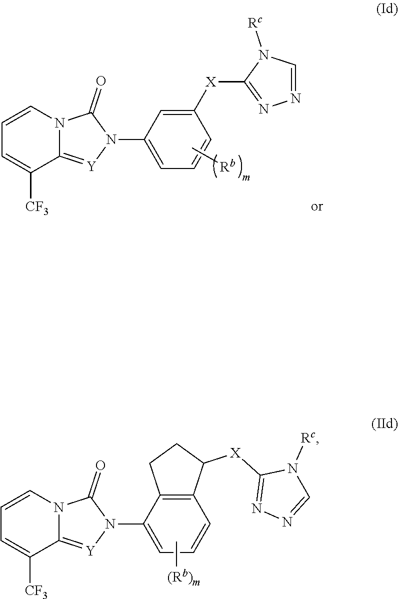

or a pharmaceutically acceptable salt thereof, wherein X, R^{b}, R^{c} and m are defined above and described in classes and subclasses herein.

In some embodiments, present disclosure includes a compound of formula (Id1) or (IId1):

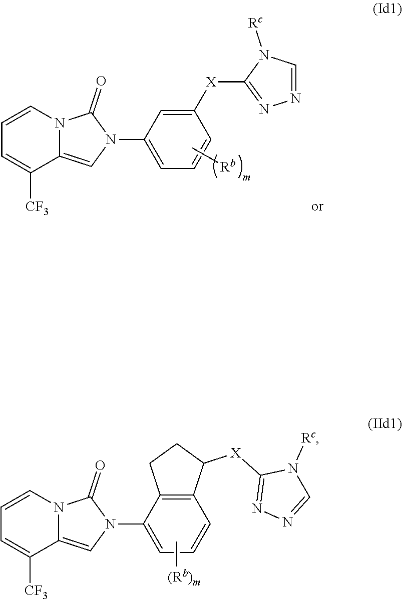

or a pharmaceutically acceptable salt thereof, wherein X, R^{b}, R^{c} and m are defined above and described in classes and subclasses herein.

### X

In some embodiments, X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl, wherein X is optionally substituted with an optionally substituted group selected from the group consisting of halogen, C₁-C₃ aliphatic, phenyl, 3-6-membered heteroaryl, 3-6-membered heterocylyl, and —(CH₂)(3-6-membered carbocyclyl). In some embodiments, X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl. In some embodiments, X is an optionally substituted C₁-C₃ alkylene chain, wherein one or more methylene units is optionally replaced by —N(H)—, —N(R¹)—, —O—, —S—, —SO—, —SO₂—,

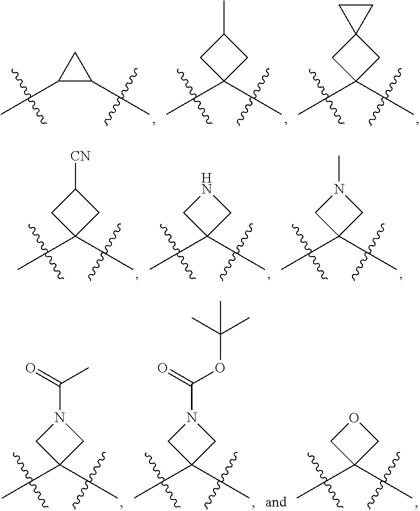

and wherein each methylene unit may be substituted with 1-2 substituents independently selected from the group consisting of halogen, optionally substituted C₁-C₃ aliphatic, optionally substituted 5-membered heteroaryl, optionally substituted phenyl, optionally substituted C₃-C₄ carbocylyl, and optionally substituted C₃-C₄ heterocyclyl. In some embodiments, In some embodiments, X is an optionally substituted C₁-C₃ alkylene chain, wherein one or more methylene units is optionally replaced by —N(H)—, —N(R¹)—, —O—, —S—,

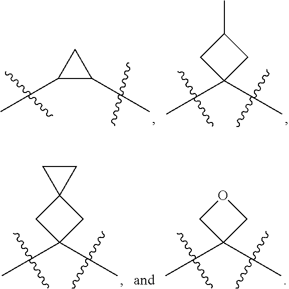

In some embodiments, X is an optionally substituted C₁-C₃ alkylene chain, wherein one or more methylene units is optionally replaced by —N(H)—, —N(R¹)—, —O—, —S—, —SO—, —SO₂—,

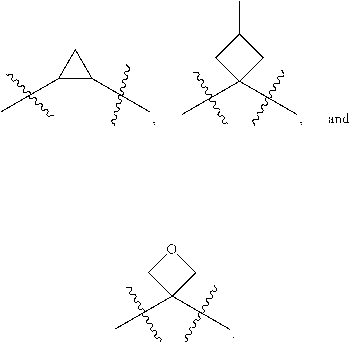

In some embodiments, X is optionally substituted C₁-C₂ alkylene. In some embodiments, X is

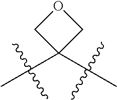 [SMILES: *C1(*)COC1]

or optionally substituted C₂ alkylene, wherein one methylene unit is replaced with

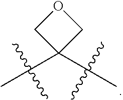 [SMILES: *C1(*)COC1]

In some embodiments, X is selected from the group consisting of

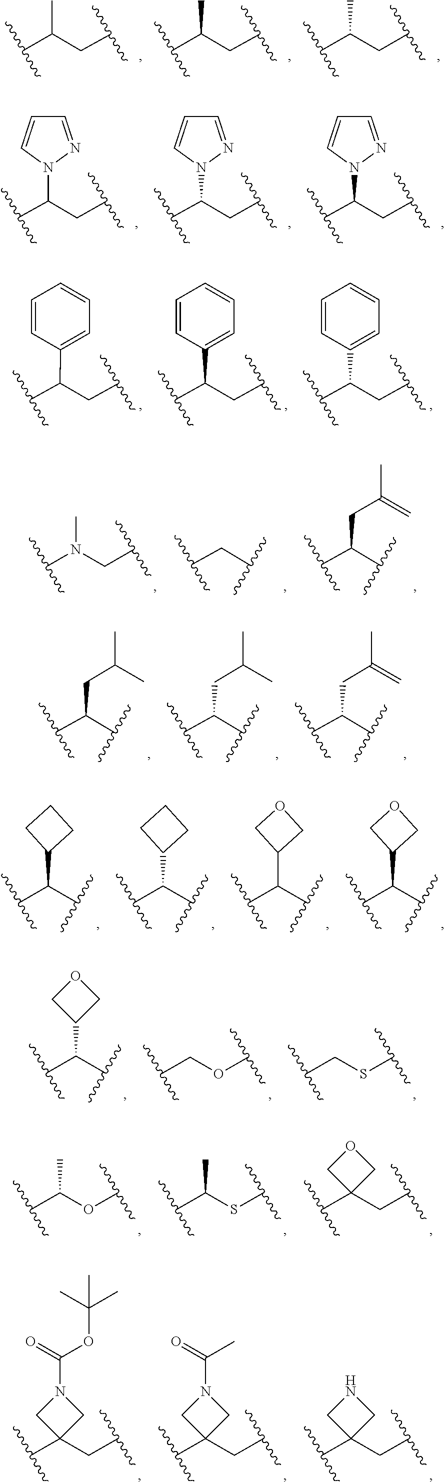

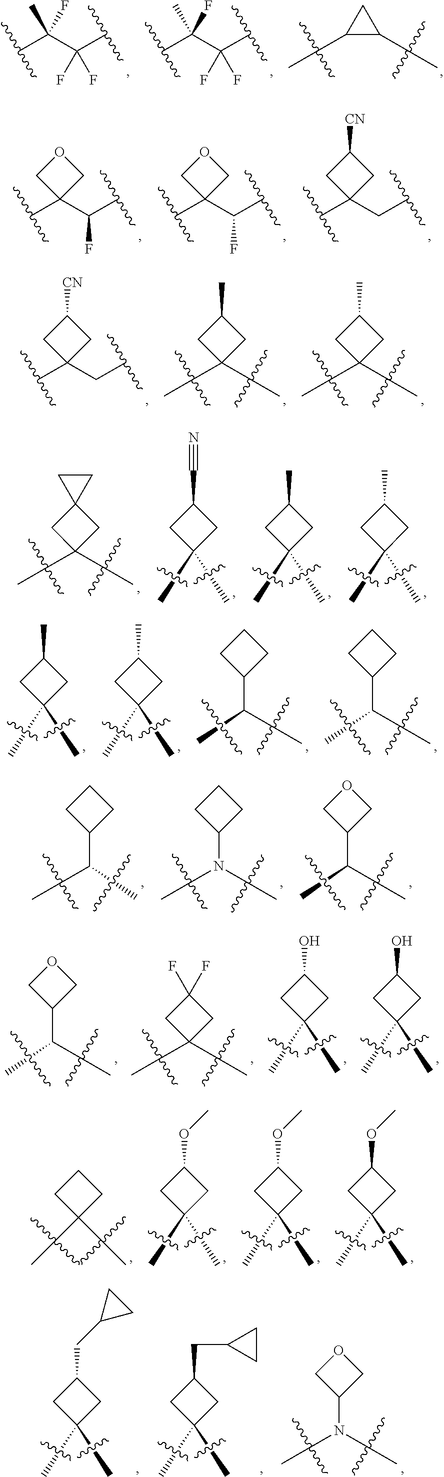

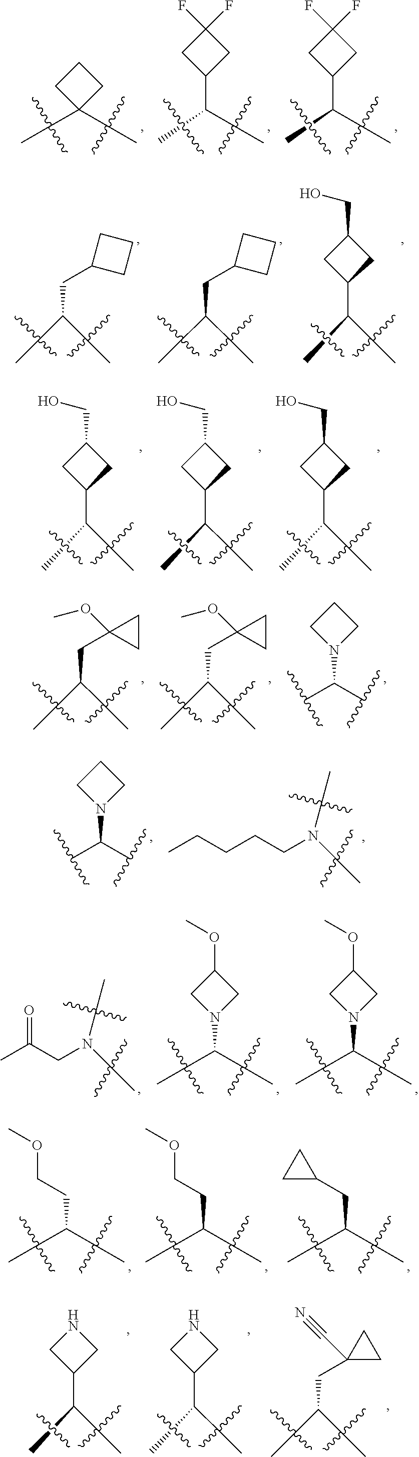

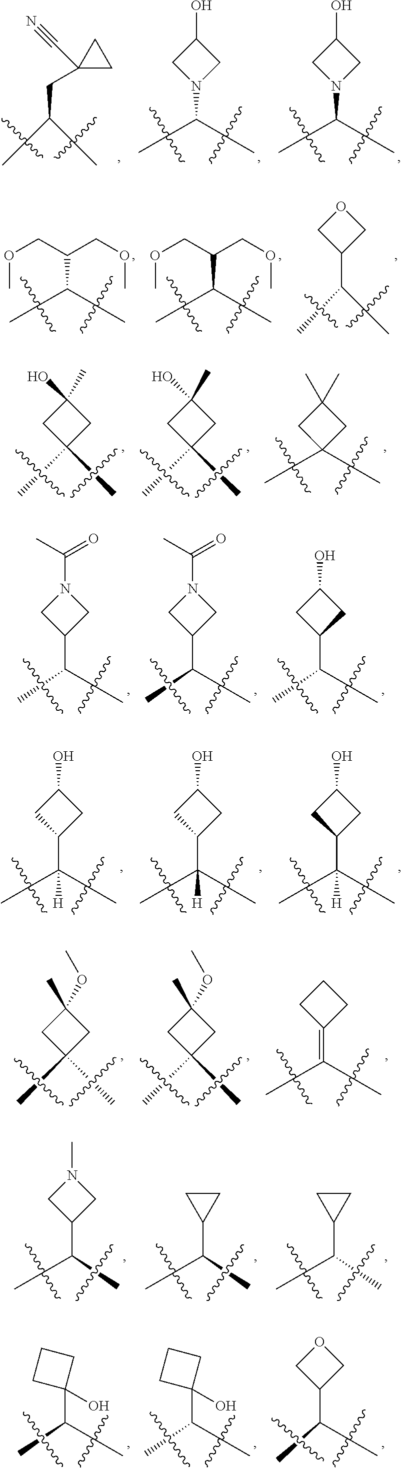

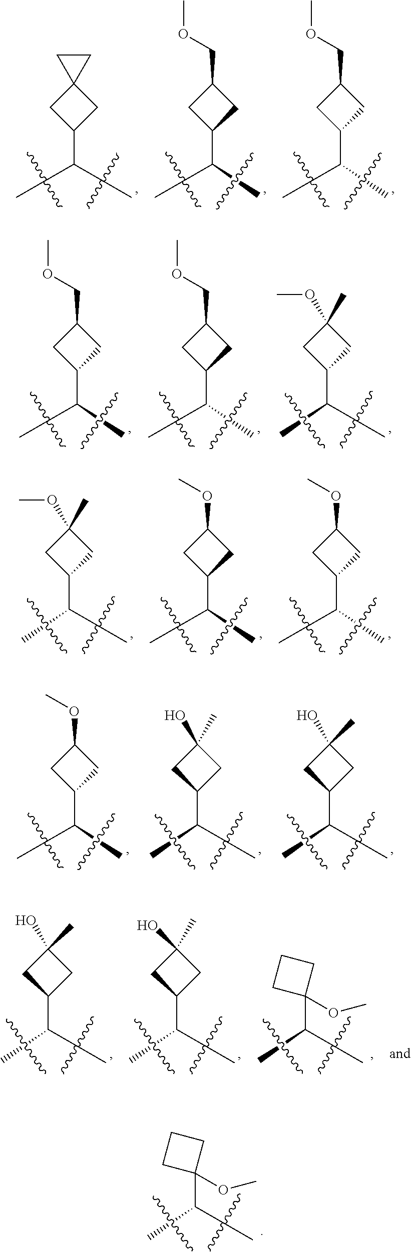

In some embodiments, wherein X is selected from the group consisting of

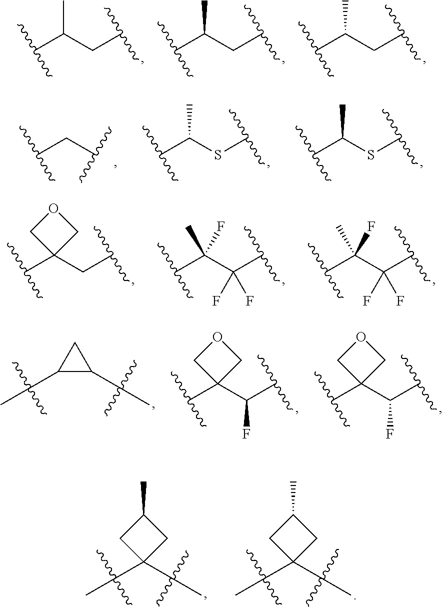

### R^{a}

In some embodiments, each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S. In some embodiments, L-A. In some embodiments, R^{a} is selected from halogen, —CN, —C(O)R¹, —CO₂H, —CONR¹R², optionally substituted C₁-C₆ aliphatic, and optionally substituted C₁-C₆ heteroalkyl. In some embodiments each R^{a} is independently selected from the group consisting of

halogen, —CN, —CO₂H, —CHO, —CHF₂, —CF₃, —OMe, —S(O)₂NHMe,

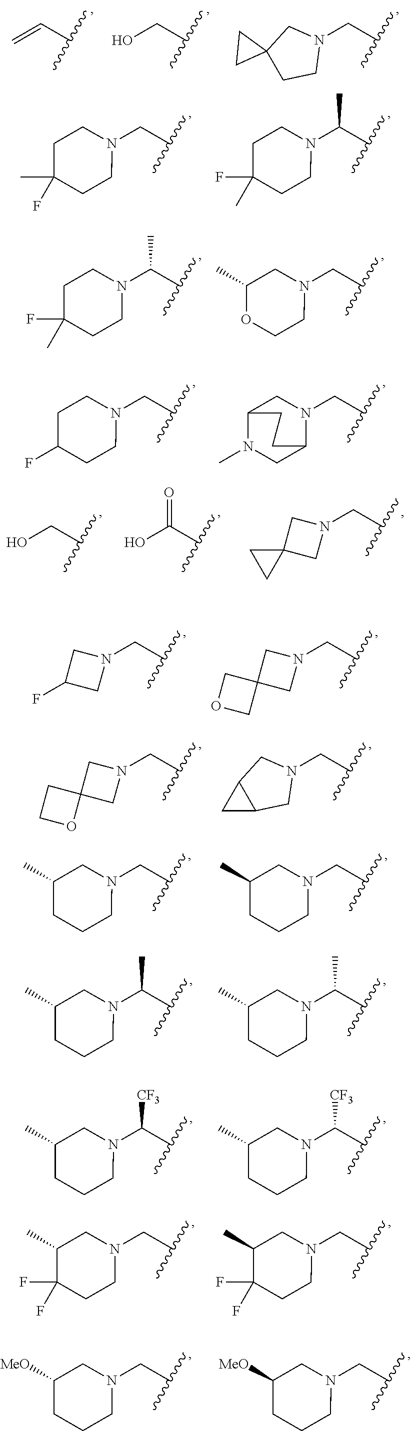

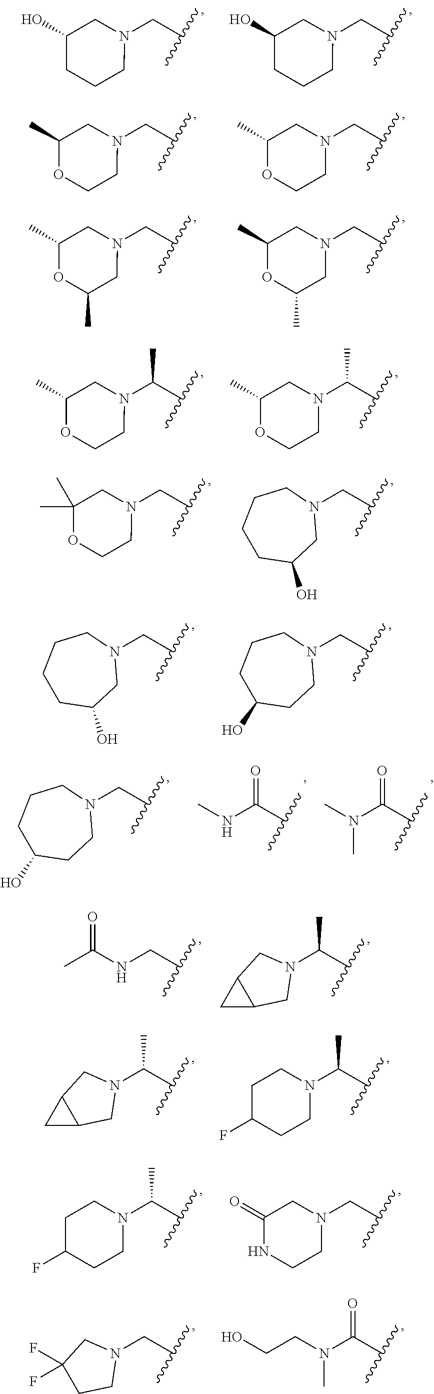

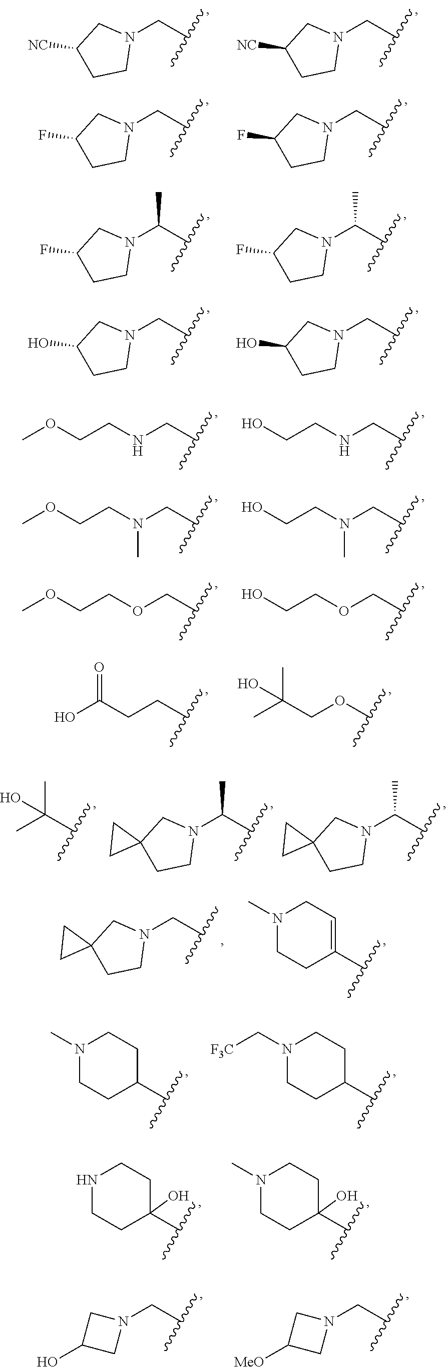

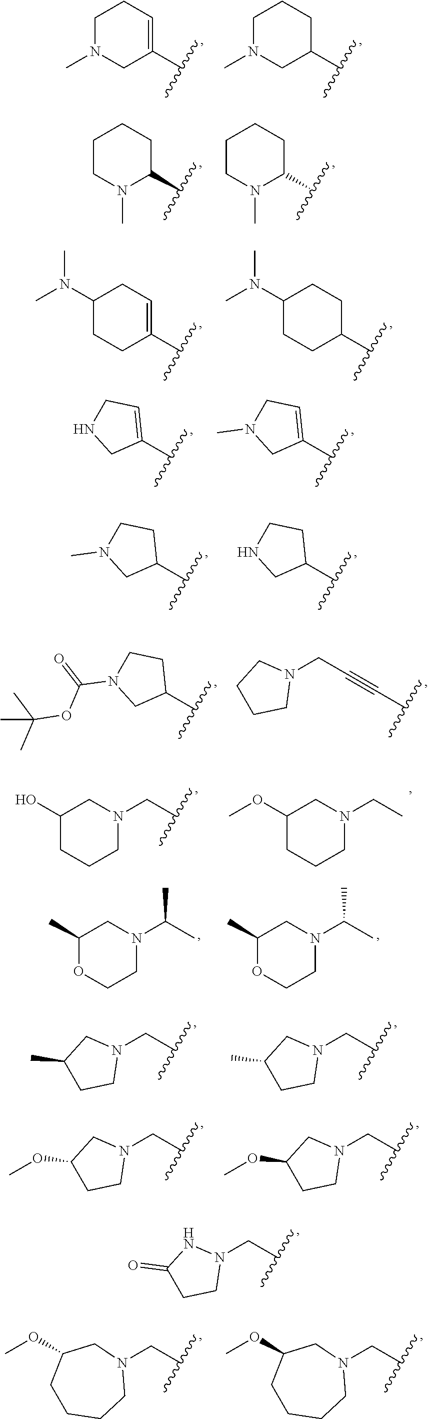

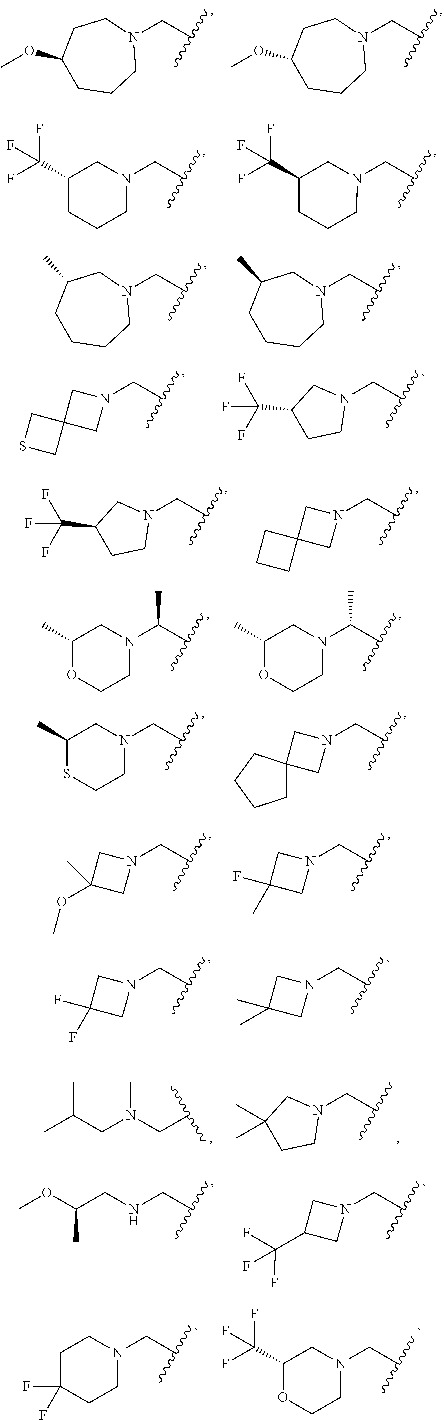

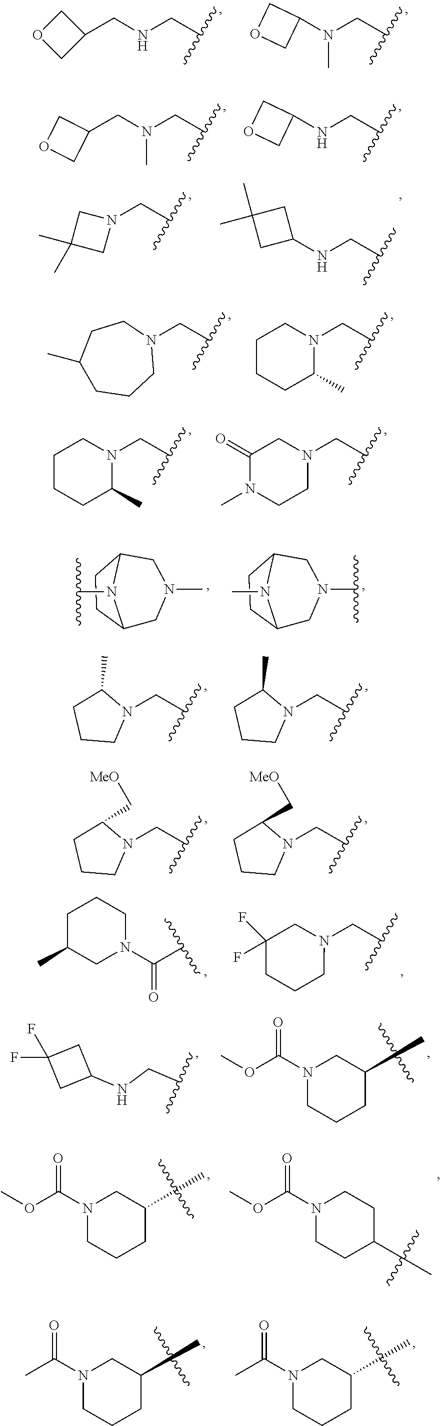

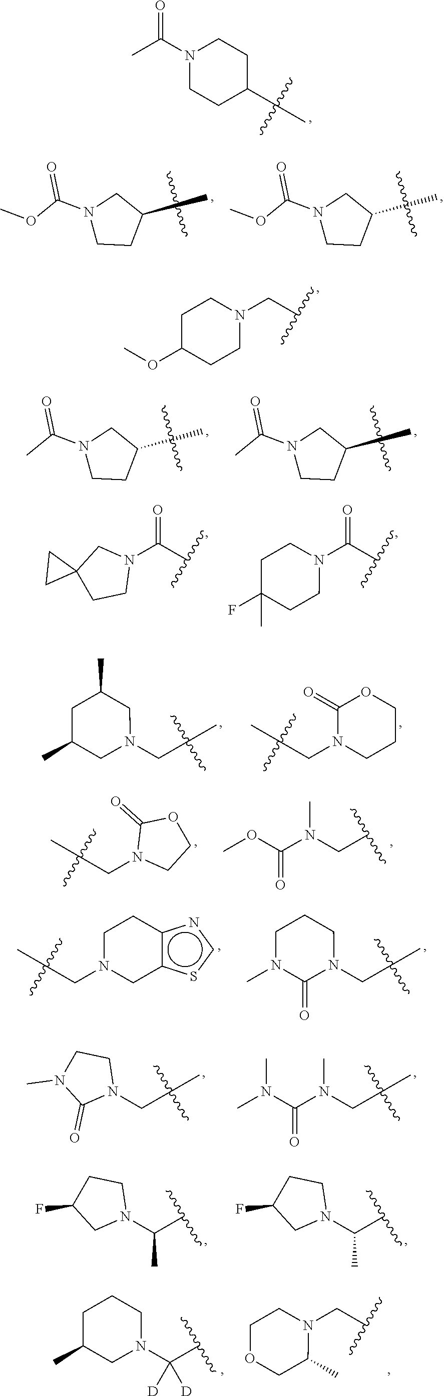

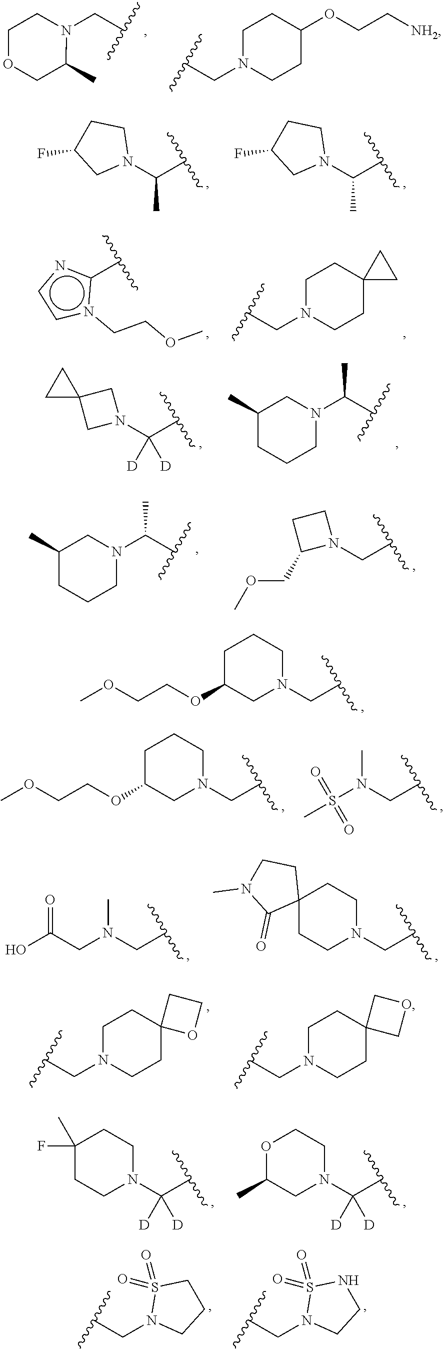

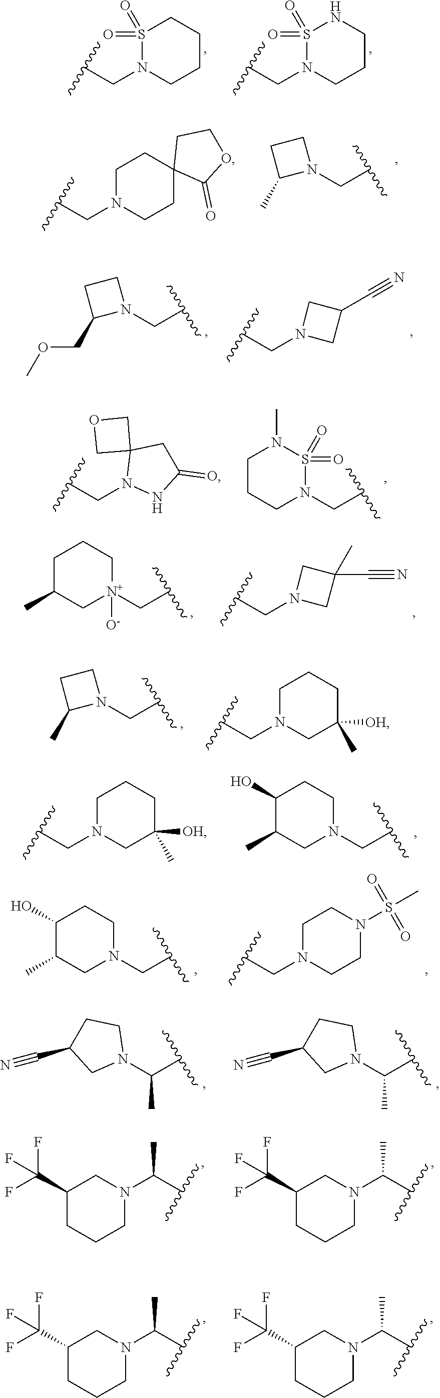

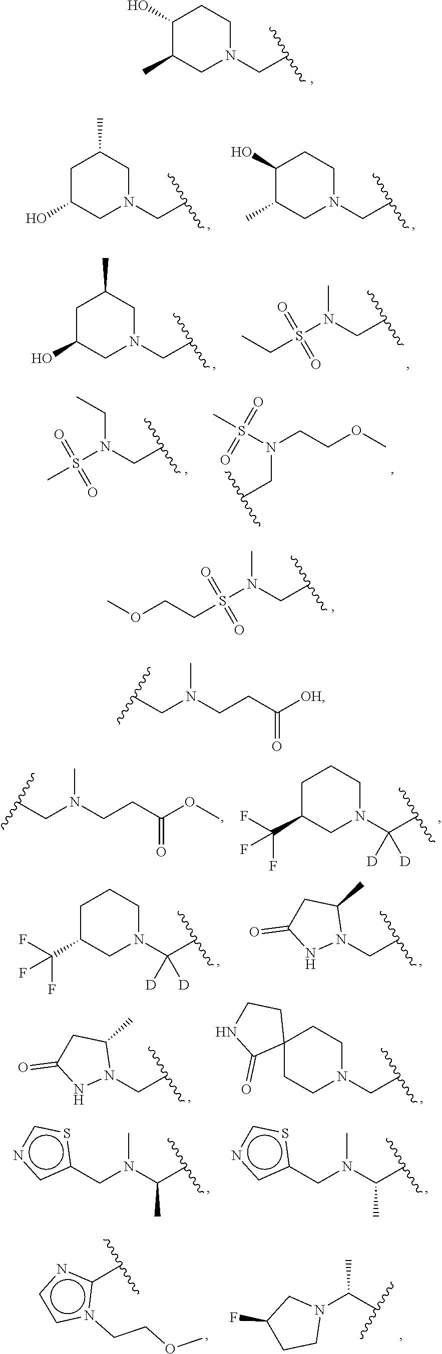

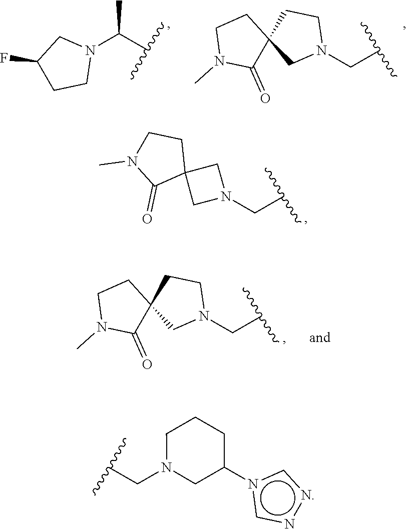

In some embodiments, R^{a} is selected from the group consisting of halogen, —CN, —CO₂H,

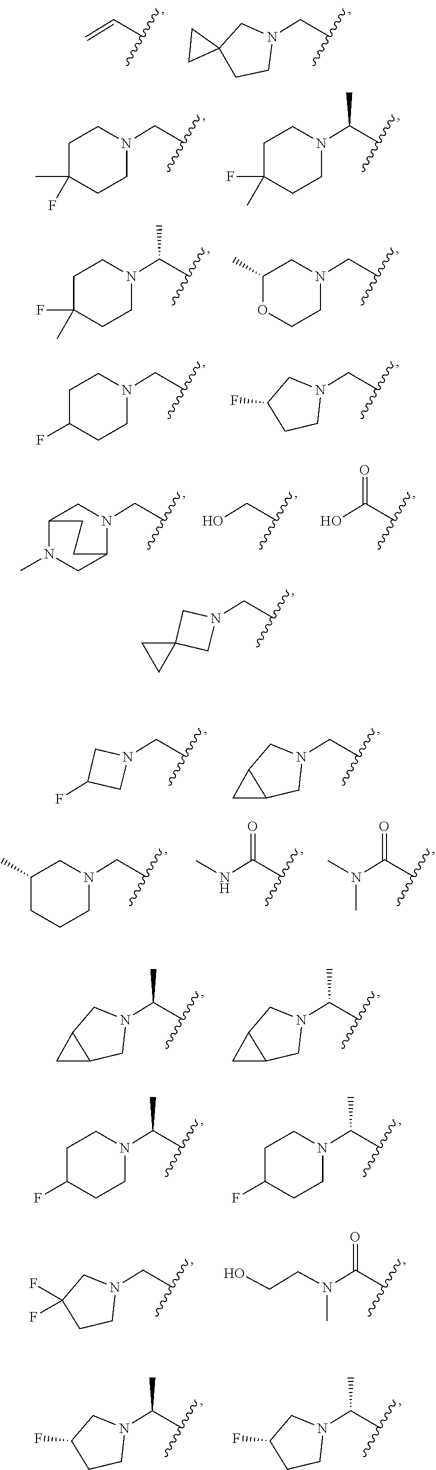

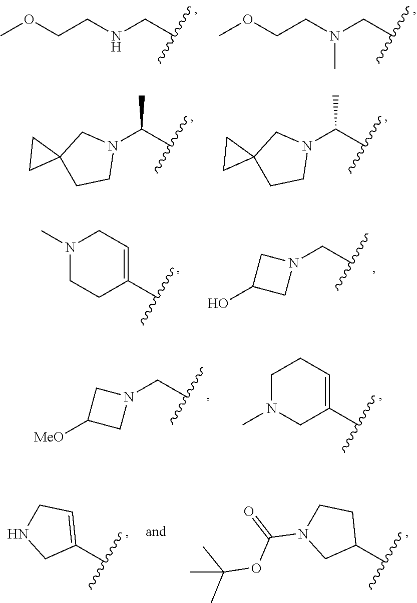

### L

In some embodiments, L is an optionally substituted C₁-C₃ alkylene chain. In some embodiments, L is —CH₂— or —CH(CH₃)—.

### A

In some embodiments, A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S. In some embodiments, A is optionally substituted 3-6 membered heterocyclyl containing 1-4 heteroatoms each selected from the group consisting of N, O, and S. In some embodiments, A is selected from optionally substituted piperidine, optionally substituted tetrahydropyridine, optionally substituted pyrrolidine, optionally substituted dihydropyrrole, optionally substituted aziridine, and optionally substituted morpholine.

### C

In some embodiments, C is optionally substituted 5-membered heteroaryl. In some embodiments, C is optionally substituted 5-membered heteroaryl containing 3 nitrogen atoms. In some embodiments, C is optionally substituted triazolyl. In some embodiments, C is optionally substituted 1,2,4 trizaolyl. In some embodiments, C is optionally substituted 1,2,3 trizaolyl. In some embodiments, C is optionally substituted 5-membered heteroaryl containing 2 nitrogen atoms. In some embodiments, C is optionally substituted pyrazolyl. In some embodiments, C is optionally substituted isoxazolyl. In some embodiments, C is optionally substituted thiazolyl. In some embodiments, C is optionally substituted thiadizolyl. In some embodiments, C is optionally substituted 1,3,4 thiadizolyl. In some embodiments, C is optionally substituted pyridinyl. In some embodiments, C is optionally substituted pyrazinyl. In some embodiments, C is optionally substituted pyrimidinyl. In some embodiments, C is optionally substituted pyridazinyl.

### R^{b}

Im some embodiments, each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S.

### R^{c}

In some embodiments, each R^{c} is independently selected from the group consisting of hydrogen, optionally substituted C₁-C₆ aliphatic, —OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, each R^{c} is independently selected from the group consisting of hydrogen, optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, R^{c} is optionally substituted C₁-C₃ aliphatic. In some embodiments, R^{c} is methyl.

### R¹

In some embodiments, each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³. In some embodiments, each R¹ is optionally substituted C₁-C₆ aliphatic. In some embodiments, each R¹ is methyl.

### R²

In some embodiments, each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.

In some embodiments, each R² is optionally substituted C₁-C₆ aliphatic. In some embodiments, each R² is methyl.

### R³

In some embodiments, each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S.

In some embodiments, the present disclosure includes compounds described in Table 1.

TABLE 1

| Cmpd | |
|---|---|
| No. | Structure |
| 1 | 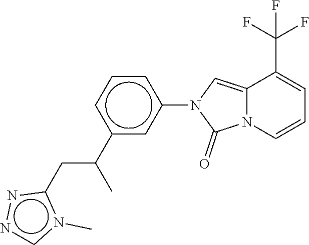 |
| 2 | 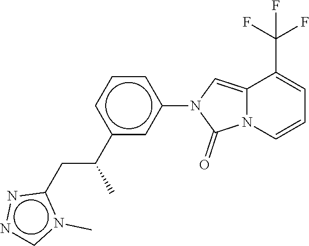 |
| 3 | 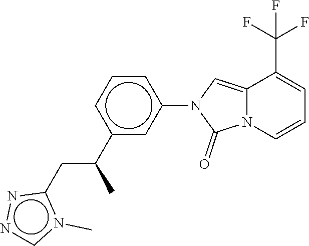 |
| 4 | 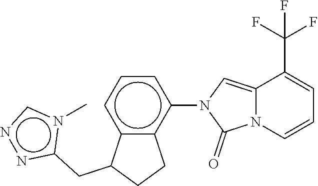 |
| 5 | 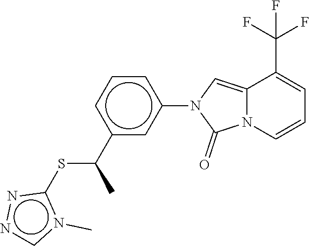 |
| 6 | 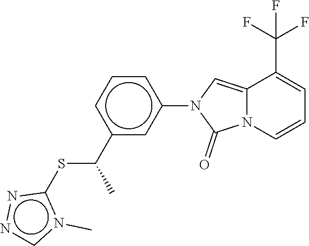 |
| 7 | 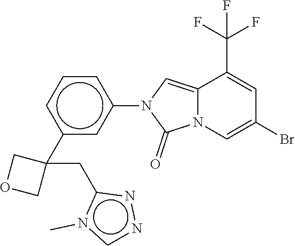 |
| 8 | 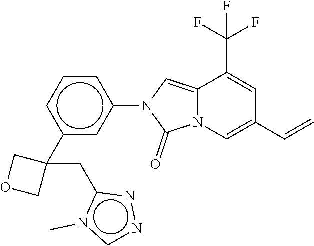 |
| 9 | 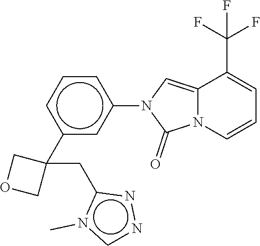 |
| 10 | 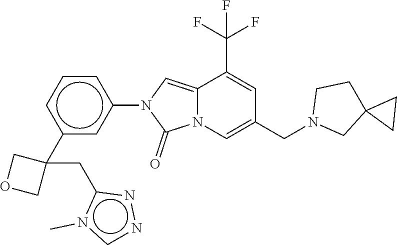 |
| 11 | 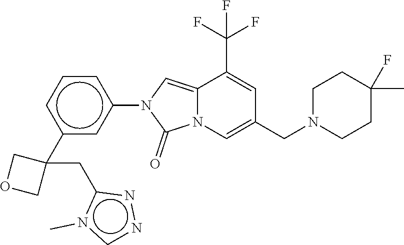 |
| 12 | 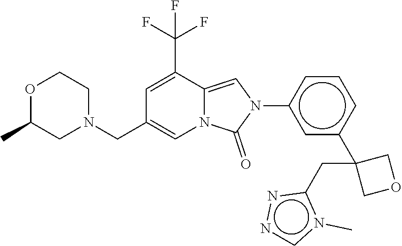 |
| 13 | 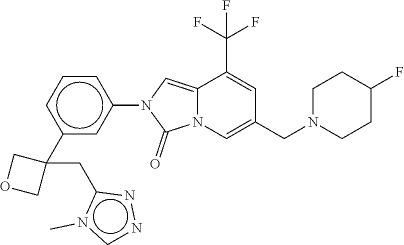 |
| 14 | 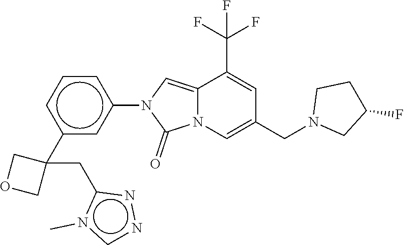 |
| 15 | 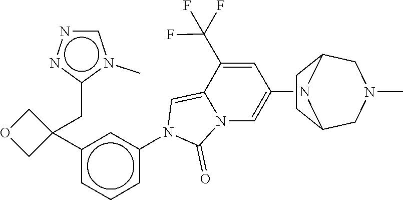 |
| 16 | 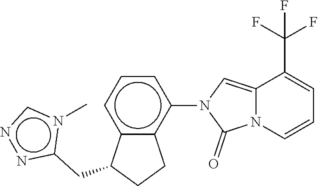 |
| 17 | 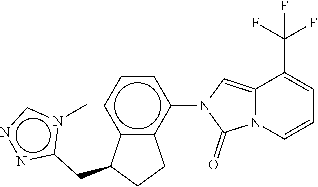 |
| 18 | 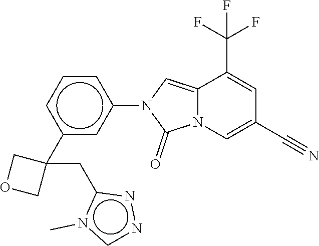 |
| 19 | 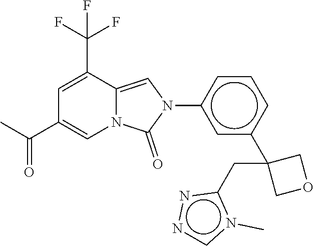 |
| 20 | 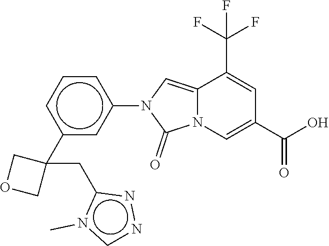 |
| 21 | 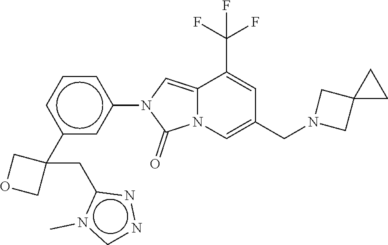 |
| 22 | 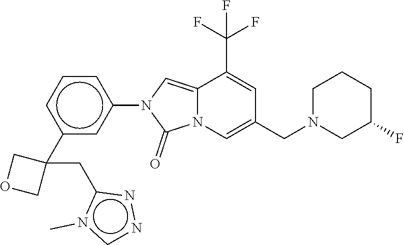 |
| 23 | 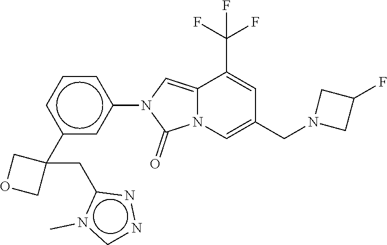 |
| 24 | 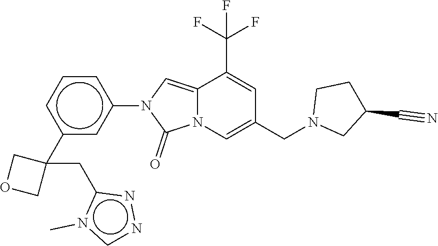 |
| 25 | 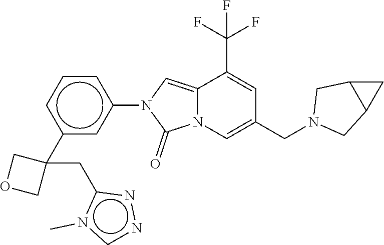 |
| 26 | 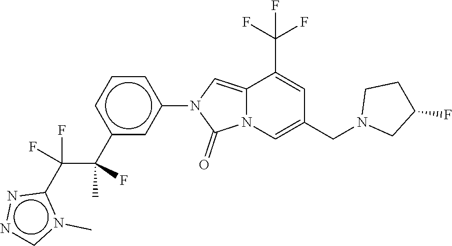 |
| 27 | 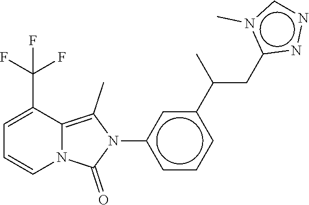 |
| 28 | 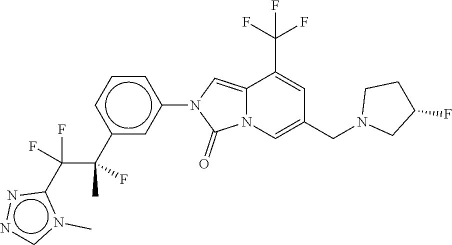 |
| 29 | 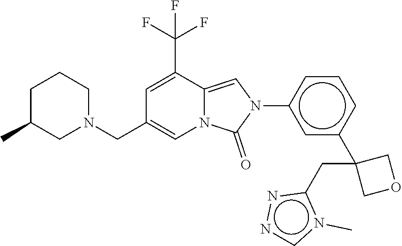 |
| 30 | 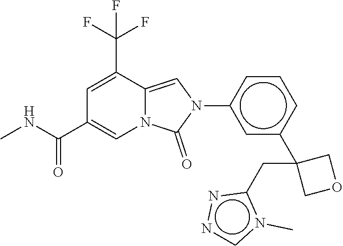 |
| 31 | 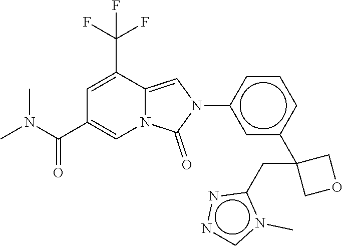 |
| 32 | 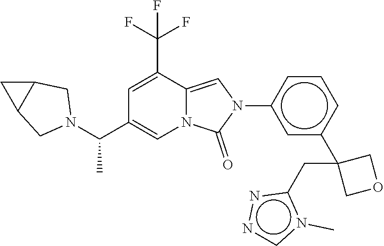 |
| 33 | 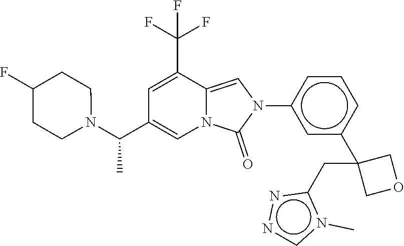 |
| 34 | 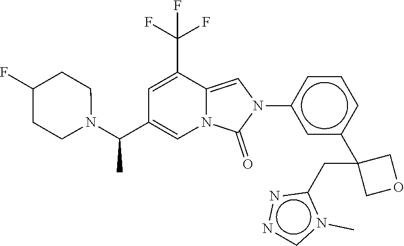 |
| 35 | 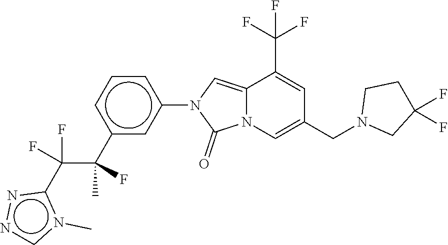 |
| 36 | 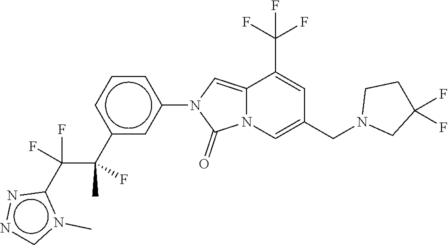 |
| 37 | 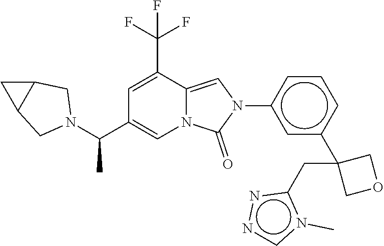 |
| 38 | 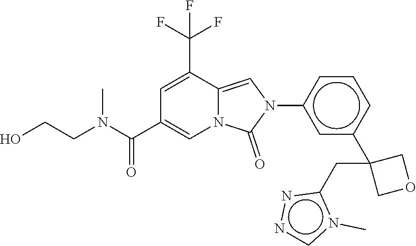 |
| 39 | 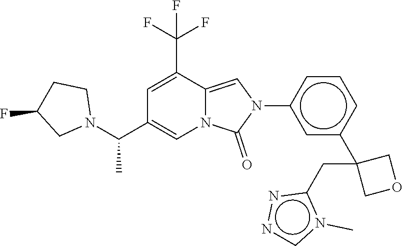 |
| 40 | 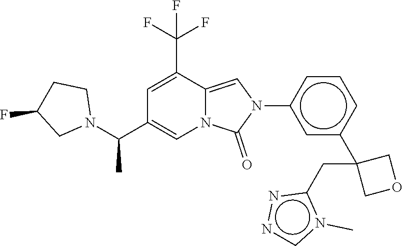 |
| 41 | 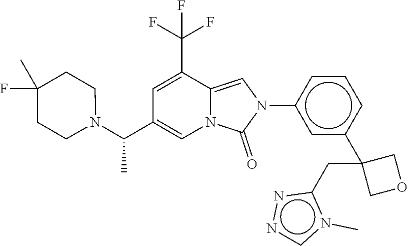 |
| 42 | 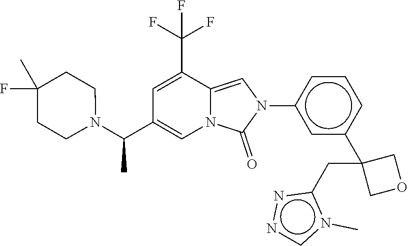 |
| 43 | 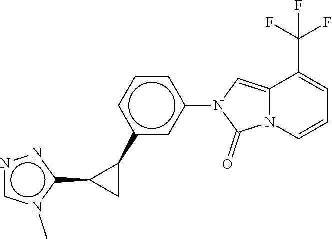 |
| 44 | 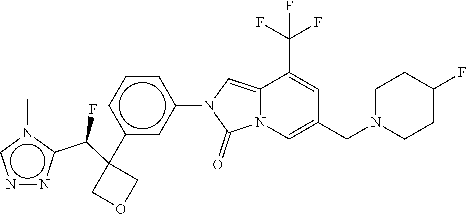 |
| 45 | 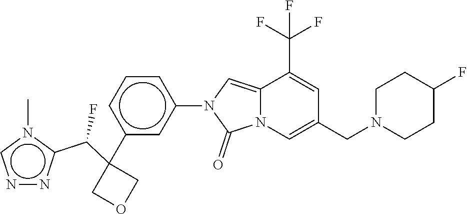 |
| 46 | 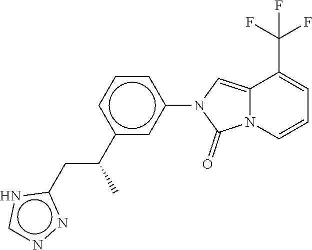 |
| 47 | 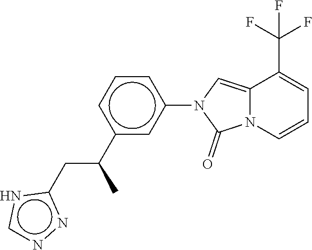 |
| 48 | 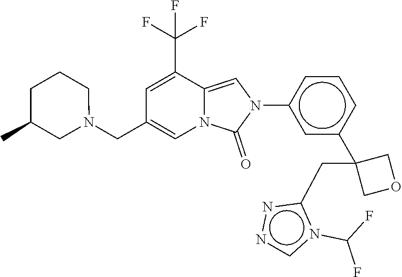 |
| 49 | 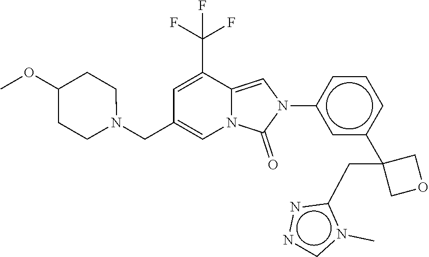 |
| 50 | 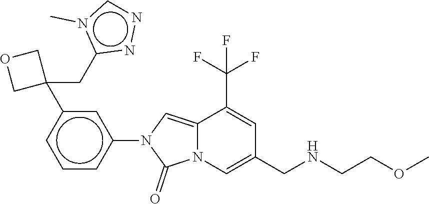 |
| 51 | 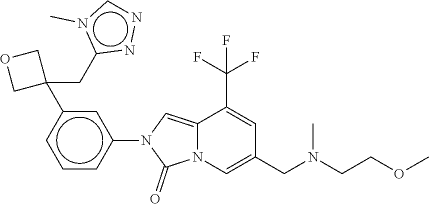 |
| 52 | 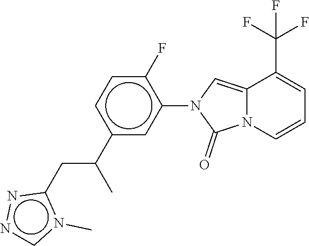 |
| 53 | 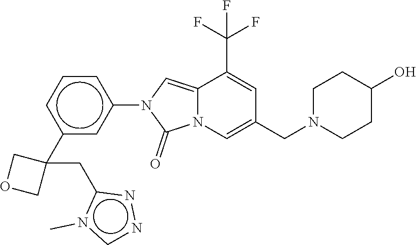 |
| 54 | 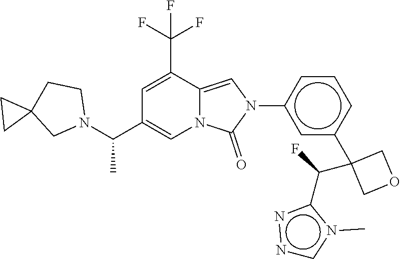 |
| 55 | 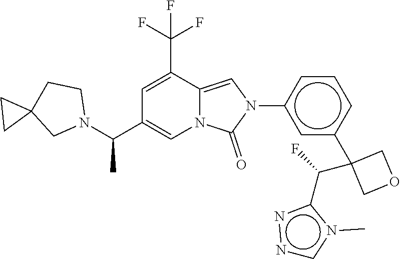 |
| 56 | 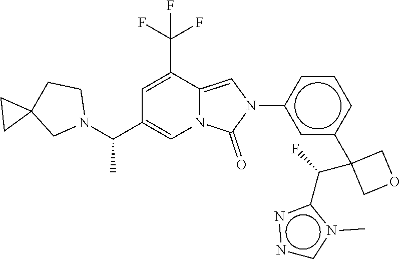 |
| 57 | 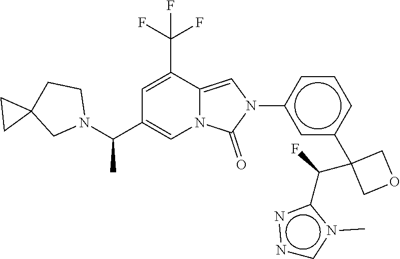 |
| 58 | 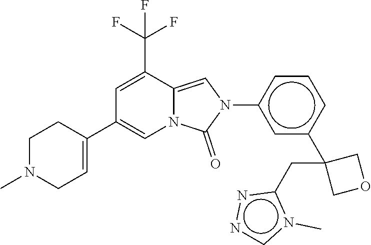 |
| 59 | 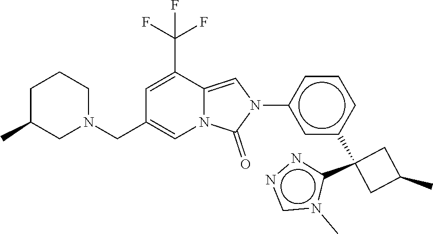 |
| 60 | 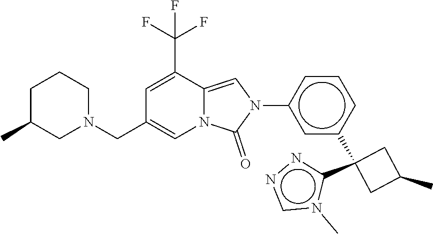 |
| 61 | 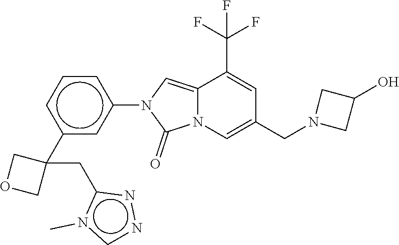 |
| 62 | 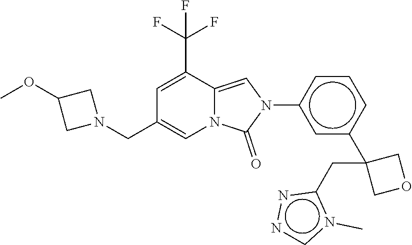 |
| 63 | 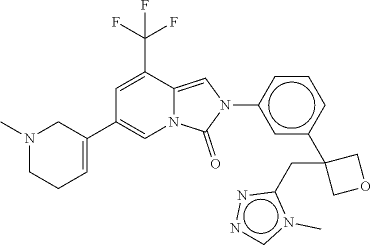 |
| 64 | 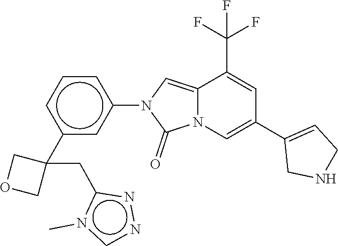 |
| 65 | 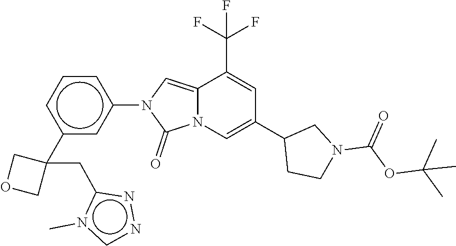 |
| 66 | 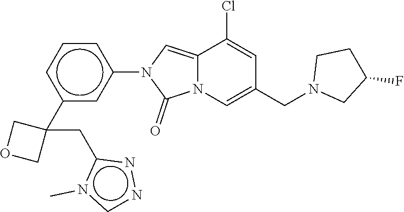 |
| 67 | 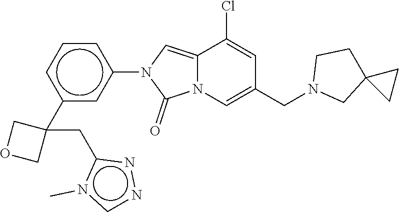 |
| 68 | 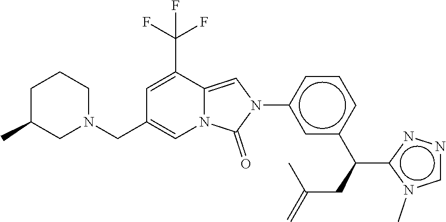 |
| 69 | 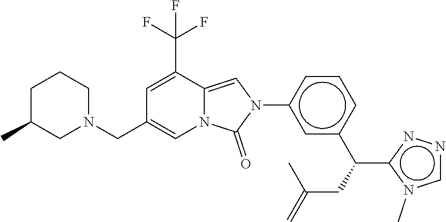 |
| 70 | 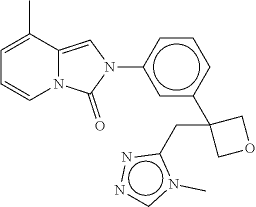 |
| 71 | 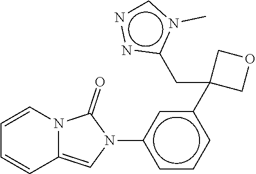 |
| 72 | 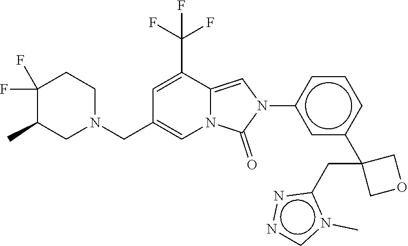 |
| 73 | 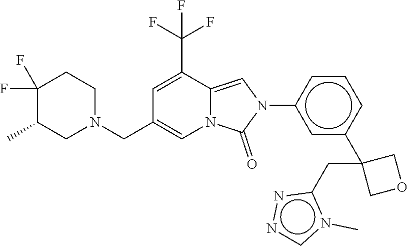 |
| 74 | 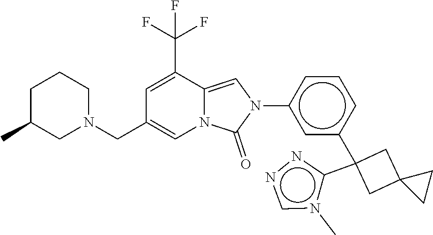 |
| 75 | 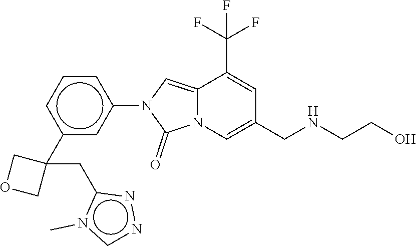 |
| 76 | 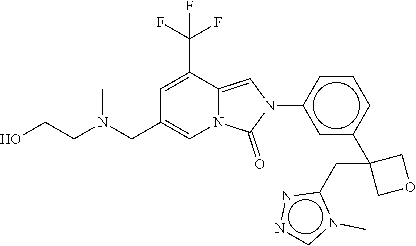 |
| 77 | 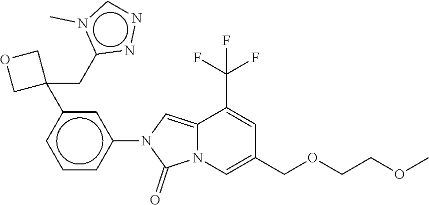 |
| 78 | 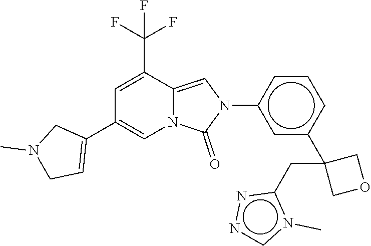 |
| 79 | 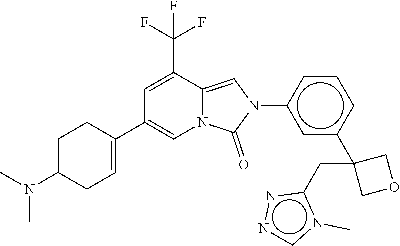 |
| 80 | 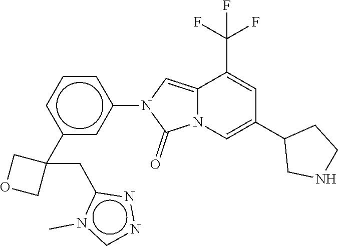 |
| 81 | 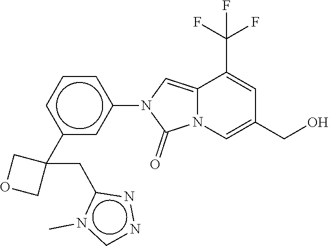 |
| 82 | 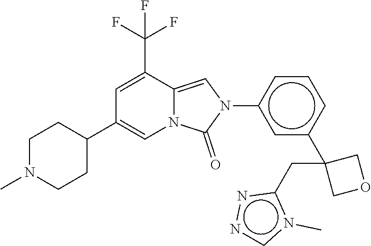 |
| 83 | 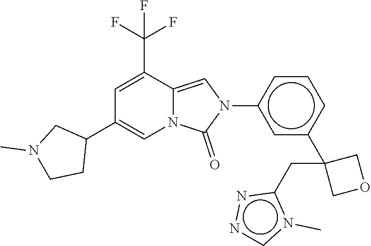 |
| 84 | 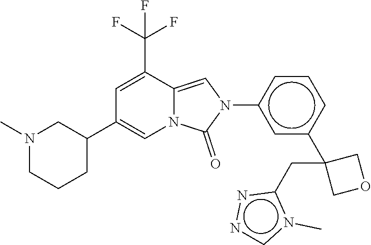 |
| 85 | 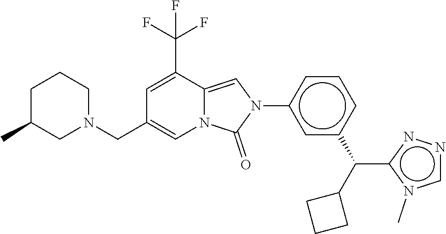 |
| 86 | 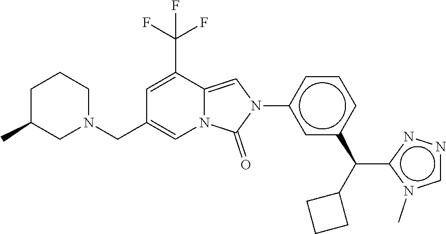 |
| 87 | 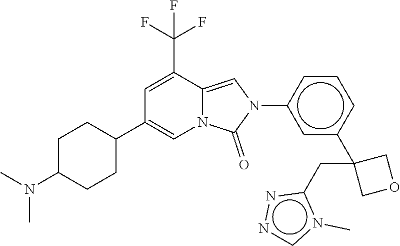 |
| 88 | 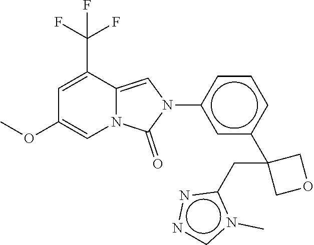 |
| 89 | 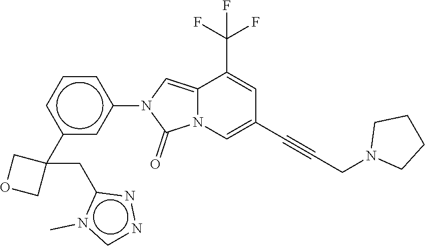 |
| 90 | 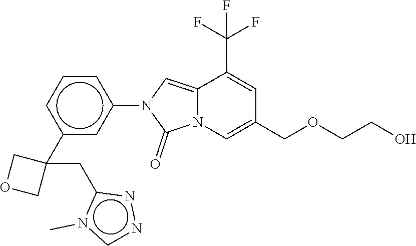 |
| 91 | 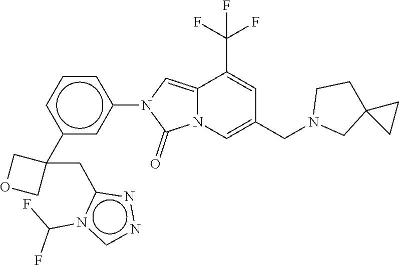 |
| 92 | 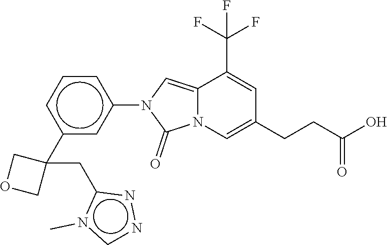 |
| 93 | 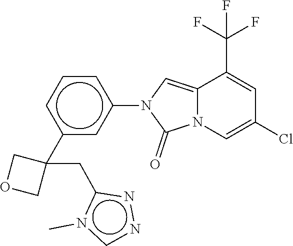 |
| 94 | 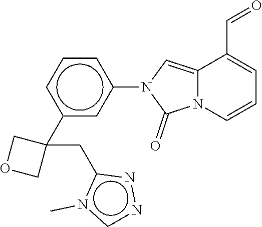 |
| 95 | 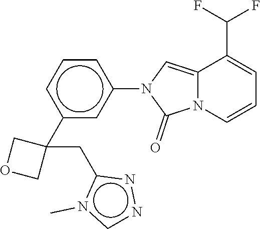 |
| 96 | 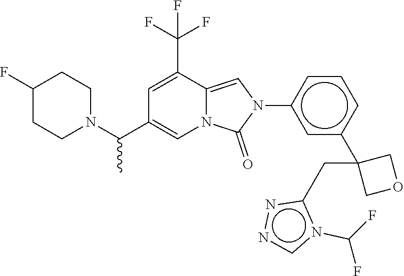 |
| | isomer 1 |
| 97 | 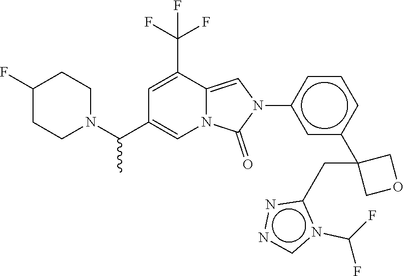 |
| | isomer 2 |
| 98 | 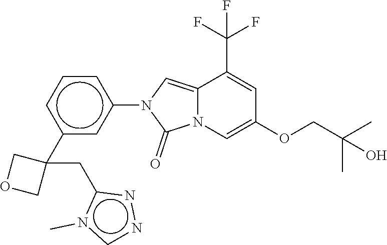 |
| 99 | 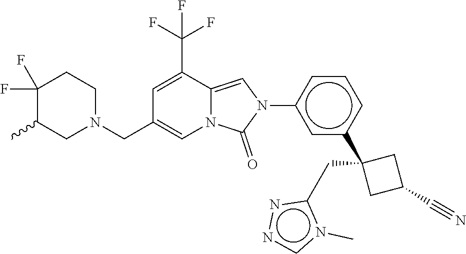 |
| | isomer 1 |
| 100 | 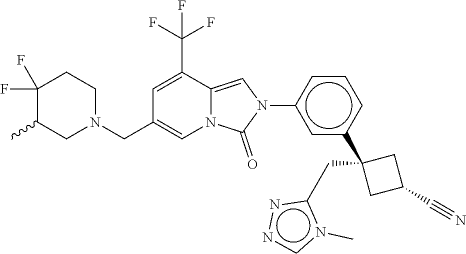 |
| | isomer 2 |
| 101 | 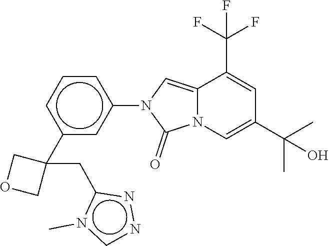 |
| 102 | 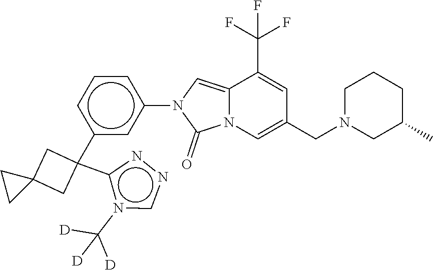 |
| 103 | 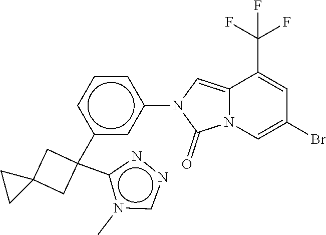 |
| 104 | 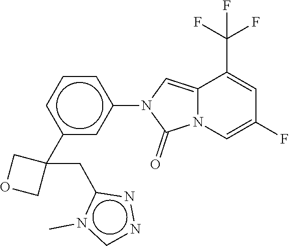 |
| 105 | 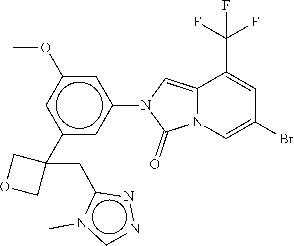 |
| 106 | 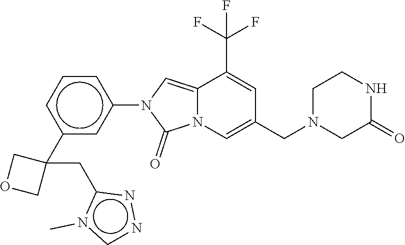 |
| 107 | 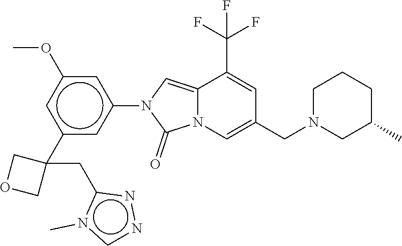 |
| 108 | 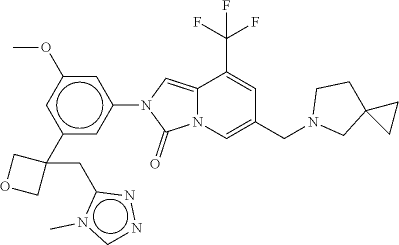 |
| 109 | 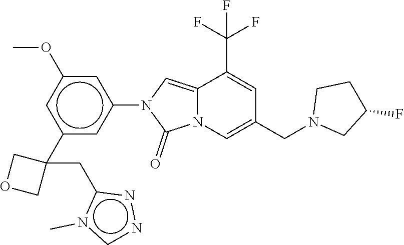 |
| 110 | 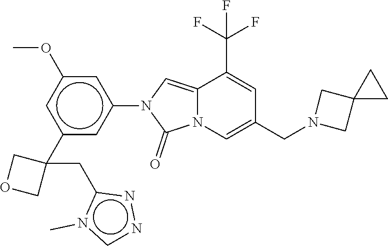 |
| 111 | 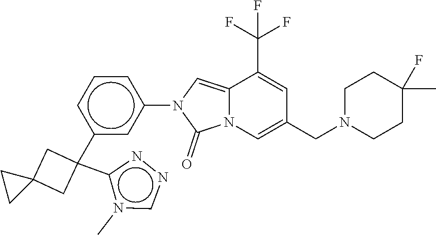 |
| 112 | 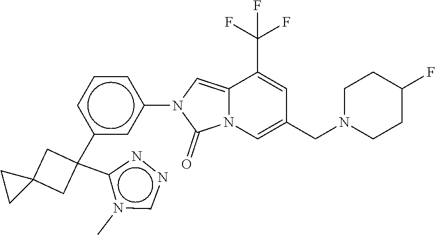 |
| 113 | 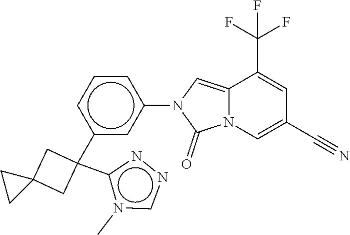 |
| 114 | 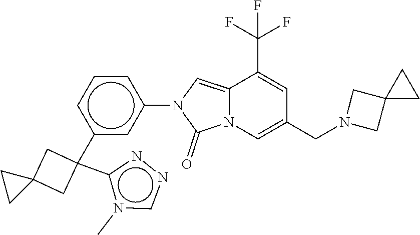 |
| 115 | 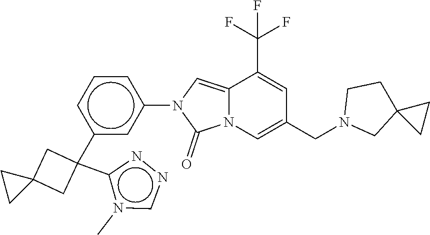 |
| 116 | 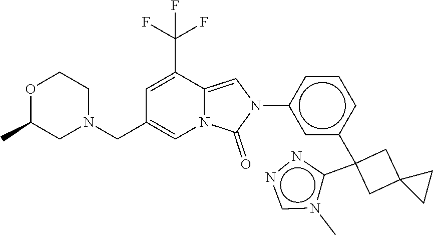 |
| 117 | 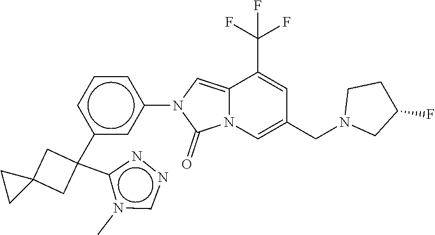 |
| 118 | 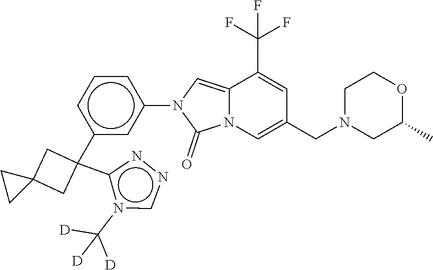 |
| 119 | 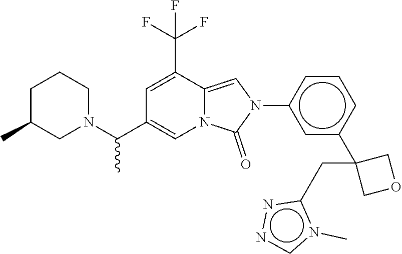 |
| | isomer 1 |
| 120 | 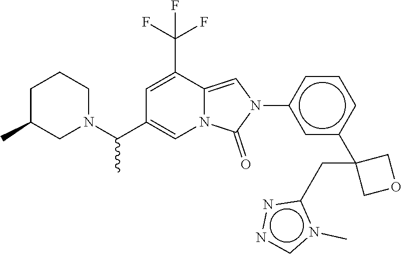 |
| | isomer 2 |
| 121 | 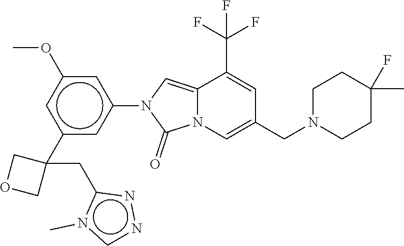 |
| 122 | 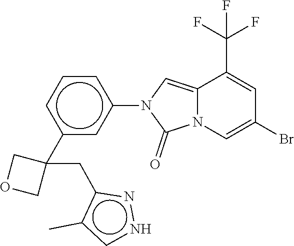 |
| 123 | 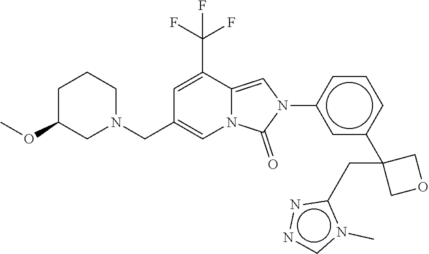 |
| 124 | 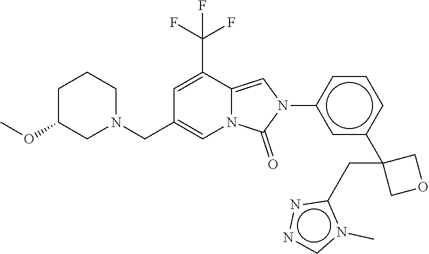 |
| 125 | 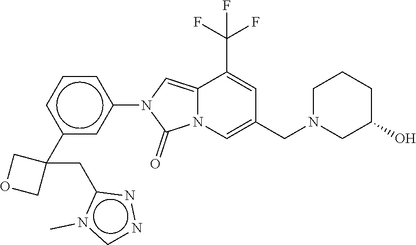 |
| 126 | 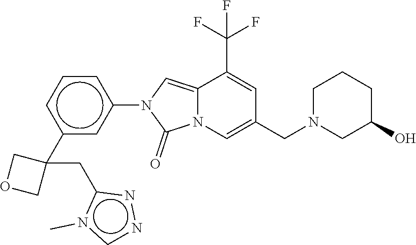 |
| 127 | 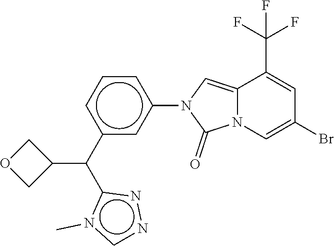 |
| 128 | 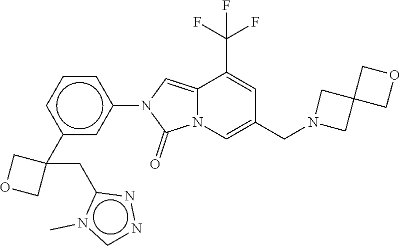 |
| 129 | 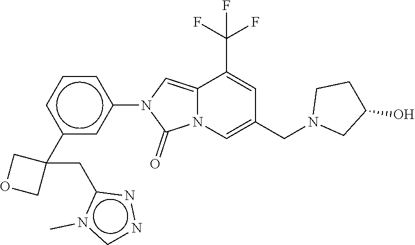 |
| 130 | 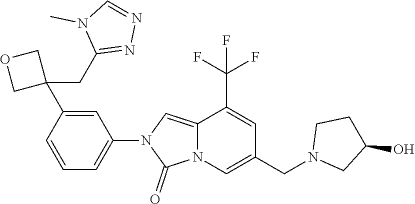 [SMILES: Cn1cnnc1CC1(c2cccc(-n3cc4c(C(F)(F)F)cc(CN5CC[C@@H](O)C5)cn4c3=O)c2)COC1] |
| 131 | 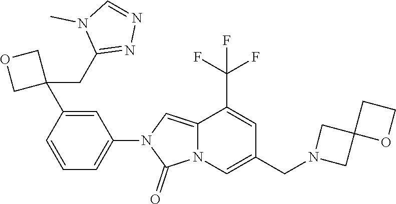 [SMILES: Cn1cnnc1CC1(c2cccc(-n3cc4c(C(F)(F)F)cc(CN5CC6(CCO6)C5)cn4c3=O)c2)COC1] |
| 132 | 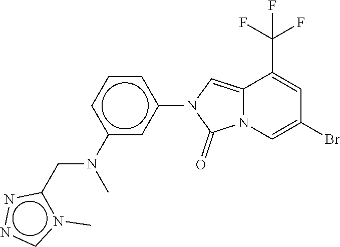 |
| 133 | 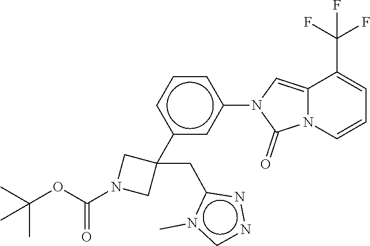 |
| 134 | 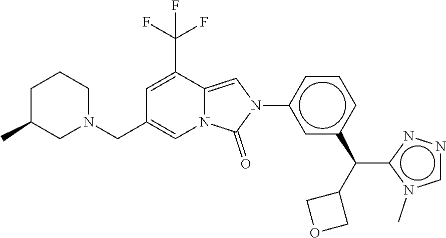 |
| 135 | 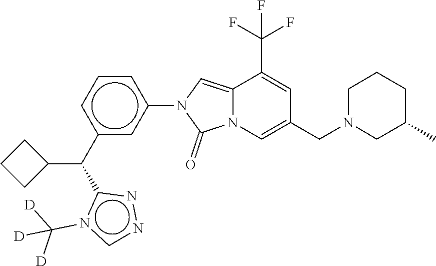 |
| 136 | 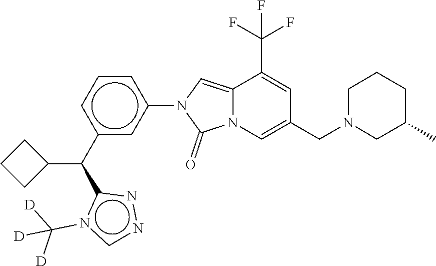 |
| 137 | 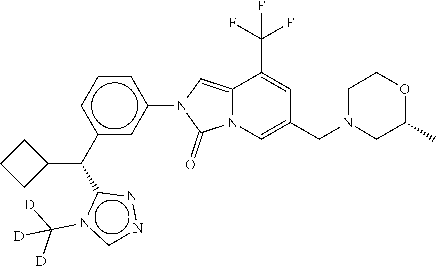 |
| 138 | 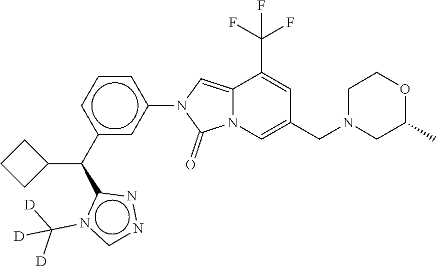 |
| 139 | 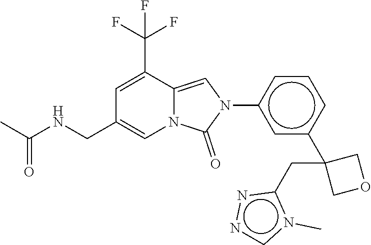 |
| 140 | 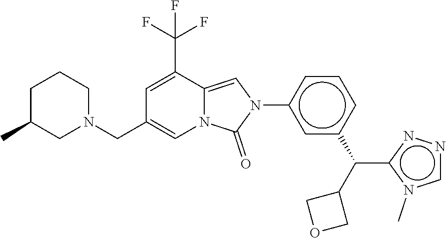 |
| 141 | 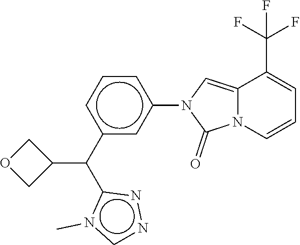 |
| 142 | 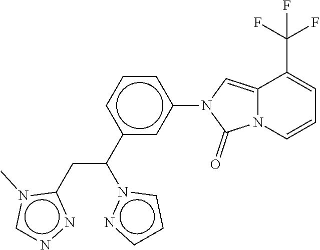 |
| 143 | 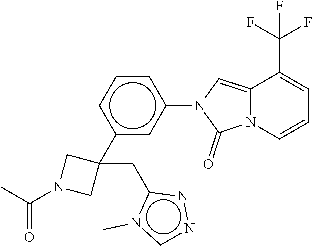 |
| 144 | 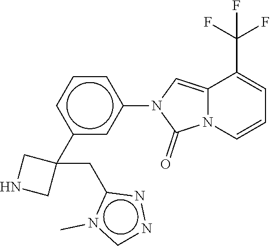 |
| 145 | 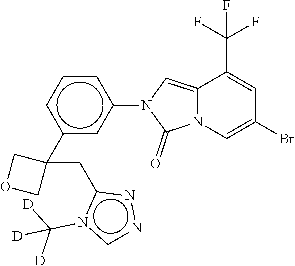 |
| 146 | 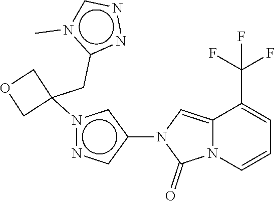 |
| 147 | 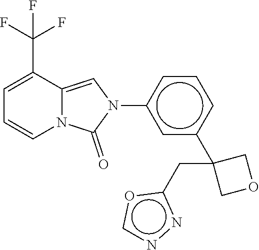 |
| 148 | 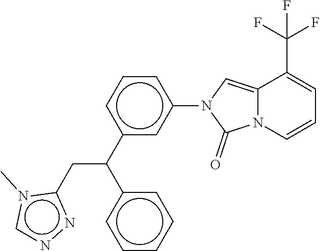 |
| 149 | 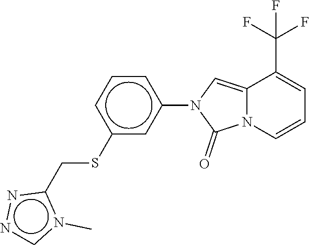 |
| 150 | 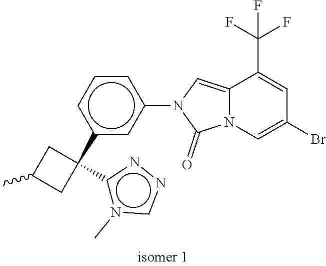 |
| 151 | 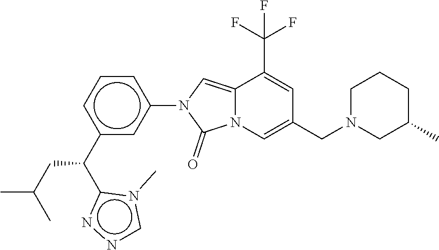 |
| 152 | 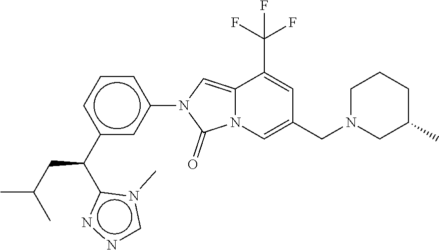 |
| 153 | 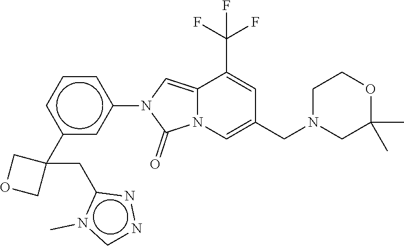 |
| 154 | 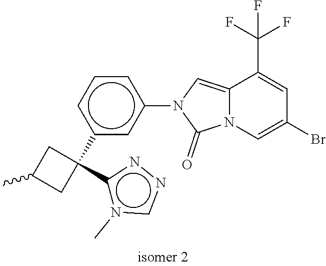 |
| 155 | 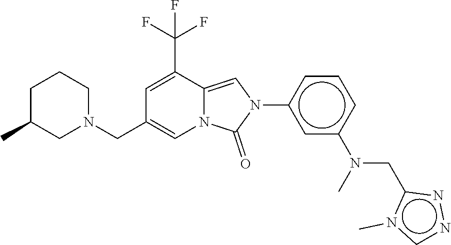 |
| 156 | 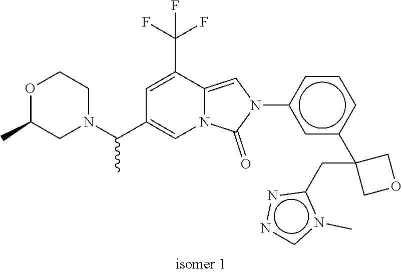 |
| 157 | 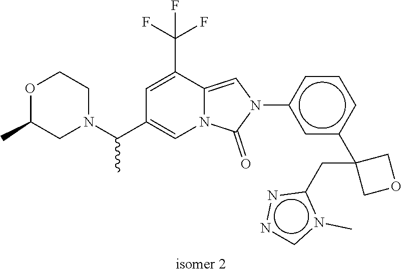 |
| 158 | 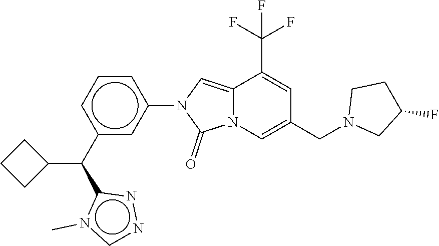 |
| 159 | 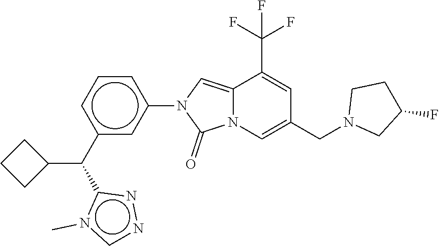 |
| 160 | 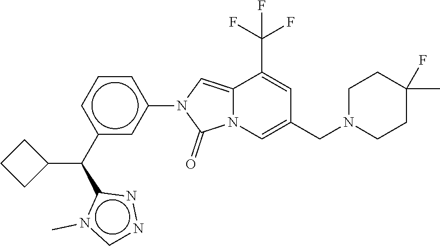 |
| 161 | 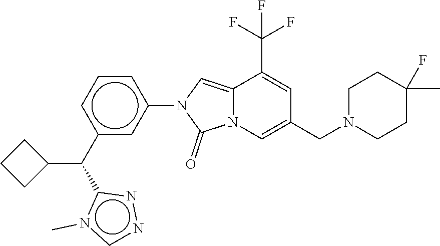 |
| 162 | 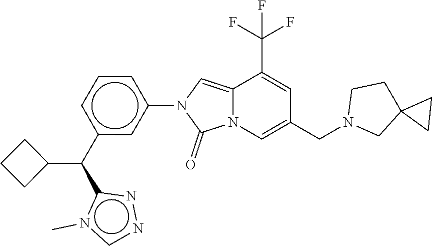 |
| 163 | 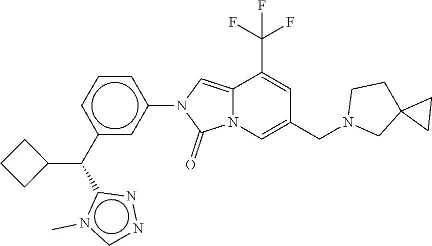 |
| 164 | 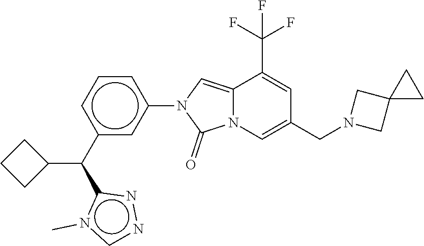 |
| 165 | 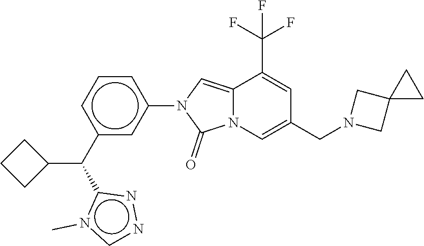 |
| 166 | 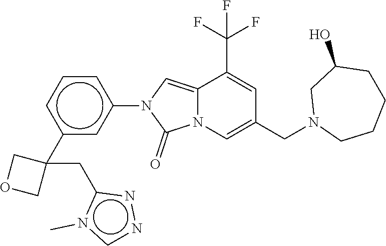 |
| 167 | 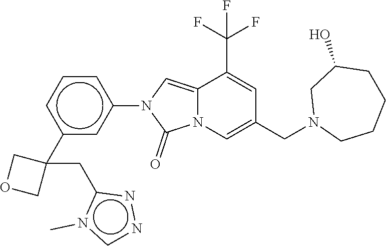 |
| 168 | 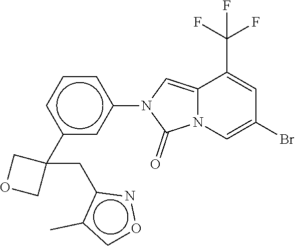 |
| 169 | 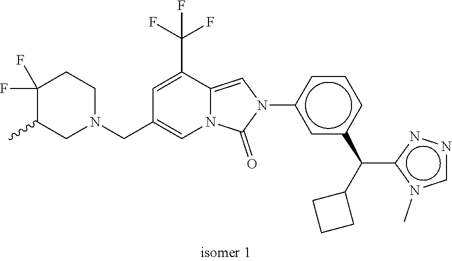 |
| 170 | 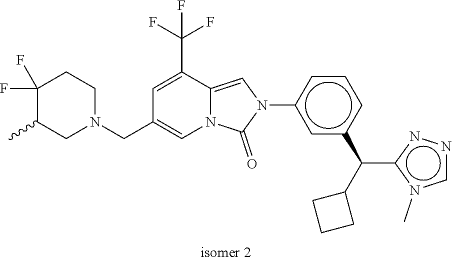 |
| 171 | 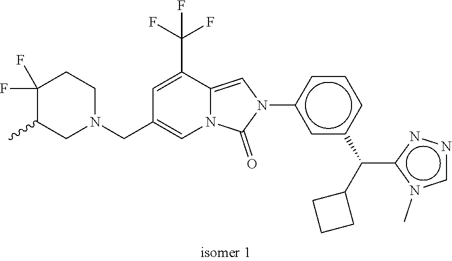 |
| 172 | 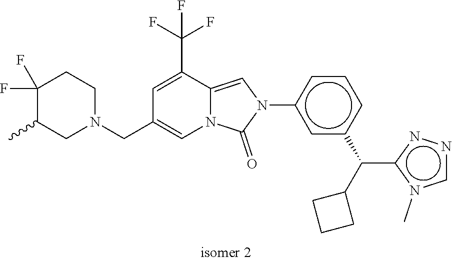 |
| 173 | 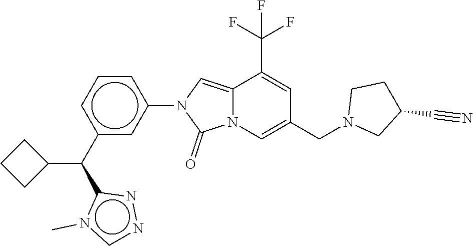 |
| 174 | 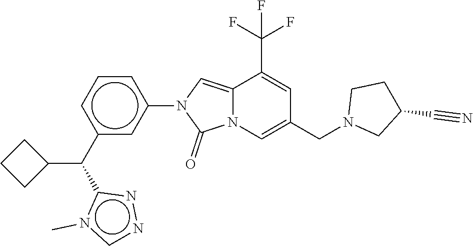 |
| 175 | 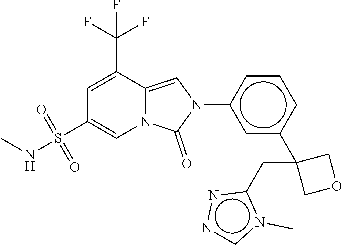 |
| 176 | 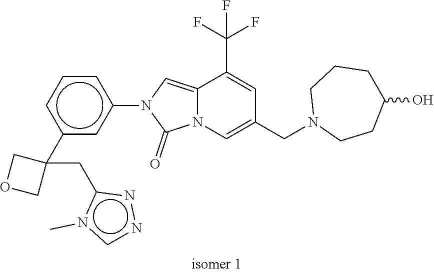 |
| 177 | 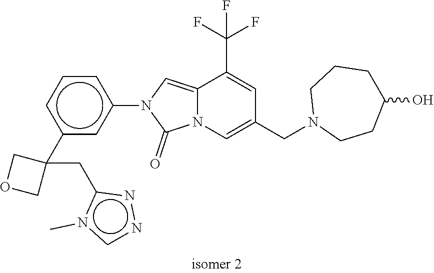 |
| 178 | 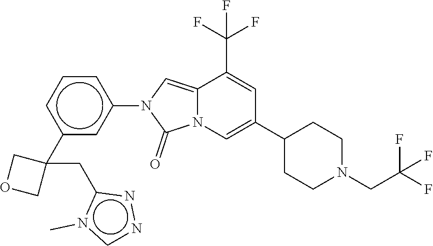 |
| 179 | 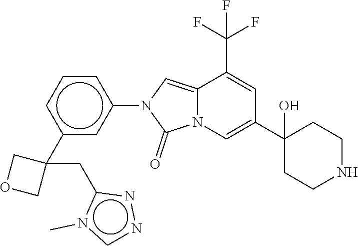 |
| 180 | 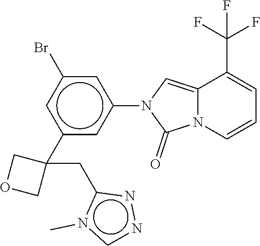 |
| 181 | 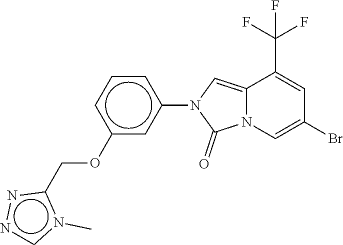 |
| 182 | 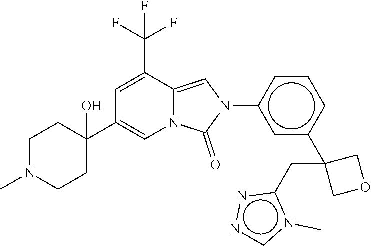 |
| 183 | 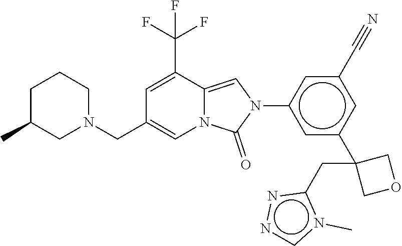 |
| 184 | 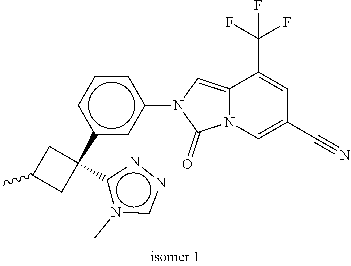 |
| 185 | 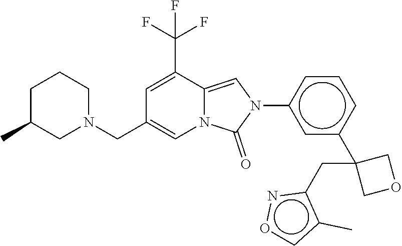 |
| 186 | 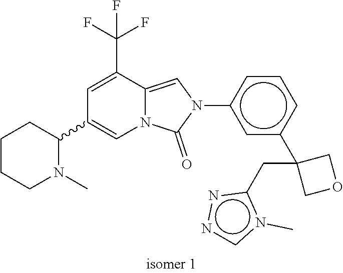 |
| 187 | 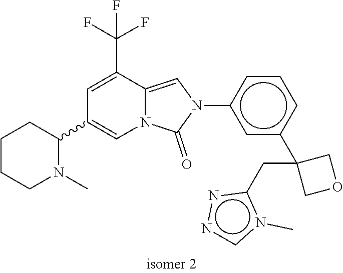 |
| 188 | 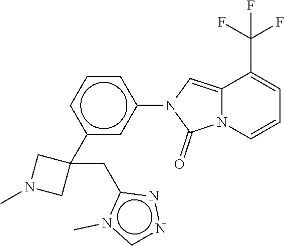 |
| 189 | 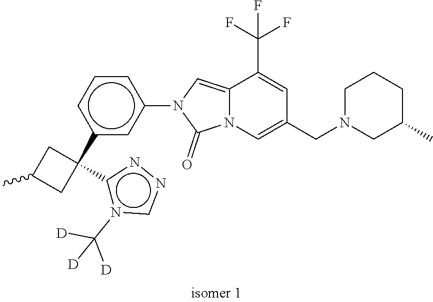 |
| 190 | 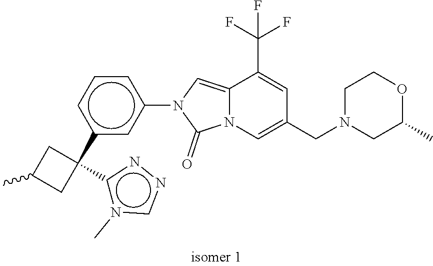 |
| 191 | 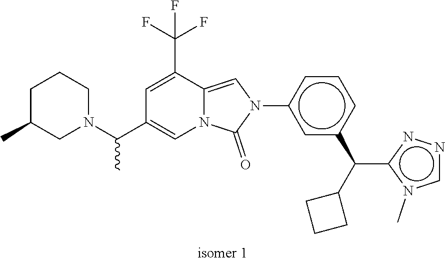 |
| 192 | 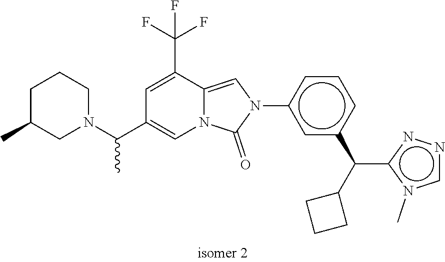 |
| 193 | 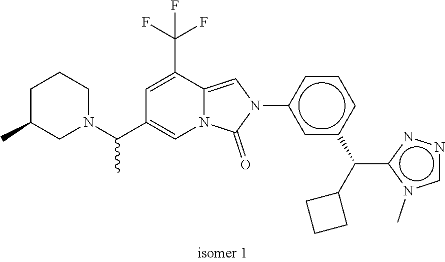 |
| 194 | 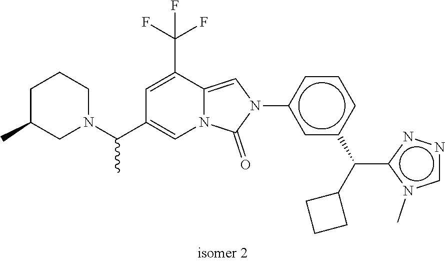 |
| 195 | 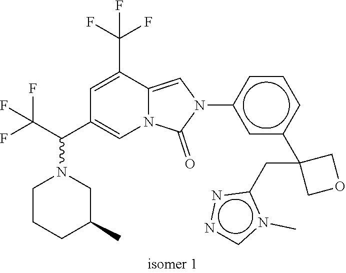 |
| 196 | 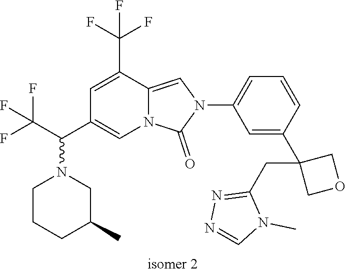 [SMILES: C[C@H]1CCCN(C(c2cc(C(F)(F)F)c3cn(-c4cccc(C5(Cc6nncn6C)COC5)c4)c(=O)n3c2)C(F)(F)F)C1] |
| 197 | 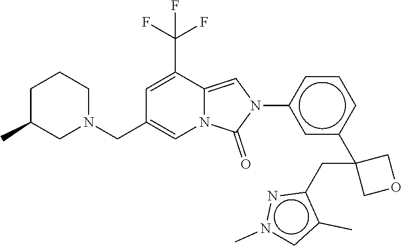 |
| 198 | 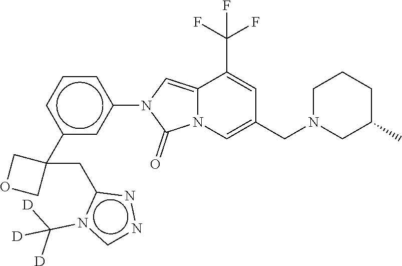 |
| 199 | 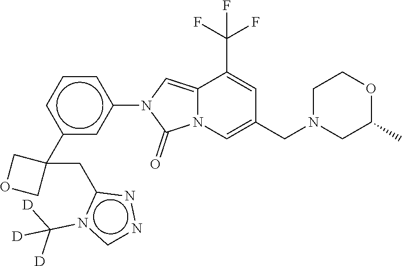 |
| 200 | 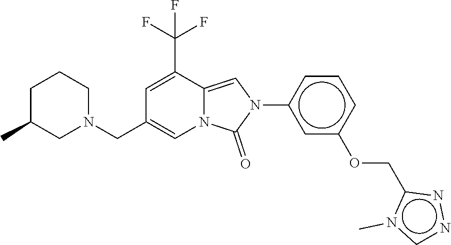 |
| 201 | 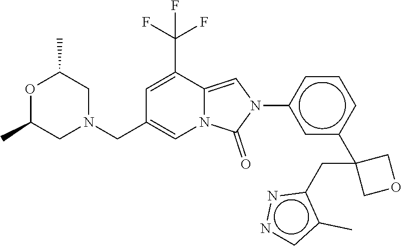 |
| 202 | 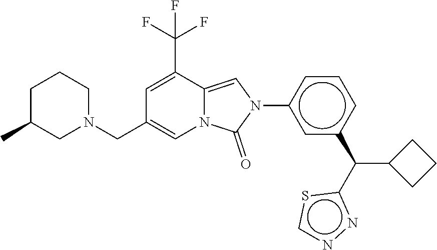 |
| 203 | 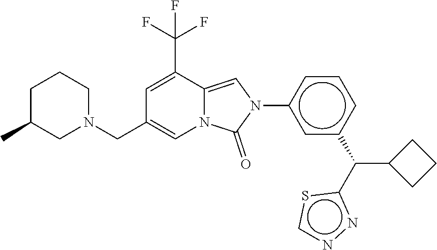 |
| 204 | 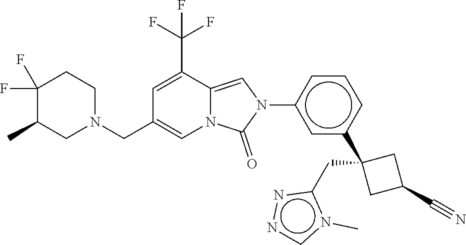 |
| 205 | 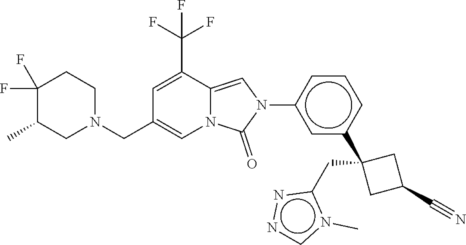 |
| 206 | 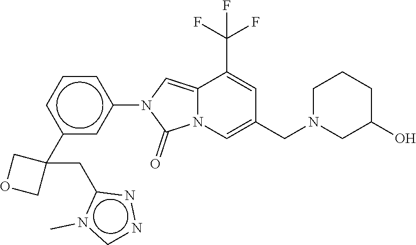 |
| 207 | 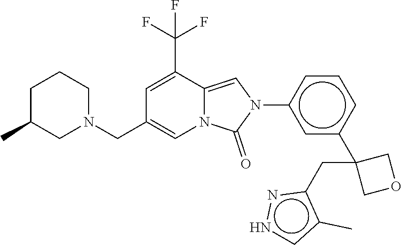 |
| 208 | 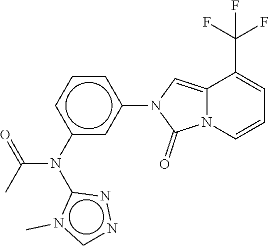 |
| 209 | 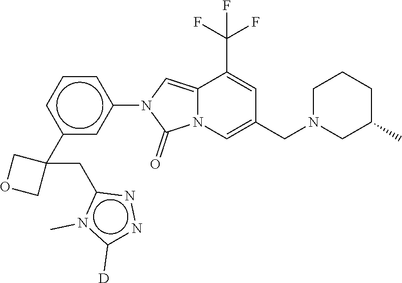 |
| 210 | 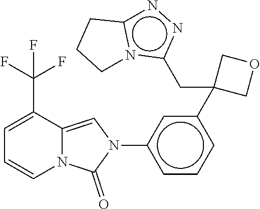 |
| 211 | 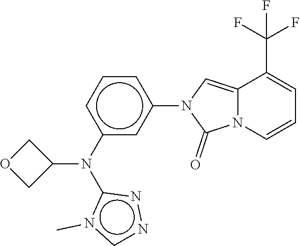 |
| 212 | 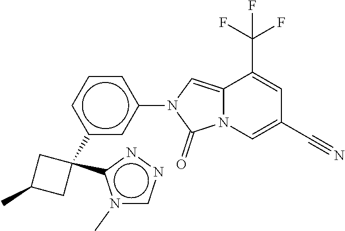 |
| 213 | 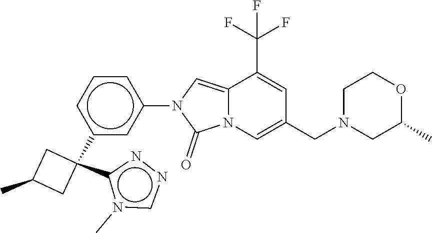 |
| 214 | 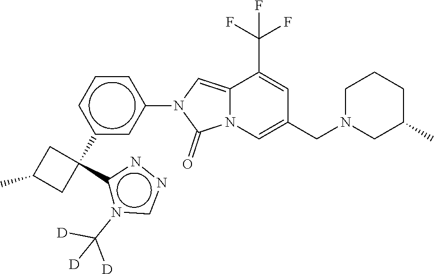 |
| 215 | 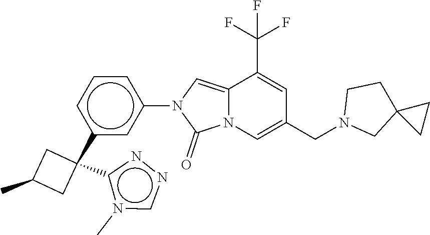 |
| 216 | 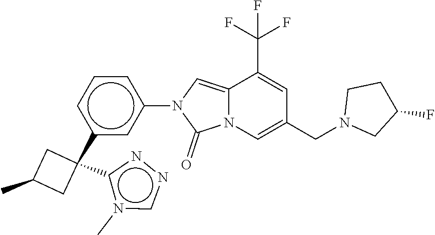 |
| 217 | 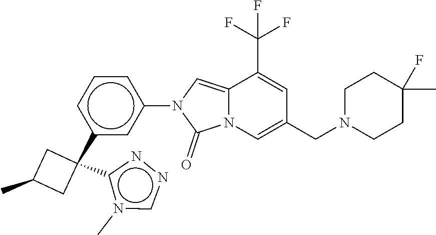 |
| 218 | 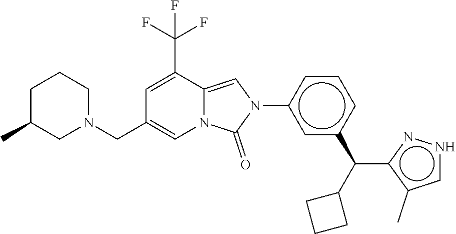 |
| 219 | 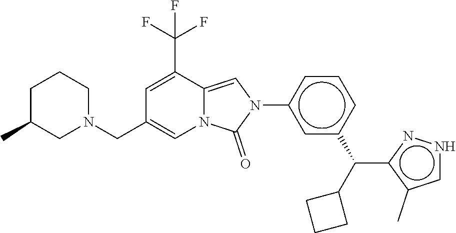 |
| 220 | 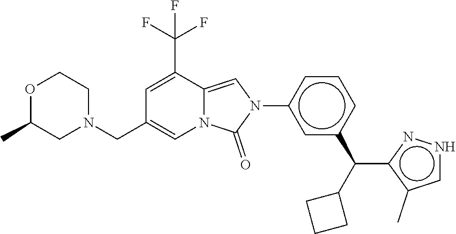 |
| 221 | 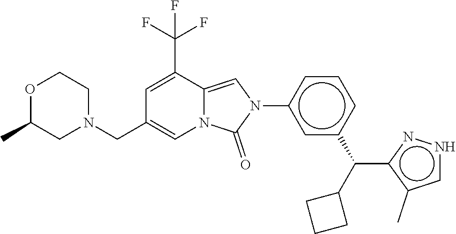 |
| 222 | 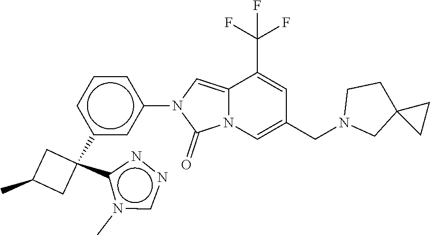 |
| 223 | 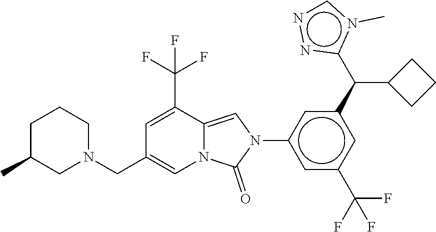 |
| 224 | 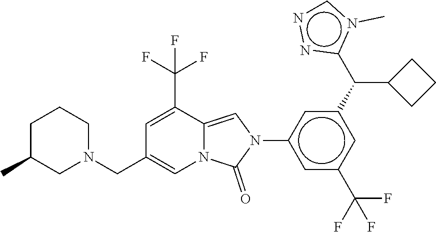 |
| 225 | 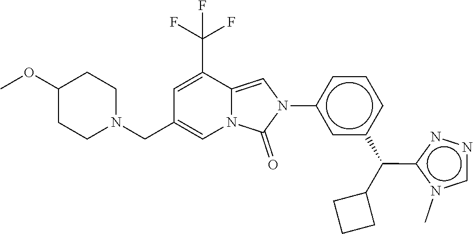 |
| 226 | 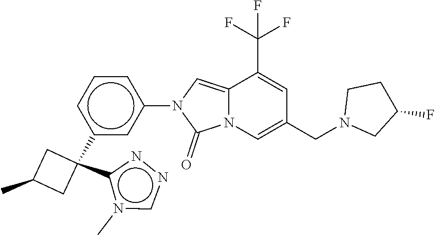 |
| 227 | 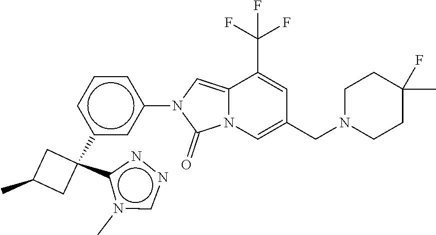 |
| 228 | 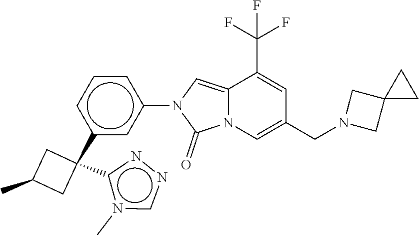 |
| 229 | 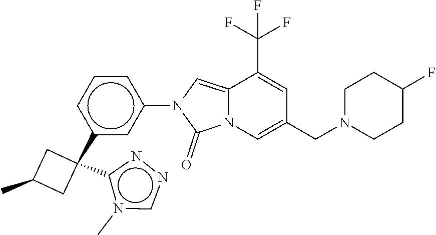 |
| 230 | 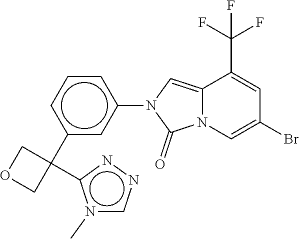 |
| 231 | 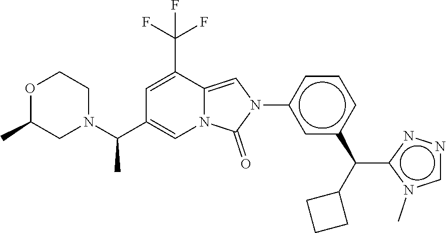 |
| 232 | 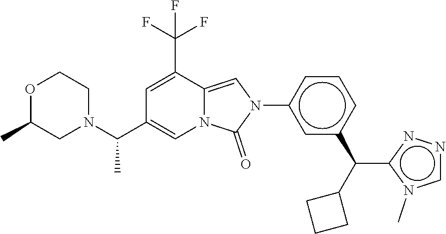 |
| 233 | 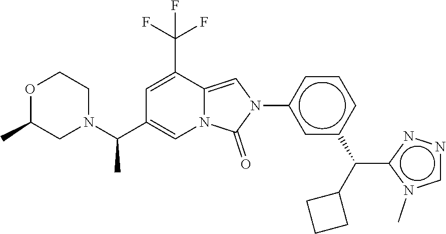 |
| 234 | 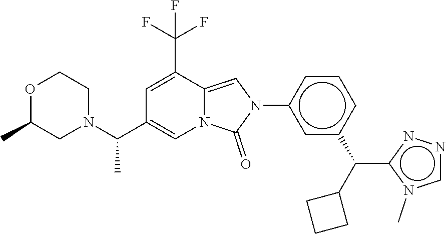 |
| 235 | 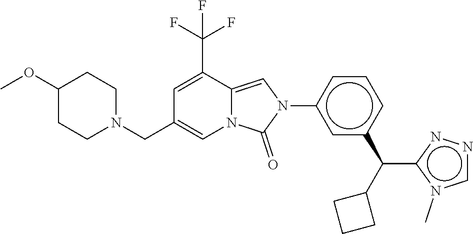 |
| 236 | 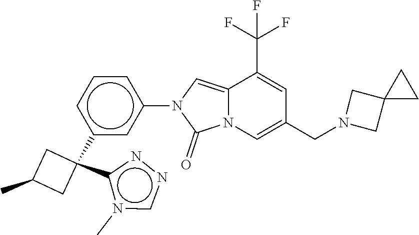 |
| 237 | 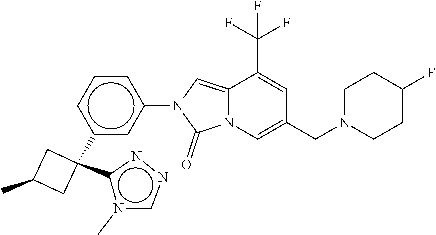 |
| 238 | 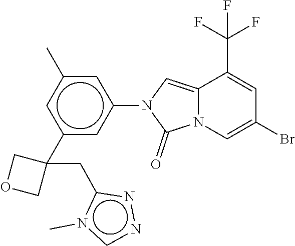 |
| 239 | 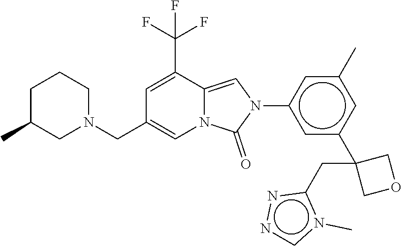 |
| 240 | 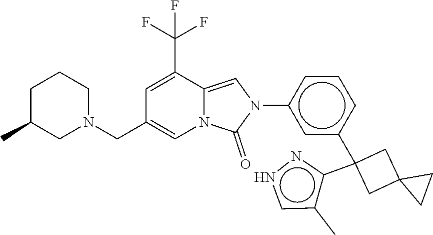 |
| 241 | 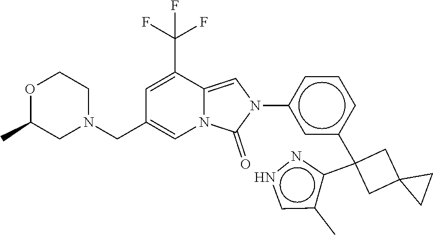 |
| 242 | 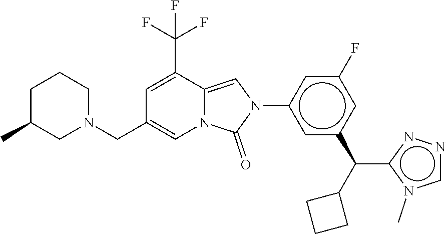 |
| 243 | 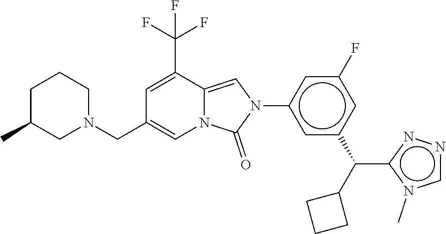 |
| 244 | 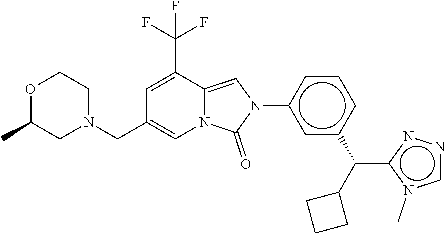 |
| 245 | 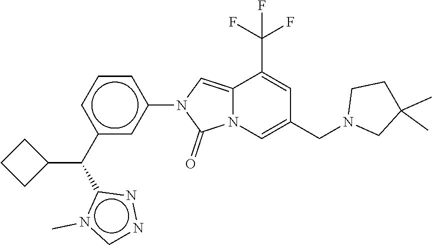 |
| 246 | 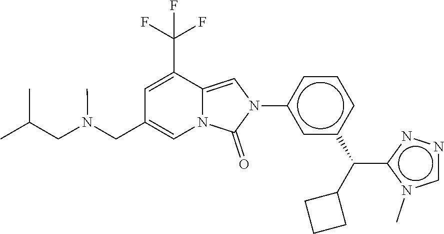 |
| 247 | 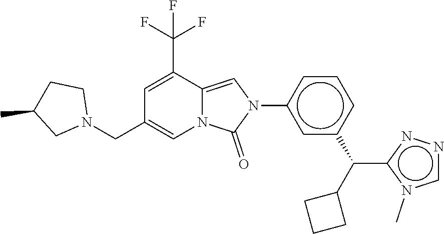 |
| 248 | 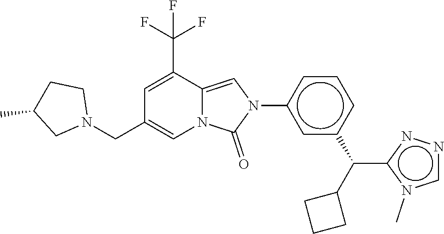 |
| 249 | 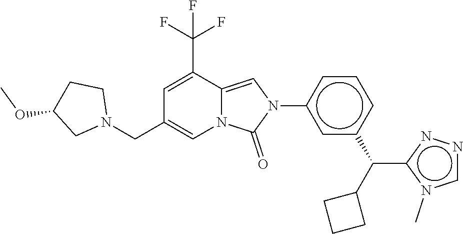 |
| 250 | 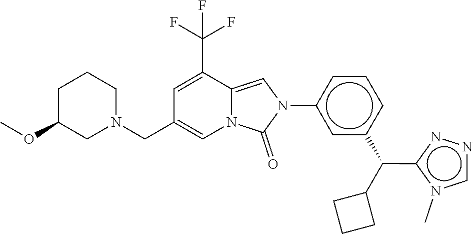 |
| 251 | 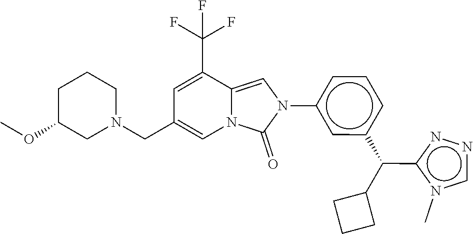 |
| 252 | 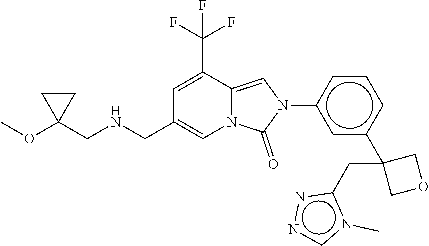 |
| 253 | 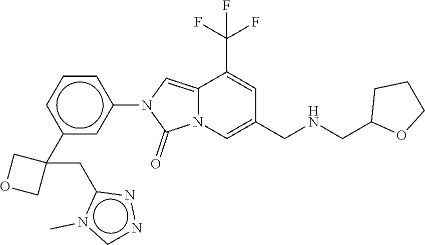 |
| 254 | 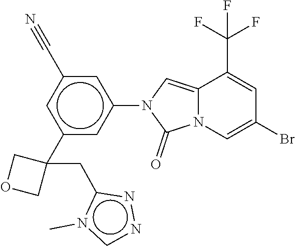 |
| 255 | 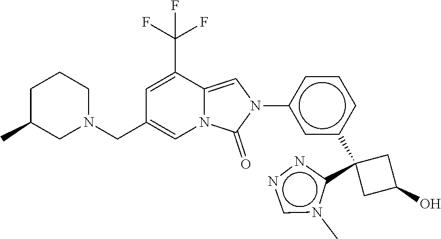 |
| 256 | 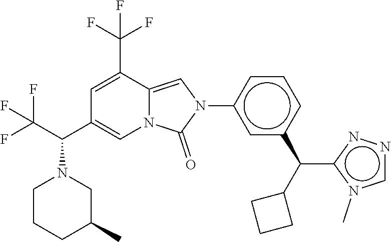 |
| 257 | 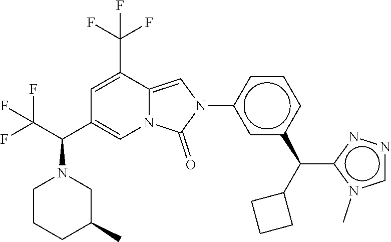 |
| 258 | 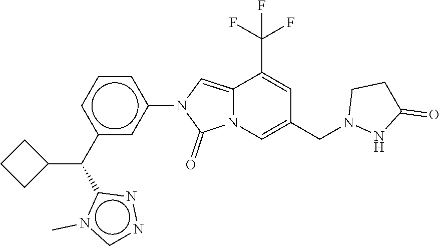 |
| 259 | 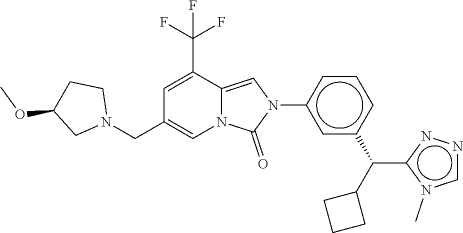 |
| 260 | 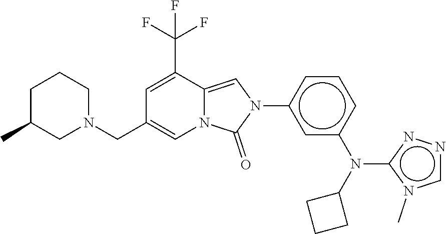 |
| 261 | 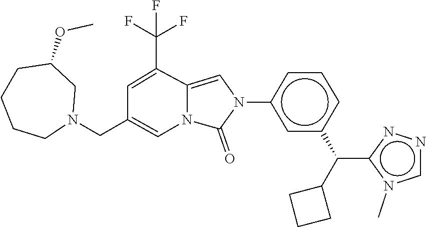 |
| 262 | 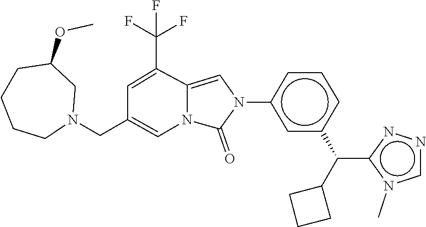 |
| 263 | 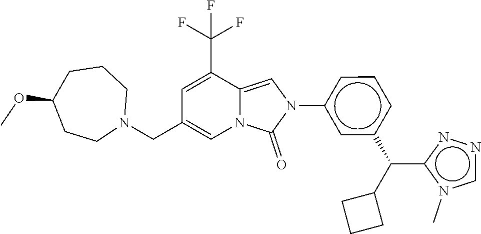 |
| 264 | 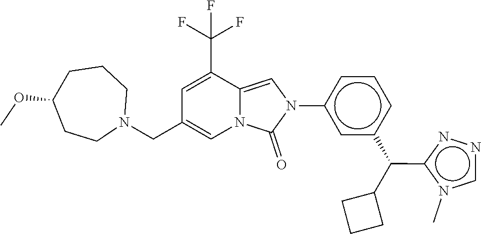 |
| 265 | 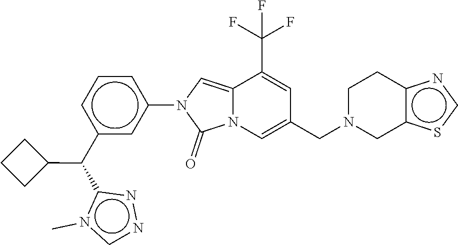 |
| 266 | 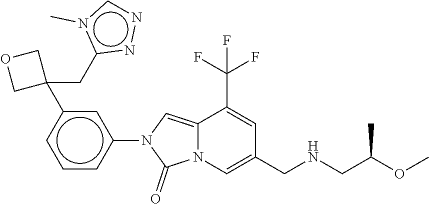 |
| 267 | 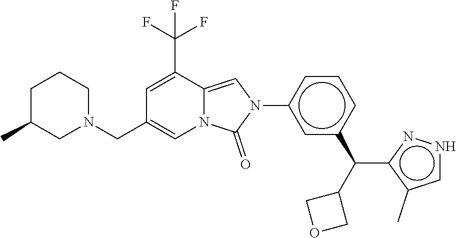 |
| 268 | 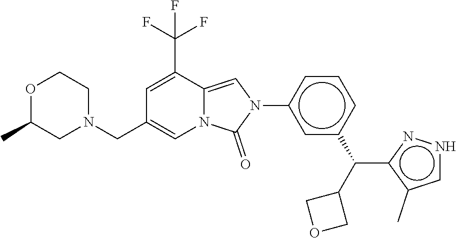 |
| 269 | 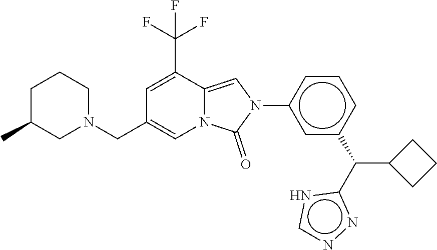 |
| 270 | 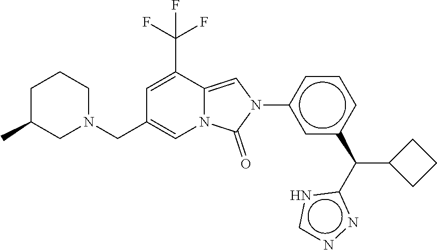 |
| 271 | 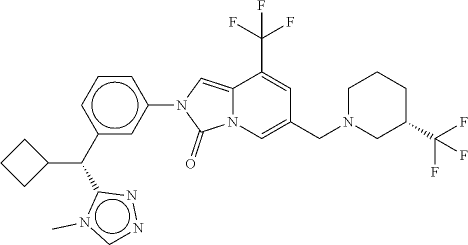 |
| 272 | 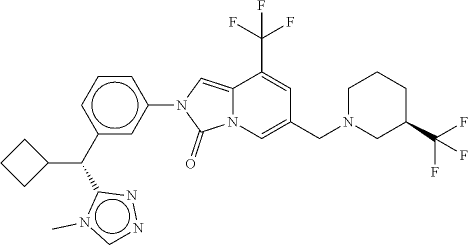 |
| 273 | 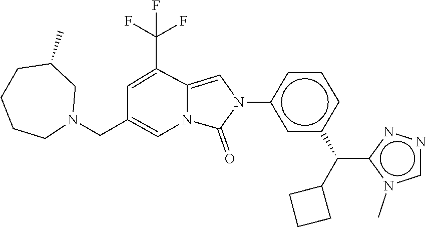 |
| 274 | 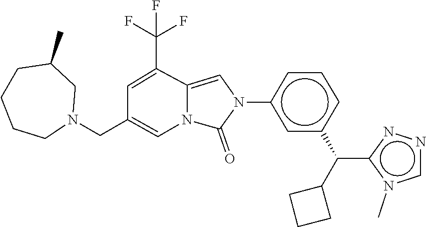 |
| 275 | 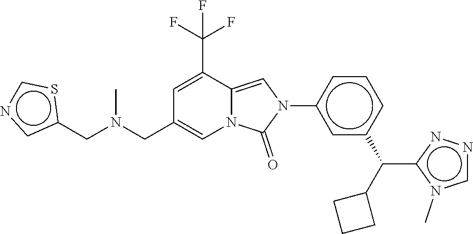 |
| 276 | 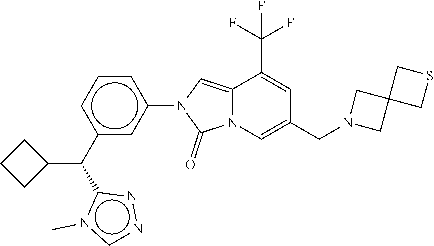 |
| 277 | 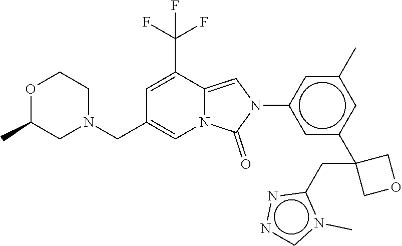 |
| 278_p1 | 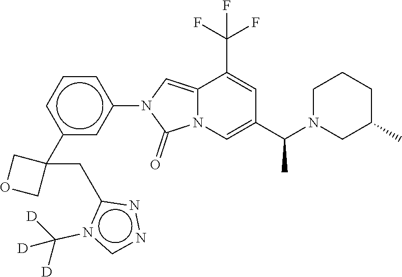 |
| 278_p2 | 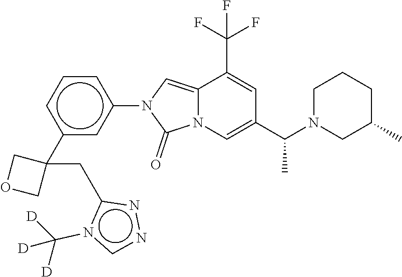 |
| 279 | 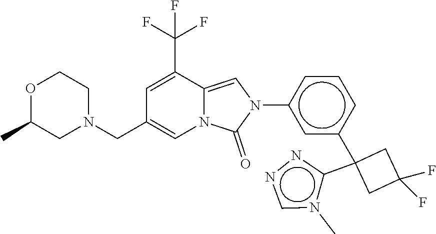 |
| 280 | 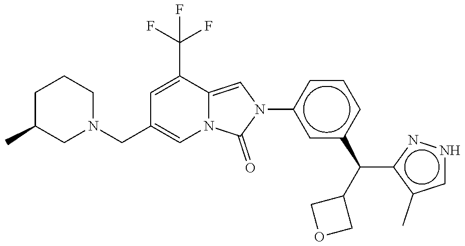 |
| 281 | 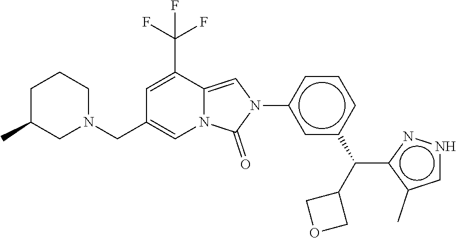 |
| 282 | 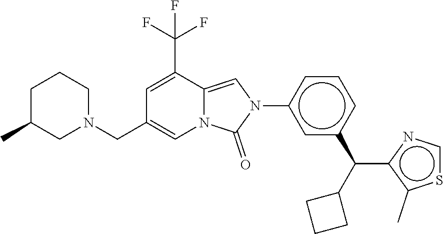 |
| 283 | 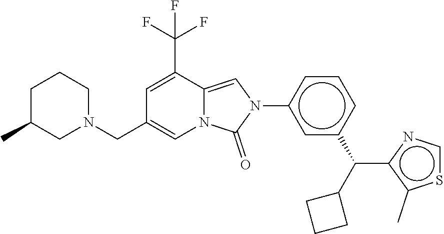 |
| 284 | 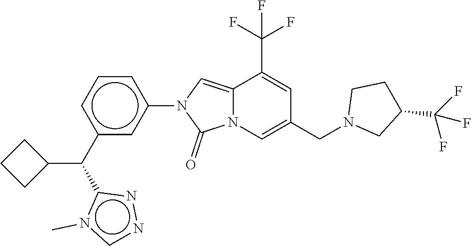 |
| 285 | 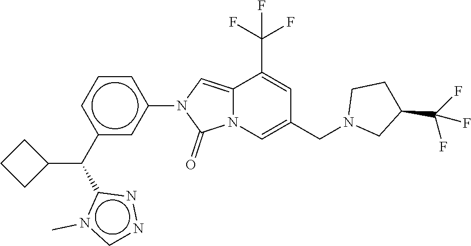 |
| 286 | 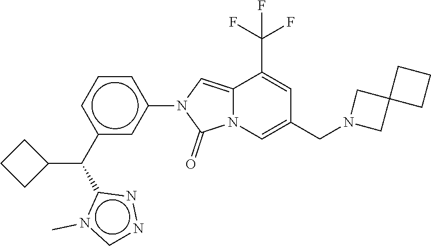 |
| 287_P1 | 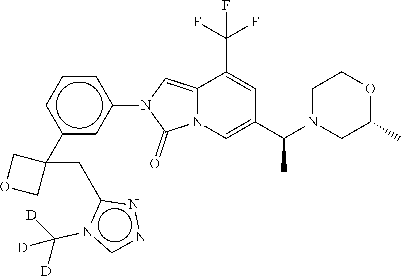 |
| 287_P2 | 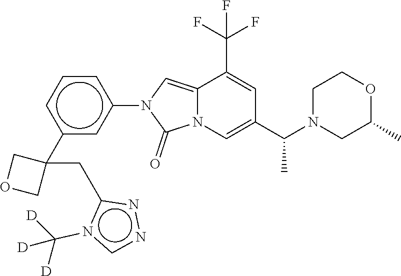 |
| 288 | 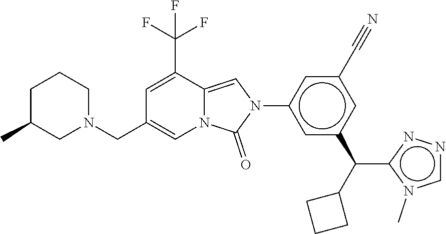 |
| 289 | 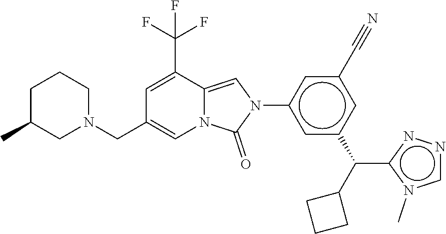 |
| 290 | 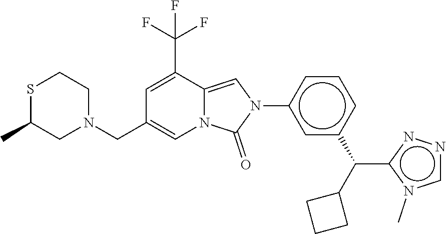 |
| 291 | 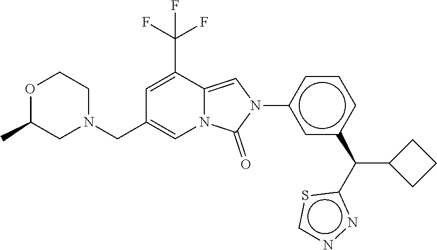 |
| 292 | 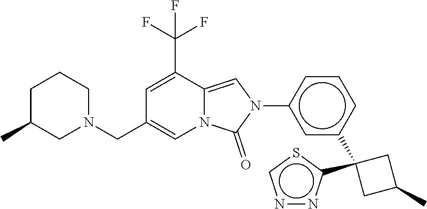 |
| 293 | 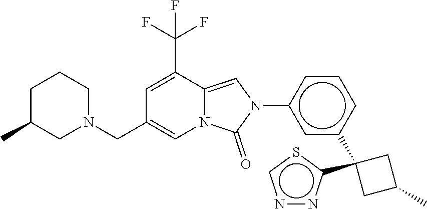 |
| 294 | 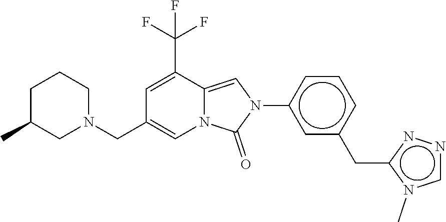 |
| 295 | 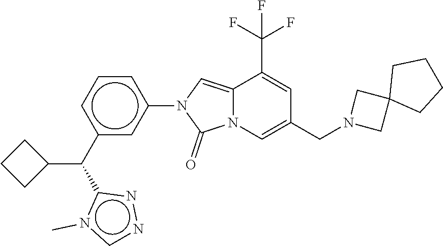 |
| 296 | 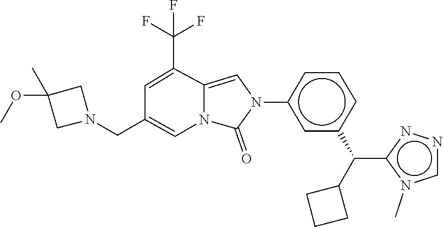 |
| 297 | 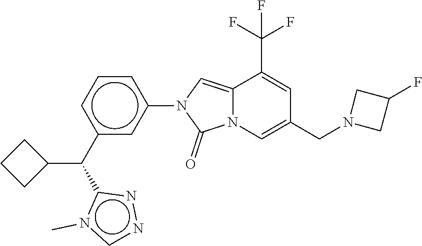 |
| 298 | 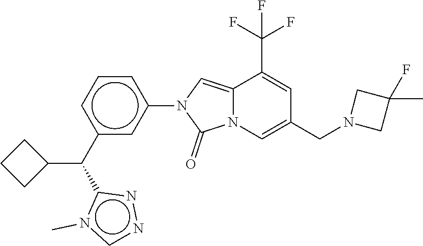 |
| 299 | 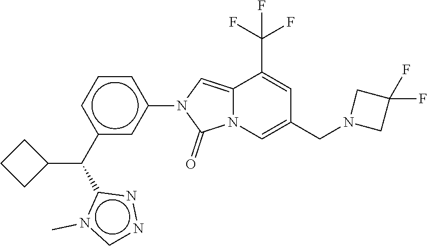 |
| 300 | 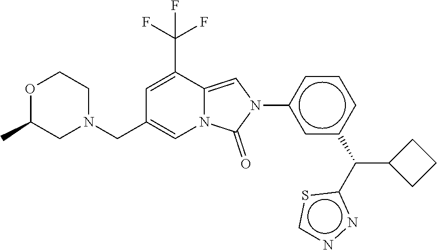 |
| 301 | 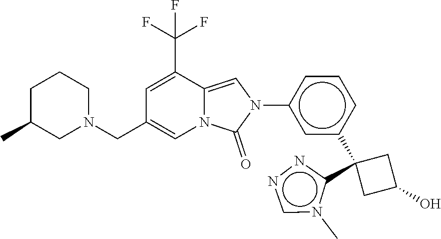 |
| 302 | 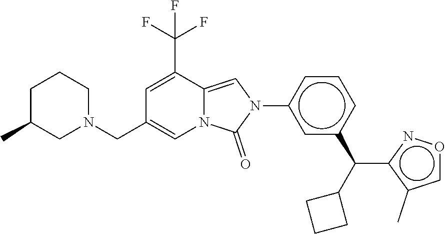 |
| 303 | 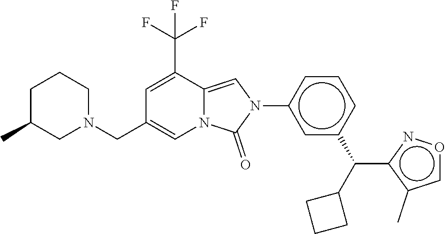 |
| 304 | 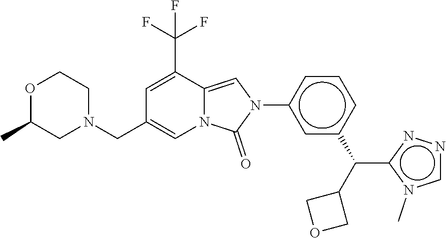 |
| 305 | 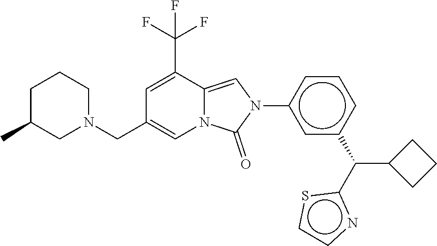 |
| 306 | 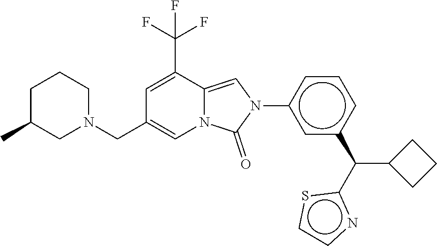 |
| 307 | 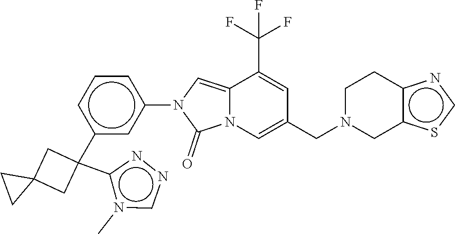 |
| 308 | 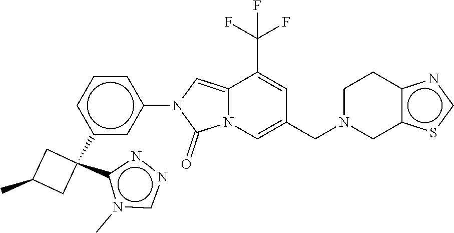 |
| 309 | 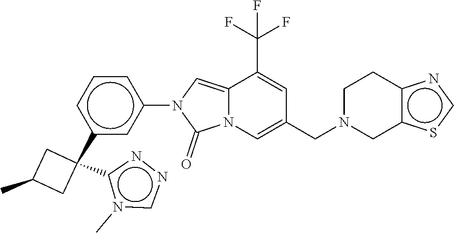 |
| 310 | 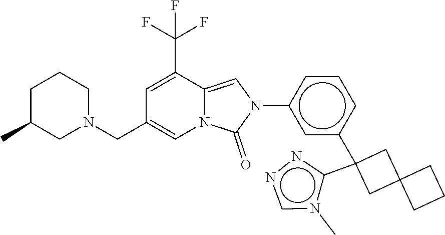 |
| 311 | 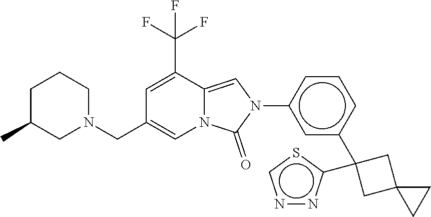 |
| 312 | 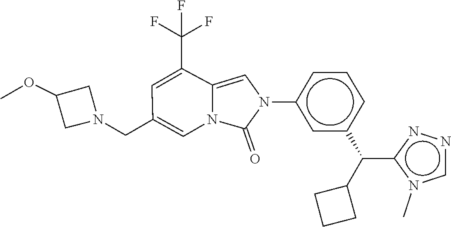 |
| 313 | 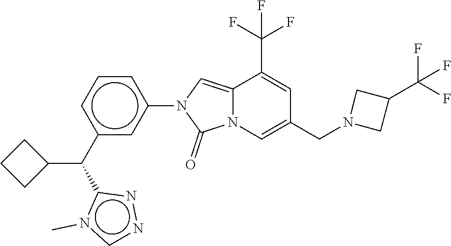 |
| 314 | 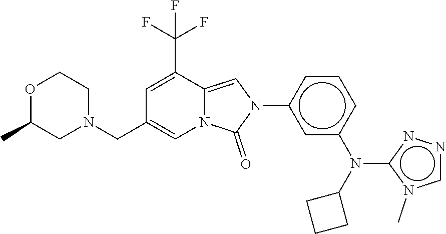 |
| 315 | 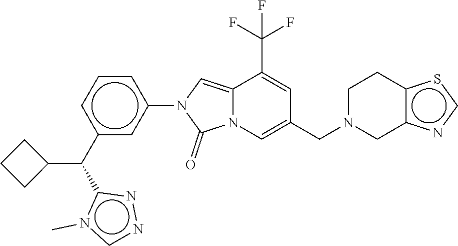 |
| 316 | 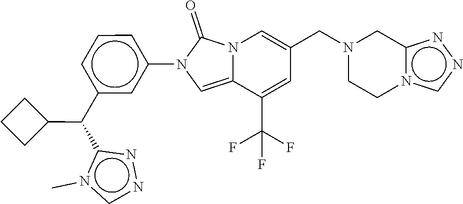 |
| 317 | 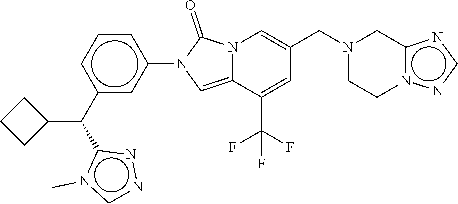 |
| 318 | 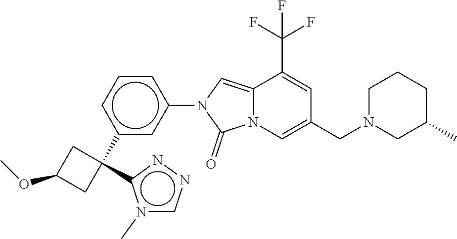 |
| 319 | 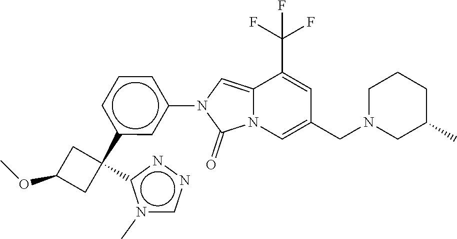 |
| 329 | 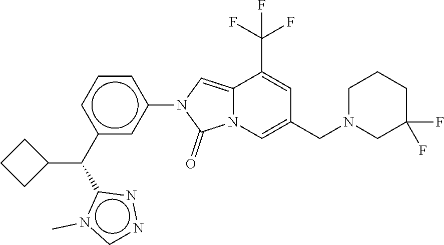 |
| 321 | 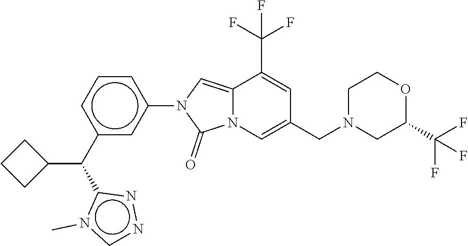 |
| 322 | 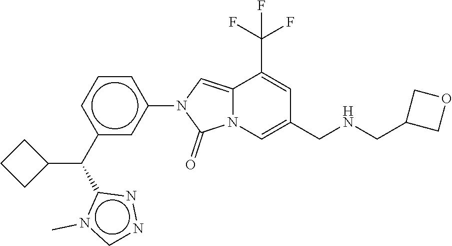 |
| 323 | 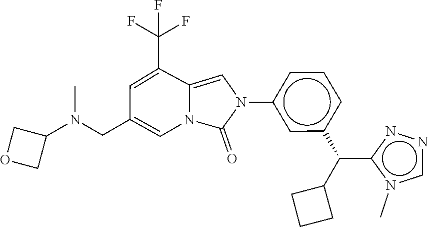 |
| 324 | 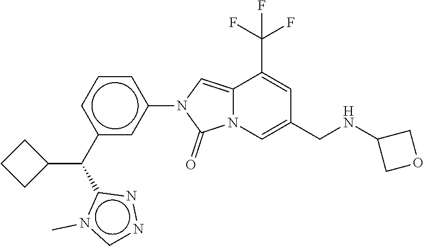 |
| 325 | 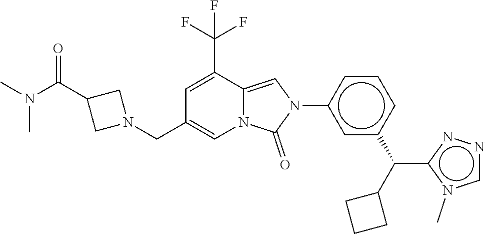 |
| 326 | 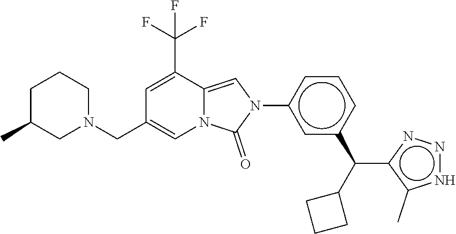 |
| 327 | 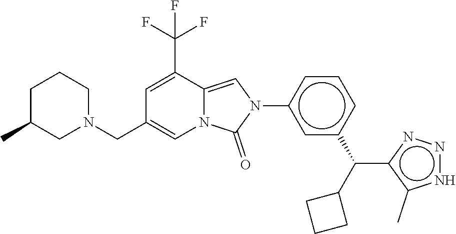 |
| 328 | 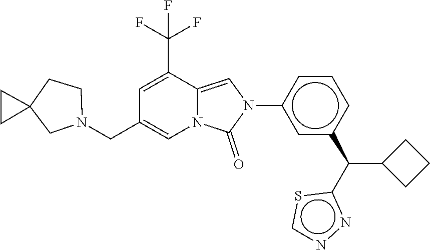 |
| 329 | 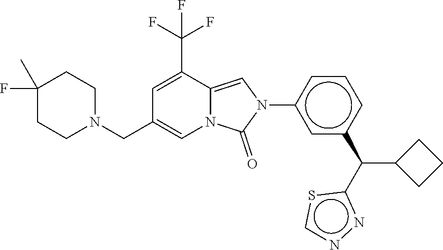 |
| 330 | 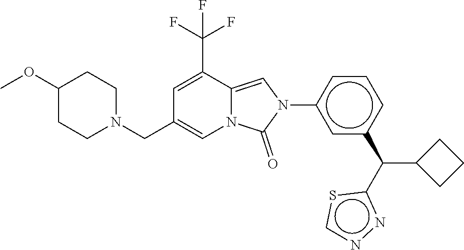 |
| 331 | 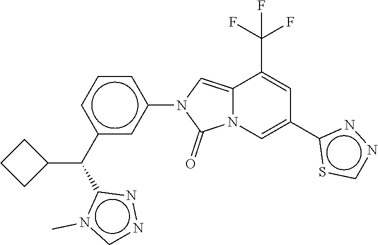 |
| 332_p1 | 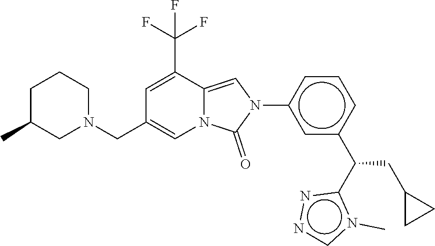 |
| 332-p2 | 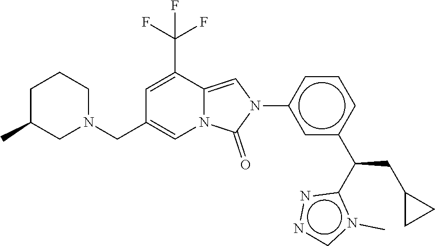 |
| 333 | 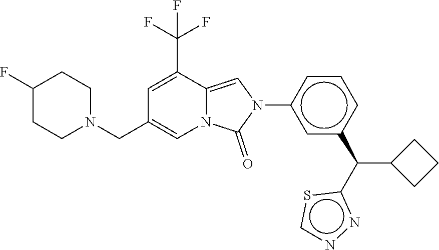 |
| 334 | 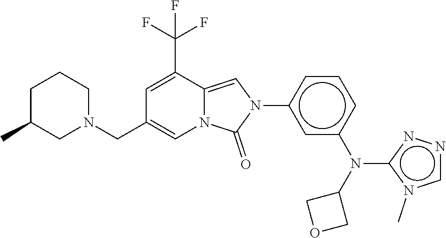 |
| 335 | 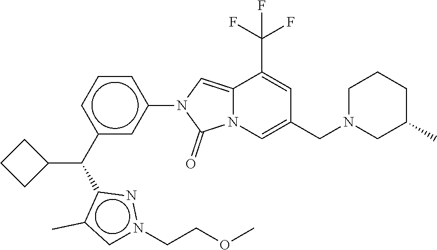 |
| 336 | 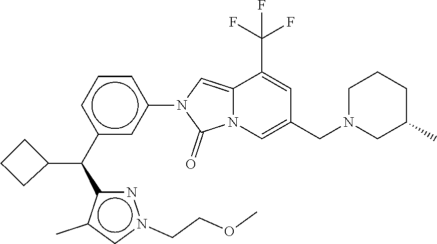 |
| 337 | 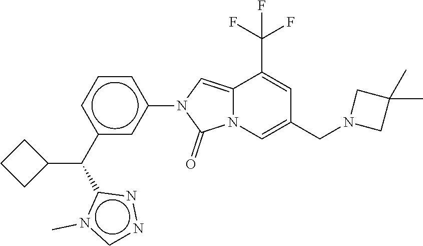 |
| 338 | 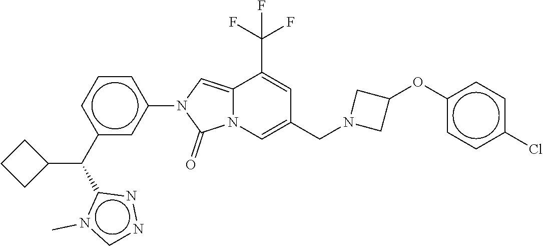 |
| 339_P1 | 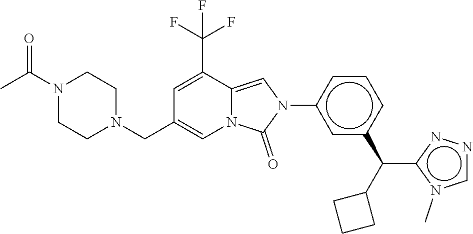 |
| 339_P2 | 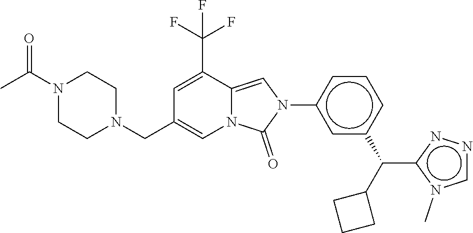 |
| 340_P1 | 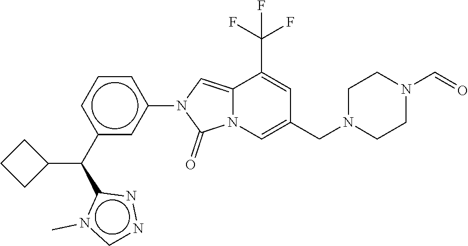 |
| 340_P2 | 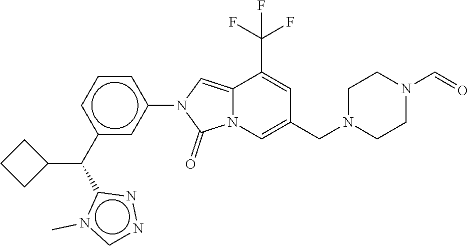 |
| 341 | 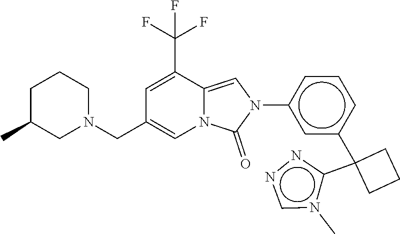 |
| 342 | 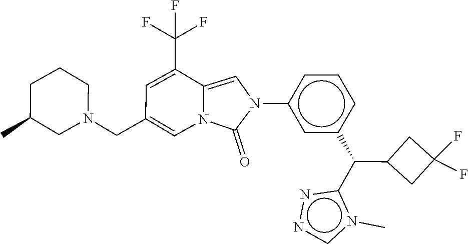 |
| 343 | 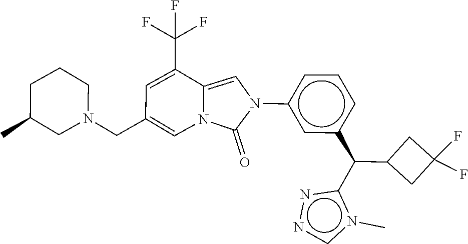 |
| 344 | 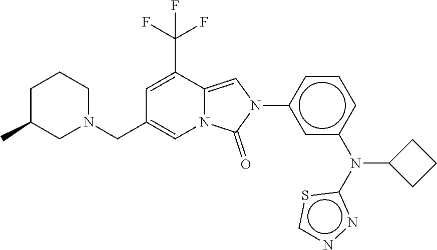 |
| 345 | 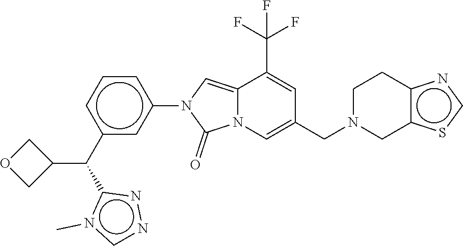 |
| 346 | 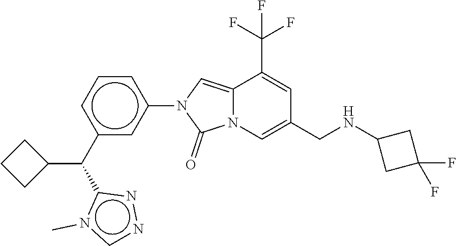 |
| 347 | 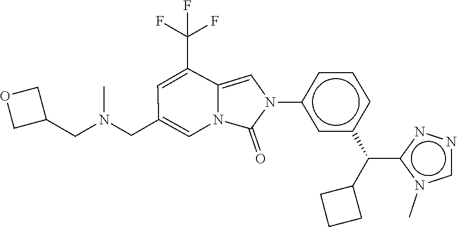 |
| 348 | 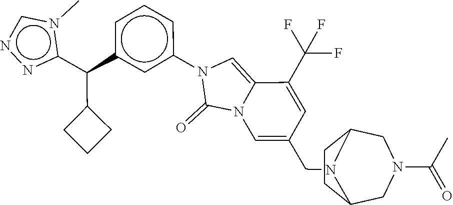 |
| 349 | 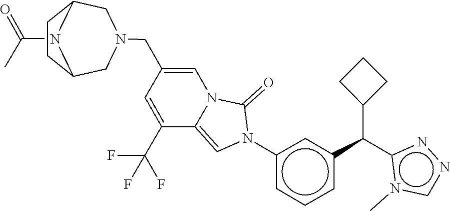 |
| 350 | 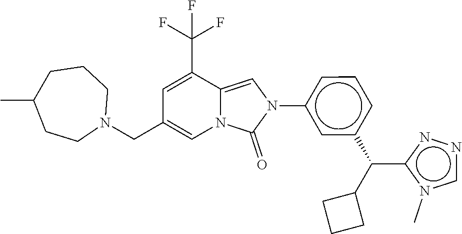 |
| 351_P1 | 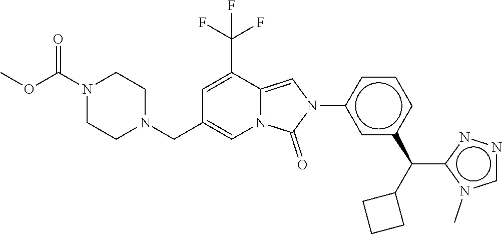 |
| 351_P2 | 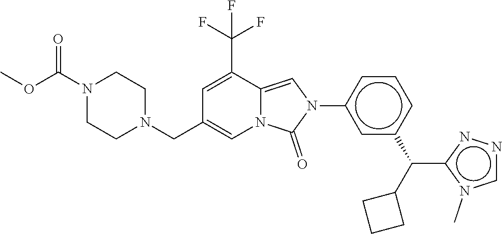 |
| 352_P1 | 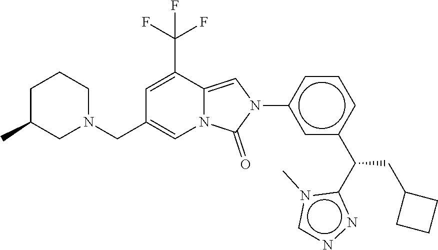 |
| 352_P2 | 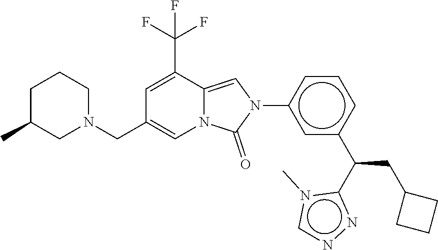 |
| 353 | 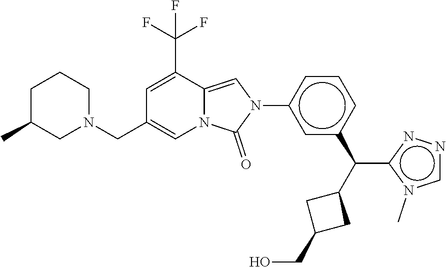 |
| 354 | 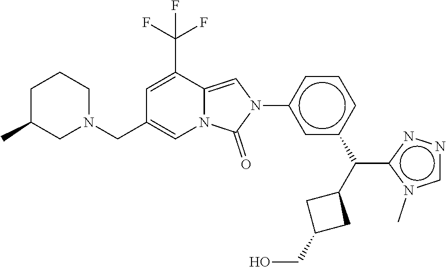 |
| 355 | 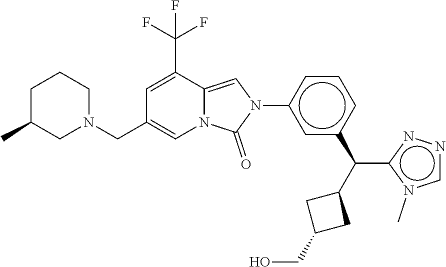 |
| 356 | 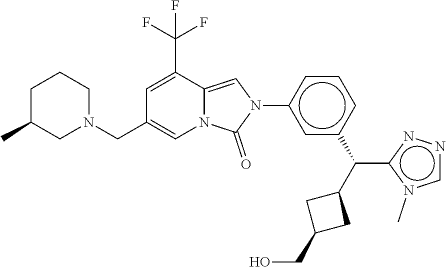 |
| 357 | 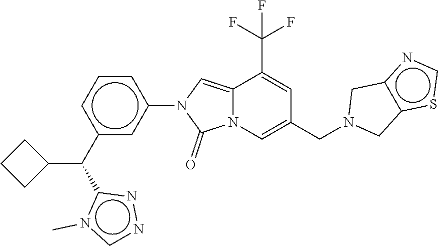 |
| 358 | 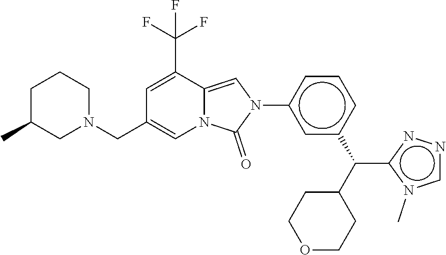 |
| 359 | 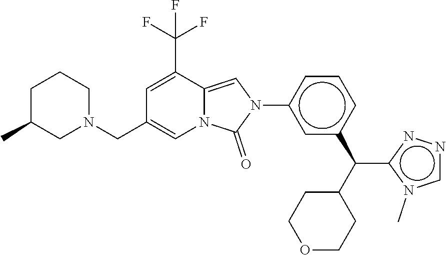 |
| 360 | 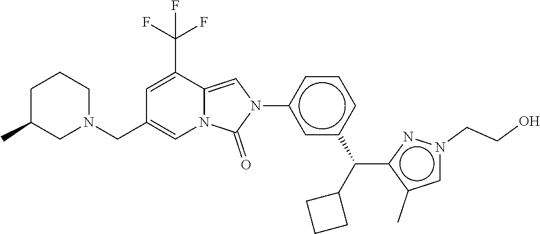 |
| 361 | 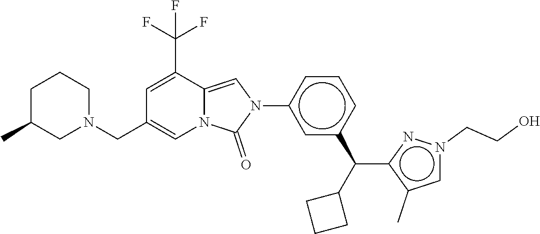 |
| 362_P1 | 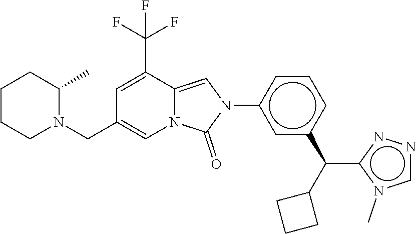 |
| 362_P2 | 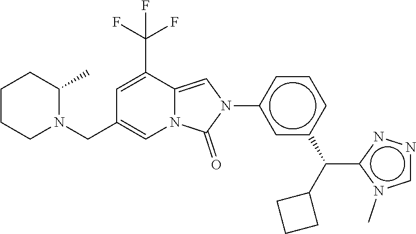 |
| 363_P1 | 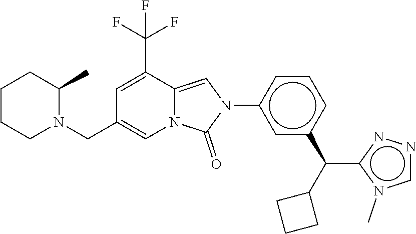 |
| 363_P2 | 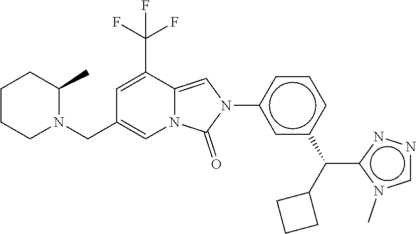 |
| 364_P1 | 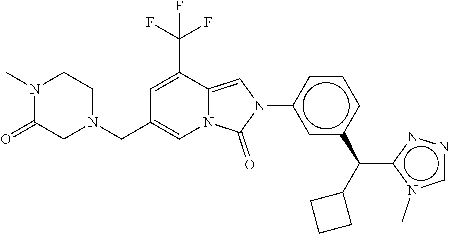 |
| 364_P2 | 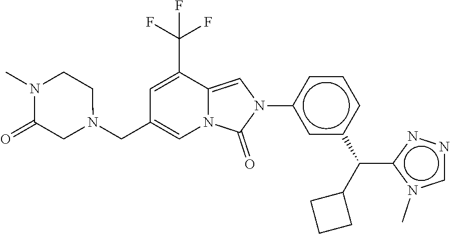 |
| 365 | 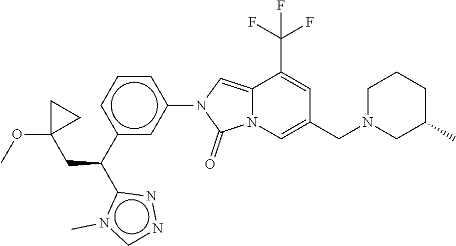 |
| 366 | 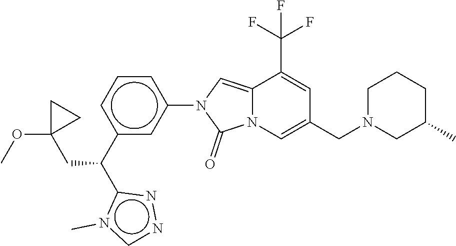 |
| 367 | 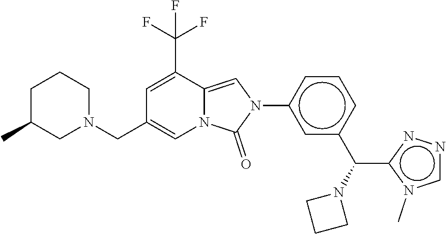 |
| 368 | 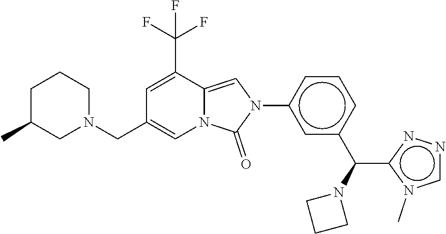 |
| 369 | 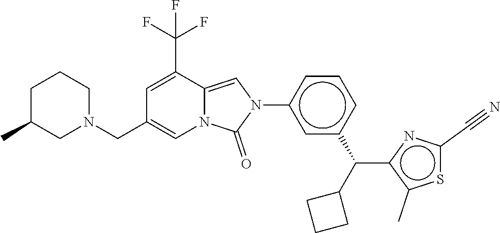 |
| 370 | 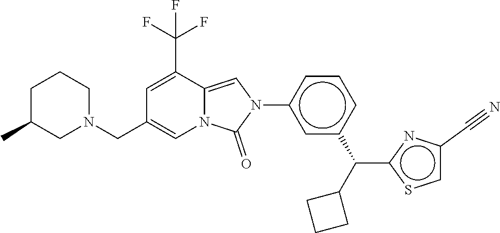 |
| 371 | 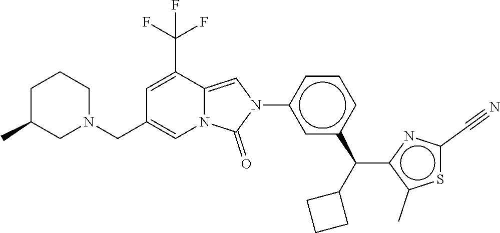 |
| 372 | 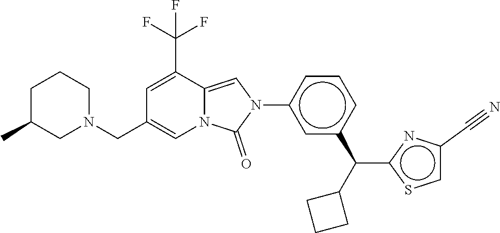 |
| 373 | 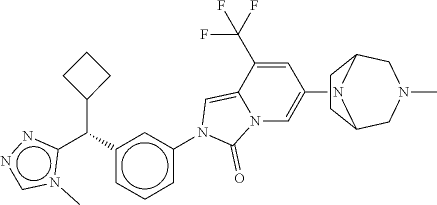 |
| 374 | 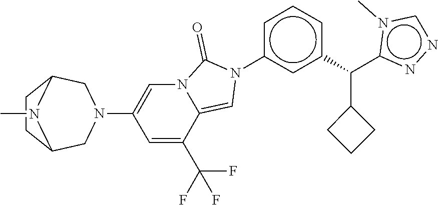 |
| 375 | 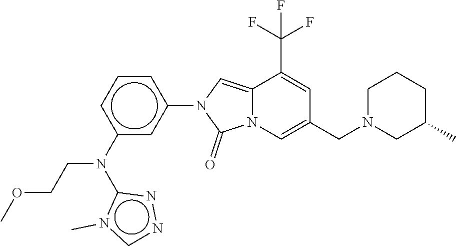 |
| 376 | 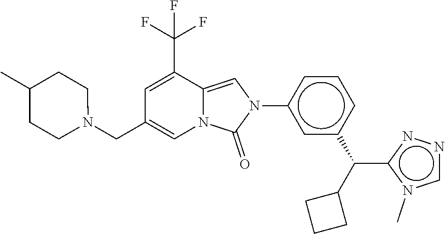 |
| 377 | 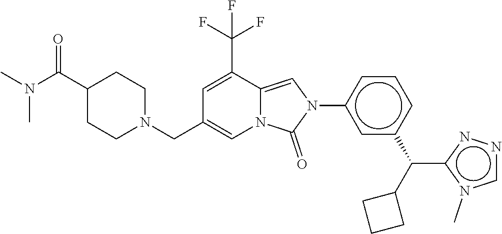 |
| 378 | 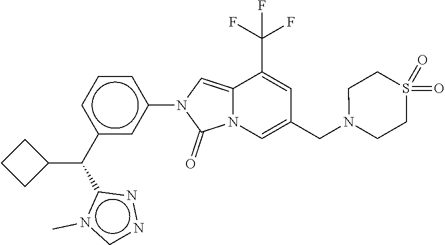 |
| 379 | 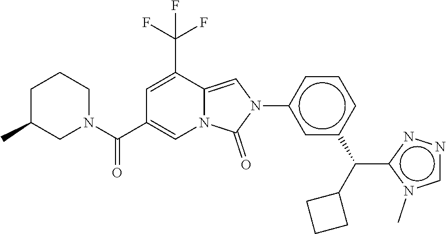 |
| 380_P1 | 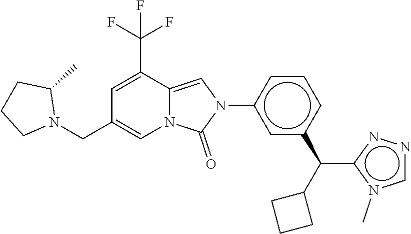 |
| 380_P2 | 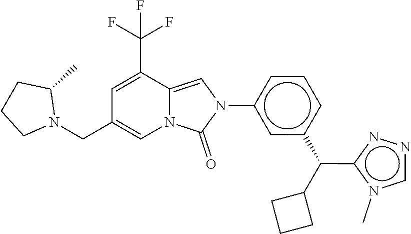 |
| 381_P1 | 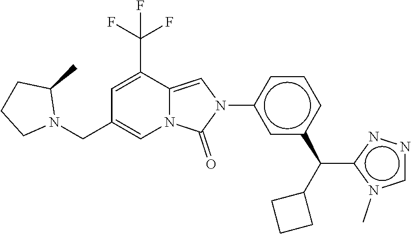 |
| 381_P2 | 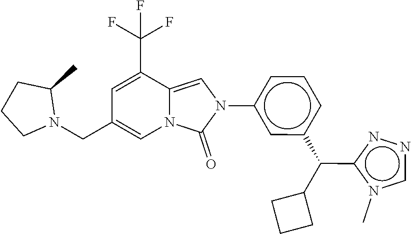 |
| | 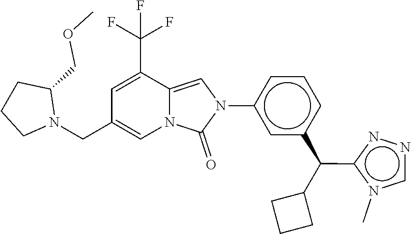 |
| | 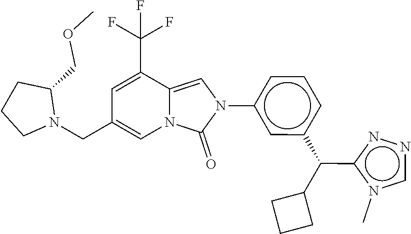 |
| 382_P1 | 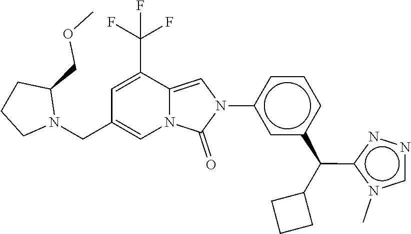 |
| 382_P2 | 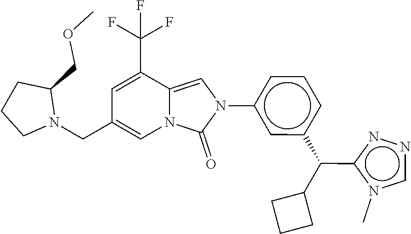 |
| 383 | 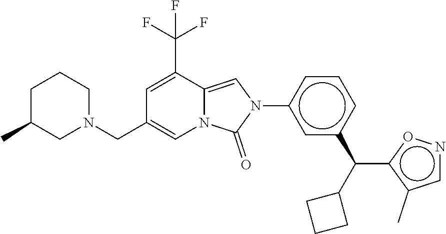 |
| 384 | 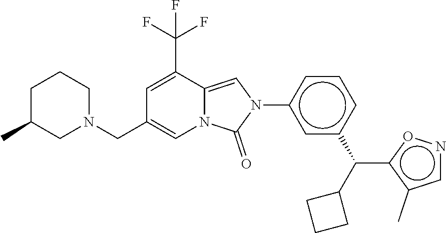 |
| 385 | 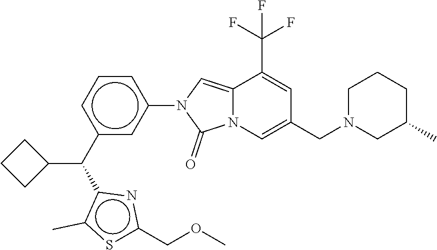 |
| 386 | 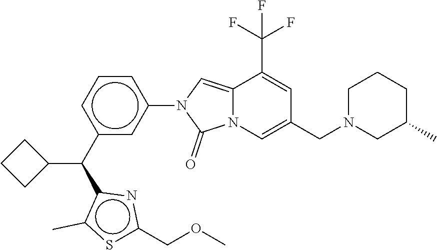 |
| 387 | 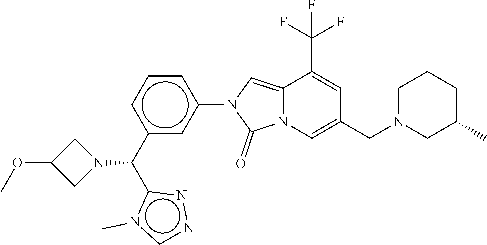 |
| 388 | 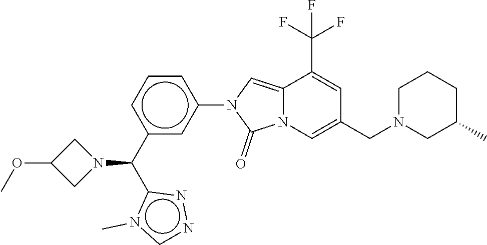 |
| 389 | 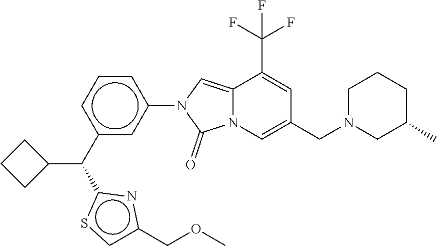 |
| 390 | 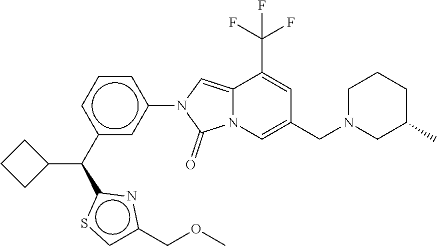 |
| 391 | 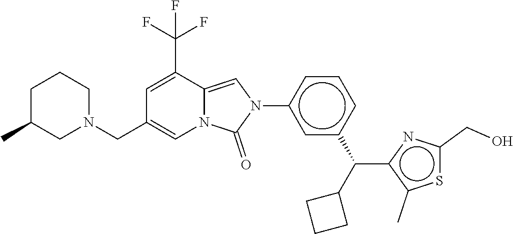 |
| 392 | 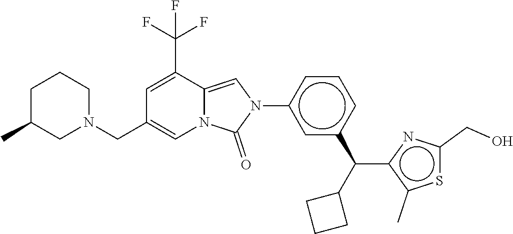 |
| 393 | 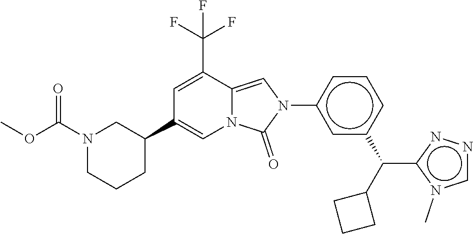 |
| 394 | 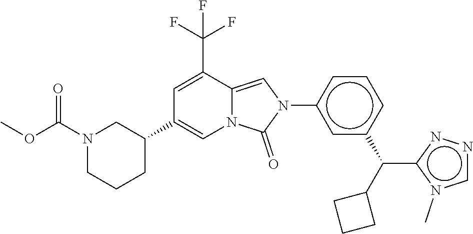 |
| 395 | 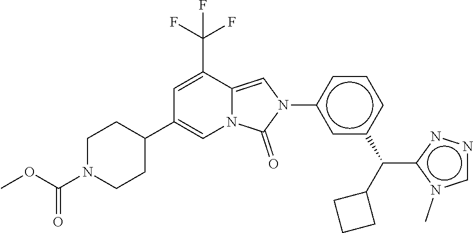 |
| 396 | 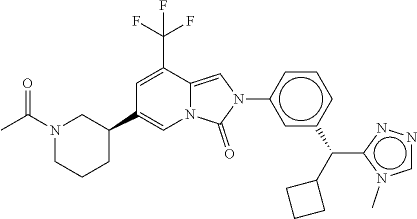 |
| 397 | 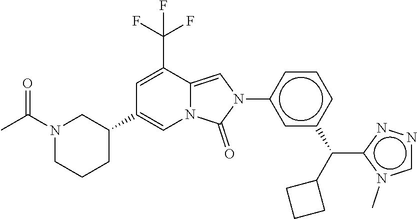 |
| 398 | 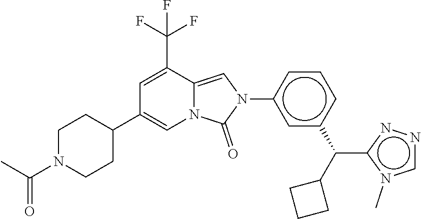 |
| 399 | 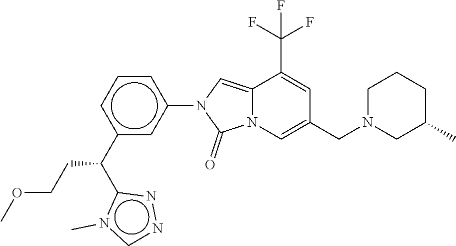 |
| 400 | 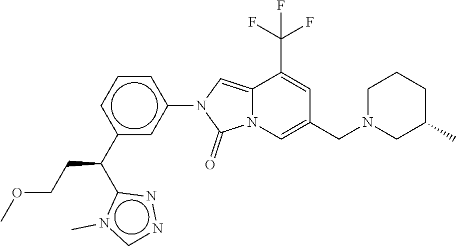 |
| 401_P1 | 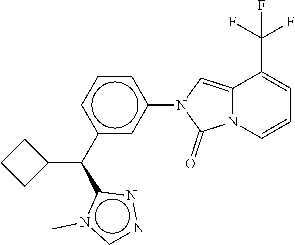 |
| 401_P2 | 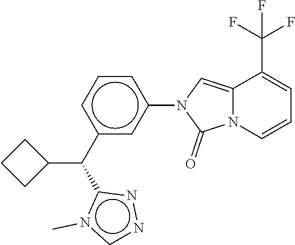 |
| 402 | 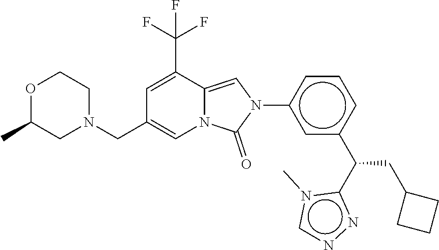 |
| 403_P1 | 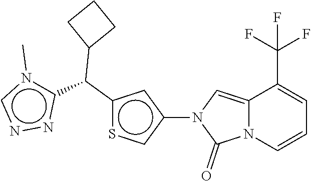 |
| 403_P2 | 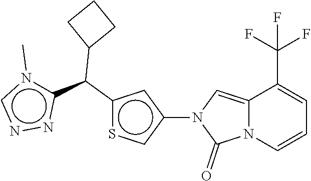 |
| 404 | 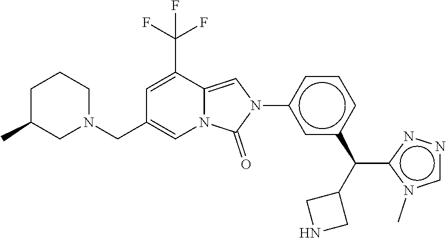 |
| 403 | 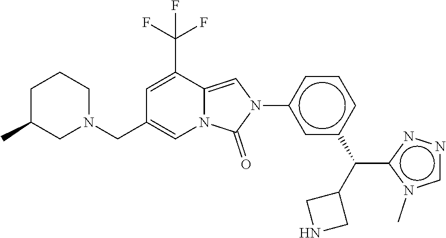 |
| 404 | 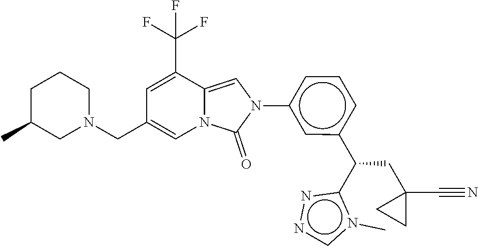 |
| 405 | 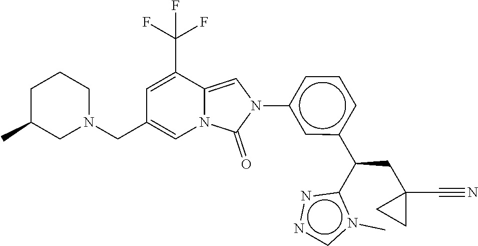 |
| 406 | 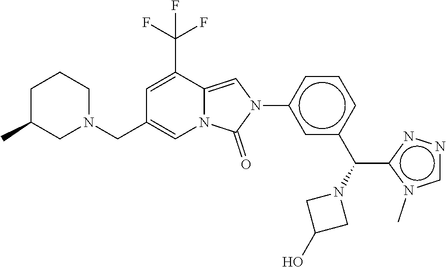 |
| 407 | 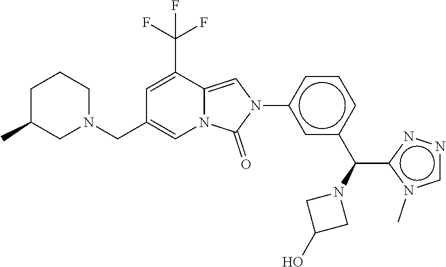 |
| 408 | 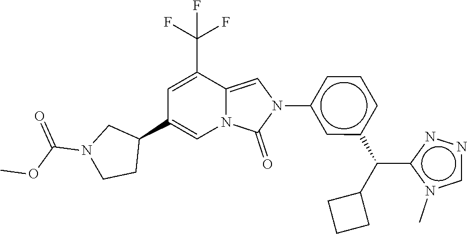 |
| 409 | 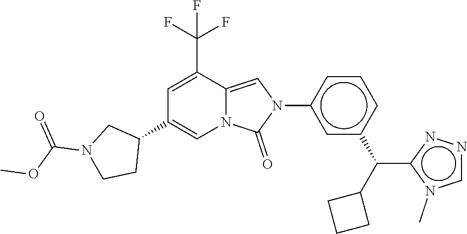 |
| 410 | 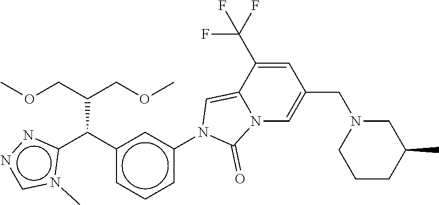 |
| 411 | 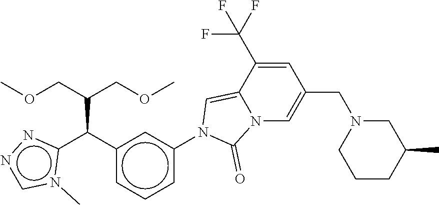 |
| 412 | 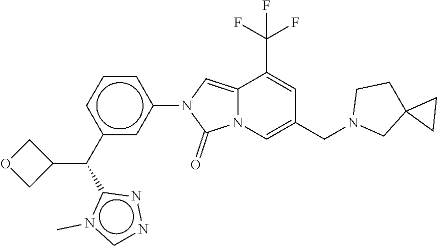 |
| 413 | 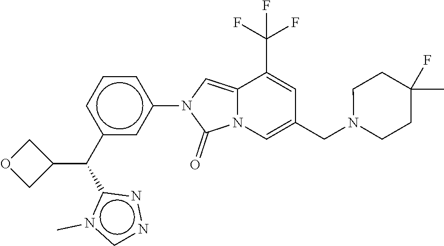 |
| 414 | 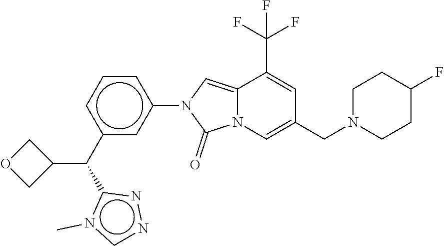 |
| 415 | 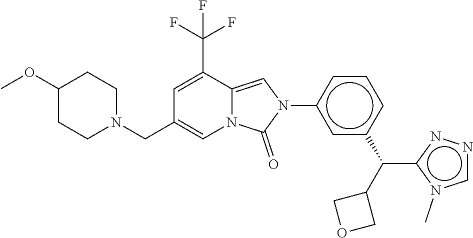 |
| 416 | 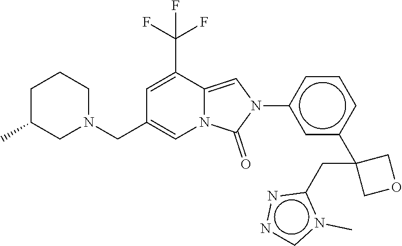 |
| 417 | 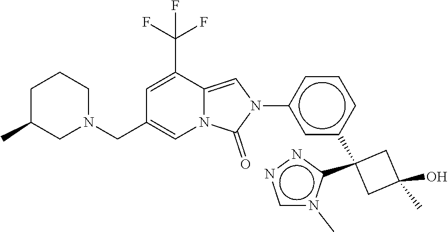 |
| 418 | 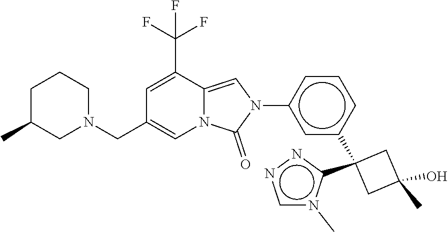 |
| 419 | 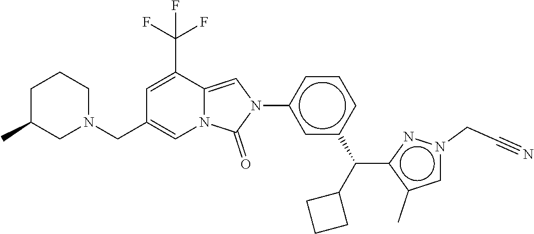 |
| 420 | 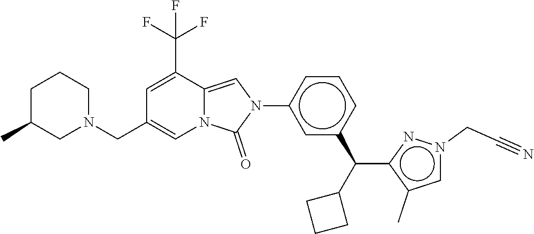 |
| 421 | 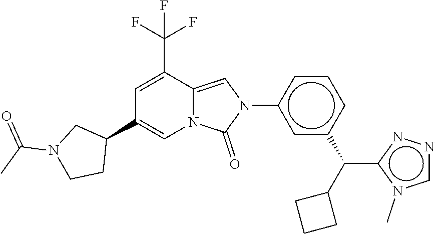 |
| 422 | 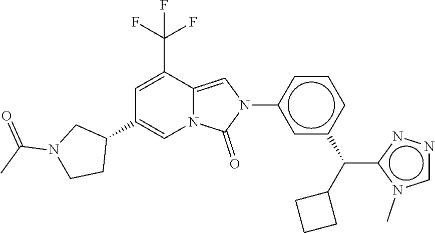 |
| 423 | 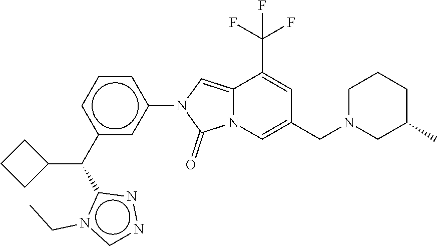 |
| 424 | 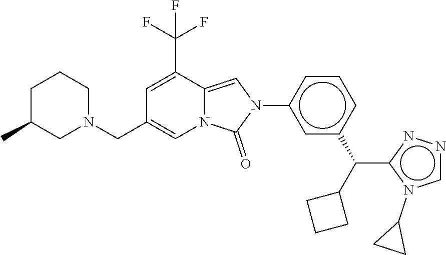 |
| 425 | 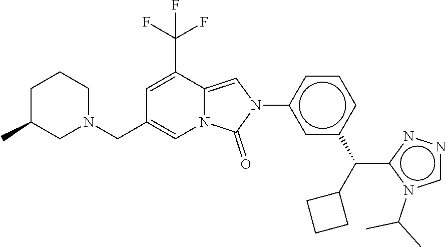 |
| 426 | 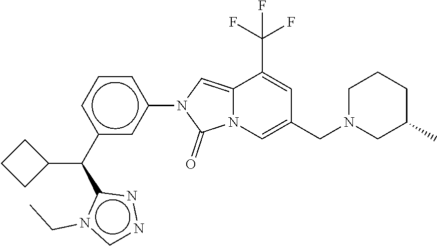 |
| 427 | 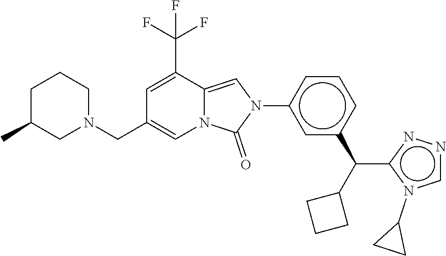 |
| 428 | 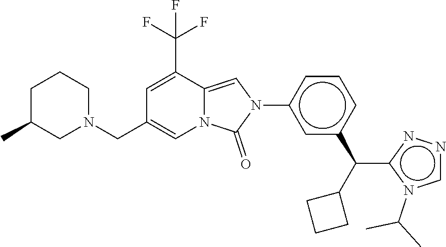 |
| 429 | 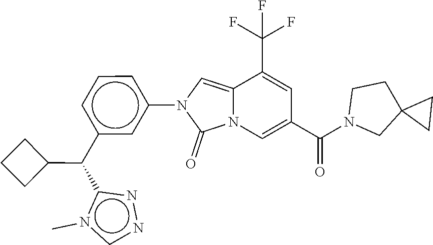 |
| 430 | 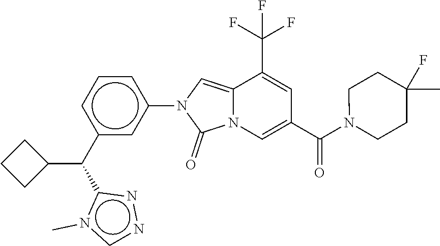 |
| 431 | 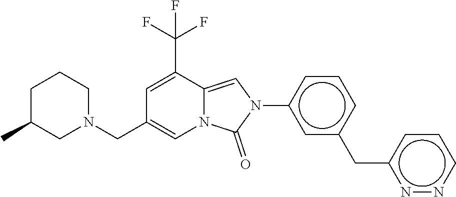 |
| 432 | 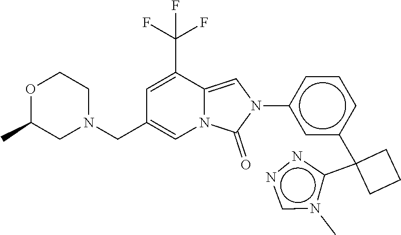 |
| 433 | 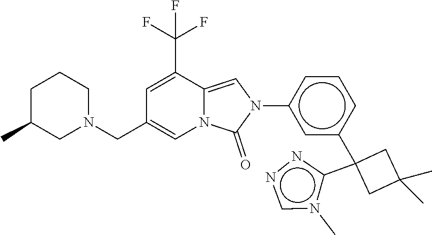 |
| 434_P1 | 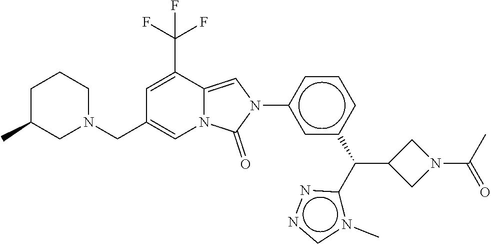 |
| 434_P2 | 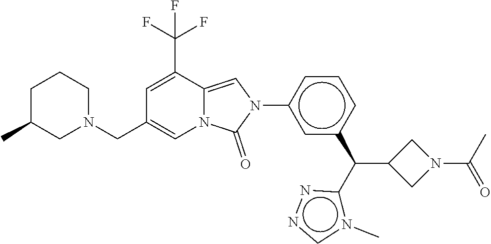 |
| 435 | 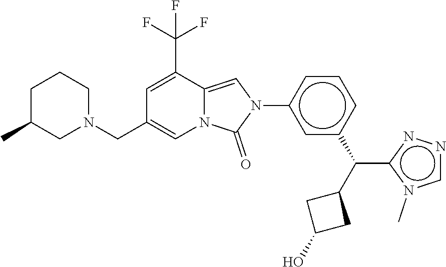 |
| 436 | 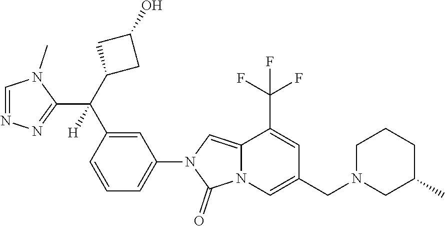 [SMILES: C[C@H]1CCCN(Cc2cc(C(F)(F)F)c3cn(-c4cccc([C@@H](c5nncn5C)[C@H]5C[C@@H](O)C5)c4)c(=O)n3c2)C1] |
| 437 | 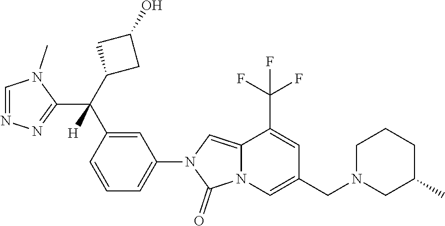 [SMILES: C[C@H]1CCCN(Cc2cc(C(F)(F)F)c3cn(-c4cccc([C@H](c5nncn5C)[C@H]5C[C@@H](O)C5)c4)c(=O)n3c2)C1] |
| 438 | 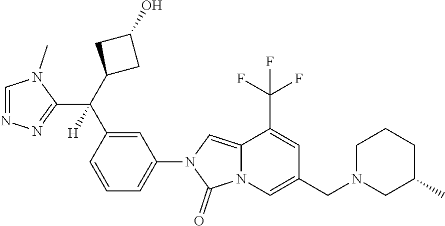 [SMILES: C[C@H]1CCCN(Cc2cc(C(F)(F)F)c3cn(-c4cccc([C@@H](c5nncn5C)[C@H]5C[C@H](O)C5)c4)c(=O)n3c2)C1] |
| 439 | 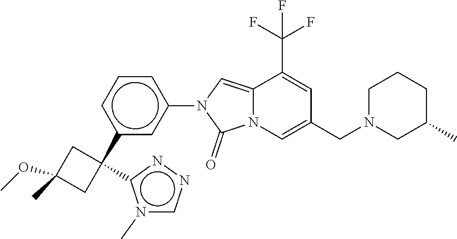 |
| 440 | 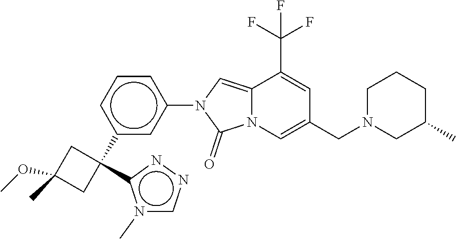 |
| 441 | 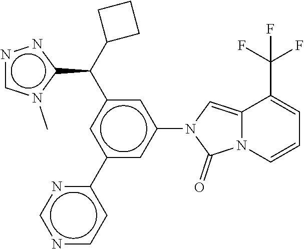 |
| 442 | 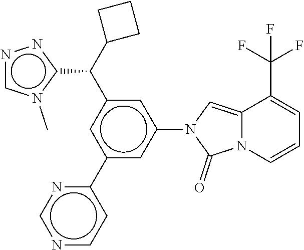 |
| 443 | 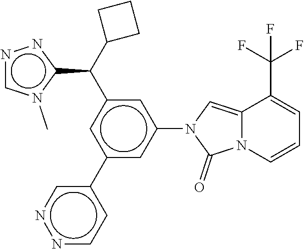 |
| 444 | 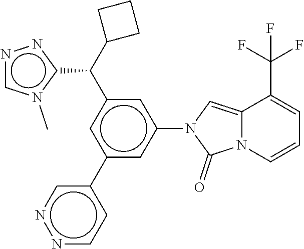 |
| 445 | 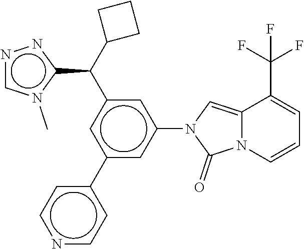 |
| 446 | 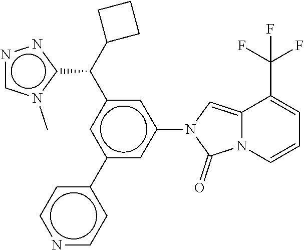 |
| 447 | 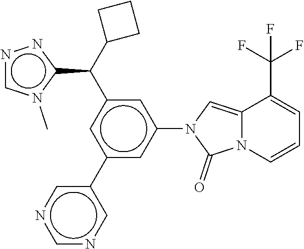 |
| 448 | 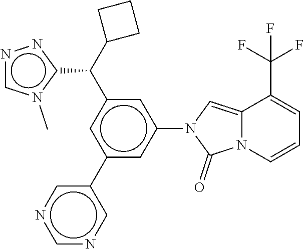 |
| 449 | 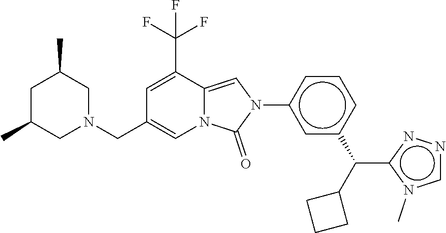 |
| 450 | 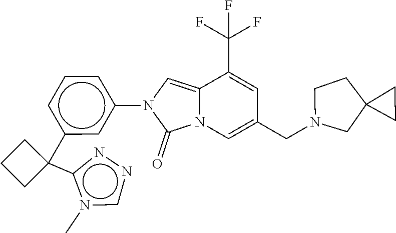 |
| 451 | 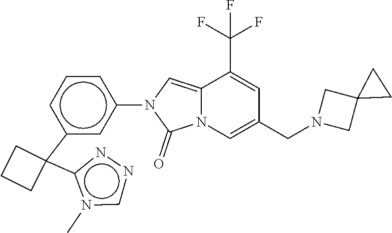 |
| 452 | 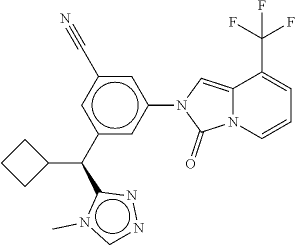 |
| 453 | 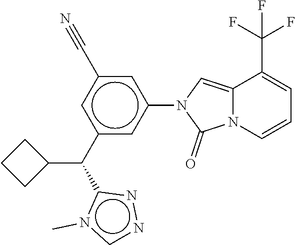 |
| 454 | 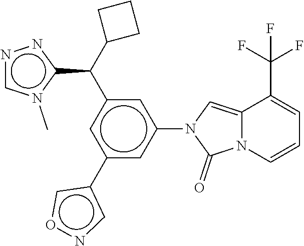 |
| 455 | 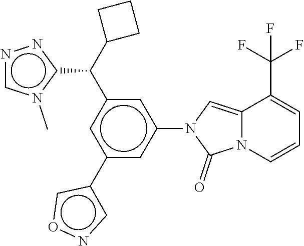 |
| 456 | 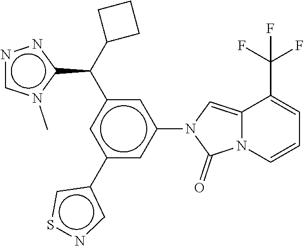 |
| 457 | 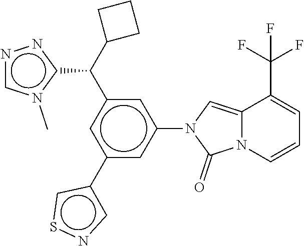 |
| 458 | 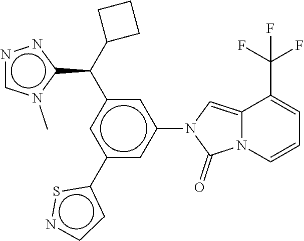 |
| 459 | 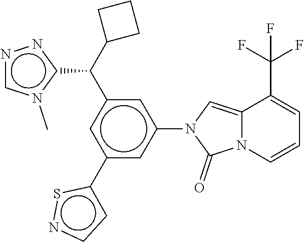 |
| 460 | 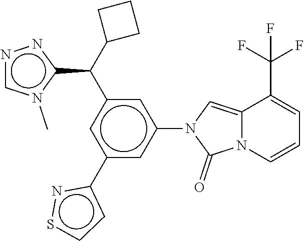 |
| 461 | 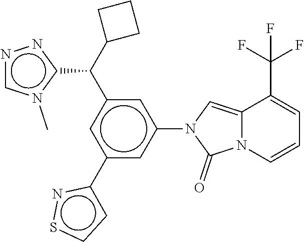 |
| 462 | 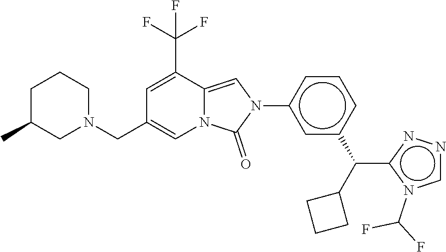 |
| 463 | 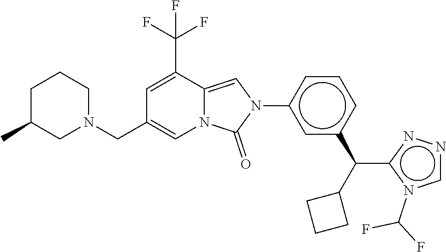 |
| 464 | 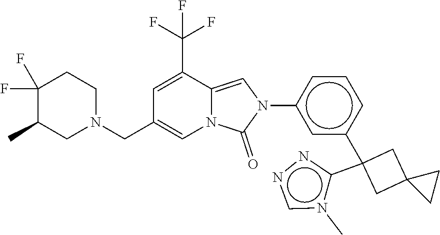 |
| 465 | 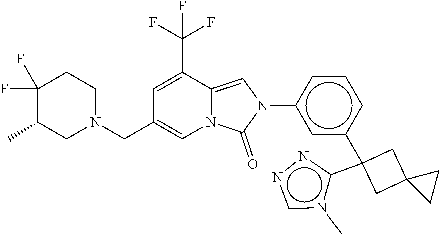 |
| 466 | 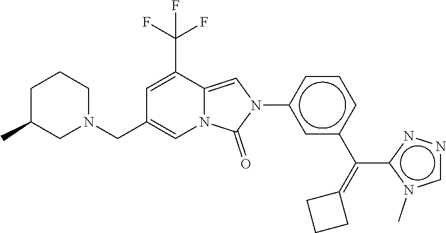 |
| 467 | 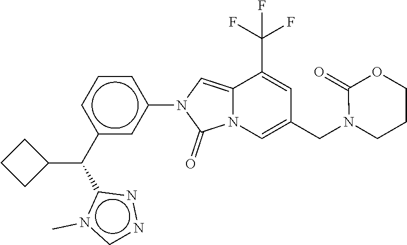 |
| 468 | 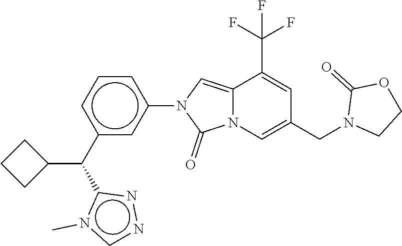 |
| 469 | 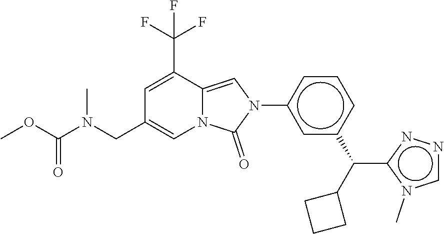 |
| 470_P1 | 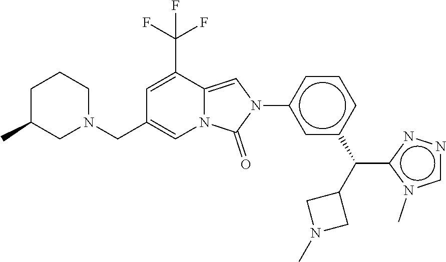 |
| 470_P2 | 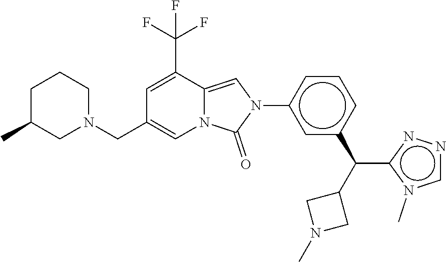 |
| 471 | 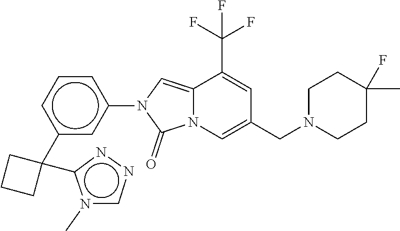 |
| 472_P1 | 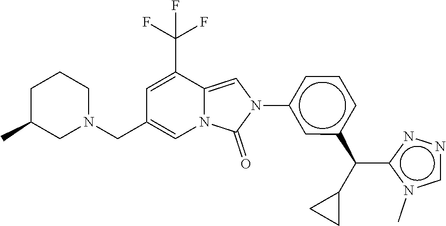 |
| 472_P2 | 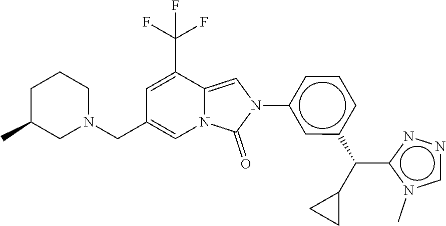 |
| 473 | 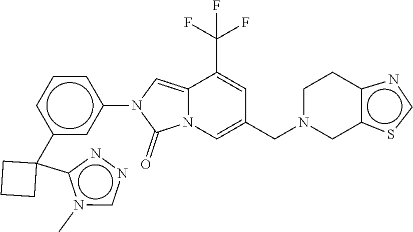 |
| 474 | 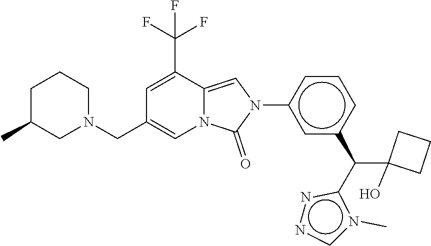 |
| 475 | 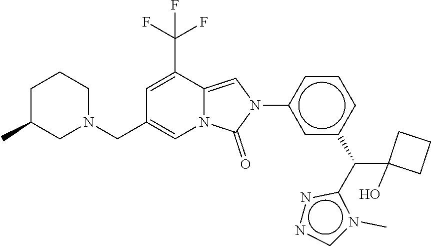 |
| 476 | 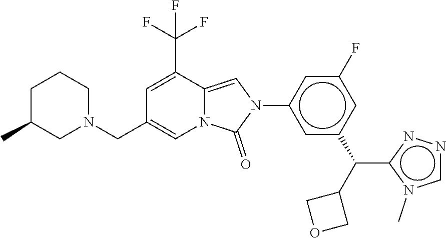 |
| 477 | 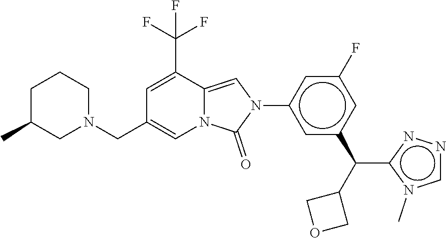 |
| 478 | 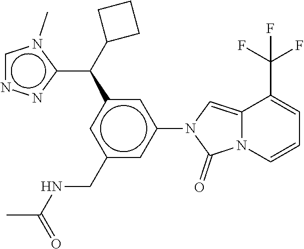 |
| 479 | 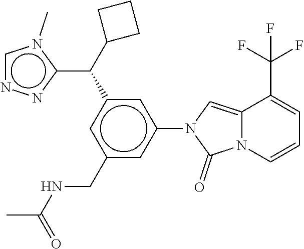 |
| 480 | 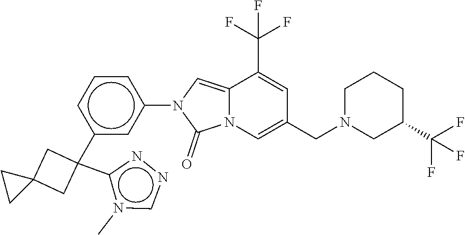 |
| 481 | 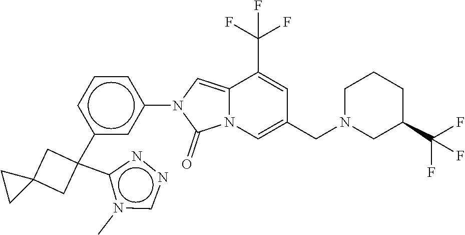 |
| 482 | 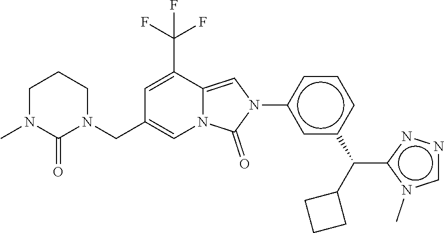 |
| 483 | 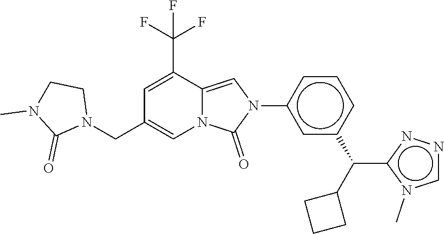 |
| 484 | 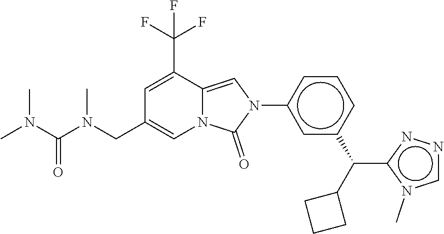 |
| 485 | 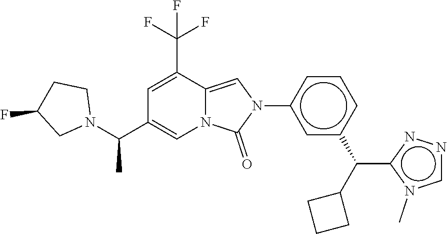 |
| 486 | 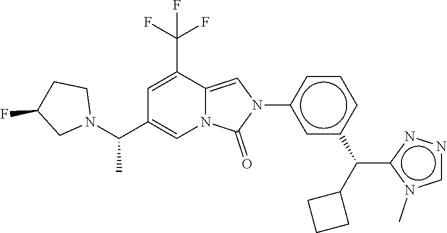 |
| 487 | 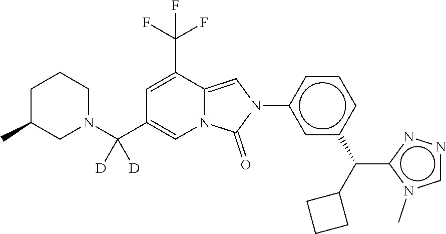 |
| 488 | 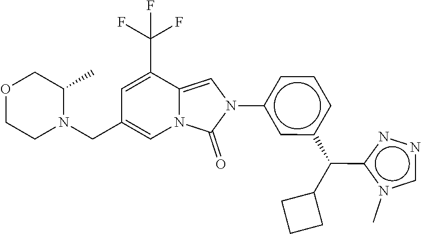 |
| 489 | 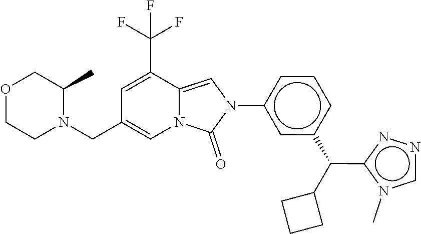 |
| 490 | 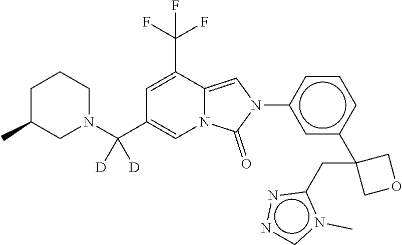 |
| 491 | 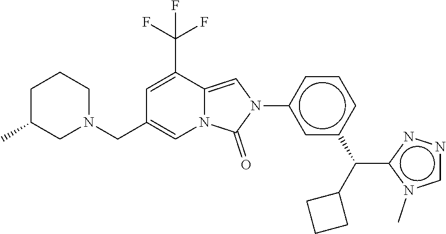 |
| 492 | 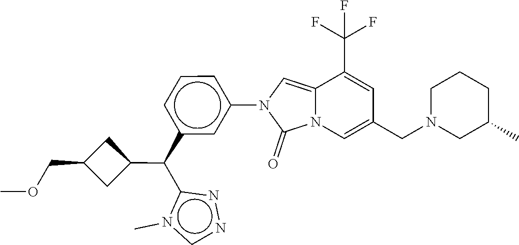 |
| 493 | 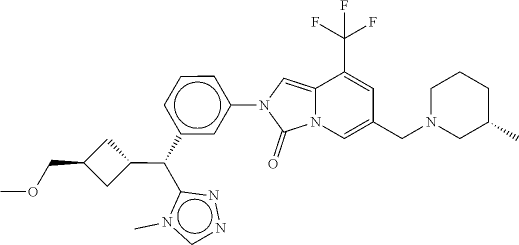 |
| 494 | 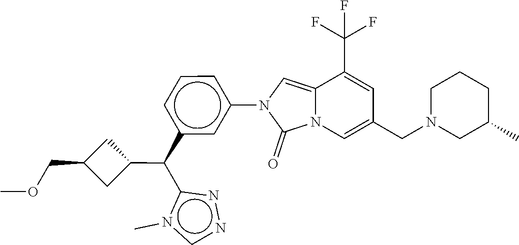 |
| 495 | 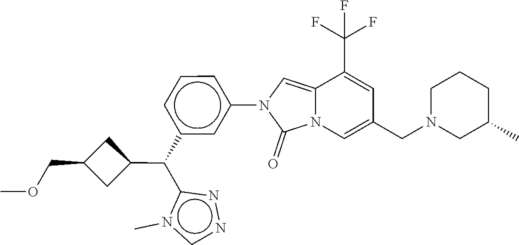 |
| 496 | 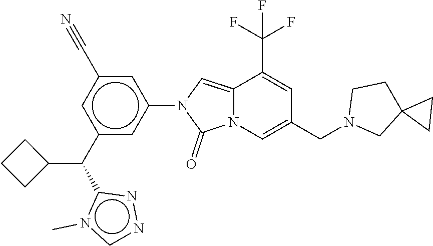 |
| 497 | 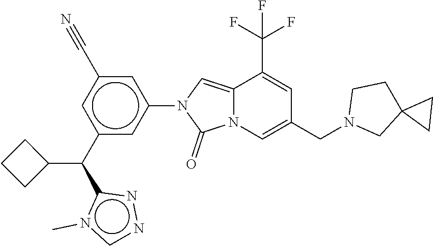 |
| 498 | 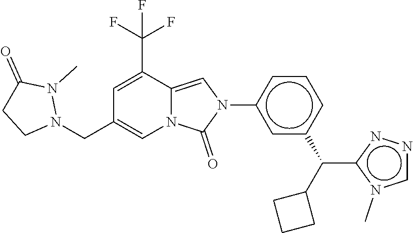 |
| 499 | 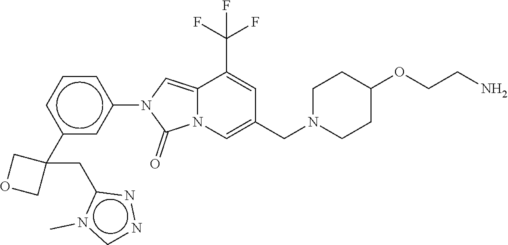 |
| 500 | 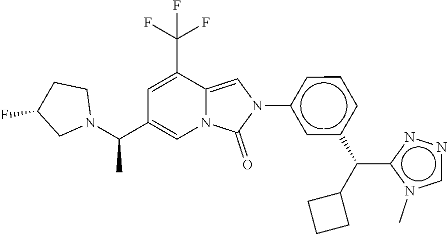 |
| 501 | 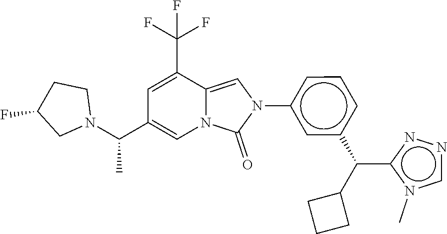 |
| 502 | 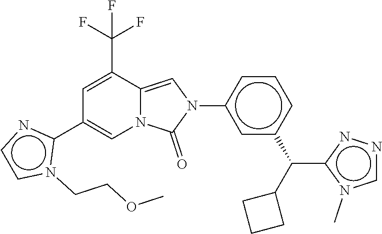 |
| 503 | 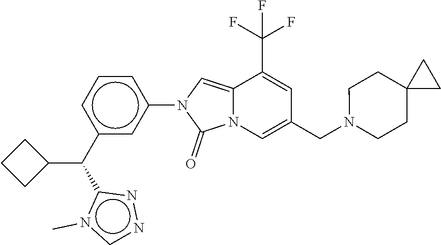 |
| 504 | 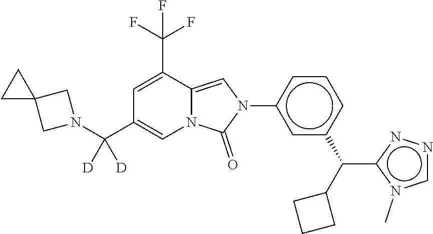 |
| 505 | 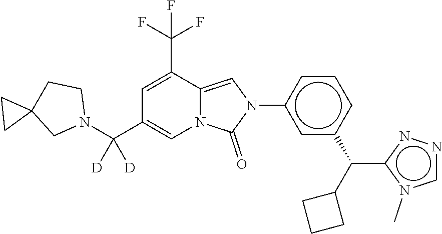 |
| 506_P1 | 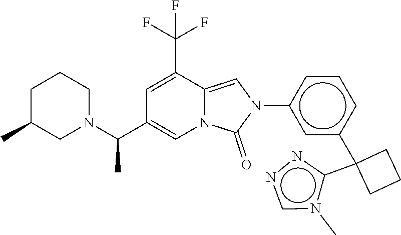 |
| 506_P2 | 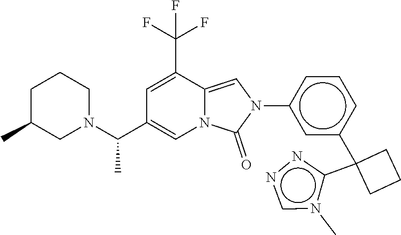 |
| 507 | 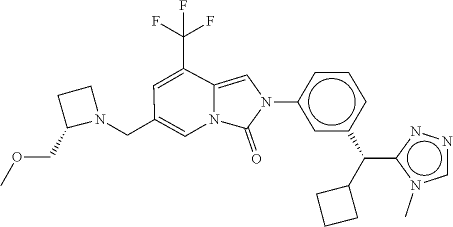 |
| 508 | 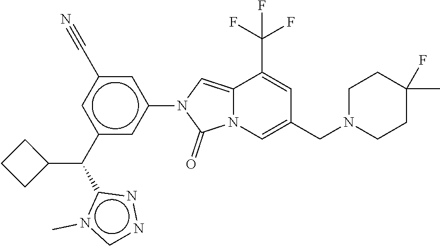 |
| 509 | 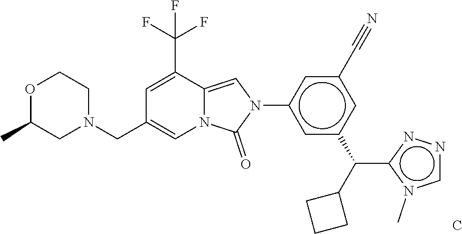 |
| 510 | 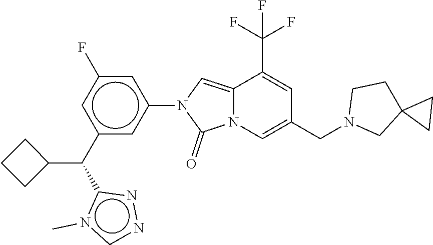 |
| 511 | 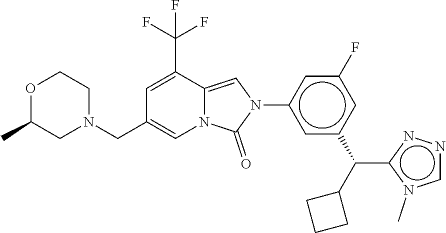 |
| 512 | 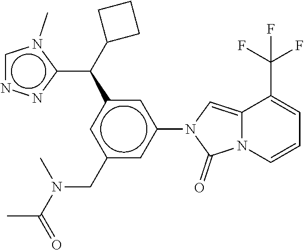 |
| 513 | 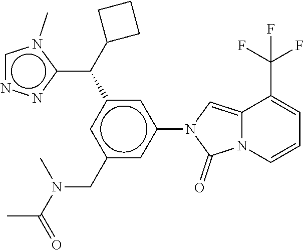 |
| 514 | 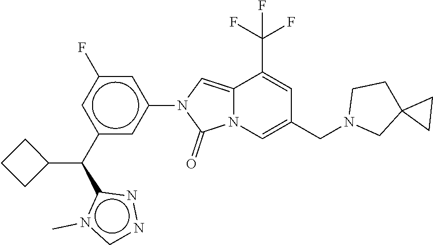 |
| 515 | 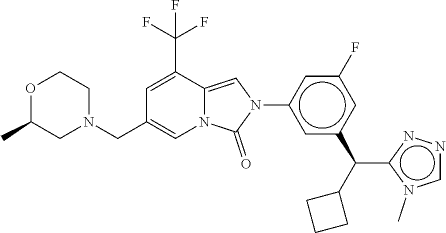 |
| 516 | 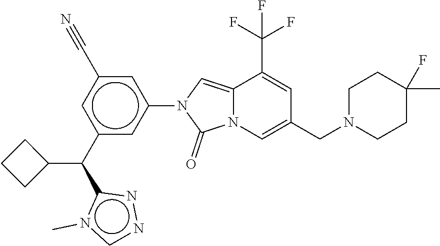 |
| 517 | 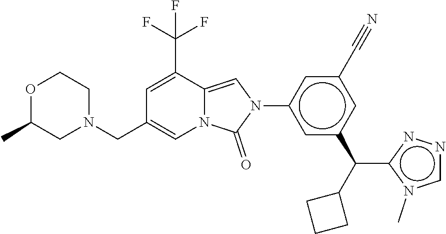 |
| 518 | 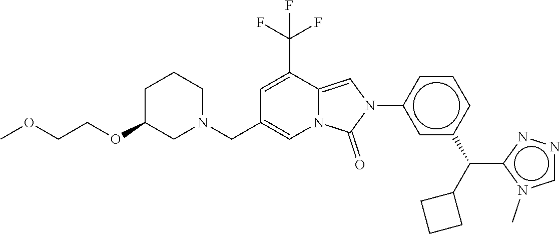 |
| 519 | 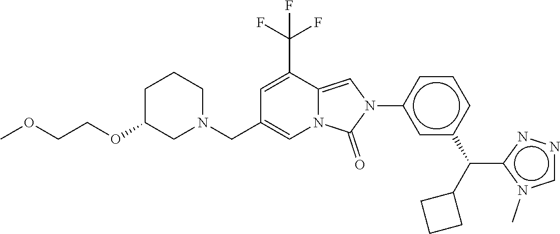 |
| 520 | 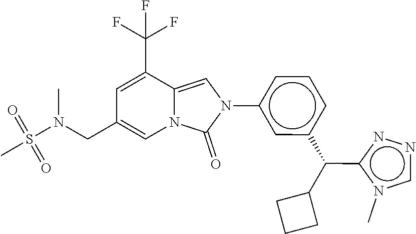 |
| 521 | 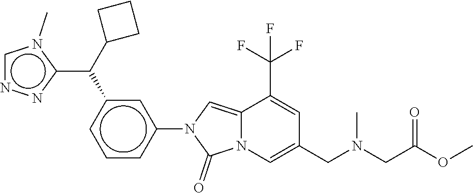 |
| 522 | 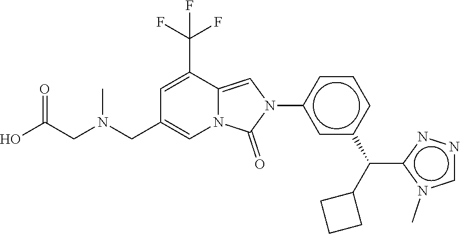 |
| 523 | 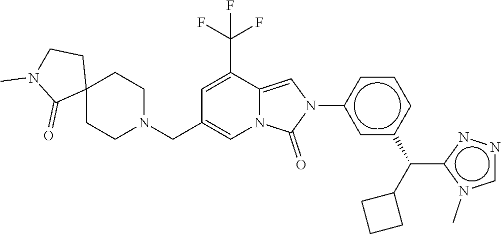 |
| 524 | 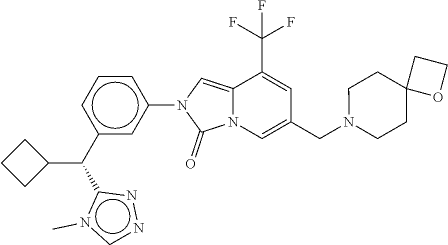 |
| 525 | 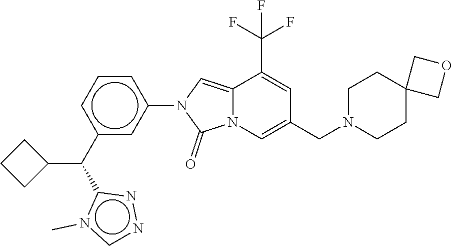 |
| 526 | 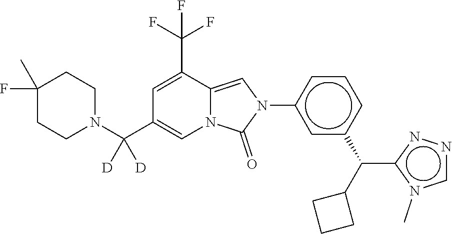 |
| 527 | 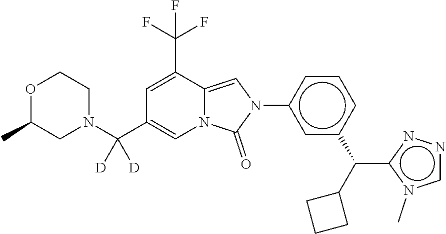 |
| 528 | 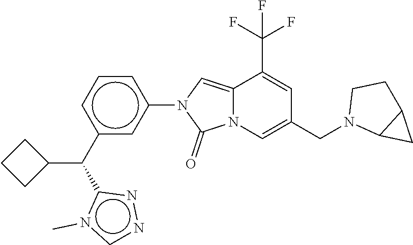 |
| 529 | 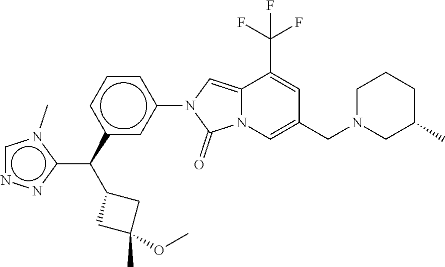 |
| 530 | 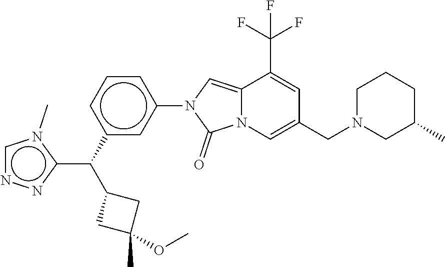 |
| 531 | 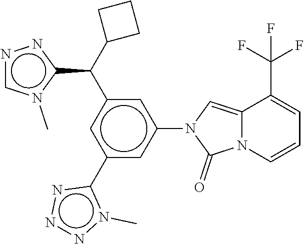 |
| 532 | 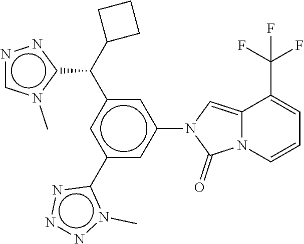 |
| 533 | 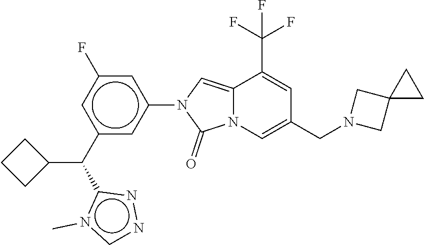 |
| 534 | 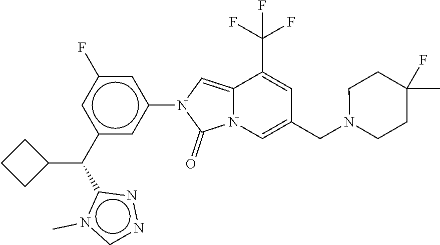 |
| 535 | 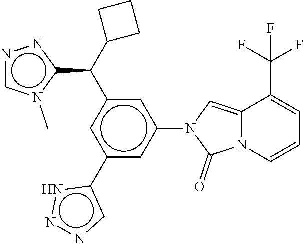 |
| 536 | 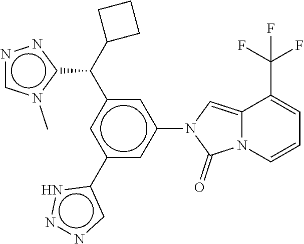 |
| 537 | 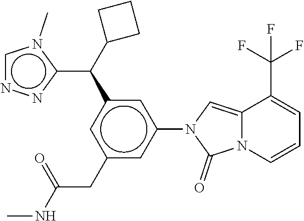 |
| 538 | 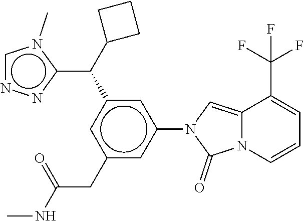 |
| 539 | 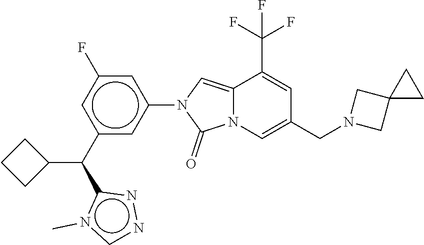 |
| 540 | 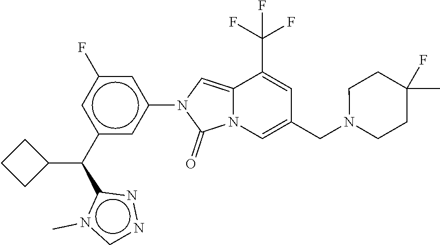 |
| 541 | 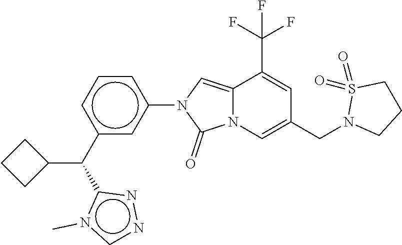 |
| 542 | 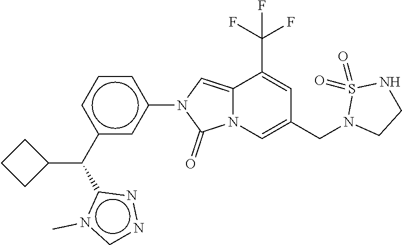 |
| 543 | 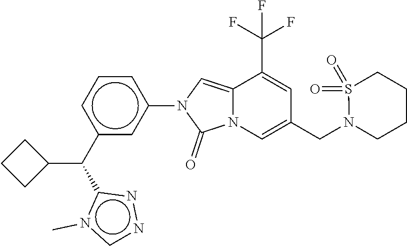 |
| 544 | 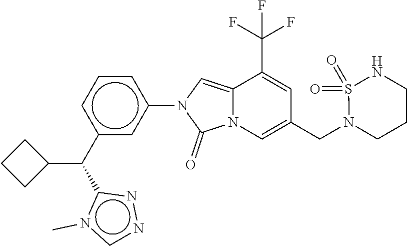 |
| 545 | 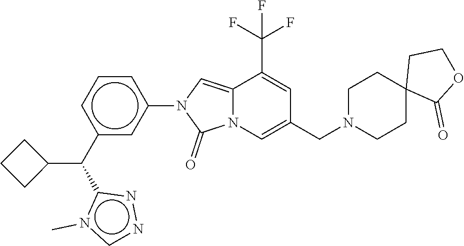 |
| 546 | 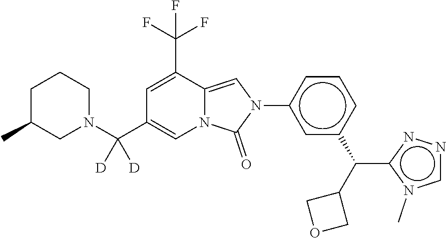 |
| 547 | 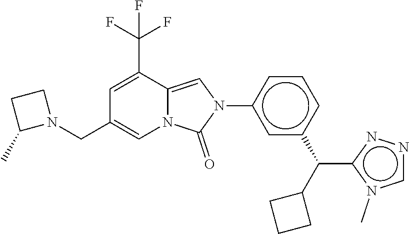 |
| 548 | 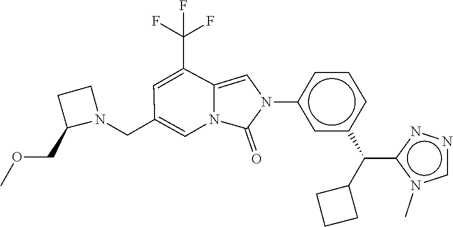 |
| 549 | 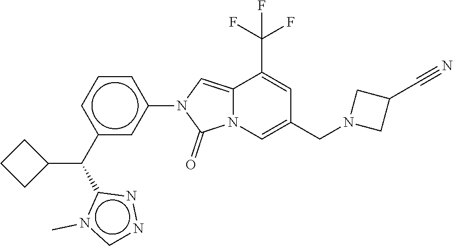 |
| 550_P1 | 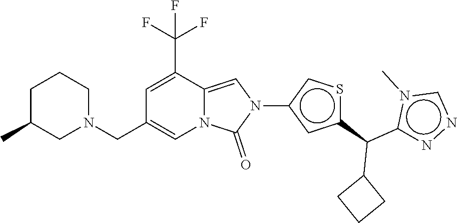 |
| 550_P2 | 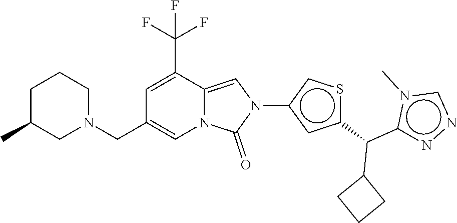 |
| 551 | 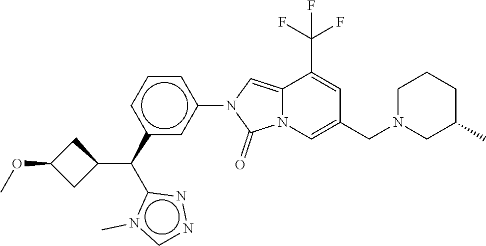 |
| 552 | 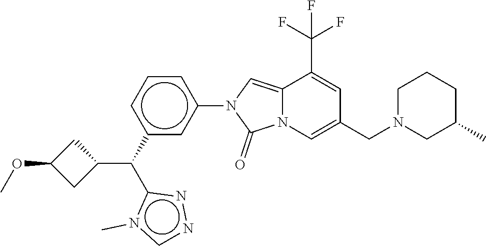 |
| 553 | 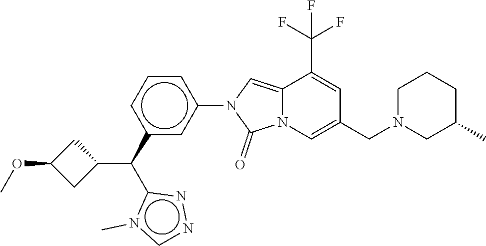 |
| 554 | 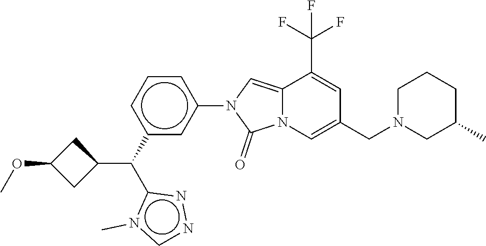 |
| 555 | 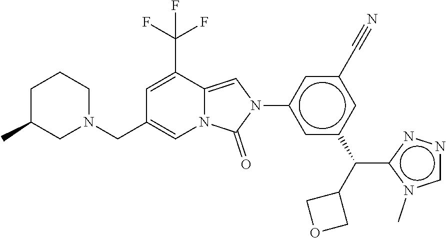 |
| 556 | 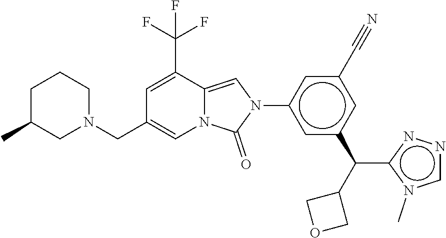 |
| 557 | 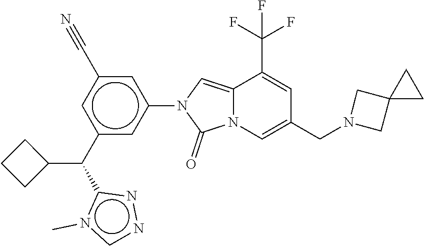 |
| 558 | 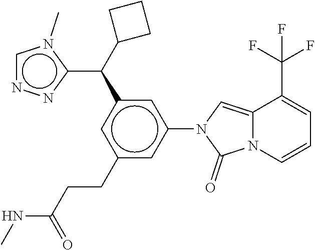 |
| 559 | 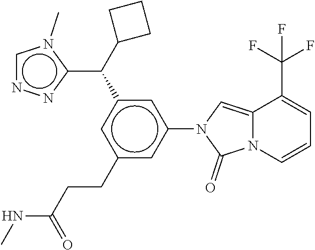 |
| 560 | 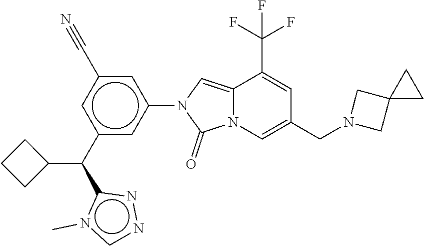 |
| 561 | 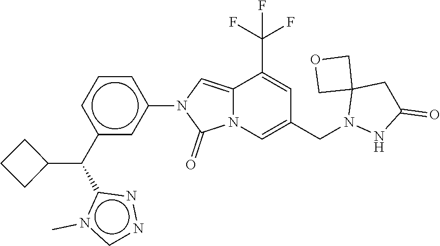 |
| 562 | 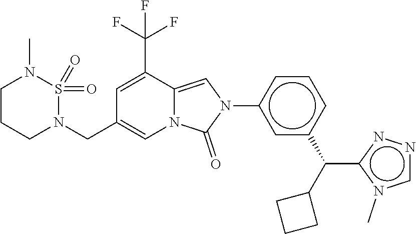 |
| 563 | 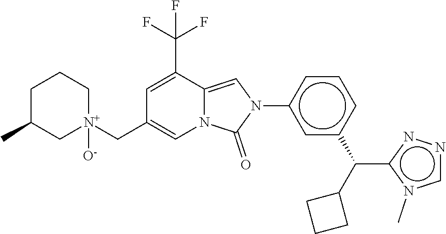 |
| 564 | 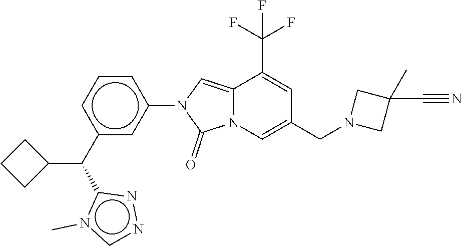 |
| 565_P1 | 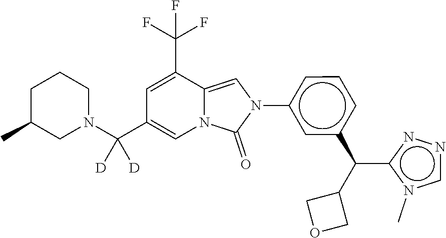 |
| 565_P2 | 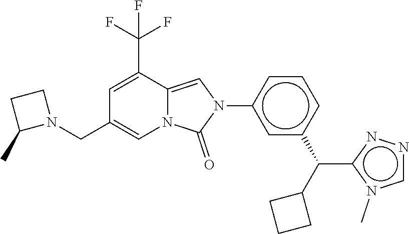 |
| 566 | 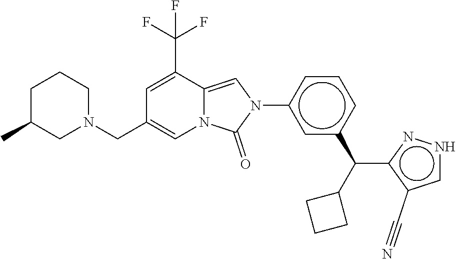 |
| 567 | 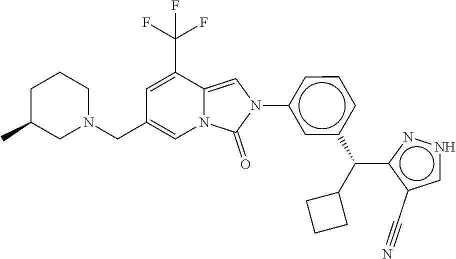 |
| 568 | 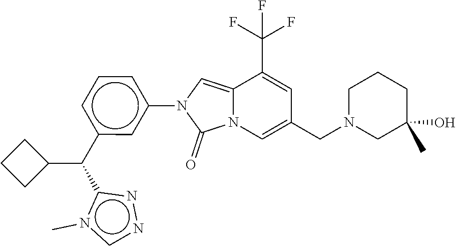 |
| 569 | 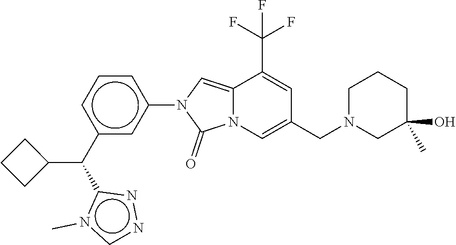 |
| 570 | 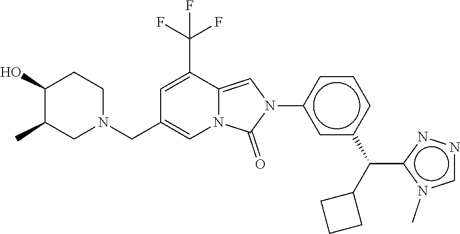 |
| 571 | 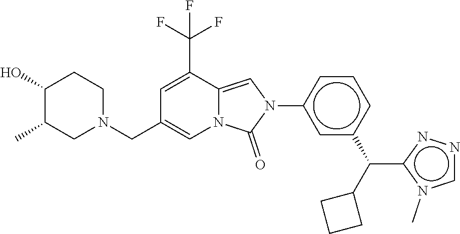 |
| 572 | 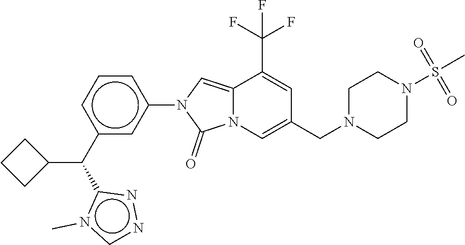 |
| 573_P1 | 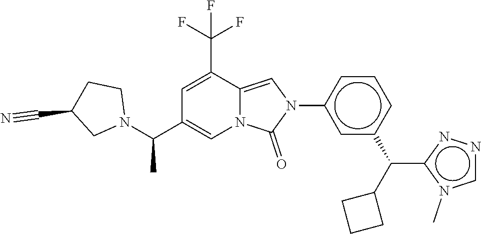 |
| 573_P2 | 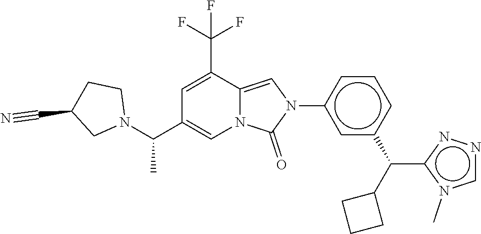 |
| 574 | 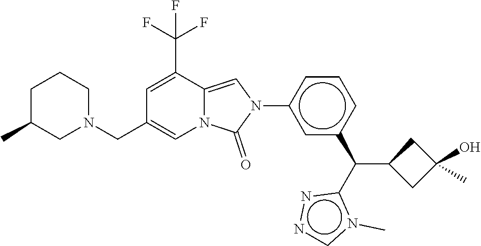 |
| 575 | 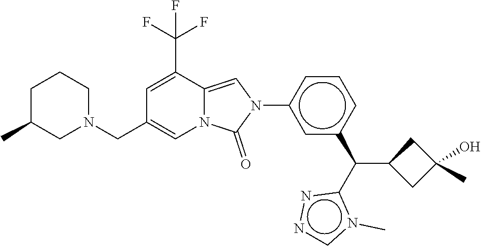 |
| 576 | 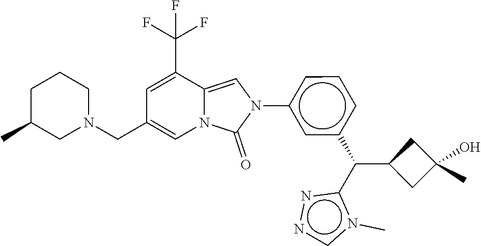 |
| 577 | 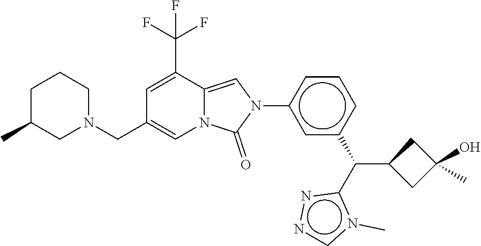 |
| 578 | 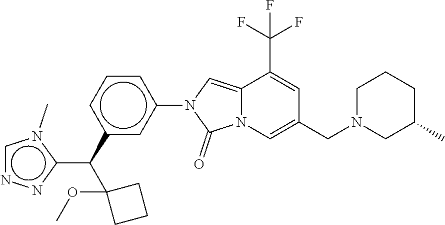 |
| 579 | 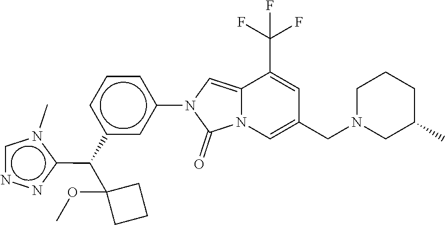 |
| 580 | 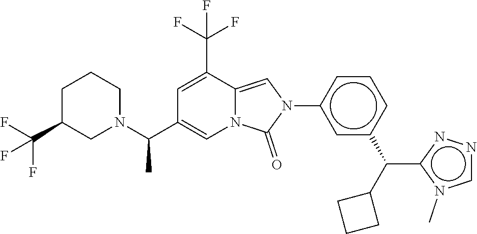 |
| 581 | 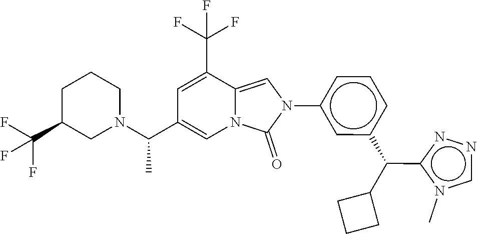 |
| 582 | 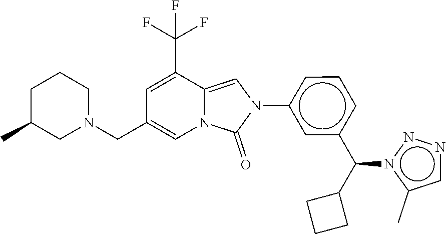 |
| 583 | 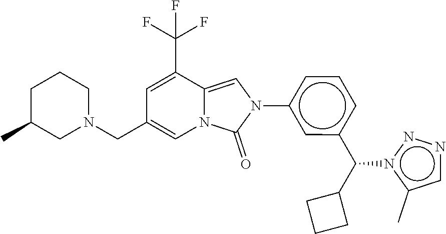 |
| 584 | 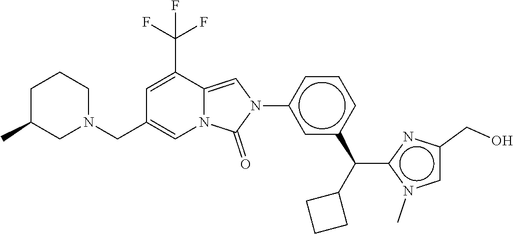 |
| 585 | 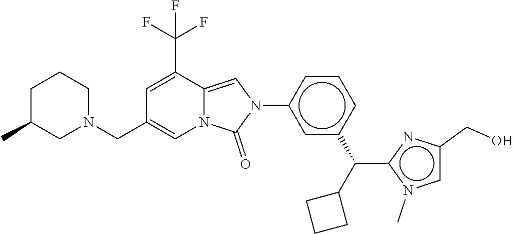 |
| 586 | 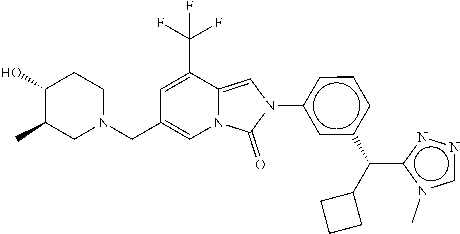 |
| 587 | 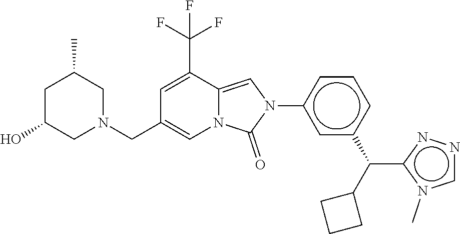 |
| 588 | 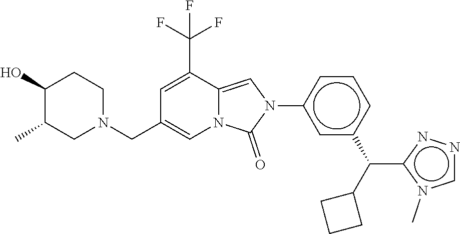 |
| 589 | 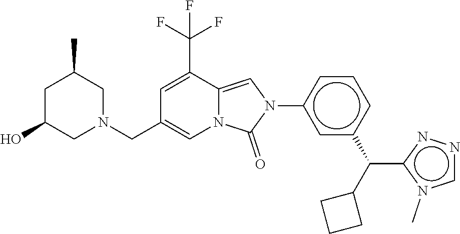 |
| 590 | 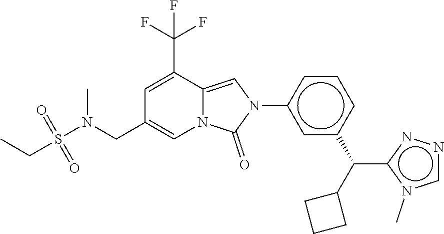 |
| 591 | 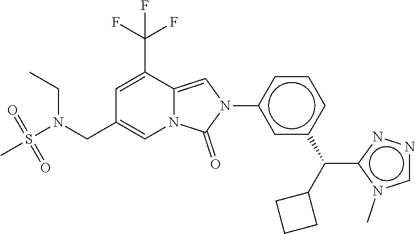 |
| 592 | 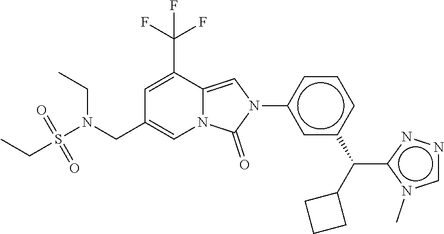 |
| 593 | 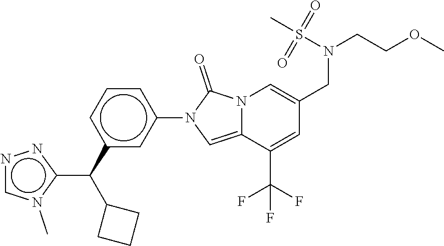 |
| 594 | 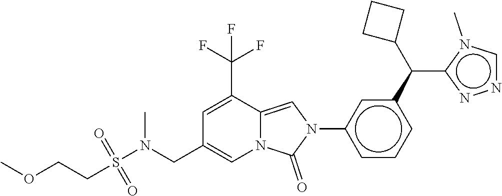 |
| 595 | 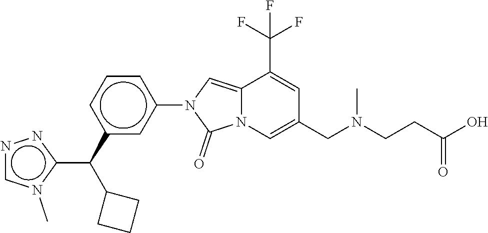 |
| 596 | 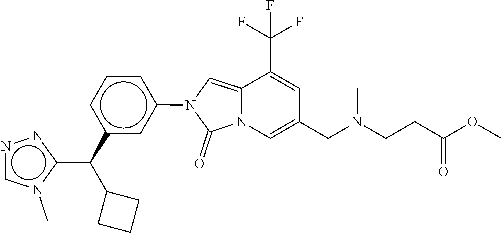 |
| 597_P1 | 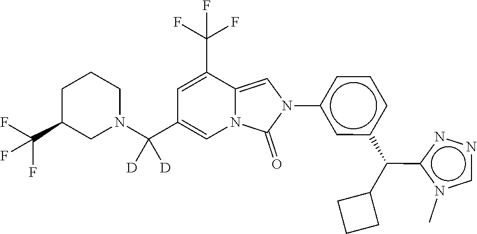 |
| 597_P2 | 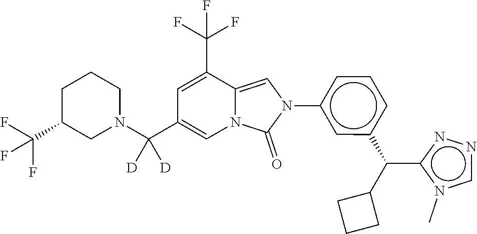 |
| 598 | 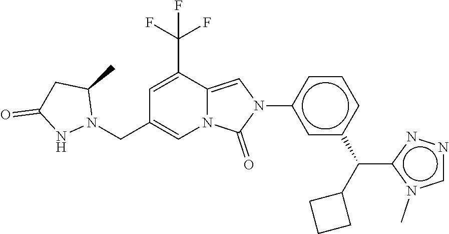 |
| 599 | 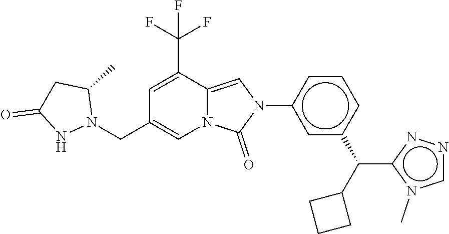 |
| 600 | 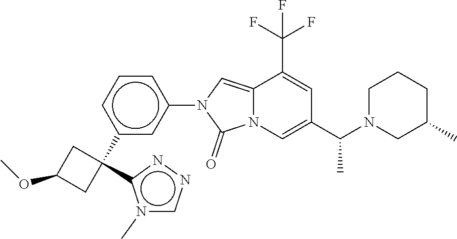 |
| 601 | 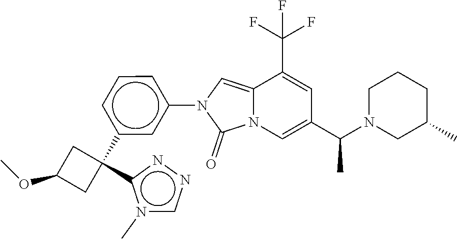 |
| 602 | 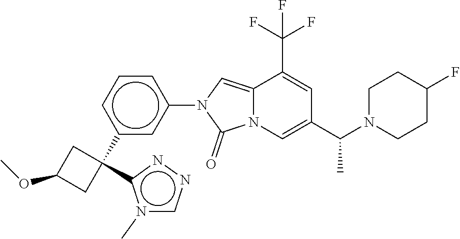 |
| 603 | 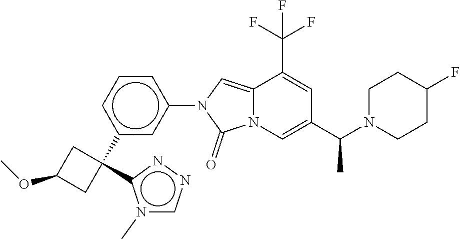 |
| 604 | 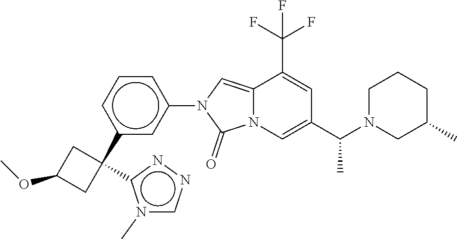 |
| 605 | 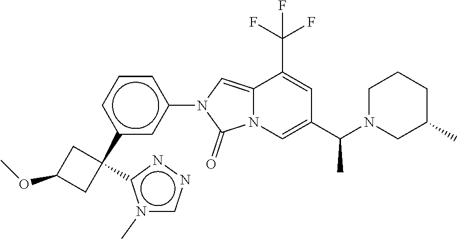 |
| 606 | 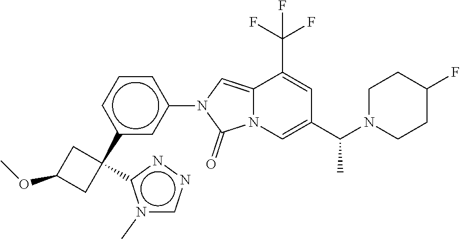 |
| 607 | 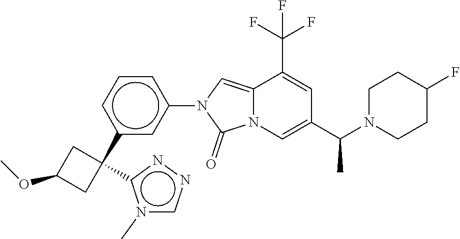 |
| 608 | 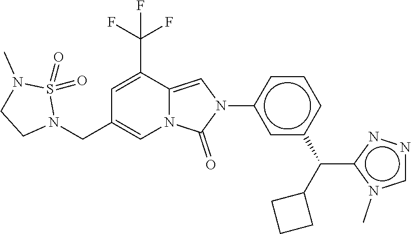 |
| 609 | 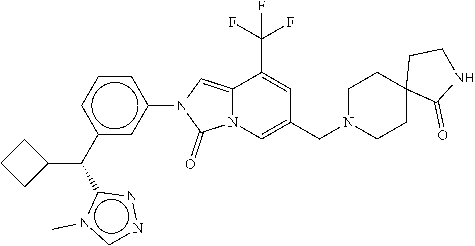 |
| 610_P1 | 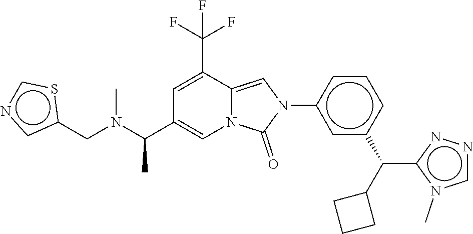 |
| 610_P2 | 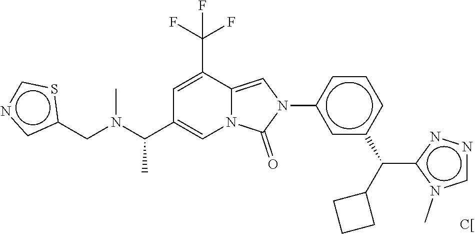 |
| 611 | 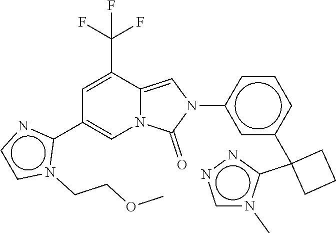 |
| 612_P1 | 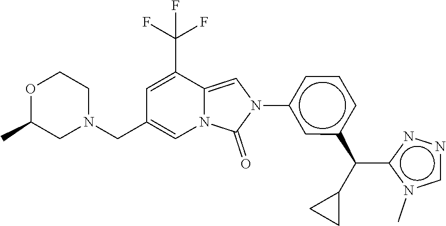 |
| 612_P2 | 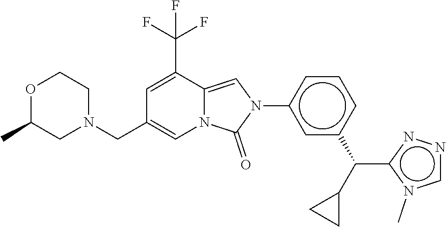 |
| 613 | 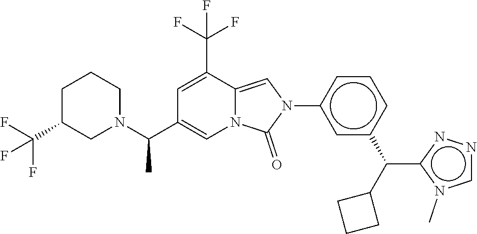 |
| 614 | 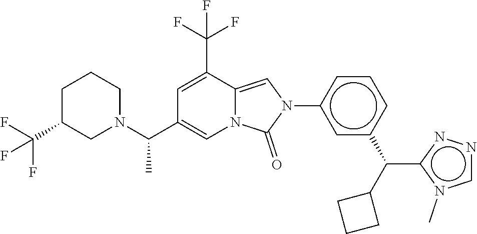 |
| 615 | 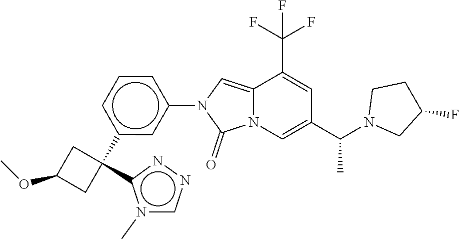 |
| 616 | 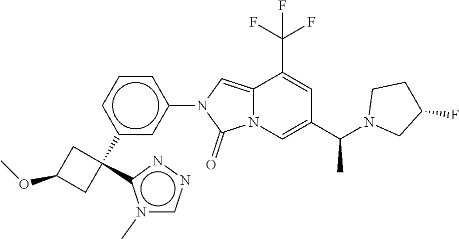 |
| 617 | 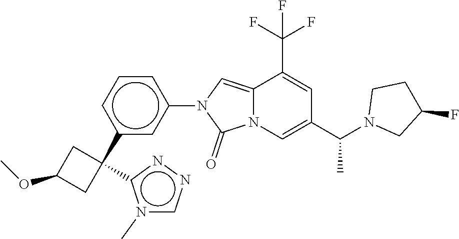 |
| 618 | 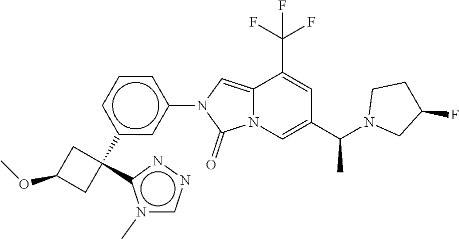 |
| 619 | 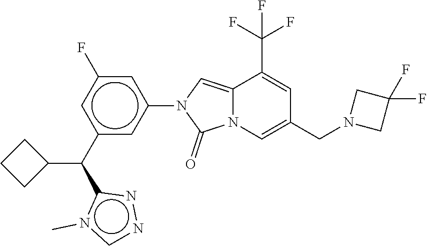 |
| 620 | 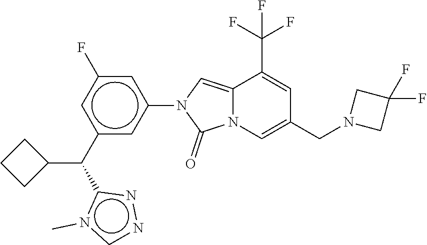 |
| 621 | 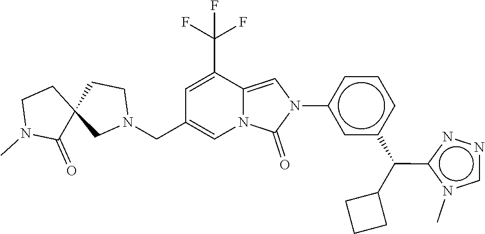 |
| 622 | 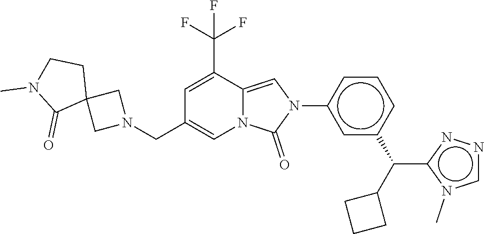 |
| 623 | 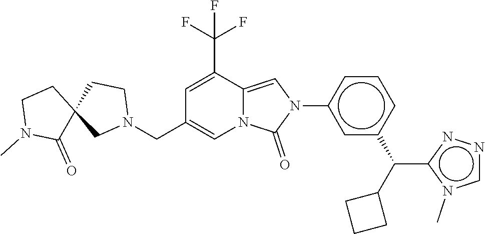 |
| 624 | 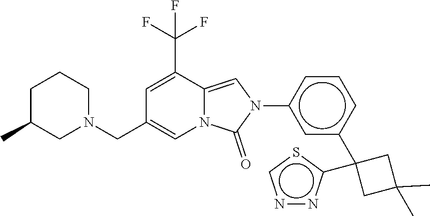 |
| 625 | 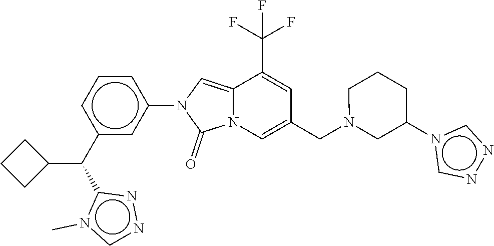 |
| 626 | 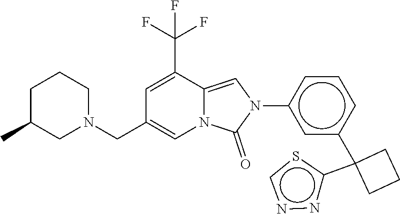 |
| 627_P1 | 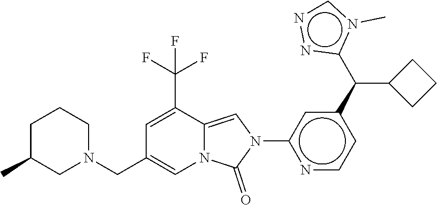 |
| 627_P2 | 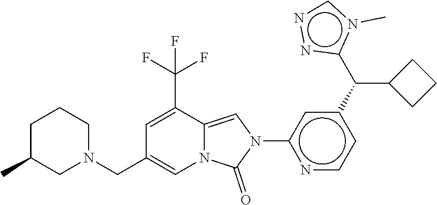 |
| 628_P1 | 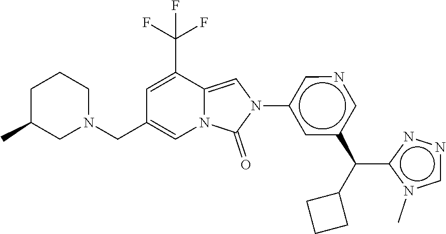 |
| 628_P2 | 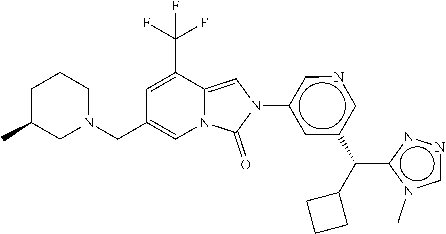 |

or a pharmaceutically acceptable salt thereof

A person of skill in the art will understand the present disclosure includes compounds with the stereochemistry which are the opposite of how they have been drawn. Additionally, the present disclosure contemplates tautomers of the compounds as drawn herein.

The present disclosure includes the racemate of any compound disclosed herein.

### Additional Therapeutic Agent

In some embodiments, an additional therapeutic agent is a chemotherapeutic agent. In some embodiments, an additional therapeutic agent is selected from the group consisting of pemetrexed, carboplatin, paclitaxel/nab-paclitaxel, cisplatin, 5-fluorouracil, trastuzumab, capecitabine, oxaliplatin, leucovorin, platinum, bevacizumab, and etoposide.

In some embodiments, an additional therapeutic agent is radiotherapy. In some embodiments, an additional therapeutic agent is SBRT or Concurrent chemoradiation.

In some embodiments, an additional therapeutic agent is an angiogenesis inhibitor. In some embodiments, an additional therapeutic agent is selected from the group consisting of axitinib, Lenvatinib, bevacizumab, cabozantinib, anlotinib, IBI305, and apatinib.

In some embodiments, an additional therapeutic agent is checkpoint inhibitor. In some embodiments, an additional therapeutic agent is selected from the group consisting of Ipilimumab, Tremelimumab, LY3321367, Sabatolimab, Relatlimab, COM701, Tiragolumab, PF-05082566, APX005M, KY1044, and GWN323.

In some embodiments, an additional therapeutic agent is a targeted therapy. In some embodiments, an additional therapeutic agent is selected from the group consisting of erlotinib, osimertinib, crizotinib, alectinib, crizotinib, pamiparib, niraparib, olaparib, cobimetinib, AMG 510, MK-8353, dabrafenib, trametinib, encorafenib, cetuximab, copanlisib, ipatasertib, pemigatinib, B-701, savolitinib, abemaciclib, and TNO155.

In some embodiments, an additional therapeutic agent is an anti-body drug conjugate. In some embodiments, an additional therapeutic agent is selected from the group consisting of trastuzumab emtansine (T-DM1), trastuzumab deruxtecan (T-DXd), enfortumab veotin (EV), and sacituzumab govitecan.

In some embodiments, an additional therapeutic agent is an antibody product. In some embodiments, an additional therapeutic agent is selected from the group consisting of

| Antibody Product | Antibody Product |
|---|---|
| TGF-β × PD-L1 | M7824 |
| | YM101 |
| | SHR-1701 |
| CTLA-4 × PD-L1 | KN046 |
| CTLA-4 × PD-1 | MGD019 |
| | MEDI5752 |
| LAG-3 × PD-L1 | IBI323 |
| LAG-3 × PD-1 | Tebotelimab |
| TIM-3 × PD-L1 | LY3415244 |
| TIGIT × PD-L1 | |
| 4-1BB × PD-L1 | MCLA-145 |
| | ABL503 |
| | PM1003 |
| CD27 × PD-L1 | CDX-527 |
| c-Met × PD-1 | |
| EGFR × PD-L1 | |
| PD-1 × PD-L1 | LY3434172 |
| CD47 × PD-L1 | IBI322 |

In some embodiments, an additional therapeutic agent is chimeric antigen receptor T cells (CAR-T). In some embodiments, CAR-T cells are selected from the group consisting of CD19/C19CAR-28-zeta T cells, GD2/iC9-GD2-CD28-OX40 (iC9-GD2) T cells, anti-CD19 CAR T cells, autologous anti-C19CAR-4-1BB-CD3ζ-EGFRt-expressing CD4⁺/CD8⁺ central memory T lymphocytes CAR014 T cells, CD19/KTE-C19 T cells, JCAR017 T cells, and CART-EGFRvII T cells.

In some embodiments, an additional therapeutic agent is an oncolytic virus. In some embodiments, an additional therapeutic agent is herpes virus (e.g., HSV1716, G47Δ-mIL-12), adenovirus (e.g., hTertAd, ISF35, D24-RGDOX, ADV-TK, rHu-hDCT, ChAdOx I-STEAP I, ChAdOx I-h5T4), vaccinia virus (e.g., WR-mAb I), and myxoma virus (e.g., vPD1).

In some embodiments, methods disclosed herein comprise administering two or more additional therapeutic agents to a subject in need thereof. In some embodiments, two or more additional therapeutic agents are selected from the group consisting of:

Pemetrexed and carboplatin
Paclitaxel/nab-paclitaxel and carboplatin
Paclitaxel; nab-paclitaxel;
gemcitabine and carboplatin
Cisplatin and 5-fluorouracil
Trastuzumab plus either 5-fluorouracil plus cisplatin
or capecitabine plus oxaliplatin
5-fluorouracil and leucovorin plus oxaliplatin;
capecitabine and oxaliplatin
Pemetrexed and platinum
Gemcitabine and platinum
Carboplatin and pemetrexed
Gemcitabine and cisplatin
Platinum and pemetrexed
Bevacizumab plus paclitaxel and carboplatin
Carboplatin and etoposide
Nab-paclitaxel
Carboplatin and nab-paclitaxel
Etoposide and carboplatin/cisplatin

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and tert-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)_{0- 4}C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and =S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{∘}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}2, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein.

In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

Unless specifically stated or otherwise apparent from context, as used herein the term “about” is understood as within a range of normal tolerance in the art, for example within 2 standard deviations of the mean. About can be understood as within ±10% of the stated value.

Unless otherwise clear from context, all numerical values provided herein are modified by the term about.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Diseases and Disorders

Provided herein are methods for modulating activity of an immune cell (e.g., a T-cell, a B-cell, or a NK-cell) such as by contacting the immune cell with an effective amount of a Cbl-b inhibitor described herein or a composition thereof. Also provided are in vitro methods of producing said immune cells with modulated activity, referred to herein as “modified immune cells,” wherein said modified immune cells can be administered to an individual in need thereof (e.g., an individual having cancer) by ex vivo methods. Further provided are in vivo methods of modulating a response in an individual in need thereof (e.g., an individual with cancer), wherein the method comprises administration of an effective amount of a Cbl-b inhibitor described herein or a composition thereof. Moreover, the present disclosure provides in vitro methods of producing an expanded population of lymphocytes after in vivo lympho-conditioning in an individual, wherein the lympho-conditioning occurs as a result of administration of an effective amount of a Cbl-b inhibitor described herein or a composition thereof to the individual. In addition, the expanded population of lymphocytes can then be administered to the individual with cancer. In some embodiments, the modified immune cells or the expanded population of lymphocytes are produced from a biological sample comprising immune cells obtained from the individual, such as a blood sample comprising peripheral blood mononuclear cells or a tumor biopsy comprising tumor infiltrating lymphocytes (TILs).

Additionally, provided are Cbl-b inhibitors for use as therapeutic active substances. A Cbl-b inhibitor for use in treating or preventing a disease or condition associated with Cbl-b activity is provided. Also, a Cbl-b inhibitor for use in treating cancer is provided. Further provided is the use of a Cbl-b inhibitor in the manufacture of a

medicament for treating or preventing a disease or condition associated with Cbl-b activity.

Also provided is the use of a Cbl-b inhibitor in the manufacture of a medicament for treating cancer. Moreover, the present disclosure provides treatment methods, medicaments, and uses comprising a Cbl-b inhibitor as part of a combination therapy for treating cancer involving one or more of an immune checkpoint inhibitor, an antineoplastic agent, and radiation therapy.

In some embodiments of the treatment methods, medicaments, and uses of the present disclosure, the cancer is a hematologic cancer such as lymphoma, a leukemia, or a myeloma. In other embodiments of the treatment methods, medicaments, and uses of the present disclosure, the cancer is a non-hematologic cancer such as a sarcoma, a carcinoma, or a melanoma.

Hematologic cancers include, but are not limited to, one or more leukemias such as B-cell acute lymphoid leukemia (“BALL”), T-cell acute lymphoid leukemia (“TALL”), acute lymphoid leukemia (ALL); one or more chronic leukemias including, but not limited to, chronic myelogenous leukemia (CML) and chronic lymphocytic leukemia (CLL); additional hematologic cancers or hematologic conditions including, but not limited to, B-cell prolymphocytic leukemia, blastic plasmacytoid dendritic cell neoplasm, Burkitt's lymphoma, diffuse large B-cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and“preleukemia,” which are a diverse collection of hematological conditions united by ineffective production (or dysplasia) of myeloid blood cells.

Non-hematologic cancers include but are not limited to, a neuroblastoma, renal cell carcinoma, colon cancer, colorectal cancer, breast cancer, epithelial squamous cell cancer, melanoma, stomach cancer, brain cancer, lung cancer (e.g., NSCLC), pancreatic cancer, cervical cancer, ovarian cancer, liver cancer, bladder cancer, prostate cancer, testicular cancer, thyroid cancer, uterine cancer, adrenal cancer, and head and neck cancer.

In some aspects, the effectiveness of administration of a Cbl-b inhibitor in the treatment of a disease or disorder such as cancer is measured by assessing clinical outcome, such as reduction in tumor size or number of tumors, and/or survival. In some embodiments, “treating cancer” comprises assessing a patient's response to the treatment regimen according to the Response Evaluation Criteria in Solid Tumors (RECIST version 1.1) as described (see, e.g., Eisenhauer et al., Eur J Cancer, 45:228-247, 2009; and Nishino et al., Am J Roentgenol, 195: 281-289, 2010). Response criteria to determine objective anti-tumor responses per RECIST 1.1 include: complete response (CR); partial response (PR); progressive disease (PD); and stable disease (SD).

In some embodiments, a disease or disorder is selected from the group consisting of non-squamous NSCLC, squamous NSCLC, triple negative breast cancer (TNBC), esophageal cancer, gastroesophageal junction adenocarcinoma, and HER+ gastric adenocarcinoma. In some embodiments, a disease or disorder is selected from the group consisting of a solid tumor, NSCLC, head and neck squamous cell carcinoma (HNSCC), and SCLC. In some embodiments, a disease or disorder is selected from the group consisting of renal cell cancer (RCC), a solid tumor, endometrial cancer, non-squamous NSCLC, hepatocellular carcinoma (HCC), gastric cancer, SCLC, cervical cancer, TNBC, ostreosarcoma, kidney cancer, and melanoma. In some embodiments, a disease or disorder is selected from the group consisting of urothelial cancer, colorectal cancer, HNSCC, NSCLC, gastric or GEJ adenocarcinoma, germ cell tumors, mesothelioma, HCC, pancreatic ductal adenocarcinoma, melanoma, RCC, a solid tumor, and lymphoma. In some embodiments, a disease or disorder is NSCLC, a solid tumor (e.g. KRAS p.G12C mutant solid tumor), TNBC, ovarian cancer, colorectal cancer, melanoma, breast cancer, and lymphoma. In some embodiments, a disease or disorder is selected from the group consisting of HER2+ metastatic breast cancer, urothelial cancer, and triple-negative metastatic breast cancer. In some embodiments, a disease or disorder is selected from the group consisting of B cell lymphoma, chronic lymphocytic, leukemia, acute lymphocytic leukemia, neurobastoma, CD19⁺ diffuse large B cell lymphoma, follicular lymphoma, mantle cell lymphoma, glioblastoma, and non-Hodgkin lymphoma. In some embodiments, a disease or disorder is selected from the group consisting of rhabdomyosarcoma, glioblastoma, melanoma brain metastases, lung adenocarcinoma, melanoma, and prostate cancer.

### Formulations

The present disclosure also provides pharmaceutical compositions comprising a compound disclosed herein, or a pharmaceutically acceptable salt and solvate thereof, and at least one pharmaceutically acceptable carrier, diluent, excipient and/or adjuvant.

The present disclosure also provides a medicament comprising at least one compound disclosed herein, or a pharmaceutically acceptable salt and solvate thereof, as active ingredient.

Generally, for pharmaceutical use, a compound disclosed herein may be formulated as a pharmaceutical preparation comprising at least one compound disclosed and at least one pharmaceutically acceptable carrier, diluent, excipient and/or adjuvant, and optionally one or more further pharmaceutically active compounds. Details regarding the presence of further pharmaceutically active compounds are provided hereafter.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. Formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. Compositions may also be formulated so as to provide rapid, sustained or delayed release of the active compound(s) contained therein.

Pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, a compound disclosed may be administered as a single daily dose, divided over one or more daily doses, or essentially continuously, e.g. using a drip infusion.

Another object of this disclosure is the use of the combination as a medicament, i.e. for medical use. Thus, in one embodiment, the disclosure provides the use of the combination of the disclosure for the manufacturing of a medicament. Especially, the disclosure provides the use of the combined pharmaceutical composition of the disclosure or the kit of the disclosure for the manufacturing of a medicament.

### Dosing

In some embodiments, a CBL-B inhibitor is administered at a dose of 60-600 mg. In some embodiments, a CBL-B inhibitor is administered at a dose of 100-500 mg. In some embodiments, a CBL-B inhibitor is administered at a dose of 125-475 mg. In some embodiments, a CBL-B inhibitor is administered at a dose of 150-450 mg. In some embodiments, a CBL-B inhibitor is administered at a dose of 200-400 mg. In some embodiments, a CBL-B inhibitor is administered at a dose of about 60 mg. In some embodiments, a CBL-B inhibitor is administered at a dose of about 80 mg. In some embodiments, a CBL-B inhibitor is administered at a dose of about 100 mg.

In some embodiments, a CBL-B inhibitor is administered at a dose of about 120 mg. In some embodiments, a CBL-B inhibitor is administered at a dose of about 140 mg. In some embodiments, a CBL-B inhibitor is administered at a dose of about 160 mg. In some embodiments, a CBL-B inhibitor is administered at a dose of about 180 mg. In some embodiments, a CBL-B inhibitor is administered at a dose of about 200 mg. In some embodiments, a CBL-B inhibitor is administered at a dose of about 220 mg. In some embodiments, a CBL-B inhibitor is administered at a dose of about 240 mg. In some embodiments, a CBL-B inhibitor is administered at a dose of about 260 mg. In some embodiments, a CBL-B inhibitor is administered at a dose of about 300 mg. In some embodiments, a CBL-B inhibitor is administered at a dose of about 325 mg. In some embodiments, a CBL-B inhibitor is administered at a dose of about 350 mg. In some embodiments, a CBL-B inhibitor is administered at a dose of about 375 mg. In some embodiments, a CBL-B inhibitor is administered at a dose of about 400 mg. In some embodiments, a CBL-B inhibitor is administered at a dose of about 425 mg. In some embodiments, a CBL-B inhibitor is administered at a dose of about 450 mg. In some embodiments, a CBL-B inhibitor is administered at a dose of about 475 mg. In some embodiments, a CBL-B inhibitor is administered at a dose of about 500 mg. In some embodiments, a CBL-B inhibitor is administered at a dose of about 525 mg. In some embodiments, a CBL-B inhibitor is administered at a dose of about 550 mg. In some embodiments, a CBL-B inhibitor is administered at a dose of about 575 mg. In some embodiments, a CBL-B inhibitor is administered at a dose of about 600 mg.

In some embodiments, a CBL-B inhibitor is administered at a dose of 2.0-60 mg/kg. In some embodiments, a CBL-B inhibitor is administered at a dose of 2.0-60 mg/kg. In some embodiments, a CBL-B inhibitor is administered at a dose of 2.0-10 mg/kg. In some embodiments, a CBL-B inhibitor is administered at a dose of 10-20 mg/kg. In some embodiments, a CBL-B inhibitor is administered at a dose of 20-30 mg/kg. In some embodiments, a CBL-B inhibitor is administered at a dose of 30-40 mg/kg. In some embodiments, a CBL-B inhibitor is administered at a dose of 40-50 mg/kg. In some embodiments, a CBL-B inhibitor is administered at a dose of 50-60 mg/kg.

In some embodiments, a CBL-B inhibitor is administered at a dose of about 2.0 mg/kg. In some embodiments, a CBL-B inhibitor is administered at a dose of about 6.0 mg/kg. In some embodiments, a CBL-B inhibitor is administered at a dose of about 10 mg/kg. In some embodiments, a CBL-B inhibitor is administered at a dose of about 20 mg/kg. In some embodiments, a CBL-B inhibitor is administered at a dose of about 30 mg/kg. In some embodiments, a CBL-B inhibitor is administered at a dose of about 40 mg/kg. In some embodiments, a CBL-B inhibitor is administered at a dose of about 50 mg/kg. In some embodiments, a CBL-B inhibitor is administered at a dose of about 60 mg/kg.

In some embodiments, a compound or composition disclosed herein may be administered at specified intervals. For example, during treatment a patient may be administered a compound or composition at intervals of every, e.g., 1 year, 6 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In some embodiments, a CBL-B inhibitor is administered once daily. In some embodiments, a CBL-B inhibitor is administered twice daily (BID). In some embodiments, a CBL-B inhibitor is administered three times daily (TID). In some embodiments, a CBL-B inhibitor is administered four time daily (QID). In some embodiments, a CBL-B inhibitor is administered once weekly. In some embodiments, a CBL-B inhibitor is administered twice weekly. In some embodiments, a CBL-B inhibitor is administered three times weekly. In some embodiments, a CBL-B inhibitor is administered four times weekly. In some embodiments, a CBL-B inhibitor is administered once monthly. In some embodiments, a CBL-B inhibitor is administered twice monthly. In some embodiments, a CBL-B inhibitor is administered three times monthly. In some embodiments, a CBL-B inhibitor is administered four times monthly.

In some embodiments, a compound or composition disclosed herein may be administered at specified intervals. For example, during treatment a patient may be administered a compound or composition at intervals of every, e.g., 1 year, 6 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

In some embodiments, an additional therapeutic agent is administered once daily. In some embodiments, an additional therapeutic agent is administered twice daily (BID). In some embodiments, an additional therapeutic agent is administered three times daily (TID). In some embodiments, an additional therapeutic agent is administered four time daily (QID). In some embodiments, an additional therapeutic agent is administered once weekly. In some embodiments, an additional therapeutic agent is administered twice weekly. In some embodiments, an additional therapeutic agent is administered three times weekly. In some embodiments, an additional therapeutic agent is administered four times weekly. In some embodiments, an additional therapeutic agent is administered once monthly. In some embodiments, an additional therapeutic agent is administered twice monthly. In some embodiments, an additional therapeutic agent is administered three times monthly. In some embodiments, an additional therapeutic agent is administered four times monthly.

In some embodiments, doxorubicin is administered at a dose of 60-75 mg/m². In some embodiments, doxorubicin is administered at a dose of 60-75 mg/m² every 3 weeks. In some embodiments, doxorubicin is administered at a dose of 40-75 mg/m². In some embodiments, doxorubicin is administered at a dose of 40-75 mg/m² every 3-4 weeks. In some embodiments, doxorubicin is administered at a dose of 60 mg/m². In some embodiments, doxorubicin is administered at a dose of 60 mg/m² every 14 days. In some embodiments, doxorubicin is administered at a dose of 20 mg/m². In some embodiments, doxorubicin is administered at a dose of 20 mg/m² every once a week.

In some embodiments, gemcitabine is administered at a dose of about 1000 mg/m². In some embodiments, gemcitabine is administered at a dose of about 1000 mg/m² once weekly. In some embodiments, gemcitabine is administered at a dose of about 1000 mg/m² once weekly on days 1 and 8 of each 21-day cycle. In some embodiments, gemcitabine is administered at a dose of about 1250 mg/m². In some embodiments, gemcitabine is administered at a dose of about 1250 mg/m² once weekly on days 1 and 8 of each 21-day cycle.

In some embodiments, oxaliplatin is administered at a dose of about 65-100 mg/m². In some embodiments, oxaliplatin is administered at a dose of about 65 mg/m². In some embodiments, oxaliplatin is administered at a dose of about 75 mg/m². In some embodiments, oxaliplatin is administered at a dose of about 85 mg/m².

### EXEMPLIFICATION

### Example 1. Synthesis of Compound 29

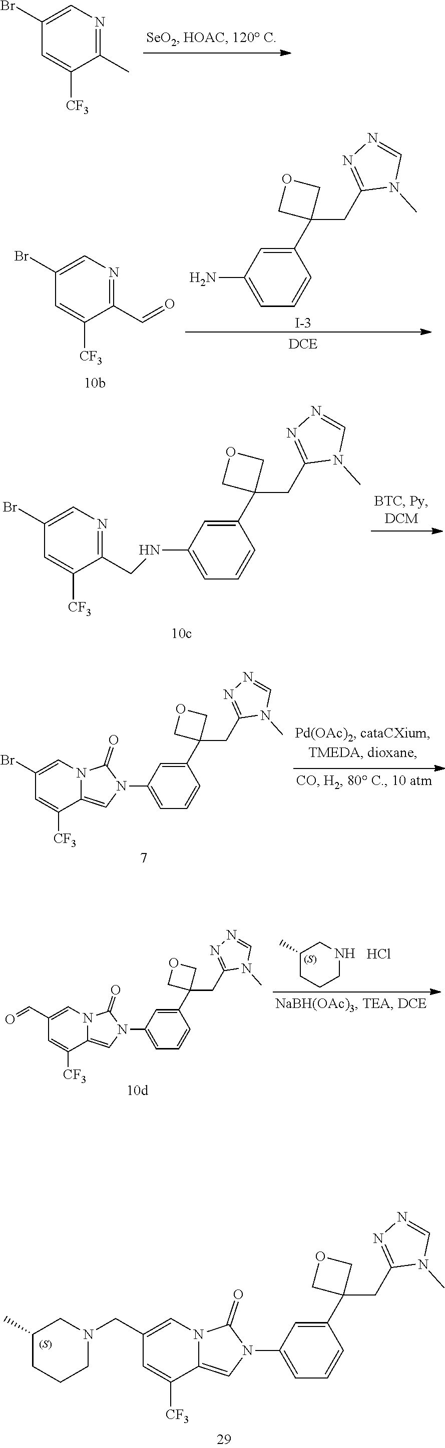

### 1. Synthesis of 10b

A mixture of 5-bromo-2-methyl-3-(trifluoromethyl)pyridine (100 g, 416.627 mmol, 1 equiv) and SeO₂ (92.47 g, 833.254 mmol, 2 equiv) in AcOH (500 mL) was stirred for overnight at 120° C. The resulting mixture was diluted with water (1000 mL). The aqueous layer was extracted with methyl tert-butyl ether (2×500 mL). The resulting mixture was concentrated under reduced pressure. The residue was purified by trituration with hexane (100 mL). This resulted in 5-bromo-3-(trifluoromethyl)pyridine-2-carbaldehyde (62 g, 58.81%) as a off-white solid.

### 2. Synthesis of 10c

To a stirred solution of 10b (100 g, 395.26 mmol, 1.20 equiv) and I-3 (80 g, 329.38 mmol, 1.00 equiv) in DCE (1000.00 mL) were added NaBH(OAc)₃ (139.65 g, 658.761 mmol, 3.00 equiv) at room temperature under nitrogen atmosphere. The resulting mixture was stirred for overnight at room temperature. The resulting mixture was diluted with water (2500.00 mL). The aqueous layer was extracted with EtOAc (3×1000.00 mL). The organic layers were concentrated under reduced pressure. The residue was purified by trituration with MTBE (2×300.00 mL). This resulted in 10c (116 g, 61.01%) as a white solid.

### 3. Synthesis of 7

To a stirred solution of 10c (130 g, 269.539 mmol, 1 equiv) and Pyridine (127.92 g, 1617.234 mmol, 6 equiv) in DCM (2600 mL) was added Triphosgene (26.39 g, 94.594 mmol, 0.35 equiv) at 0° C. The resulting mixture was stirred for 1 h at 0° C. The reaction was quenched by the addition of NaHCO₃(aq.) (1500 mL). The resulting mixture was extracted with CH₂Cl₂/MeOH=10/1 (2×300 mL). The combined organic layers were dried over anhydrous Na₂SO₄. After filtration, the filtrate was concentrated under reduced pressure. The residue was purified by trituration with methyl tert-butyl ether (600 mL). This resulted in 7 (110 g, 80.29%) as a yellow solid.

(ES, m/z): [M+H]⁺: 508

### 4. Synthesis of 10d

To a solution of 7 (110 g, 216.408 mmol, 1 equiv), TMEDA (50.30 g, 432.816 mmol, 2 equiv) in dioxane (4400 mL) was added bis(adamantan-1-yl)(butyl)phosphane (15.52 g, 43.282 mmol, 0.2 equiv) and Pd(OAc)₂ (4.86 g, 21.641 mmol, 0.1 equiv) in an autoclave. After flushing the autoclave three times with CO/H₂ (1:1), the mixture was pressurized to 10 atm with CO/H₂ (1:1) at 80 degrees overnight. The resulting mixture was concentrated under vacuum. The residue was purified by silica gel column chromatography, eluted with CH₂Cl₂/MeOH (20:1) to afford 10d (58 g, 58.59%) as a yellow solid.

(ES, m/z): [M+H]⁺: 458

### 5. Synthesis of 29

To a stirred mixture of 10d (58 g, 126.800 mmol, 1.00 equiv) and (s)-3-methylpiperidine hydrochloride (34.40 g, 253.600 mmol, 2 equiv) in DCE (1600 mL) was added TEA (38.49 g, 380.400 mmol, 3 equiv). The resulting mixture was stirred for 2 h at room temperature. To the above mixture was added NaBH(OAc)₃ (53.75 g, 253.600 mmol, 2 equiv). The resulting mixture was stirred overnight at room temperature. The reaction was quenched by the addition of Water (1000 mL). The resulting mixture was extracted with CH₂Cl₂/MEOH=10/1 (2×1000 mL). The combined organic layers were concentrated under reduced pressure. The residue was purified by reverse flash chromatography with the following conditions: column, C18 silica gel; mobile phase, MeCN in water (10 mmoL/L NH₄HCO₃), 15% to 80% gradient in 40 min; detector, UV 254 nm. This resulted in 29 (30.9 g, 45.08%) as a yellow solid.

LC-MS-29: (ES, m/z): [M+H]+ 541. H-NMR-29: (400 MHz, CD₃OD, δ ppm): 0.75-0.95 (m, 4H), 1.43-1.49 (m, 1H), 1.49-1.66 (m, 4H), 1.86-1.91 (m, 1H), 2.67-2.76 (m, 2H), 2.96 (s, 3H), 3.31 (s, 2H), 3.53 (s, 2H), 4.90-4.95 (m, 1H), 6.88-9.90 (d, 1H), 7.00 (s, 1H), 7.29 (s, 1H), 7.37-7.40 (d, 1H), 7.64 (s, 1H), 7.74-7.76 (d, 1H), 8.19 (s, 1H).

### Example 2. Synthesis of Compound 85

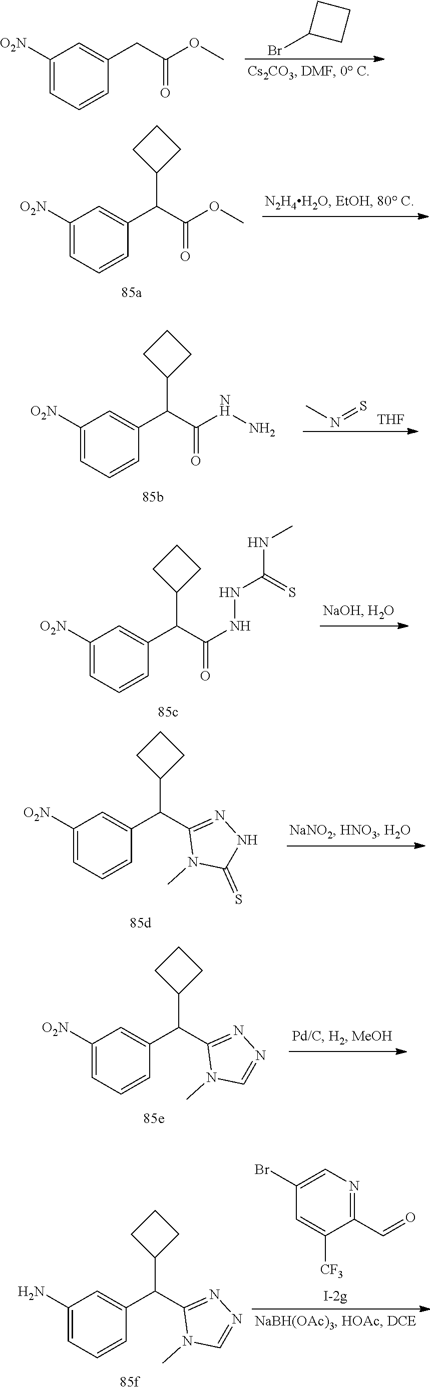

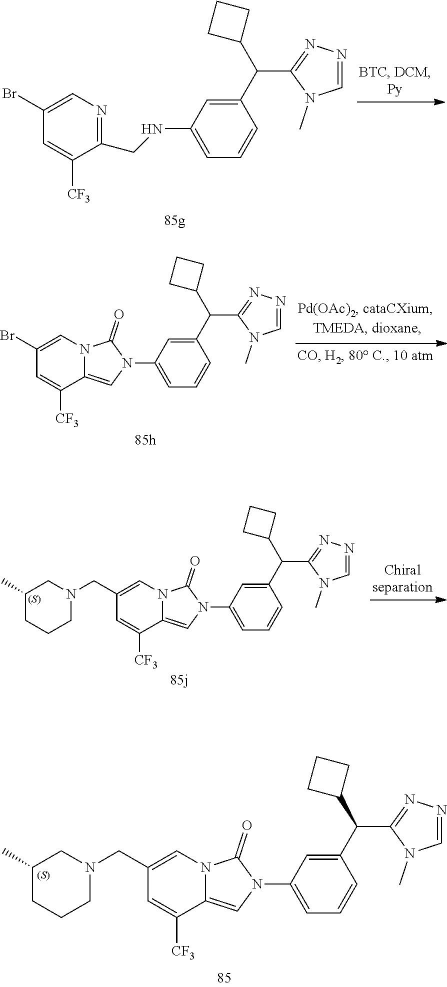

### Synthesis of 85a

A mixture of methyl 2-(3-nitrophenyl)acetate (48.1 g, 246.447 mmol, 1 equiv) and Cs₂CO₃ (401.49 g, 1232.235 mmol, 5 equiv) in DMF (500 mL) was stirred for 3 h at 0° C. under nitrogen atmosphere. To the above mixture was added bromocyclobutane (99.81 g, 739.341 mmol, 3 equiv) at room temperature. The resulting mixture was stirred overnight at room temperature. The reaction was diluted with NH₄Cl (aq.) (3 L) at 0° C. The aqueous layer was extracted with EtOAc (3×500 mL). The resulting mixture was concentrated under reduced pressure. The residue was purified by silica gel column chromatography, eluted with PE/EA (150:1) to afford 85a (49 g, 73.38%) as a off-white solid

### Synthesis of 85b

To a stirred solution of 85a (49 g, 196.577 mmol, 1 equiv) in EtOH (500 mL) was added hydrazine hydrate (98%) (251.04 g, 4914.425 mmol, 25 equiv, 98%) at room temperature. The resulting mixture was stirred overnight at 80° C. The reaction was diluted by the addition of water (500 mL) at room temperature. The aqueous layer was extracted with CH₂Cl₂/MEOH (10:1) (3×500 mL). The resulting mixture was concentrated under reduced pressure. The residue was purified by silica gel column chromatography, eluted with CH₂Cl₂/MeOH (100:1) to afford 85b (43 g, 79.86%) as a yellow oil.

### Synthesis of 85c

To a stirred solution of 85b (45 g, 180.527 mmol, 1 equiv) in THF (450 mL) was added methyl isothiocyanate (33.00 g, 451.317 mmol, 2.5 equiv) at room temperature under nitrogen atmosphere. The resulting mixture was stirred for 3 h at room temperature. The resulting mixture was diluted with water (280 mL). The resulting mixture was filtered, the filter cake was washed with water (3×50 mL). The resulting solid was dried under vacuum. This resulted in 85c (55 g, 86.00%) as a white solid.

### Synthesis of 85d

To a stirred solution of NaOH (66 g, 1650.120 mmol, 9.67 equiv) in H₂O (1.65 L) was added 85c (55 g, 170.606 mmol, 1 equiv) at room temperature. The resulting mixture was stirred overnight at room temperature. The mixture was acidified to pH 5 with HCl (1 M). The resulting mixture was filtered, the filter cake was washed with water (3×50 mL). The resulting solid was dried under vacuum. This resulted in 85d (50 g, 86.66%) as a off-white solid.

### Synthesis of 85e

To a stirred mixture of 85d (50 g, 164.274 mmol, 1 equiv) in EtOAc (190 mL) and H₂O (760 mL) was added NaNO₂ (113.3 g 1642.74 mmol, 10 equiv) at room temperature. To the above mixture was added HNO₃ (1642 mL, 1642.74 mmol, 10.00 equiv, 1 M) dropwise at 0 degrees C. The resulting mixture was stirred overnight at room temperature. The mixture was neutralized to pH 7 with saturated NaHCO₃(aq.). The aqueous layer was extracted with CH₂Cl₂/MeOH (10:1) (3×500 mL). The resulting mixture was concentrated under reduced pressure. The residue was purified by silica gel column chromatography, eluted with CH₂Cl₂/MeOH (50:1) to afford 85e (40 g, 85.84%) as a yellow solid.

### Synthesis of 85f

To a solution of 85e (40 g, 146.892 mmol, 1 equiv) in 1.2 L MeOH was added Pd/C (20%, 8 g) in a 2 L round-bottom flask. The mixture was hydrogenated at room temperature overnight under hydrogen atmosphere using a hydrogen balloon, filtered through a Celite pad, and concentrated under reduced pressure. This resulted in 85f (35 g, 94.39%) as a off-white solid.

### Synthesis of 85g

To a stirred solution of 85f (31.45 g, 123.800 mmol, 1.2 equiv) and I-2g (31.45 g, 123.800 mmol, 1.2 equiv) in DCE (300 mL) were added NaBH(OAc)₃ (43.73 g, 206.334 mmol, 2 equiv) and HOAc (6.20 g, 103.167 mmol, 1 equiv) at room temperature under nitrogen atmosphere. The resulting mixture was stirred overnight at room temperature under nitrogen atmosphere. The reaction was quenched by the addition of water (500 mL) at room temperature. The aqueous layer was extracted with EtOAc (3×500 mL), dried over anhydrous Na₂SO₄. After filtration, the filtrate was concentrated under reduced pressure. The residue was purified by trituration with MTBE (2×50 mL). This resulted in 85g (34 g, 65.18%) as a white solid.

### Synthesis of 85h

To a stirred solution of 85g (34 g, 70.784 mmol, 1 equiv) and pyridine (33.59 g, 424.704 mmol, 6 equiv) in DCM (400 mL) were added Triphosgene (7.35 g, 24.774 mmol, 0.35 equiv) dropwise at 0° C. under nitrogen atmosphere. The resulting mixture was stirred for 10 min at room temperature under nitrogen atmosphere. The reaction was quenched by the addition of water (500 mL) at room temperature. The resulting mixture was extracted with CH₂Cl₂ (3×500 mL) and dried over anhydrous CaCl₂). After filtration, the filtrate was concentrated under reduced pressure. The residue was purified by trituration with MTBE (2×100 mL). This 85h (33 g, 87.47%) as a yellow solid.

### Synthesis of 85i

To a solution of 85h (33 g, 65.175 mmol, 1 equiv) and TMEDA (15.15 g, 130.350 mmol, 2 equiv) in dioxane (1000 mL) was added bis(adamantan-1-yl)(butyl)phosphane (4.67 g, 13.035 mmol, 0.2 equiv) and Pd(OAc)₂ (1.46 g, 6.518 mmol, 0.1 equiv) in an autoclave. After flushing the autoclave three times with CO/H₂ (1:1), the mixture was pressurized to 10 atm with CO/H₂ (1:1) and run overnight at 80 degrees C. The resulting mixture was concentrated under vacuum. The residue was purified by silica gel column chromatography, eluted with CH₂Cl₂/MeOH (20:1) to CH₂Cl₂/MeOH (5:1) to afford 85i (20 g, 67.38%) as a yellow solid.

### Synthesis of 85j

To a stirred mixture of 85i (10 g, 21.957 mmol, 1 equiv) and (3S)-3-methylpiperidine hydrochloride (8.93 g, 65.871 mmol, 3 equiv) in DCE (150 mL) was added Et₃N (8.89 g, 87.828 mmol, 4 equiv). The resulting mixture was stirred for 2 h at room temperature under nitrogen atmosphere. To the above mixture was added NaBH(OAc)₃ (6.98 g, 32.936 mmol, 1.5 equiv). The resulting mixture was stirred overnight at room temperature. The reaction was quenched by the addition of water (200 mL) at room temperature. The resulting mixture was extracted with CH₂Cl₂/MeOH=10/1 (2×200 mL). The combined organic layers were concentrated under reduced pressure. The residue was purified by reverse flash chromatography with the following conditions: column, C18 silica gel; mobile phase, MeCN in water (10 mol/L NH₄HCO₃), 15% to 60% gradient in 40 min; detector, UV 254 nm. This resulted in 85j (6.5 g, 54.96%) as a yellow solid.

### Synthesis of 85

The 85j (6.5 g) was purified by Prep-Chiral-SFC with the following conditions (Column: CHIRAL ART Cellulose-SB, 5*25 cm, 10 μm; Mobile Phase A: CO₂, Mobile Phase B: MEOH (0.1% 2M NH₃-MeOH); Flow rate: 200 mL/min; Gradient: isocratic 30% B; Column Temperature (° C.): 35; Back Pressure (bar): 100; Wave Length: 220 nm; RT2 (min): 6.26; Sample Solvent: MEOH (0.1% 2M NH₃-MEOH); Injection Volume: 1 mL; Number Of Runs: 30). This resulted in Compound 85 (3.0062g) as a yellow solid.

LC-MS: (ES, m/z): [M+H]⁺ 540

H-NMR: (400 MHz, DMSO-d6, ppm): δ 0.84-0.91 (m, 4H), δ 1.38-1.95 (m, 12H), δ 2.08-2.10 (m, 1H), δ 2.68-2.77 (m, 2H), δ 3.19-3.25 (m, 3H), δ 3.43 (s, 3H), δ 4.25-4.28 (d, 1H), δ 7.01 (s, 1H), δ 7.19-7.21 (d 1H), δ 7.32 (s, 1H), δ 7.43-7.46 (t, 1H), δ 7.66-7.75 (m, 3H), δ 8.34 (s, 1H).

### Example 3. Synthesis of Compound 86

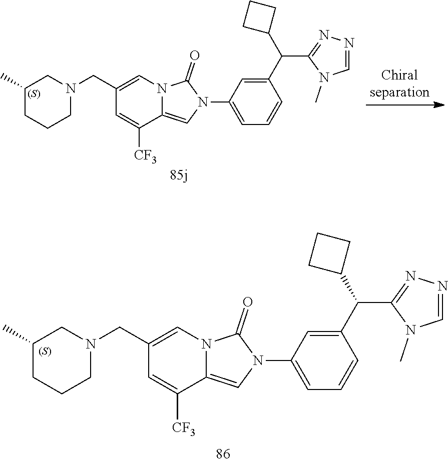

### Synthesis of 86

85j (85.00 mg, 0.158 mmol, 1.00 equiv) was purified by Prep-Chiral-HPLC with the following conditions (Column: CHIRAL ART Cellulose-SB, 2*25 cm, 5 μm; Mobile Phase A: Hex (0.5% 2M NH3-MeOH), Mobile Phase B: EtOH; Flow rate: 20 mL/min; Gradient: 30% B to 30% B in 8.5 min; Wave Length: 220/254 nm; RT1 (min): 5.19) to afford Compound 86 (24.1 mg, 27.90%) as a yellow solid.

LC-MS: (ES, m/z): [M+H]⁺ 539

H-NMR: (400 MHz, CDCl₃, ppm): δ 0.81-0.83 (d, 4H), δ 1.66-1.78 (m, 11H), δ 2.04-2.11 (m, 1H), 62.65-3.69 (m, 1H), δ 2.72-3.73 (m, 1H), δ 3.22-3.35 (m, 3H), δ 3.42 (s, 3H), δ 4.24-4.28 (d, 1H), δ 7.00 (s, 1H), δ 7.19-7.22 (d, 1H), δ 7.33 (s, 1H), δ 7.44-7.47 (m, 1H), δ 7.70-7.72 (m, 3H), δ 8.32 (s, 1H).

### Example 4. In Vivo Efficacy Study in the Treatment of Subcutaneous Murine Colorectal Cancer Model CT26.WT in Female BALB/c Mice

The objective of this study was to evaluate preclinically the in vivo therapeutic efficacy of HotSpot Therapeutics's Test Articles in the treatment of the subcutaneous murine Colorectal model CT26.WT in female BALB/c mice. Compound 29 test particles were delivered via PO. Compound 29 was assessed at various dose levels in combination with Oxaliplatin or Cabozantinib. Tumor volumes and body weight were measured three times per week after the initiation of treatment. The mice were broken into several groups, summarized in Table 2.

TABLE 2

| | | | Dose | Dosing | Dosing | | |
|---|---|---|---|---|---|---|---|
| | No. of | | level | Solution | Volume | | Dosing Frequency & |
| Group | mice | Treatment | (mg/kg) | (mg/ml) | (μL/g) | ROA | Duration |
| 1 | 10 | Vehicle | — | — | 10 | p.o. | BID × 17 days |
| | | | | | | | (day 0~day 16) |
| 2 | 10 | Compound | 60 | 6 | 10 | p.o. | BID × 21 days |
| | | 29 | | | | | (day 0~day 20) |
| 3 | 10 | Oxaliplatin | 5 | 0.5 | 10 | i.p. | Q4D × 5 doses |
| | | | | | | | (day 0, day 4, day 8, |
| | | | | | | | day 12, day 16) |
| 4 | 10 | Cabozantinib | 10 | 1 | 10 | p.o. | QD × 21 days |
| | | | | | | | (day 0~day 20) |
| 5 | 10 | Compound | 60 | 6 | 10 | p.o. | BID × 21 days |
| | | 29 | | | | | (day 0~day 20) |
| | | Oxaliplatin | 5 | 0.5 | 10 | i.p. | Q4D × 6 doses |
| | | | | | | | (day 0, day 4, day 8, |
| | | | | | | | day 12, day 16 and |
| | | | | | | | day 20) |
| 6 | 10 | Compound | 60 | 6 | 10 | p.o. | BID × 21 days |
| | | 29 | | | | | (day 0~day 20) |
| | | Cabozantinib | 10 | 1 | 10 | p.o. | QD × 21 days |
| | | | | | | | (day 0~day 20) |

The test articles of this study were formulated as outlined in Table 3

TABLE 3

| | Dose | Dosing | Dosing | |
|---|---|---|---|---|
| | level | Conc. | Volume | |
| Test article | (mg/kg) | (mg/mL) | (μL/g) | Formulation |
| 0.5% methyl | N/A | N/A | 10 | 0.5% methyl cellulose (400 cP) |
| cellulose | | | | Weighed 1.0 g Methylcellulose A4C, added |
| (400 cP) | | | | sterile water to 200.0 ml to make 0.5% |
| | | | | methylcellulose solution. |
| Vehicle | N/A | N/A | 10 | Taken 50 ml of 0.5% methyl cellulose (400 cP) |
| | | | | pH = 3.36 ± 0.3. Aliquot into 3 ml/vial × |
| | | | | 14vials for Group 1. |
| Compound 29 | 60 | 6 | 10 | Dissolved 54.22 mg (54.0 mg × salt factor 1.004) |
| | | | | mg Compound 29 with 9 ml 0.5% MC, adjust |
| | | | | pH with 1M HCl to 3.36 ± 0.3, followed by |
| | | | | vortexed and sonification. When make the 5pm |
| | | | | dosing solution, leave it at room temperature to |
| | | | | prevent any precipitation and vortex thoroughly |
| | | | | before PM dosing |
| Oxaliplatin | 5 | 0.5 | 10 | Weighted 3.0 mg of Oxaliplatin into 5% USP |
| | | | | Dextrose to make 6.0 ml of 1 mg/ml dosing |
| | | | | solution; |
| Cabozantinib | 10 | 1 | 10 | Weighed 6 mg Cabozantinib into a vial. |
| | | | | Added 0.12 mL of 2% DMSO into the vial. |
| | | | | Vortexed to make it as solution. |
| | | | | Added 1.8 mL 30% PEG300 into the vial. |
| | | | | Vortexed to mix well. |
| | | | | Added 0.3 mL 5% Tween80 into the vial. |
| | | | | Vortexed to mix well. |
| | | | | Added 3.78 mL 63% sterile water into the vial. |
| | | | | Vortexed to mix well. |

### Experimental Methods

### Cell Culture

The CT26.WT tumor cells were maintained in vitro with RPMI-1640 medium supplemented with 10% fetal bovine serum at 37° C. in an atmosphere of 5% CO2 in air. The cells in exponential growth phase were harvested and quantitated by cell counter before tumor inoculation.

### Tumor Inoculation

Each mouse was inoculated subcutaneously it the right lower flank region with 5×10⁵ tumor cells in 0.1 ml of PBS mixed with Matrigel (1:1) for tumor development.

### Randomization

The randomization started when the mean tumor size reached approximately 86.42 mm³. 60 mice were enrolled in the study. All animals were randomly allocated to 6 study groups, 10 mice in each group. Randomization was performed based on “Matched distribution” method (StudyDirector™ software, version 3.1.399.19)

The date of randomization was denoted as day 0.

### Test Article Administration

The treatment was initiated on the same day of randomization (day 0) per study design.

### Observation and Data Collection

After tumor cells inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm³ using the formula: “V=(L×W×W)/2, where V was tumor volume, L was tumor length (the longest tumor dimension) and W was tumor width (the longest tumor dimension perpendicular to L). Dosing as well as tumor and body weight measurements were conducted in a Laminar Flow Cabinet.

The body weights and tumor volumes were measured by using StudyDirector™ software (version 3.1.399.19).

### Study Termination

For Group 01 and Group 03 were terminated when the mean tumor volume of the vehicle control group reaches 2000 mm\hat{ }3 on day 16.

For Group 02, Group 04, Group 05 and Group 06 were performed for 3 weeks. The study was terminated on day 21 (Sep. 21, 2022).

### Statistical Analysis Method

To compare tumor volumes of different groups at a pre-specified day, we first used Bartlett's test to check the assumption of homogeneity of variance across all groups. When the p-value of Bartlett's test was >=0.05, we ran one-way ANOVA to test overall equality of means across all groups. If the p-value of the one-way ANOVA was <0.05, we further performed post hoc testing by running Tukey's HSD (honest significant difference) tests for all pairwise comparisons, and Dunnett's tests for comparing each treatment group with the vehicle group. When the p-value of Bartlett's test was <0.05, we ran Kruskal-Wallis test to test overall equality of medians among all groups. If the p-value the Kruskal-Wallis test was <0.05, we further performed post hoc testing by running Conover's non-parametric test for all pairwise comparisons or for comparing each treatment group with the vehicle group, both with single-step p-value adjustment.

In addition, we performed pairwise comparisons without multiple comparison correction and report nominal/uncorrected p-values directly from Welch's t-test or Mann-Whitney U test at each time point. Specifically, we first used Shapiro Wilk test to check the assumption of normal distribution for each group. When p-values of Shapiro Wilk tests of both groups in a pair are >0.05, we ran Welch's t-test, otherwise we ran Mann-Whitney U test, to obtain nominal p-values.

All statistical analyses had been done in R-a language and environment for statistical computing and graphics (version 3.3.1). All tests have two-sided unless otherwise specified, and p-values of <0.05 were regarded as statistically significant.

For the optional survival analysis, Kaplan-Meier model with Log-rank method was applied.

### Result

### Tumor Volume Growth Curve

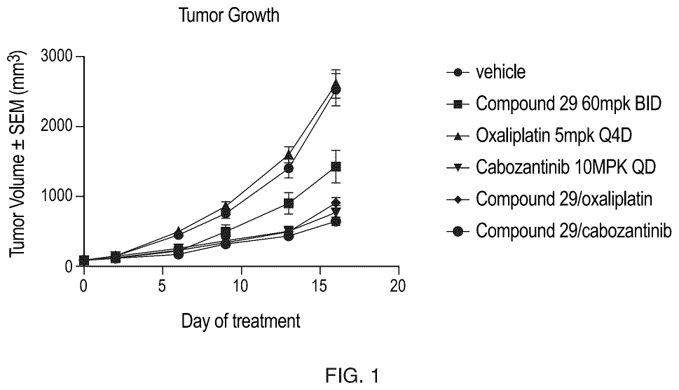

The tumor volume growth curves between randomization grouping and study termination are shown below. The mean tumor volume curve of vehicle and treatment group with SEM is shown in FIG. 1. The treatment was performed for 21 days. The study was terminated on Day 21.

TGI and T/C calculation is based on the tumor size data of Day 16, which is the last dosing day of the treatment (Table 4).

TABLE 4
TGI and T/C calculation on day 16

| | | | Day 16 | | | |
|---|---|---|---|---|---|---|
| | | Dose Level | Tumor Volume | T/C | TGI | P |
| Group | Treatment | (mg/kg) | (mean ± SEM) | (%) | (%) | Value |
| 1 | Vehicle | — | 2526.75 ± 229.97 | — | — | — |
| 2 | Compound 29 | 60 | 1425.65 ± 232.85 | 56.42 | 43.58 | 1.12e−02 |
| 3 | Oxaliplatin | 5 | 2609.42 ± 203.84 | 103.27 | −3.27 | 9.99e−01 |
| 4 | Cabozantinib | 10 | 772.01 ± 100.44 | 30.55 | 69.45 | 9.19e−08 |
| 5 | Compound 29 | 60 | 908.21 ± 76.22 | 35.94 | 64.06 | 1.75e−05 |
| | Oxaliplatin | 5 | | | | |
| 6 | Compound 29 | 60 | 643.08 ± 59.79 | 25.45 | 74.55 | 4.45e−09 |
| | Cabozantinib | 10 | | | | |

Note:
Bartlett's test was performed to test homogeneity of variance and normality, P = ~0.000, significant, non-parametric test is run to compare groups. P < 0.05 is considered to be statistically significant.

TABLE 2
Tumor growth inhibition multiple comparisons between groups

| | | |
|---|---|---|
| Test | p. values | significance. level |

Bartlett's test of homogenity of variance and normality

| | | |
|---|---|---|
| Bartlett's test | 1.08e−04 | *** |

Test of overall equality among groups

| | | |
|---|---|---|
| Kruskal-Wallis test | 1.12e−07 | *** |

Test of equality between individual groups

| | | |
|---|---|---|
| G01-G02 | 1.12e−02 | * |
| G01-G03 | 9.99e−01 | ns |
| G01-G04 | 9.19e−08 | *** |
| G01-G05 | 1.75e−05 | *** |
| G01-G06 | 4.45e−09 | *** |

Note:
ns = non-significance

### SUMMARY

In this study, mean tumor size of vehicle reached 2526.75 mm\hat{ }3 on day 16.

Treatment with Oxaliplatin (Group 03, 5 mg/kg, Q4D) as single agent didn't produce statistically anti-tumor efficacy against subcutaneous CT26.WT murine colorectal cancer model, with TGI value of −3.27%. (P>0.05 vs. Group 01).

Treatment with Compound 29 (Group 02, 60 mg/kg, BID) as single agent produced statistically anti-tumor activity against subcutaneous CT26.WT murine colorectal cancer model, with TGI value of 43.58%. (P<0.05 vs. Group 01). Treatment with Compound 29 (Group 05, 60 mg/kg, BID) in combine with Oxaliplatin (Group 05, 5 mg/kg, Q4D) produce enhanced anti-tumor efficacy against subcutaneous CT26.WT murine colorectal cancer model, with TGI value of 64.06% (P<0.01 vs. Group 01).

### Example 5. In Vivo Efficacy Study in the Treatment of Subcutaneous Murine Colorectal Cancer Model CT26.WT in Female BALB/c Mice

The objective of this study was to evaluate preclinically the in vivo therapeutic efficacy of Test Articles in the treatment of the subcutaneous murine Colorectal model CT26.WT in female BALB/c mice. Compound 29 test particles were delivered via PO. Compound 29 was assessed at various dose levels in combination with Doxorubicin, Gemcitabine or Paclitaxel. Tumor volumes and body weight were measured three times per week after the initiation of treatment. The mice were broken into several groups, summarized in Table 3

TABLE 3

| | | | Dose | Dosing | Dosing | | |
|---|---|---|---|---|---|---|---|
| | No. of | | level | Solution | Volume | | Dosing Frequency & |
| Group | mice | Treatment | (mg/kg) | (mg/ml) | (μL/g) | ROA | Duration |
| 1 | 10 | Vehicle | — | — | 10 | p.o. | BID × 21 days |
| | | | | | | | (day 0~day 20) |
| 2 | 10 | Compound 29 | 60 | 6 | 10 | p.o. | BID × 21 days |
| | | | | | | | (day 0~day 20) |
| 3 | 10 | Doxorubicin | 4 | 0.4 | 10 | i.v. | Q4D × 6 doses |
| | | | | | | | (day 0, day 4, day 8, day 12, |
| | | | | | | | day 16 and day 20) |
| 4 | 10 | Gemcitabine | 5 | 0.5 | 10 | i.p. | Q3D × 7 doses |
| | | | | | | | (day 0, day 3, day 6, day 9, |
| | | | | | | | day 12, day 15 and day 18) |
| 5 | 10 | Paclitaxel | 20 | 2 | 10 | i.p. | QW × 3 doses |
| | | | | | | | (day 0, day 7 and day 14) |
| 6 | 10 | Compound 29 | 60 | 6 | 10 | p.o. | BID × 21 days |
| | | | | | | | (day 0~day 20) |
| | | Doxorubicin | 4 | 0.4 | 10 | i.v. | Q4D × 6 doses |
| | | | | | | | (day 0, day 4, day 8, day 12, |
| | | | | | | | day 16 and day 20) |
| 7 | 10 | Compound 29 | 60 | 6 | 10 | p.o. | BID × 21 days |
| | | | | | | | (day 0~day 20) |
| | | Gemcitabine | 5 | 0.5 | 10 | i.p. | Q3D × 7 doses |
| | | | | | | | (day 0, day 3, day 6, day 9, |
| | | | | | | | day 12, day 15 and day 18) |
| 8 | 10 | Compound 29 | 60 | 6 | 10 | p.o. | BID × 21 days |
| | | | | | | | (day 0~day 20) |
| | | Paclitaxel | 20 | 2 | 10 | i.p. | QW × 3 doses |
| | | | | | | | (day 0, day 7 and day 14) |

Note:
1) The day of grouping will be denoted as day 0. Dosing of all test materials will be started from the day 0;
2) Vortex dosing solution before each syringe-draw.
3) BID interval is 8 hours.
4) Dose level of Gemcitabine will be calculated based on correction factor.

The test articles of this study were formulated as outlined in Table 4.

TABLE 4

| | Dose | Dosing | Dosing | |
|---|---|---|---|---|
| | level | Conc. | Volume | |
| Test article | (mg/kg) | (mg/mL) | (μL/g) | Formulation |
| 0.5% methyl cellulose | N/A | N/A | 10 | 0.5% methyl cellulose (400 cP) |
| (400 cP) | | | | Weigh 1.0 g Methylcellulose A4C, add sterile |
| | | | | water to 200.0 ml to make 0.5% methylcellulose |
| | | | | solution. |
| Vehicle | N/A | N/A | 10 | Take 50 ml of 0.5% methyl cellulose (400 cP) |
| | | | | pH = 3.36 ± 0.3. Aliquot into 3 ml/vial × |
| | | | | 14vials for Group 1. |
| Compound 29 | 60 | 6 | 10 | Dissolve 72.29 mg (72 mg × salt factor 1.004) |
| | | | | mg Compound 29 with 12 ml 0.5% MC, adjust |
| | | | | pH with 1M HCl to 3.36 ± 0.3, followed by |
| | | | | vortex and sonification. When make the 5pm |
| | | | | dosing solution, leave it at room temperature to |
| | | | | prevent any precipitation and vortex thoroughly |
| | | | | before PM dosing |
| Doxorubicin | 4 | 0.4 | 10 | Dilute 0.48 mL stock solution |
| | | | | (5 mg/mL) by 5.52 mL Saline, then |
| | | | | mix well. |
| Paclitaxel | 20 | 2 | 10 | Dilute 2 mL stock solution |
| | | | | (6 mg/mL) by 4 mL Saline, then |
| | | | | mix well. |
| Gemcitabine | 5 | 0.5 | 10 | Dissolve 3 mg Gemcitabine by 6 mL Saline to |
| | | | | obtain 0.5 mg/mL dosing solution. |

### Experimental Methods

### Cell Culture

The CT26.WT tumor cells will be maintained in vitro with RPMI-1640 medium supplemented with 10% fetal bovine serum at 37° C. in an atmosphere of 5% CO2 in air. The cells in exponential growth phase will be harvested and quantitated by cell counter before tumor inoculation.

### Tumor Inoculation

Each ach mouse will be inoculated subcutaneously in the right lower flank region with CT26 tumor cells (5×10\hat{ }5) in 0.1 ml of PBS for tumor development.

### Randomization

The randomization will start when the mean tumor size reaches approximately 80-100 mm\hat{ }3. Totally 80 mice each model will be enrolled in the study and randomly allocated to 8 study groups as shown in Table 4, with 10 mice per group. Randomization will be performed based on “Matched distribution” method/“Stratified” method (Study Director™ software, version 3.1.399.19)/randomized block design. The date of randomization will be denoted as day 0.

### Test Article Administration

The treatment will be initiated on the same day of randomization (day 0) per study design.

### Observation and Data Collection

After tumor cells inoculation, the animals will be checked daily for morbidity and mortality. During routine monitoring, the animals will be checked for any effects of tumor growth and treatments on behavior such as mobility, food and water consumption, body weight gain/loss (Body weights will be measured twice per week after randomization), eye/hair matting and any other abnormalities. Mortality and observed clinical signs will be recorded for individual animals in detail.

Tumor volumes will be measured twice per week after randomization in two dimensions using a caliper, and the volume will be expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements will be conducted in a Laminar Flow Cabinet.

The body weights and tumor volumes will be measured by using StudyDirector™ software (version 3.1.399.19).

### Study Termination

The efficacy study was terminated when the mean tumor volume of the vehicle control group reaches 2000 mm\hat{ }3 or remain on study until last dose, whichever comes first.

The treatment was performed for 21 days. If there is no extension of the treatment, the study was terminated on day 20.

### Results

Overall, a CBL-B inhibitor in combination with Doxorubicin, Gemcitabine or Paclitaxel in CT26 tumor models show improved effect to slow tumor growth even with limited chemo dosing.

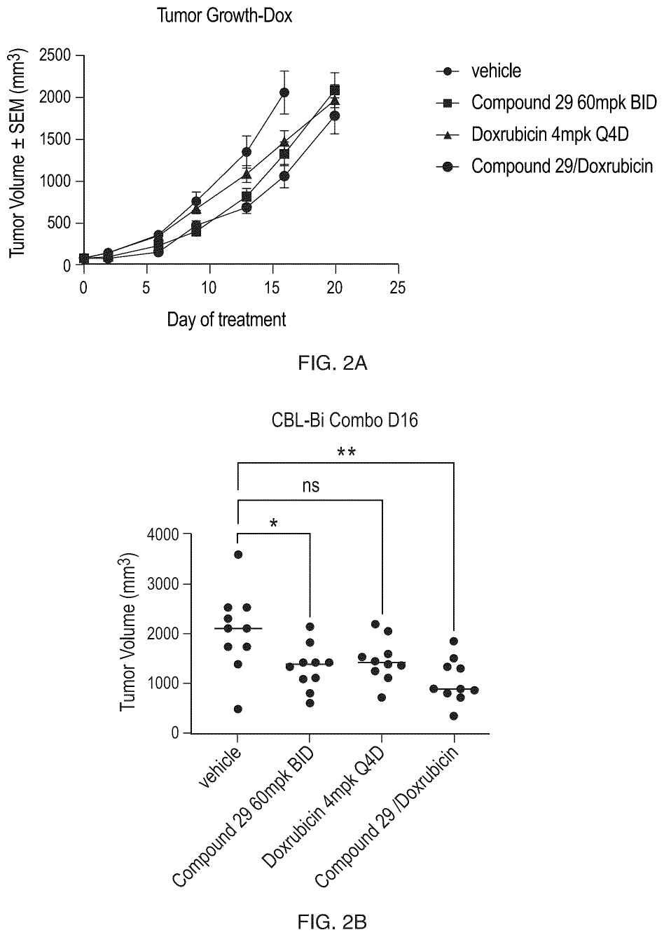

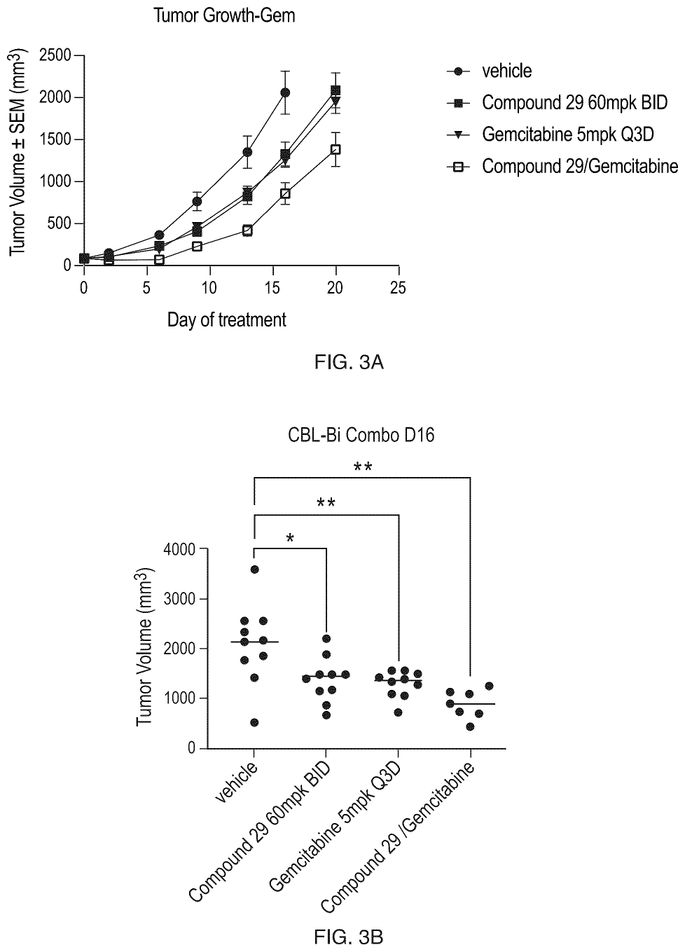

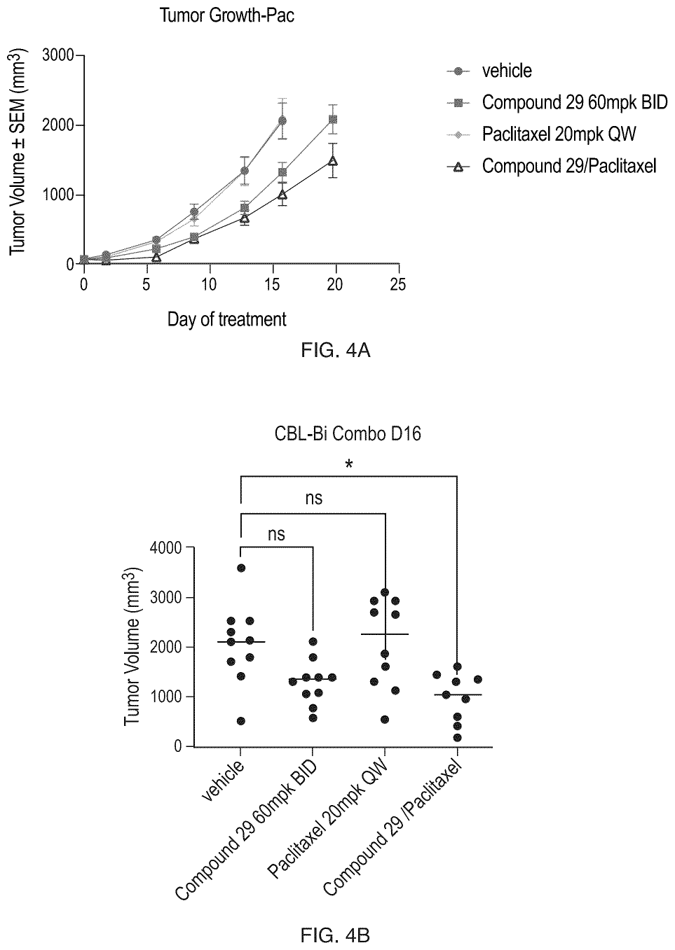

Compound 29 and Doxrubicin in combination shows improved effect on tumor inhibition as compared to each of the agents as monotherapies (See FIG. 2A and FIG. 2B). Compound 29 and Gemcitabine in combination shows improved effect on tumor inhibition as compared to each of the agents as monotherapies (See FIG. 3A and FIG. 3B). Compound 29 and Paclitaxel in combination shows improved effect on tumor inhibition as compared to each of the agents as monotherapies (See FIG. 4A and FIG. 4B).

### Example 6. In Vivo Efficacy Study in the Treatment of Subcutaneous Murine Colorectal Cancer Model CT26.WT in Female BALB/c Mice

The objective of this study was to evaluate preclinically the in vivo therapeutic efficacy of Test Articles in the treatment of the subcutaneous murine Colorectal model CT26.WT in female BALB/c mice. Compound 29 test particles were delivered via PO. Compound 29 was assessed at various dose levels in combination with ALT-803. Tumor volumes and body weight were measured three times per week after the initiation of treatment. The mice were broken into several groups, summarized in Table 5

TABLE 5

| | | | Dose | Dosing | Dosing | | |
|---|---|---|---|---|---|---|---|
| | No. of | | level | Solution | Volume | | Dosing Frequency & |
| Group | mice | Treatment | (mg/kg) | (mg/ml) | (μL/g) | ROA | Duration |
| 1 | 10 | Vehicle | — | — | 10 | p.o. | BID × 21 days |
| | | | | | | | (day 0~day 20) |
| 2 | 10 | Compound 29 | 30 | 3 | 10 | p.o. | BID × 21 days |
| | | | | | | | (day 0~day 20) |
| 3 | 10 | ALT-803 | 0.2 | 0.02 | 10 | i.p. | QW × 4 weeks |
| | | | | | | | (day 0, day 7, day 14 and day 20) |
| 4 | 10 | Compound 29 | 30 | 3 | 10 | p.o. | BID × 21 days |
| | | | | | | | (day 0~day 20) |
| | | ALT-803 | 0.2 | 0.02 | 10 | i.p. | QW × 4 weeks |
| | | | | | | | (day 0, day 7, day 14 and day 20) |

The test articles of this study were formulated as outlined in Table 6.

TABLE 6

| | Dose | Dosing | Dosing | |
|---|---|---|---|---|
| | level | Conc. | Volume | |
| Test article | (mg/kg) | (mg/mL) | (μL/g) | Formulation |
| 0.5% methyl | N/A | N/A | 10 | 0.5% methyl cellulose (400 cP) |
| cellulose | | | | Weigh 1.0 g Methylcellulose A4C, add sterile |
| (400 cP) | | | | water to 200.0 ml to make 0.5% |
| | | | | methylcellulose solution. |
| Vehicle | N/A | N/A | 10 | Take 50 ml of 0.5% methyl cellulose (400 cP) |
| | | | | pH = 3.36 ± 0.3. Aliquot into 3 ml/vial × |
| | | | | 14vials for Group 1. |
| Compound 29 | 30 | 3 | 10 | Dissolve 18.27 mg (18 mg × salt factor 1.015) |
| | | | | mg Compound 29 with 6 ml 0.5% MC, adjust |
| | | | | pH with 1M HCl to 3.36 ± 0.3, followed by |
| | | | | vortex and sonification. When make the 5pm |
| | | | | dosing solution, leave it at room temperature |
| | | | | to prevent any precipitation and vortex |
| | | | | thoroughly before PM dosing |
| ALT-803 | 0.2 | 0.02 | 10 | |

### Experimental Methods

### Cell Culture

The CT26.WT tumor cells will be maintained in vitro with RPMI-1640 medium supplemented with 10% fetal bovine serum at 37° C. in an atmosphere of 5% CO2 in air. The cells in exponential growth phase will be harvested and quantitated by cell counter before tumor inoculation.

### Tumor Inoculation

Each ach mouse will be inoculated subcutaneously in the right lower flank region with CT26 tumor cells (5×10e5) in 0.1 ml of PBS for tumor development.

### Randomization

The randomization will start when the mean tumor size reaches approximately 80-100 mm\hat{ }3. Totally 40 mice each model will be enrolled in the study and randomly allocated to 4 study groups as shown in Table 4, with 10 mice per group. Randomization will be performed based on “Matched distribution” method/“Stratified” method (Study Director™ software, version 3.1.399.19)/randomized block design. The date of randomization will be denoted as day 0.

### Test Article Administration

The treatment will be initiated on the same day of randomization (day 0) per study design.

### Observation and Data Collection

After tumor cells inoculation, the animals will be checked daily for morbidity and mortality. During routine monitoring, the animals will be checked for any effects of tumor growth and treatments on behavior such as mobility, food and water consumption, body weight gain/loss (Body weights will be measured twice per week after randomization), eye/hair matting and any other abnormalities. Mortality and observed clinical signs will be recorded for individual animals in detail.

Tumor volumes will be measured twice per week after randomization in two dimensions using a caliper, and the volume will be expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements will be conducted in a Laminar Flow Cabinet.

The body weights and tumor volumes will be measured by using StudyDirector™ software (version 3.1.399.19).

### Study Termination

The efficacy study will be terminated when the mean tumor volume of the vehicle control group reaches 2000 mm\hat{ }3 or remain on study until last dose, whichever comes first.

The treatment will be performed for 21 days. If there is no extension of the treatment, the study will be terminated on day 20.

### Results

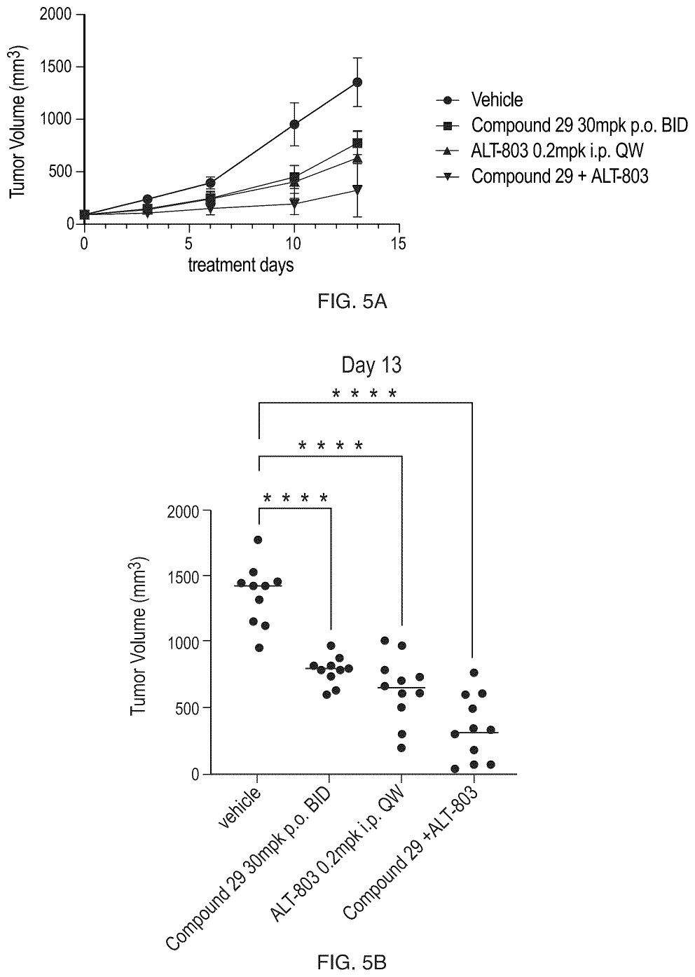

Overall, Compound 29 and ALT-803 in combination shows improved effect on tumor inhibition as compared to each of the agents as monotherapies (See FIG. 5A and FIG. 5B).

We claim:

1. A method of treating a disease or condition associated with cell proliferation comprising administering to a subject in need thereof a therapeutically effective amount of a CBL-B inhibitor and therapeutically effective amount of an additional therapeutic agent.

2. A method of treating a disease or condition associated with cell proliferation comprising administering to a subject in need thereof a therapeutically effective amount of a CBL-B inhibitor wherein the subject has previously been treated with an additional therapeutic agent.

3. A method of treating a disease or condition associated with cell proliferation comprising administering to a subject in need thereof a therapeutically effective amount of an additional therapeutic agent wherein the subject has previously been treated with a CBL-B inhibitor.

4. The method of any of claims 1-3, wherein the CBL-B inhibitor is a compound of formula (A): 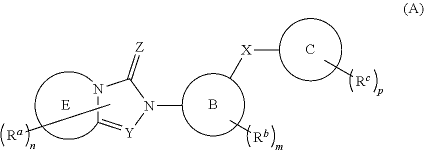
or pharmaceutically acceptable salts thereof,
wherein
Y is selected from the group ═C(H)—, ═C(R^{a})— or ═N—;
Z is ═O or ═S;
E is optionally substituted 5-6 membered heterocyclyl;
B is optionally substituted phenyl, optionally substituted 8-10 membered bicyclyl, or optionally substituted 5-6 membered heteroaryl;
C is optionally substituted 5-6 membered heterocyclyl;
X is an optionally substituted C₁-C₃ alkylene chain, wherein one or more methylene units is optionally replaced by —N(H)—, —N(R¹)—, —O—, —S—, —SO—, —SO₂—, optionally substituted 3-6-membered carbocyclyl, and optionally substituted 3-6-membered heterocylyl, wherein X is optionally substituted with an optionally substituted group selected from a group consisting of halogen, C₁-C₃ aliphatic, phenyl, 3-6-membered heteroaryl, 3-6-membered heterocylyl, and —(CH₂)(3-6-membered carbocyclyl);
each R^{a} is independently selected from the group consisting of L-A, halogen, —CN, —OH, —OR¹, —NH₂, —NR¹R², —SH, —SR¹, —SF₅, —CO₂H, —CO₂R¹, —C(O)R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein R^{a} is optionally substituted with 1-5 instances of R^{a1};
L is an optionally substituted C₁-C₃ alkylene chain;
A is selected from the group consisting of optionally substituted C₃-C₇ carbocylyl, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S, wherein A is optionally substituted with 1-5 instances of R^{a1};
each R^{a1} is independently selected from the group consisting of halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{b} is independently selected from the group consisting of, halogen, —CN, —OH, —OR¹, —NH₂, —NR¹R², —SH, —SR¹, —SF₅, —CO₂H, —CO₂R¹, —CONH₂, —CONR¹R², —SO₂NH₂, —SO₂NR¹R², —SO₂OH, —SO₂OR¹, —S(O)R¹, —S(O)₂R¹, —S(O)(NH)R¹, —S(O)(NR¹)R¹, optionally substituted C₁-C₆ aliphatic, optionally substituted C₁-C₆ heteroalkyl, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
each R^{c} is independently selected from the group consisting of hydrogen, optionally substituted C₁-C₆ aliphatic, OR¹, —NH₂, —NR¹R², optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R¹ is independently selected from the group consisting of optionally substituted C₁-C₆ aliphatic, optionally substituted phenyl, optionally substituted 3-6 membered heterocyclyl containing 1-4 heteroatoms each selected from the group consisting of N, O, and S, optionally substituted 5-6-membered heteroaryl containing 1-4 heteroatoms each selected from the group consisting of N, O and S, —C(O)R³, —CO₂R³, —C(O)NHR³, and —SO₂R³;
each R² is independently selected from the group consisting of hydrogen, optionally substituted C₁-C₆ aliphatic, optionally substituted 3-6 membered heterocyclyl containing 1-4 heteroatoms each selected from the group consisting of N, O, and S, optionally substituted phenyl, and optionally substituted 5-6-membered heteroaryl containing 1-4 heteroatoms each selected from the group consisting of N, O and S;
or R¹ and R² are taken together with their intervening atom(s) to form a 3-8-membered heterocyclyl ring containing 1-3 heteroatoms selected from the group consisting of N, O, and S, or an optionally substituted 5-6-membered heteroaryl ring containing 1-4 heteroatoms selected from the group consisting of N, O, and S.
each R³ is independently selected from the group consisting of optionally substituted C₁-C₆ aliphatic, optionally substituted 3-6 membered heterocyclyl containing 1-4 heteroatoms each selected from the group consisting of N, O, and S, optionally substituted phenyl, optionally substituted 5-6-membered heteroaryl containing 1-4 heteroatoms each selected from the group consisting of N, O and S;
n is 0, 1, 2, 3, 4, or 5;
m is 0, 1, 2, 3, or 4; and
p is 0, 1, 2, 3, or 4.

5. The method of any of the previous claims, wherein C is selected from the group consisting of optionally substituted triazolyl, optionally substituted pyrazolyl, optionally substituted isoxazolyl, optionally substituted thiazolyl, optionally substituted thiadizolyl, optionally substituted pyridinyl, optionally substituted pyrazinyl, optionally substituted pyrimidinyl, and optionally substituted pyridazinyl.

6. The method of any of the previous claims, wherein the compound is of Formula (B): 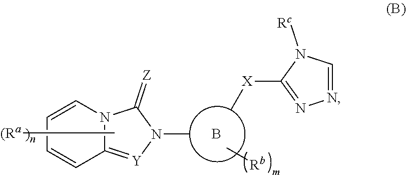
or pharmaceutically acceptable salts thereof.

7. The method of any of the previous claims, wherein the compound is of Formula (I): 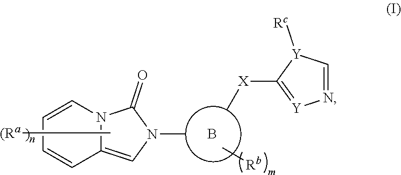
or pharmaceutically acceptable salts thereof.

8. The method of any of the previous claims, wherein the compound is of Formula (Ia) or (IIa): 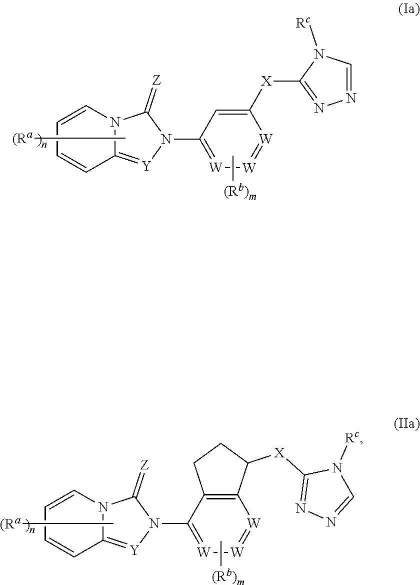
or a pharmaceutically acceptable salt thereof,
wherein each W is independently selected from N or C.

9. The method of any of the previous claims, wherein the compound is of formula (Ia1) or (IIa1): 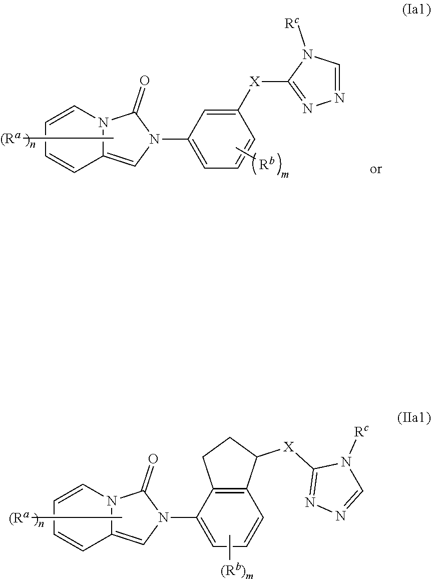
or a pharmaceutically acceptable salt thereof.

10. The method of any of the previous claims, wherein the compound is of Formula (Ia2), (Ia3), or (Ia4): 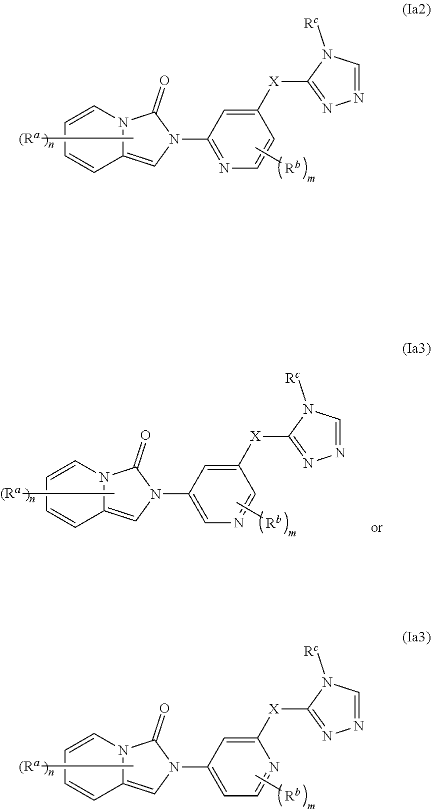
or a pharmaceutically acceptable salt thereof.

11. The method of any of the previous claims, wherein the compound is of formula (Ib) or (Ib): 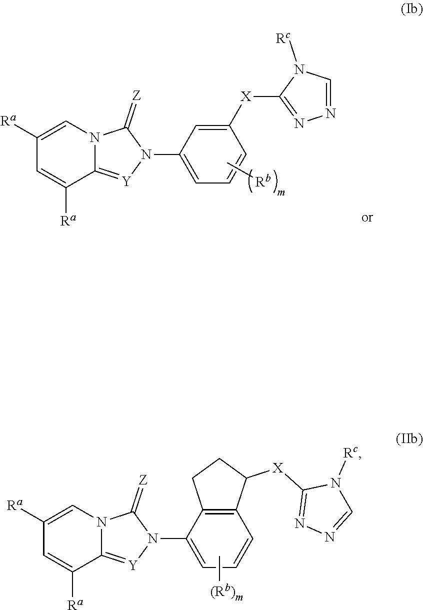
or pharmaceutically acceptable salts thereof,
wherein each W is independently selected from N or C.

12. The method of any of the previous claims, wherein the compound is of formula (Ic) or (IIc): 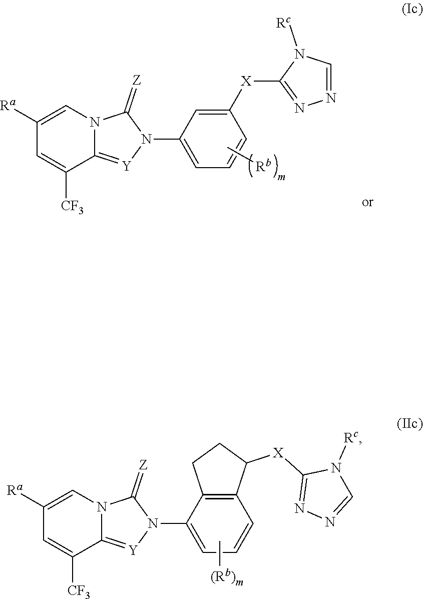
or pharmaceutically acceptable salts thereof.

13. The method of any of the previous claims, wherein R^{c} is optionally substituted C₁-C₃ aliphatic.

14. The method of any of the previous claims, wherein each R^{c} is independently selected from the group consisting of methyl, —CD₃, —CHF₂

15. The method of any of the previous claims, wherein R^{c} is methyl.

16. The method of any of the previous claims, wherein X is optionally substituted C₁-C₂ alkylene.

17. The method of any of the previous claims, wherein X is 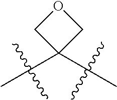 [SMILES: *C1(*)COC1]
or optionally substituted C₂ alkylene, wherein one methylene unit is replaced with

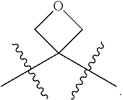 [SMILES: *C1(*)COC1]

18. The method of any of the previous claims, wherein X is selected from the group consisting of

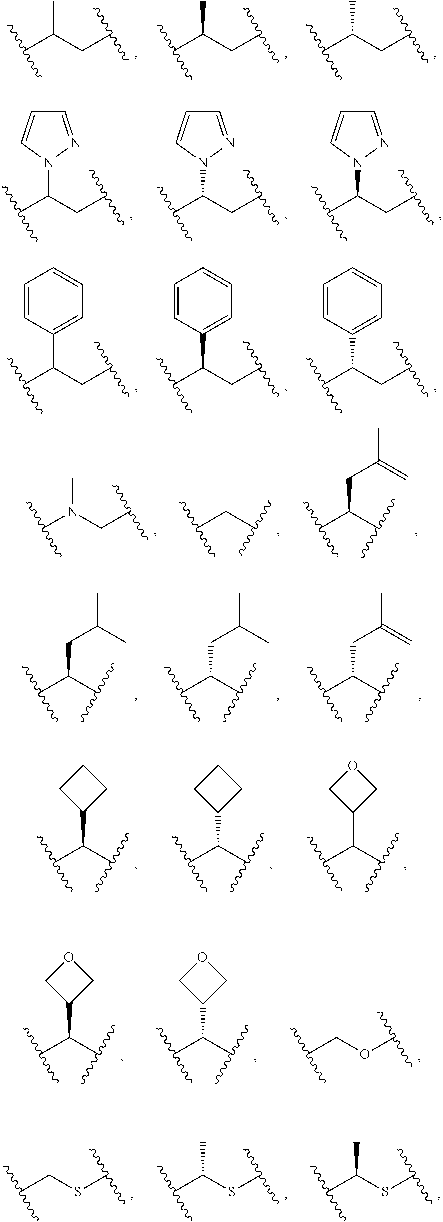

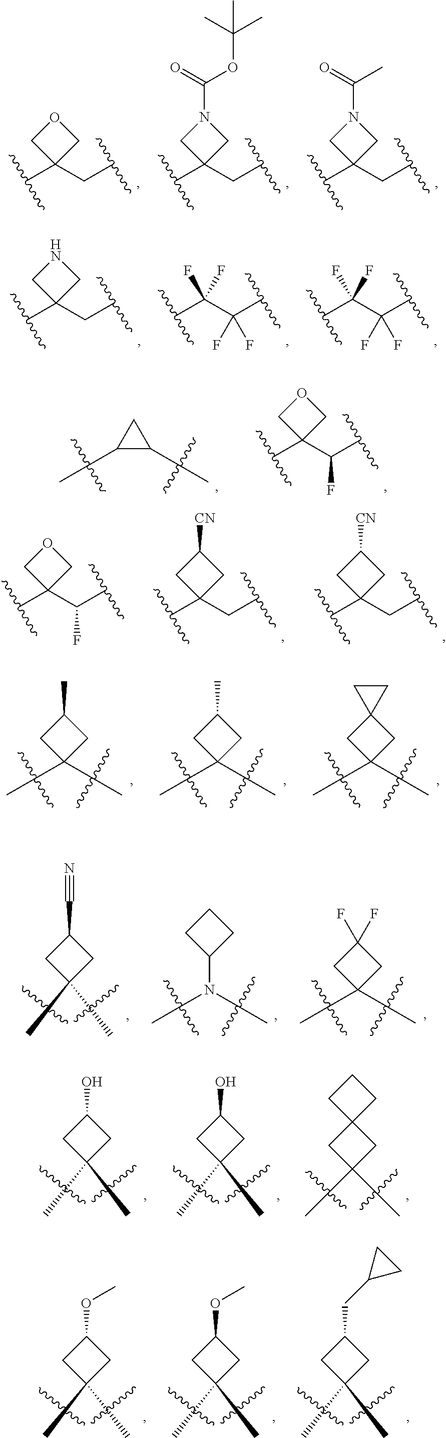

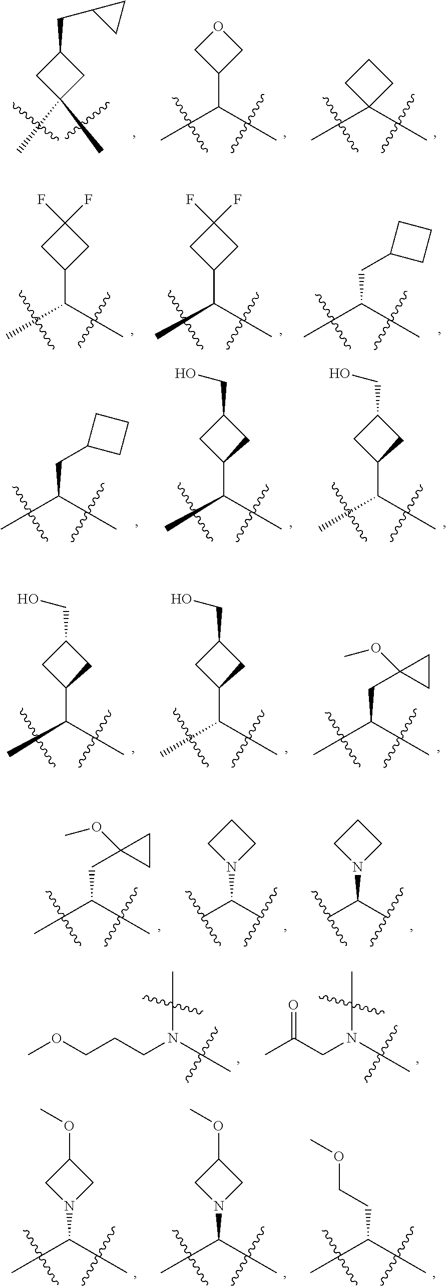

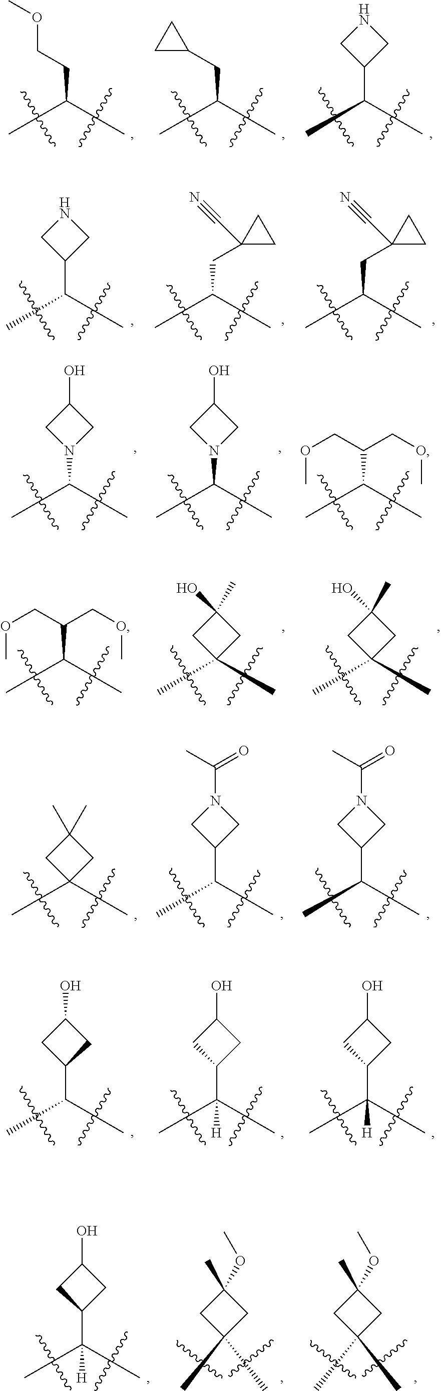

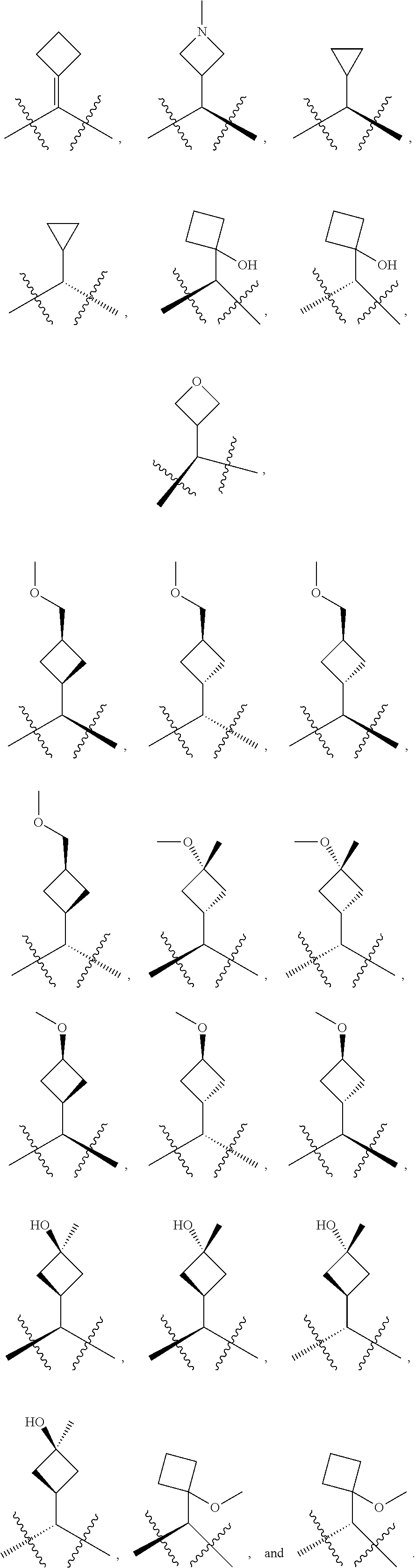

19. The method of any of the previous claims, wherein R^{a} is L-A.

20. The method of any of the previous claims, wherein L is —CH₂— or —CH(CH₃)—.

21. The method of any of the previous claims, wherein A is optionally substituted 3-6 membered heterocyclyl containing 1-4 heteroatoms each selected from the group consisting of N, O, and S.

22. The method of any of the previous claims, wherein R^{a} is selected from halogen, —CN, —C(O)R¹, —CO₂H, —CONR¹R², optionally substituted C₁-C₆ aliphatic, and optionally substituted C₁-C₆ heteroalkyl.

23. The method of any of the previous claims, wherein each R^{a} is independently selected from the group consisting of halogen, —CN, —CO₂H, —CHO, —CHF₂, —CF₃, —OMe, —S(O)₂NHMe,

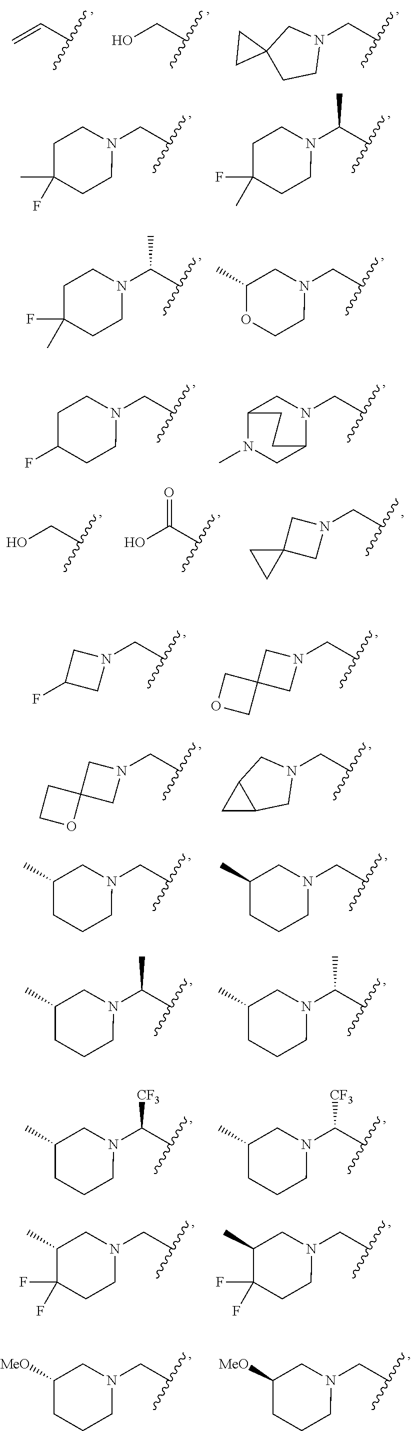

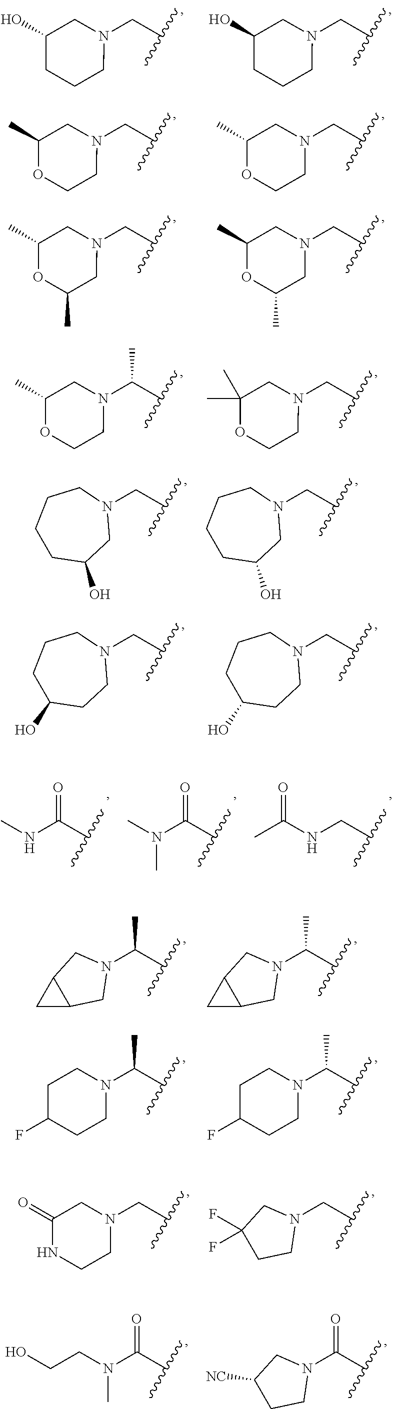

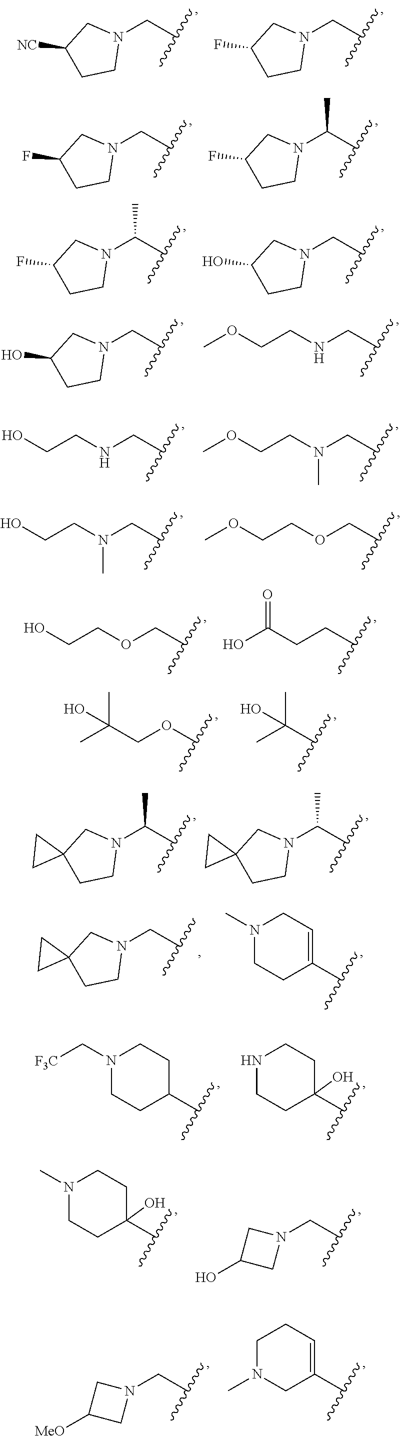

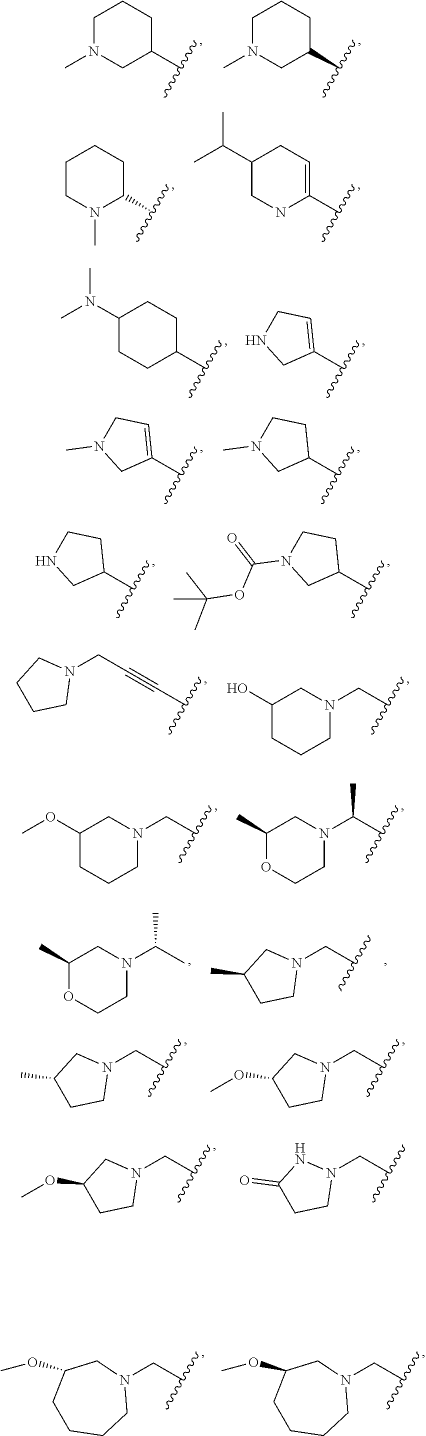

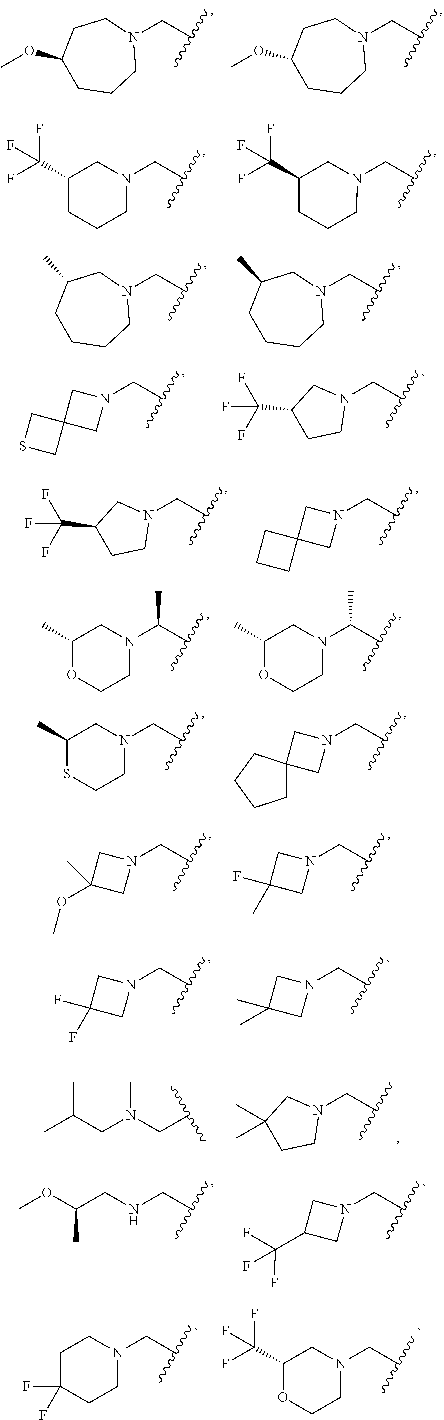

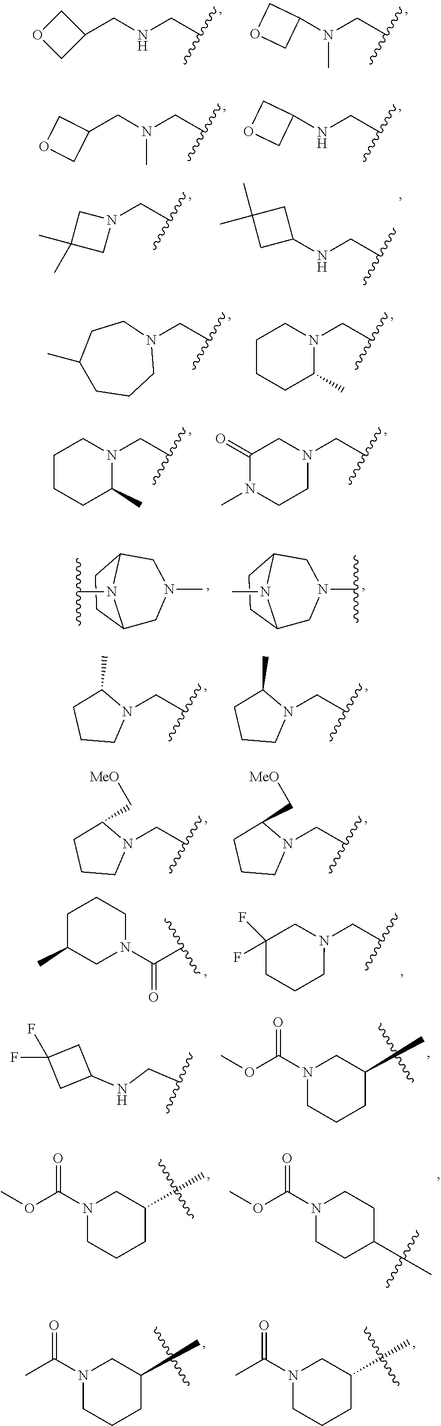

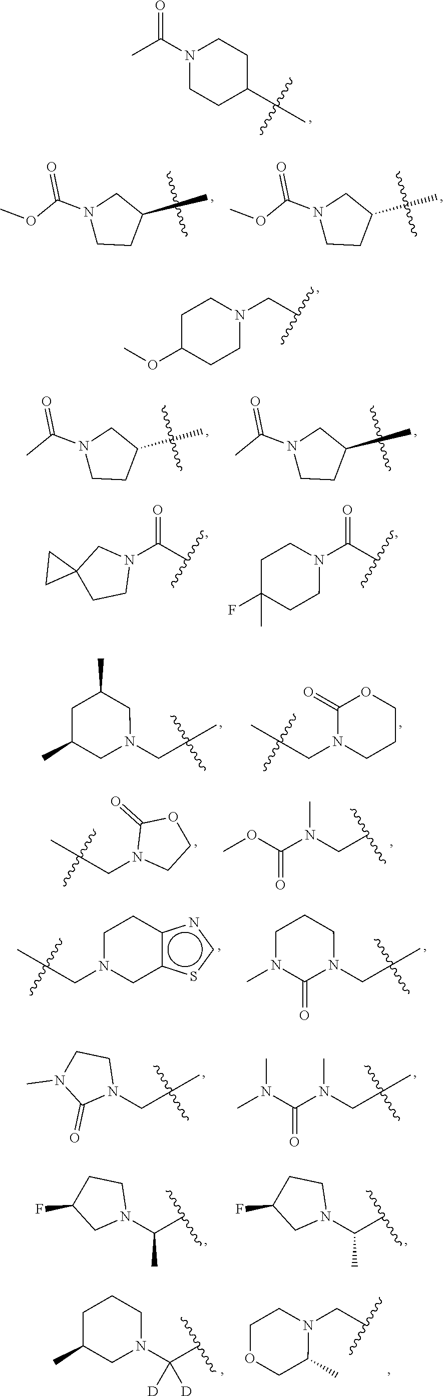

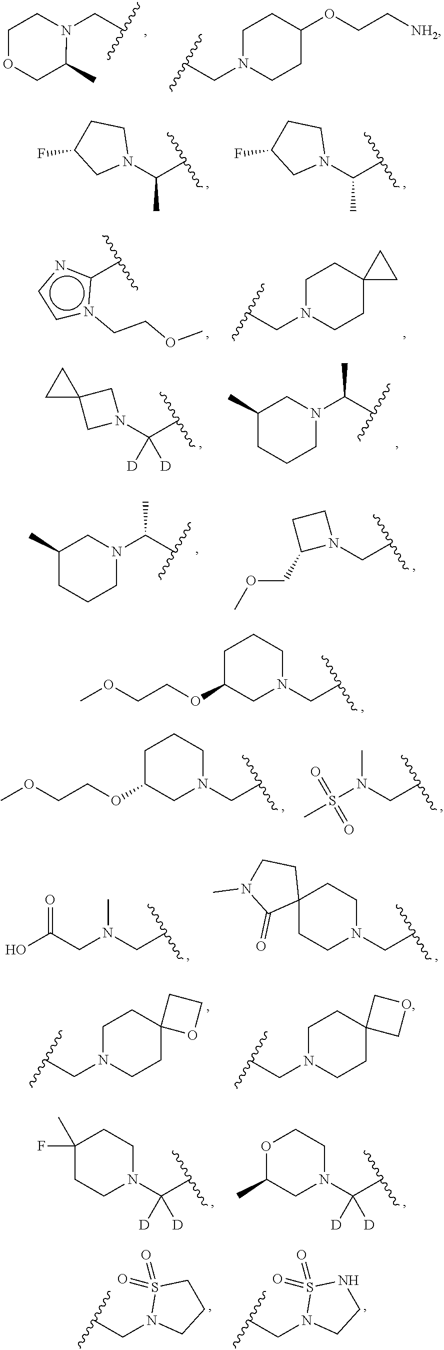

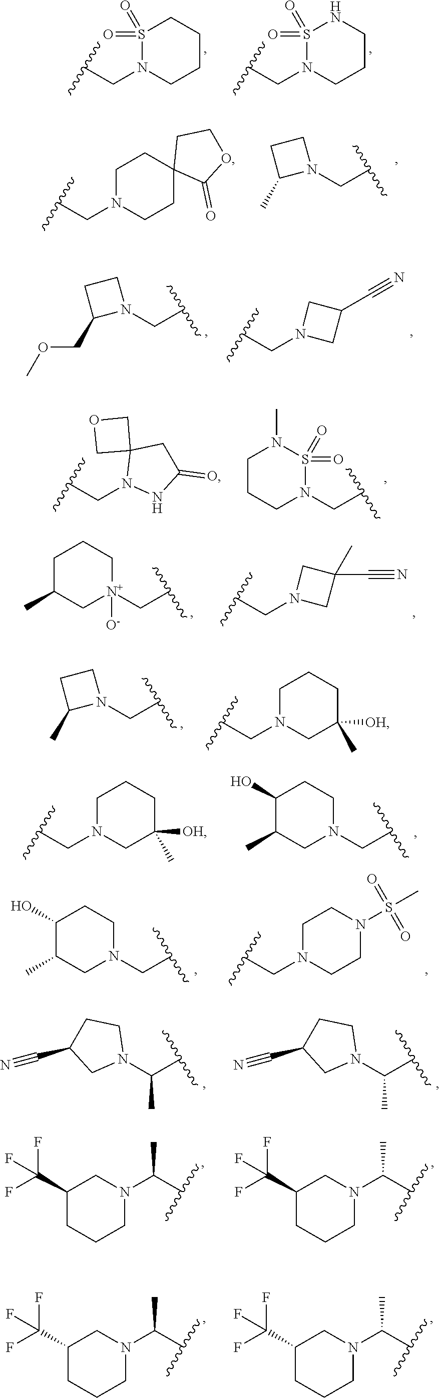

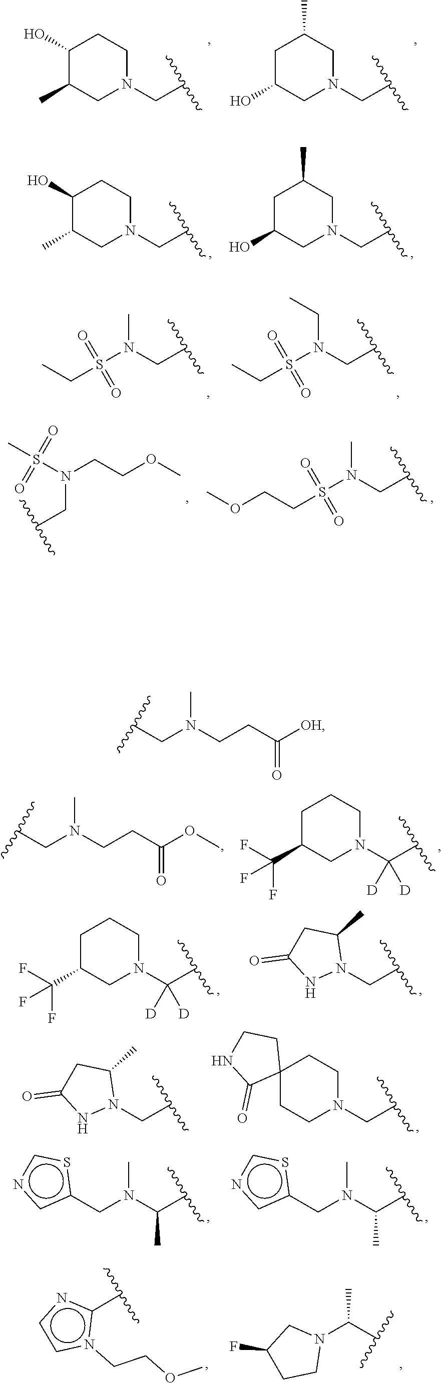

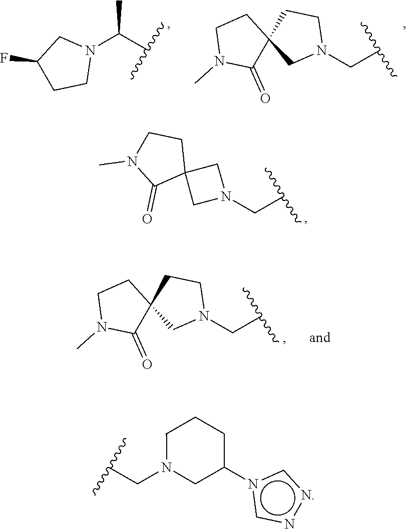

24. The method of any of the previous claims, wherein the compound is selected from the group consisting of

| Cmpd | |
|---|---|
| No. | Structure |
| 1 | 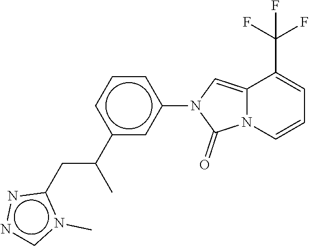 |
| 2 | 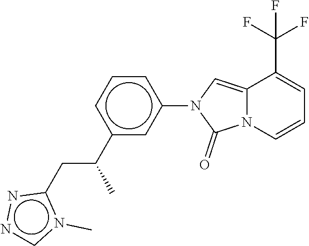 |
| 3 | 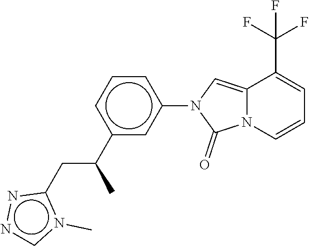 |
| 4 | 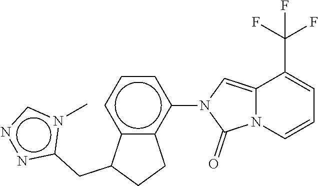 |
| 5 | 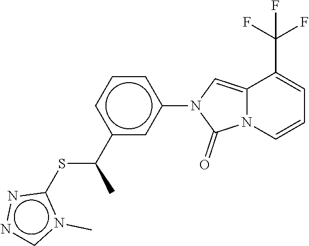 |
| 6 | 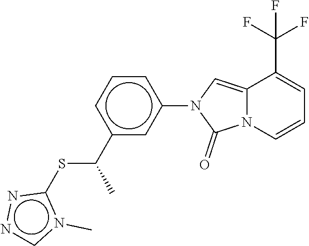 |
| 7 | 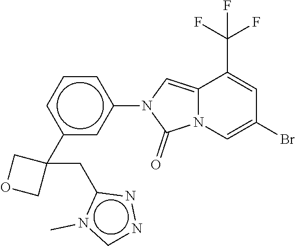 |
| 8 | 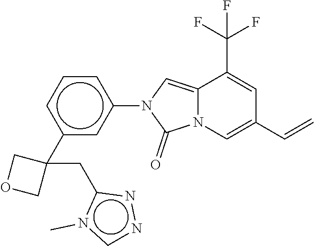 |
| 9 | 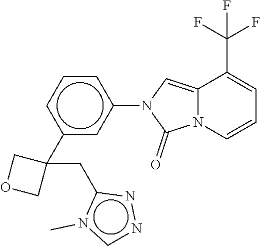 |
| 10 | 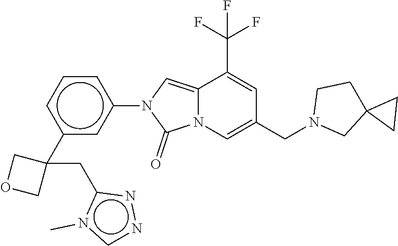 |
| 11 | 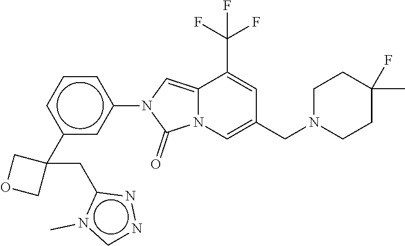 |
| 12 | 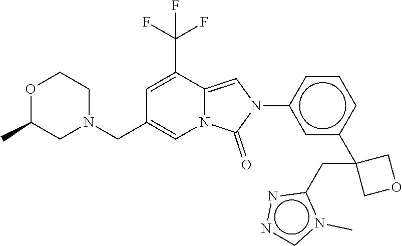 |
| 13 | 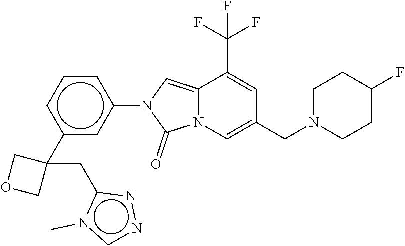 |
| 14 | 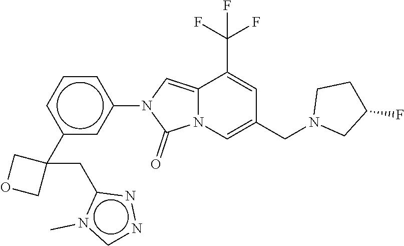 |
| 15 | 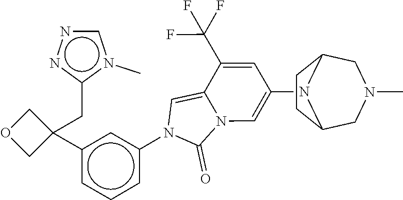 |
| 16 | 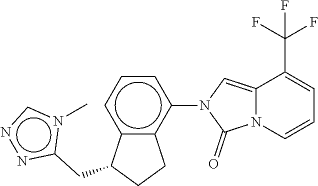 |
| 17 | 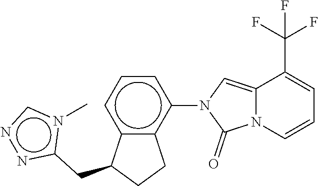 |
| 18 | 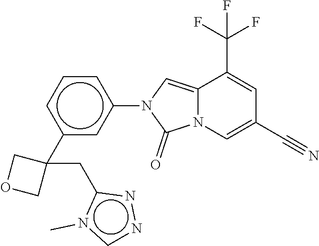 |
| 19 | 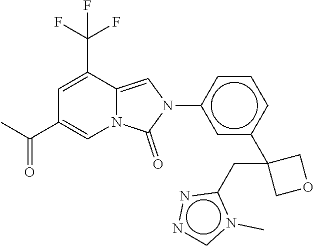 |
| 20 | 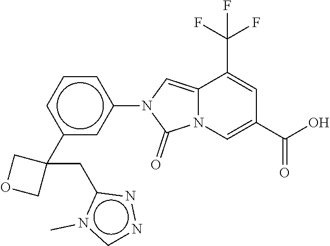 |
| 21 | 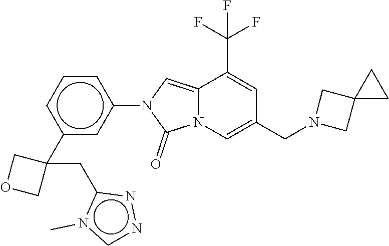 |
| 22 | 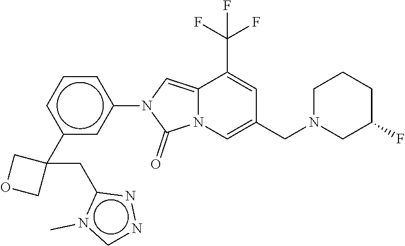 |
| 23 | 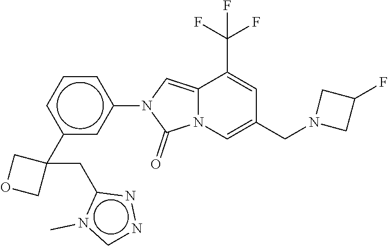 |
| 24 | 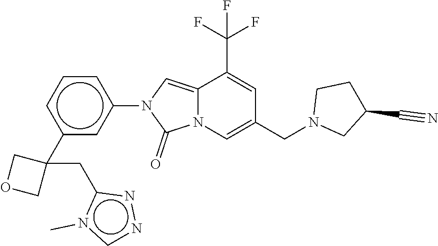 |
| 25 | 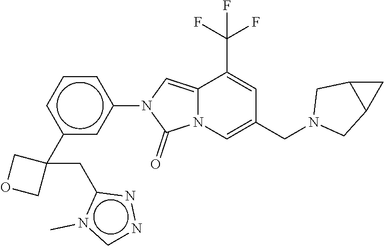 |
| 26 | 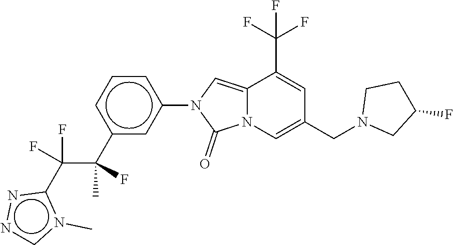 |
| 27 | 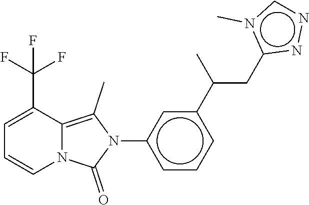 |
| 28 | 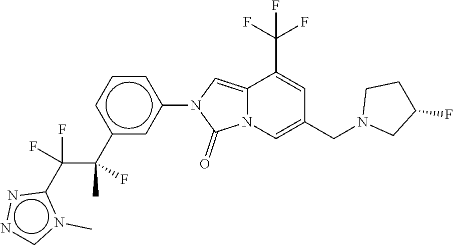 |
| 29 | 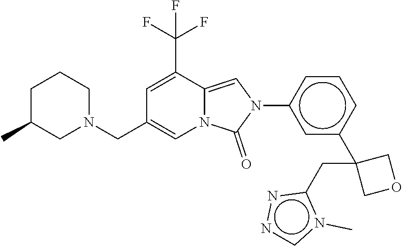 |
| 30 | 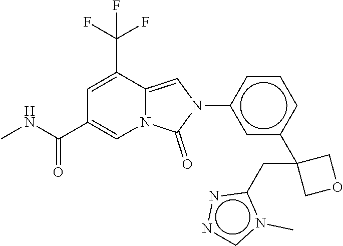 |
| 31 | 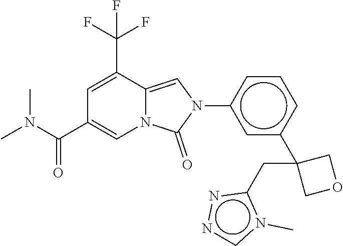 |
| 32 | 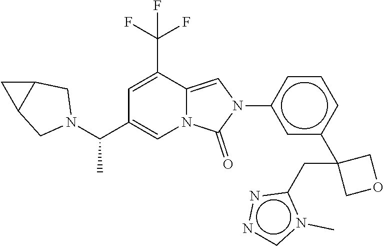 |
| 33 | 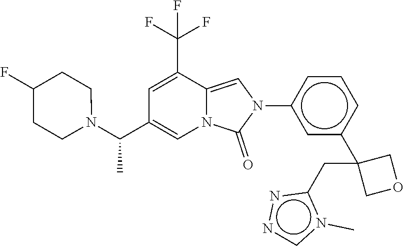 |
| 34 | 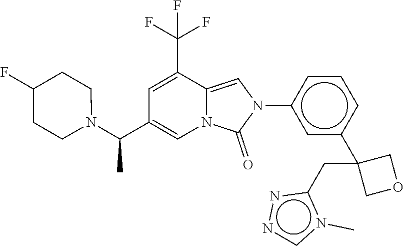 |
| 35 | 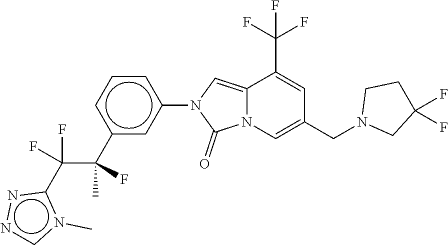 |
| 36 | 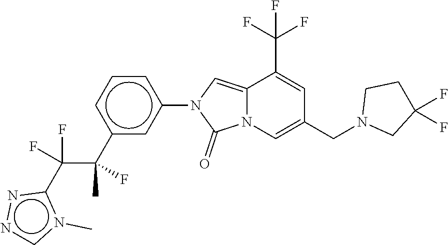 |
| 37 | 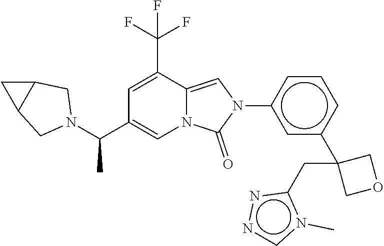 |
| 38 | 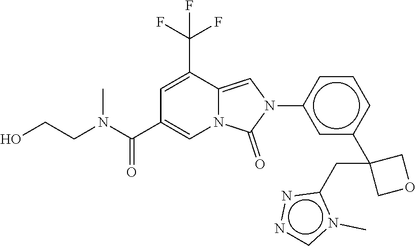 |
| 39 | 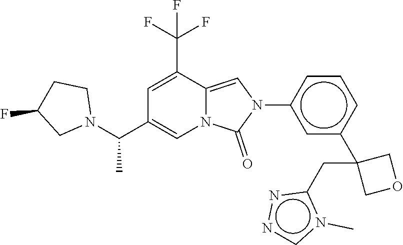 |
| 40 | 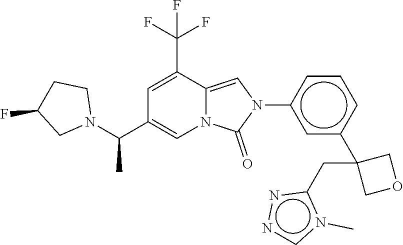 |
| 41 | 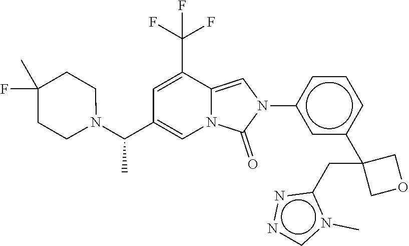 |
| 42 | 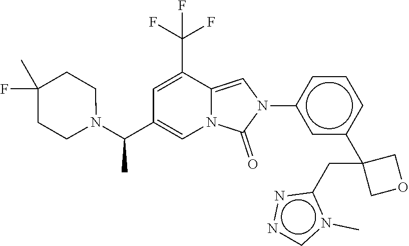 |
| 43 | 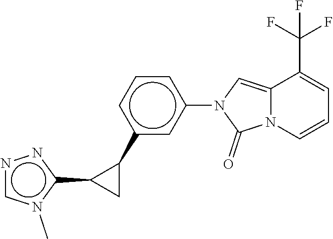 |
| 44 | 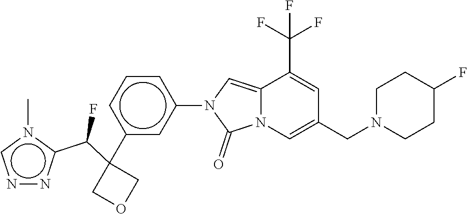 |
| 45 | 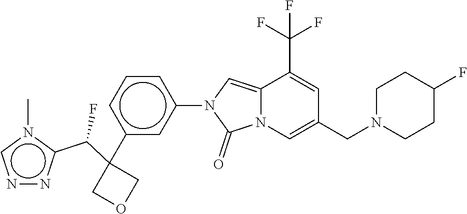 |
| 46 | 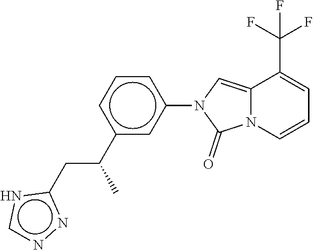 |
| 47 | 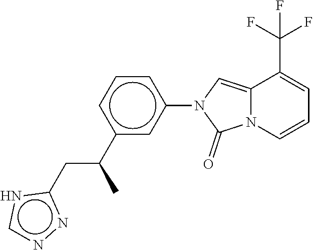 |
| 48 | 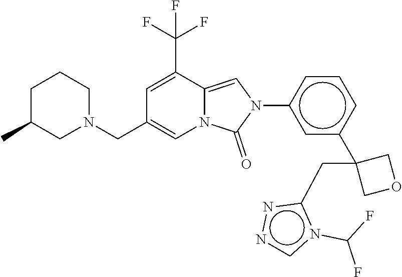 |
| 49 | 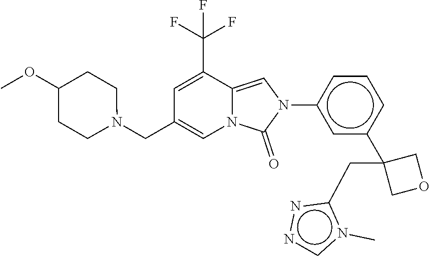 |
| 50 | 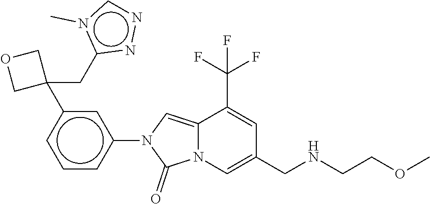 |
| 51 | 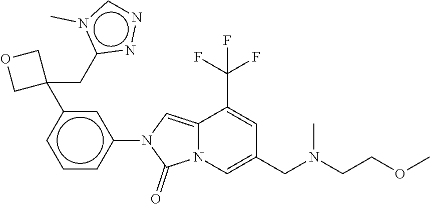 |
| 52 | 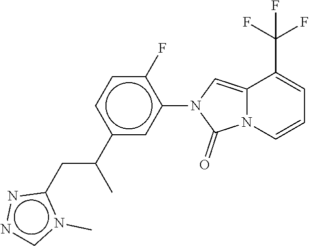 |
| 53 | 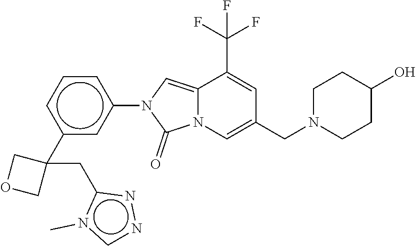 |
| 54 | 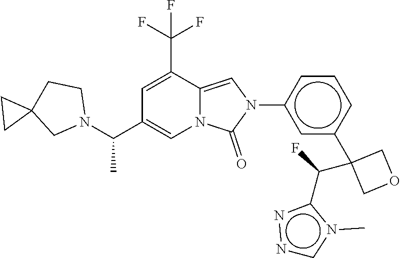 |
| 55 | 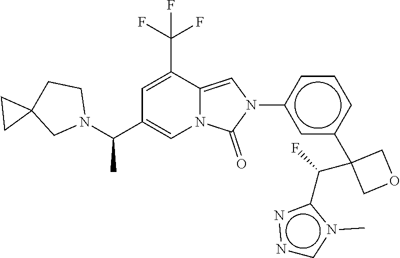 |
| 56 | 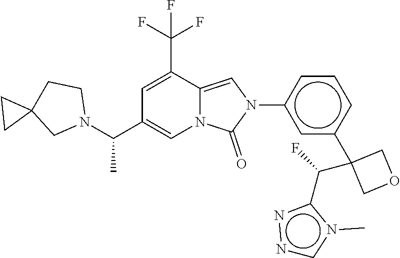 |
| 57 | 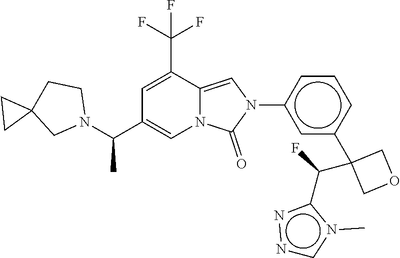 |
| 58 | 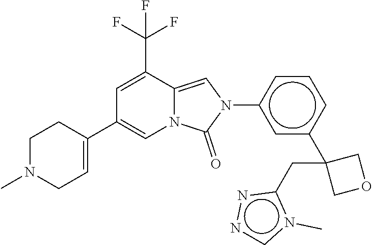 |
| 59 | 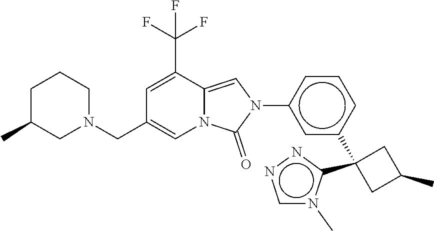 |
| 60 | 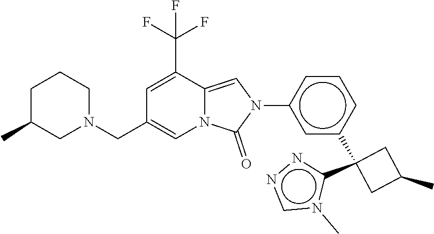 |
| 61 | 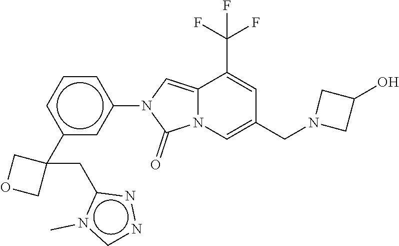 |
| 62 | 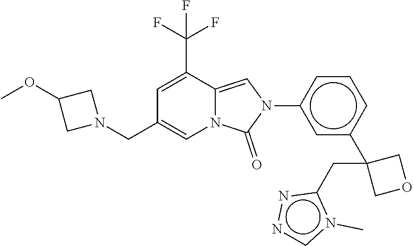 |
| 63 | 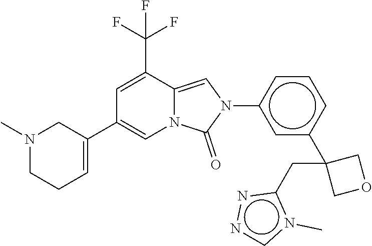 |
| 64 | 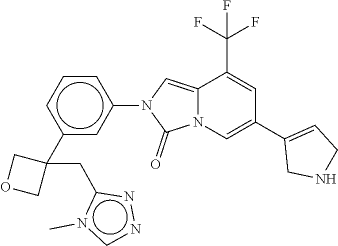 |
| 65 | 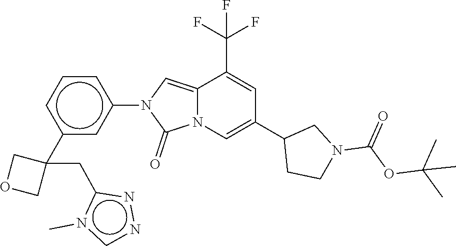 |
| 66 | 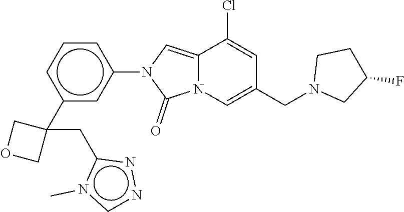 |
| 67 | 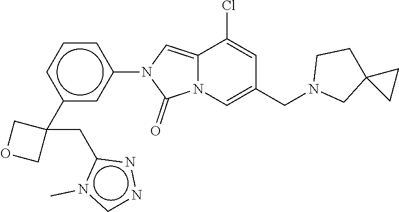 |
| 68 | 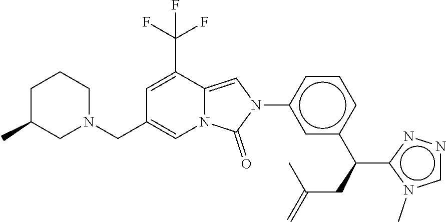 |
| 69 | 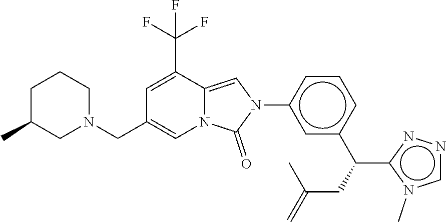 |
| 70 | 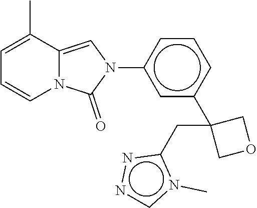 |
| 71 | 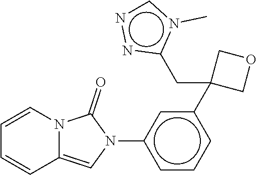 |
| 72 | 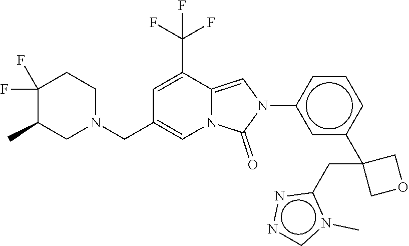 |
| 73 | 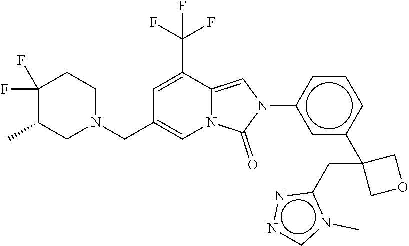 |
| 74 | 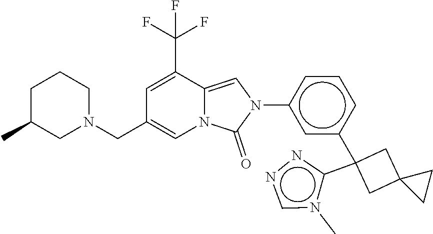 |
| 75 | 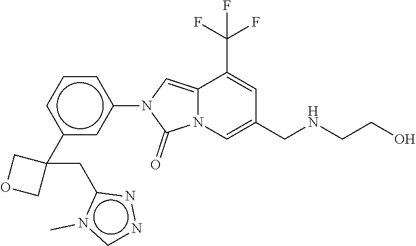 |
| 76 | 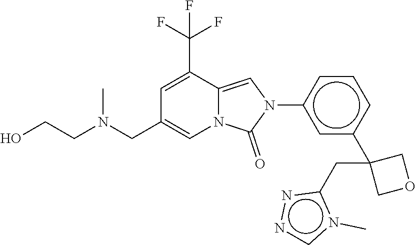 |
| 77 | 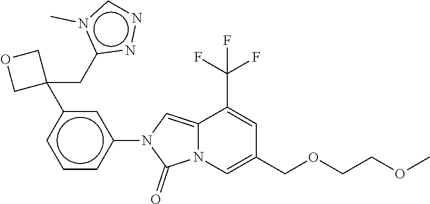 |
| 78 | 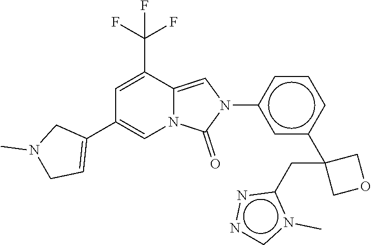 |
| 79 | 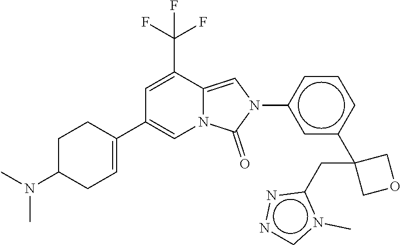 |
| 80 | 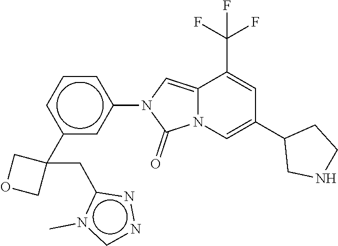 |
| 81 | 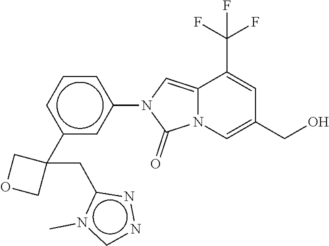 |
| 82 | 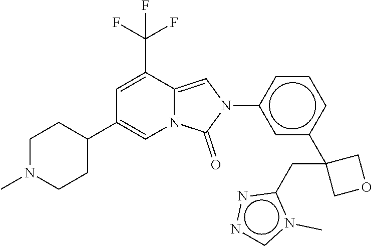 |
| 83 | 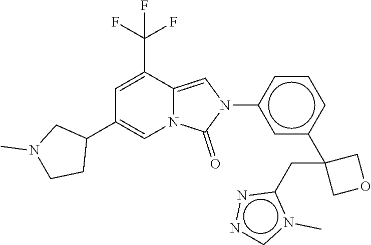 |
| 84 | 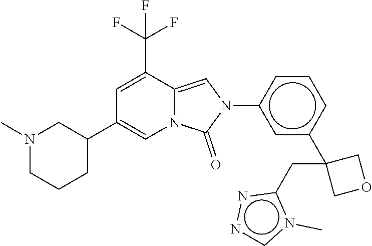 |
| 85 | 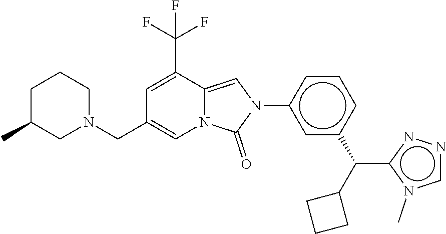 |
| 86 | 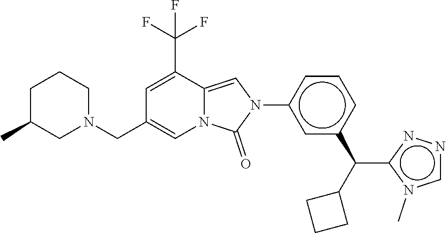 |
| 87 | 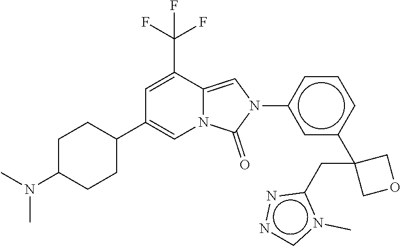 |
| 88 | 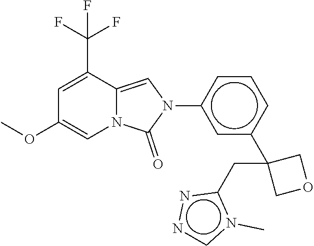 |
| 89 | 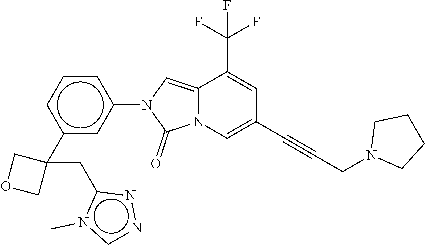 |
| 90 | 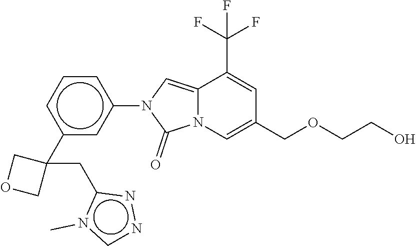 |
| 91 | 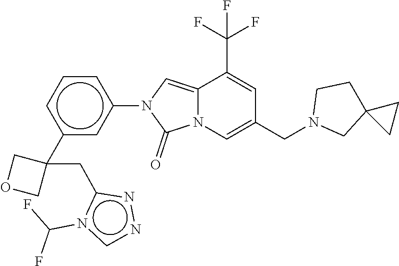 |
| 92 | 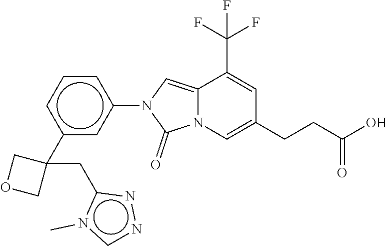 |
| 93 | 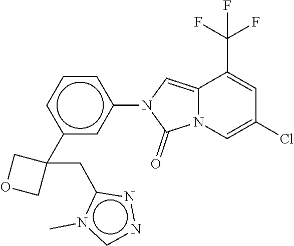 |
| 94 | 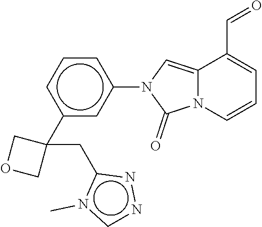 |
| 95 | 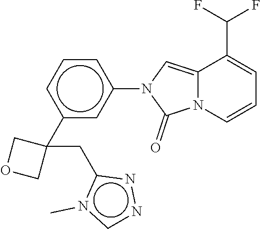 |
| 96 | 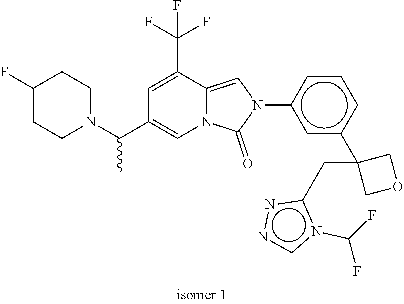 |
| 97 | 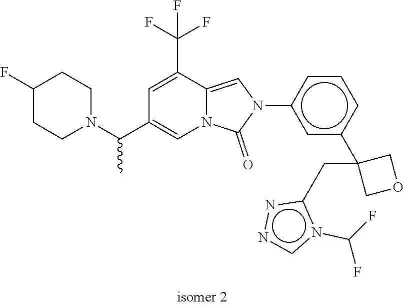 |
| 98 | 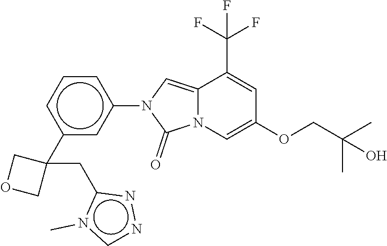 |
| 99 | 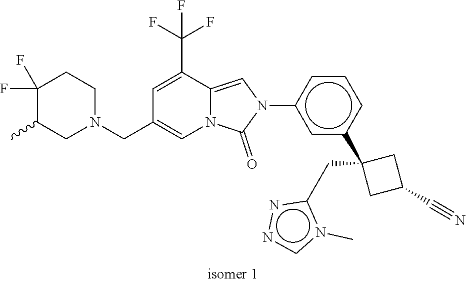 |
| 100 | 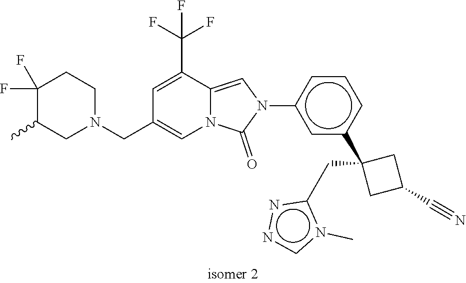 |
| 101 | 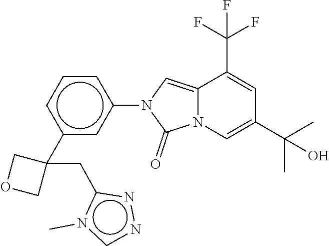 |
| 102 | 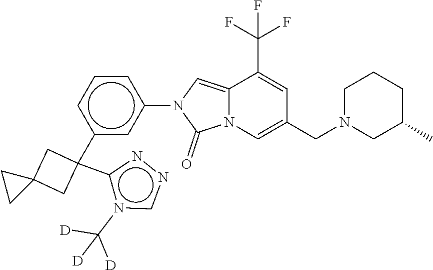 |
| 103 | 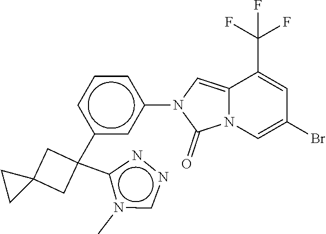 |
| 104 | 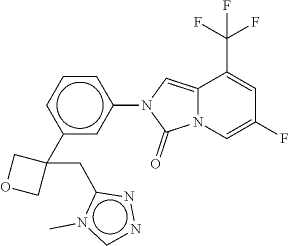 |
| 105 | 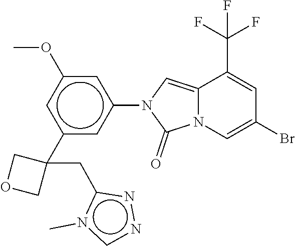 |
| 106 | 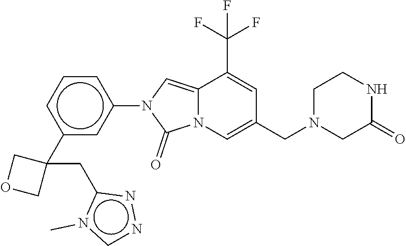 |
| 107 | 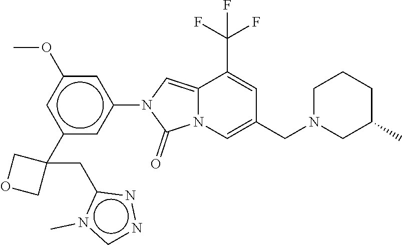 |
| 108 | 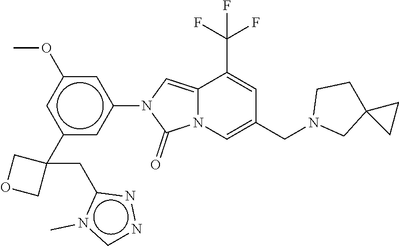 |
| 109 | 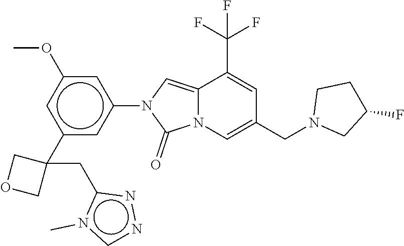 |
| 110 | 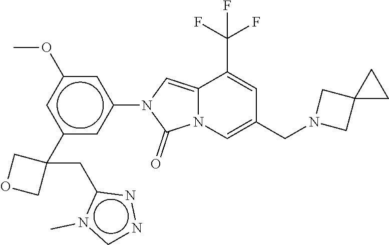 |
| 111 | 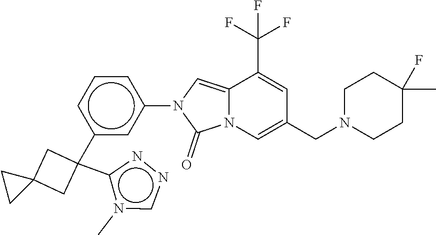 |
| 112 | 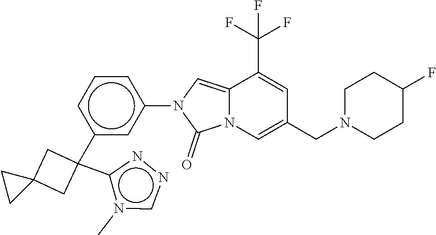 |
| 113 | 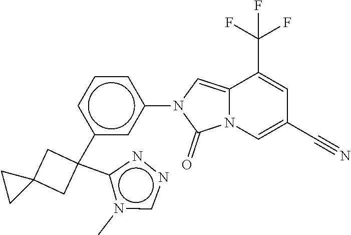 |
| 114 | 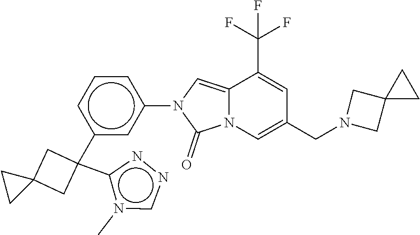 |
| 115 | 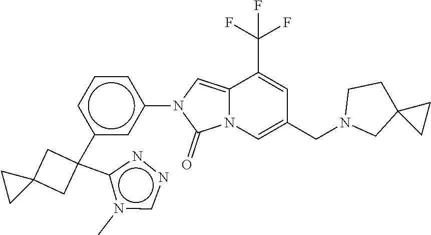 |
| 116 | 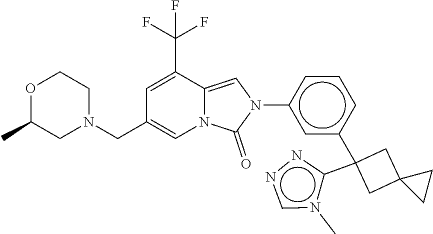 |
| 117 | 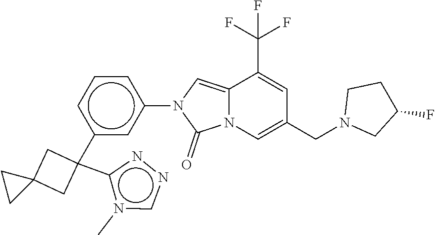 |
| 118 | 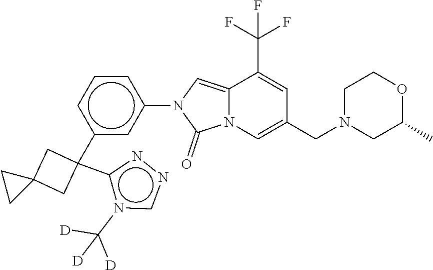 |
| 119 | 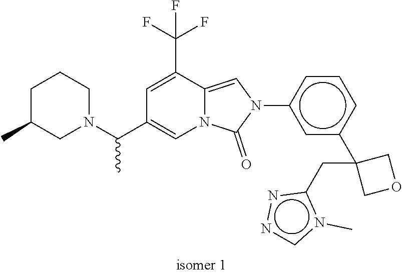 |
| 120 | 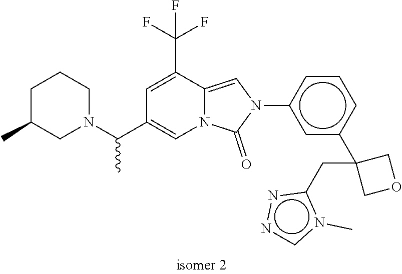 |
| 121 | 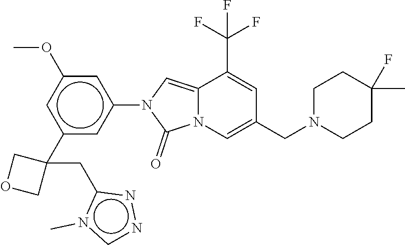 |
| 122 | 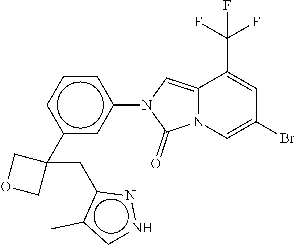 |
| 123 | 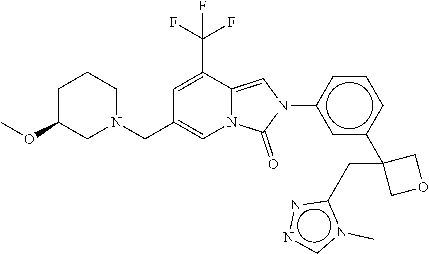 |
| 124 | 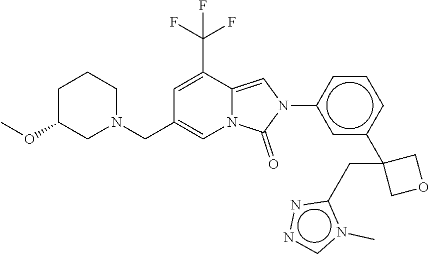 |
| 125 | 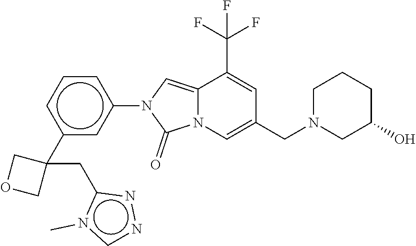 |
| 126 | 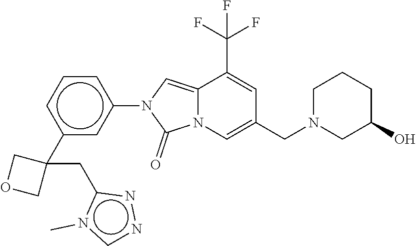 |
| 127 | 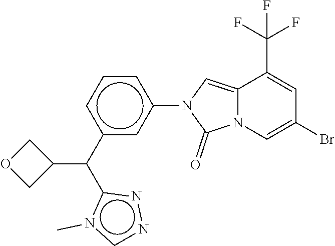 |
| 128 | 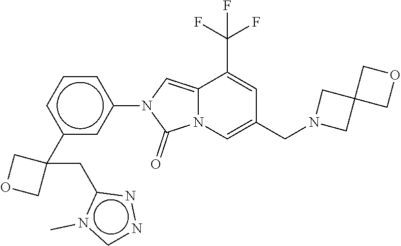 |
| 129 | 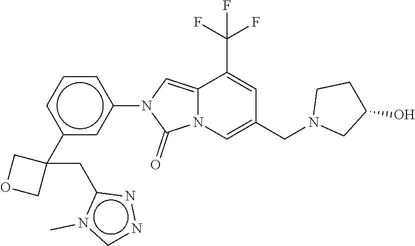 |
| 130 | 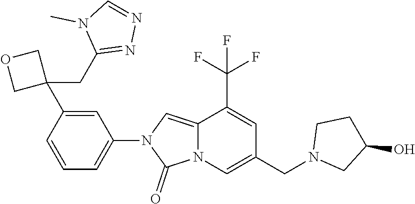 [SMILES: Cn1cnnc1CC1(c2cccc(-n3cc4c(C(F)(F)F)cc(CN5CC[C@@H](O)C5)cn4c3=O)c2)COC1] |
| 131 | 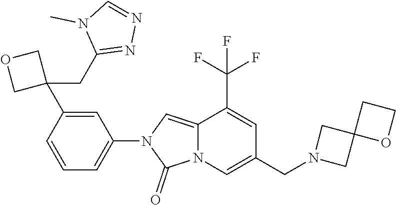 [SMILES: Cn1cnnc1CC1(c2cccc(-n3cc4c(C(F)(F)F)cc(CN5CC6(CCO6)C5)cn4c3=O)c2)COC1] |
| 132 | 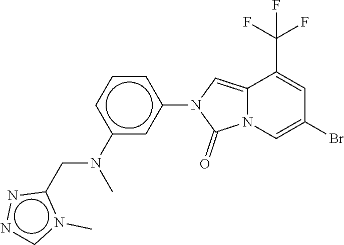 |
| 133 | 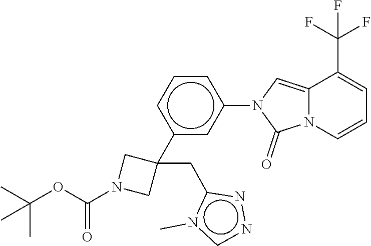 |
| 134 | 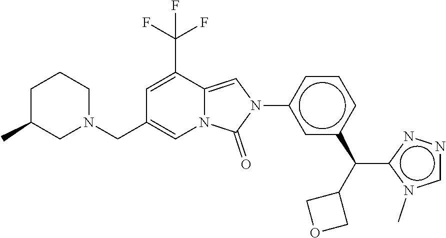 |
| 135 | 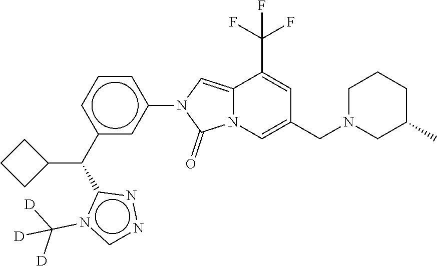 |
| 136 | 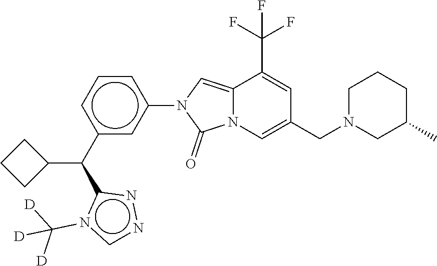 |
| 137 | 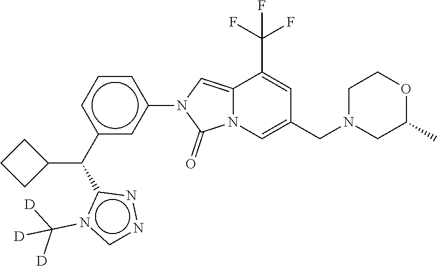 |
| 138 | 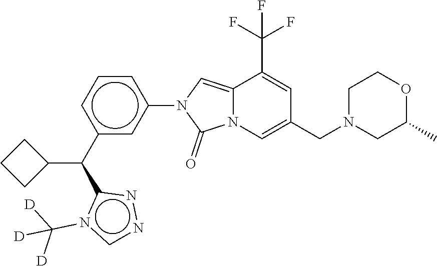 |
| 139 | 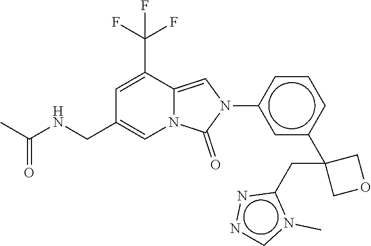 |
| 140 | 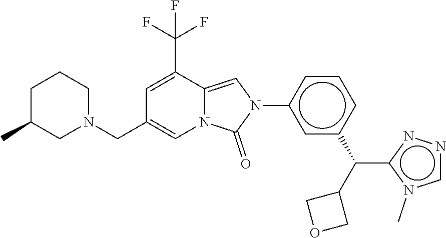 |
| 141 | 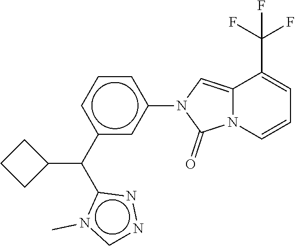 |
| 142 | 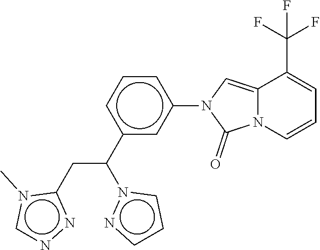 |
| 143 | 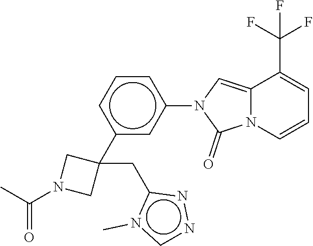 |
| 144 | 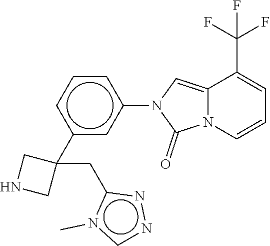 |
| 145 | 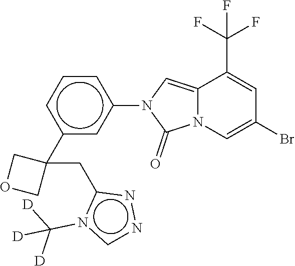 |
| 146 | 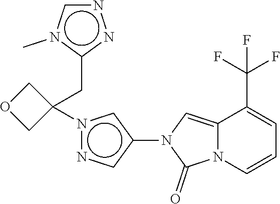 |
| 147 | 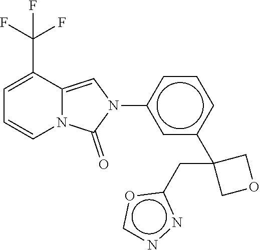 |
| 148 | 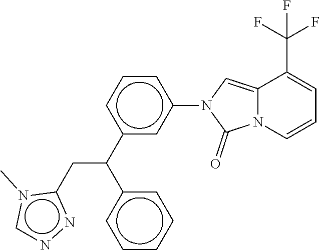 |
| 149 | 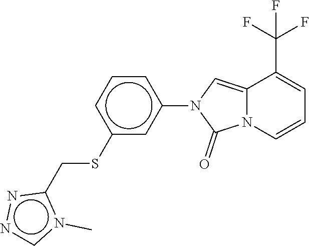 |
| 150 | 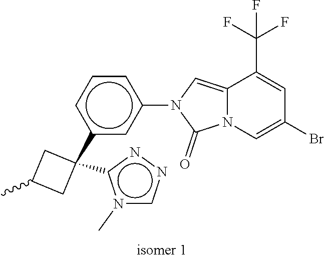 |
| 151 | 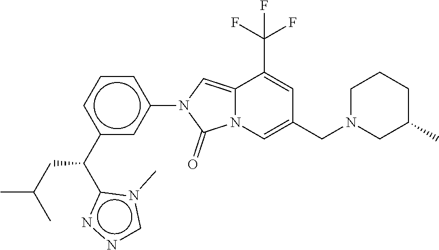 |
| 152 | 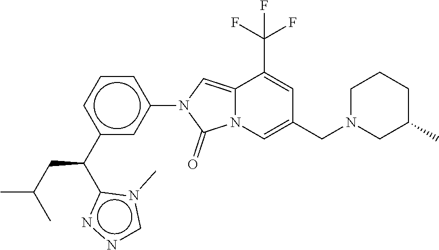 |
| 153 | 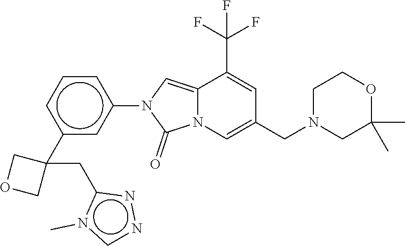 |
| 154 | 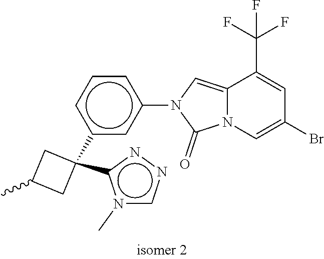 |
| 155 | 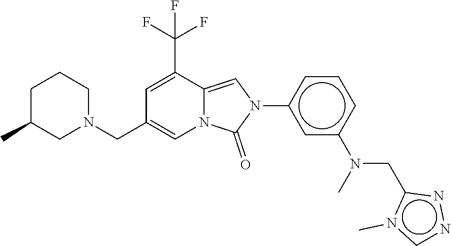 |
| 156 | 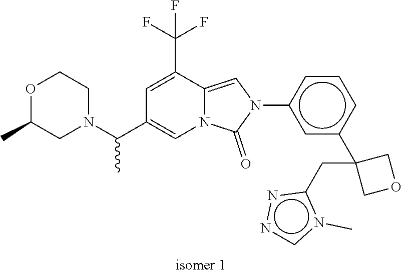 |
| 157 | 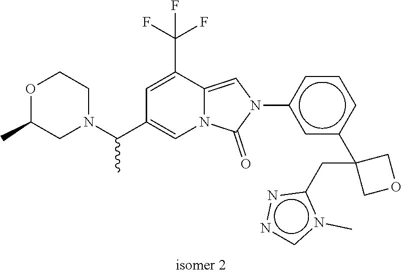 |
| 158 | 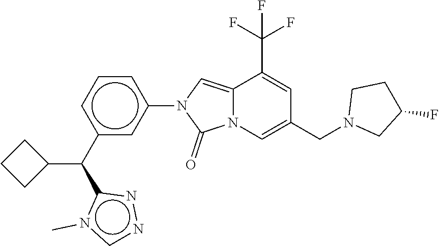 |
| 159 | 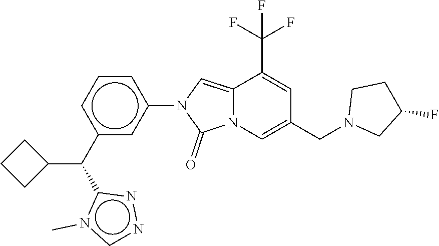 |
| 160 | 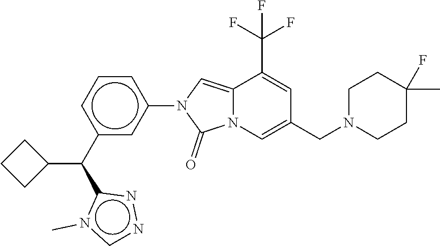 |
| 161 | 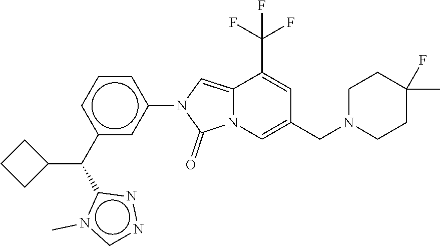 |
| 162 | 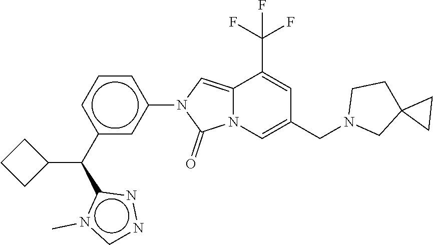 |
| 163 | 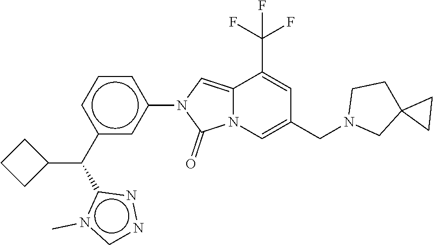 |
| 164 | 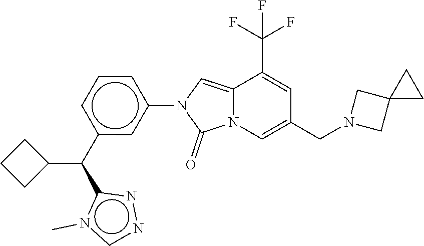 |
| 165 | 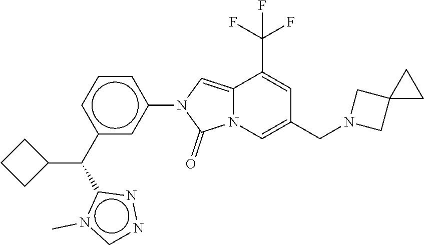 |
| 166 | 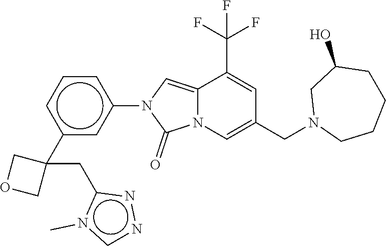 |
| 167 | 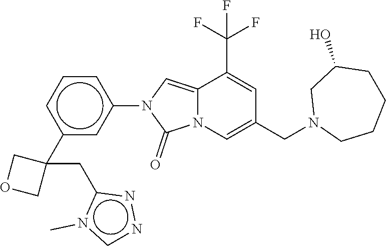 |
| 168 | 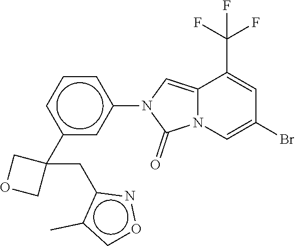 |
| 169 | 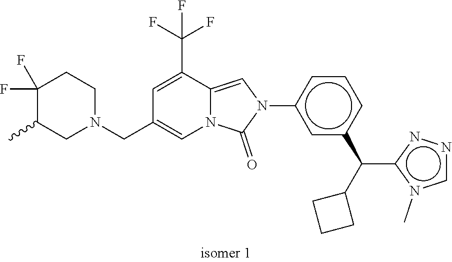 |
| 170 | 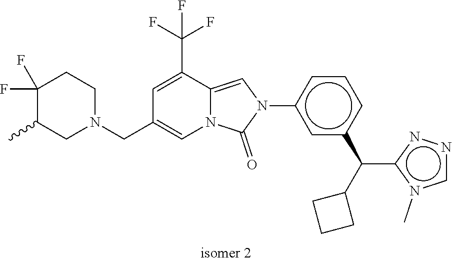 |
| 171 | 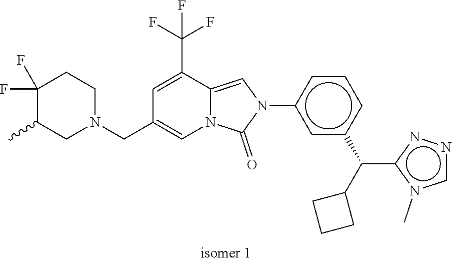 |
| 172 | 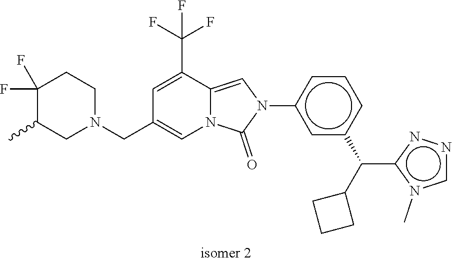 |
| 173 | 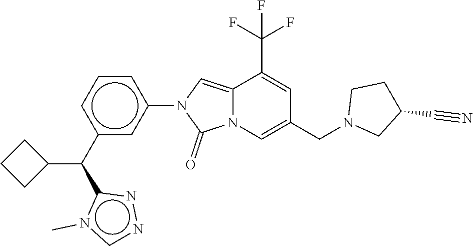 |
| 174 | 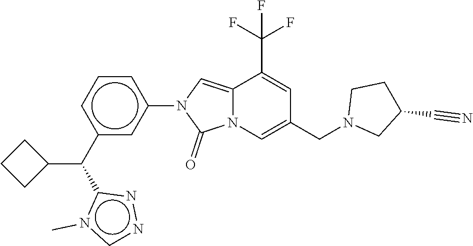 |
| 175 | 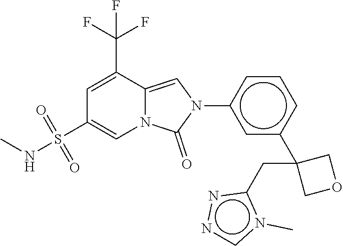 |
| 176 | 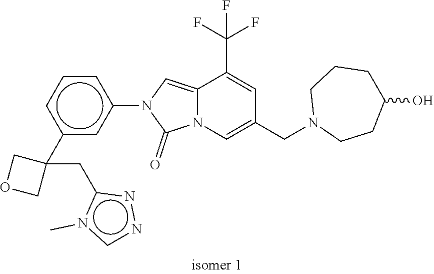 |
| 177 | 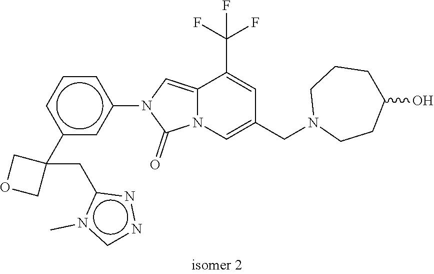 |
| 178 | 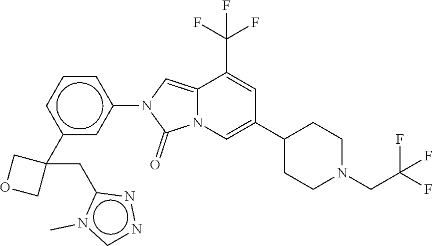 |
| 179 | 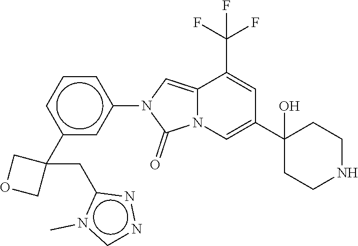 |
| 180 | 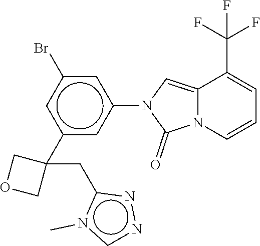 |
| 181 | 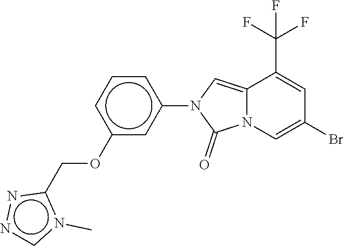 |
| 182 | 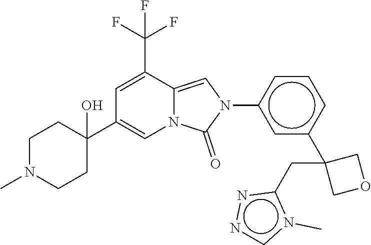 |
| 183 | 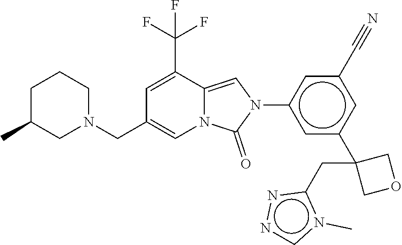 |
| 184 | 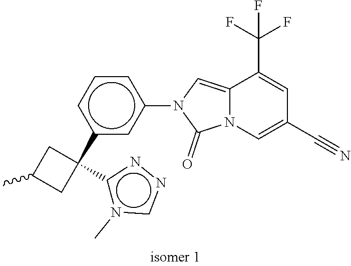 |
| 185 | 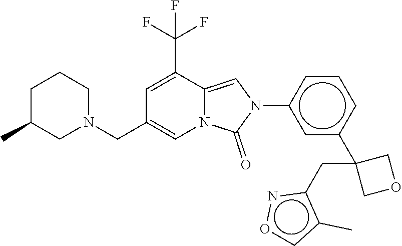 |
| 186 | 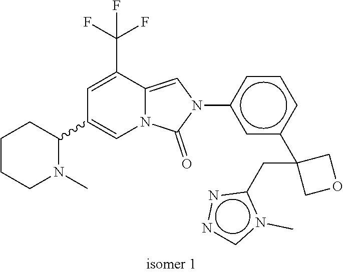 |
| 187 | 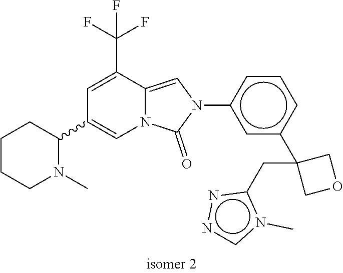 |
| 188 | 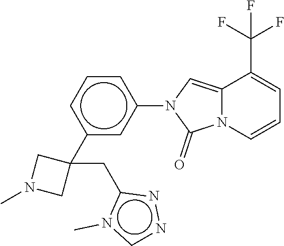 |
| 189 | 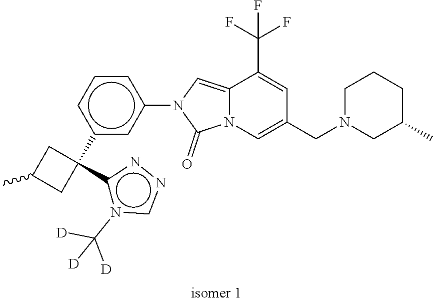 |
| 190 | 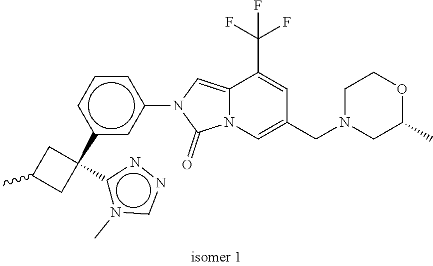 |
| 191 | 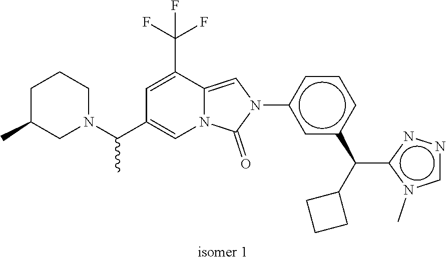 |
| 192 | 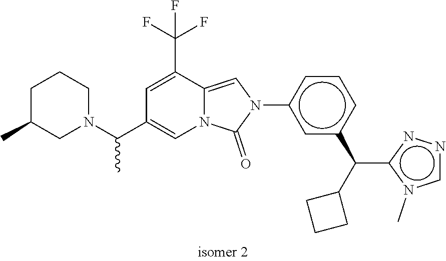 |
| 193 | 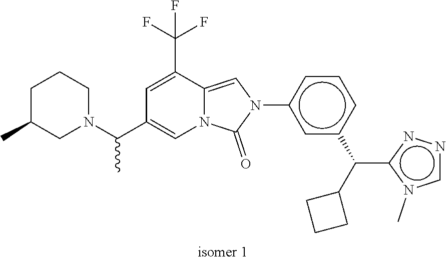 |
| 194 | 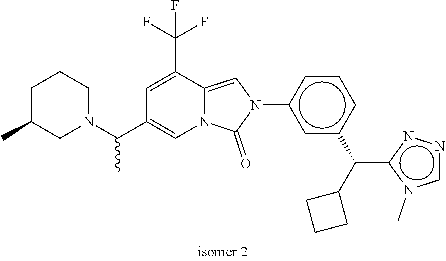 |
| 195 | 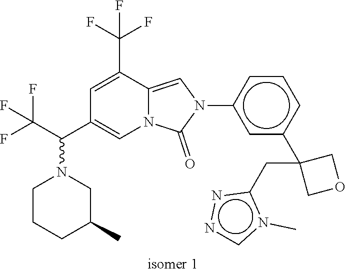 |
| 196 | 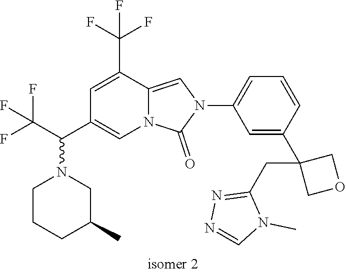 [SMILES: C[C@H]1CCCN(C(c2cc(C(F)(F)F)c3cn(-c4cccc(C5(Cc6nncn6C)COC5)c4)c(=O)n3c2)C(F)(F)F)C1] |
| 197 | 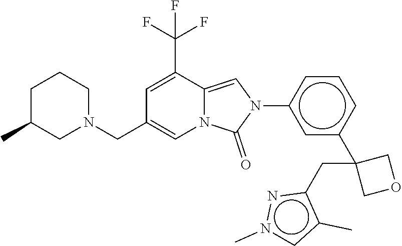 |
| 198 | 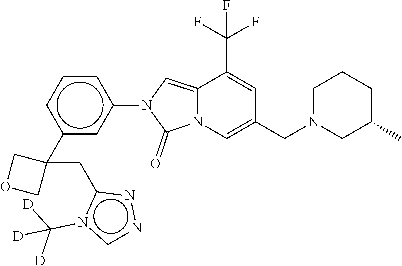 |
| 199 | 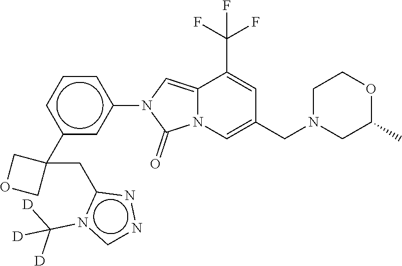 |
| 200 | 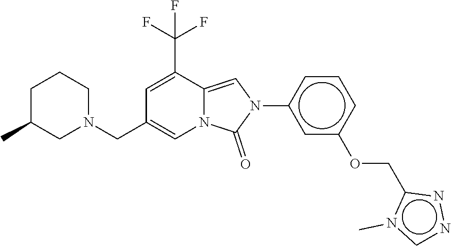 |
| 201 | 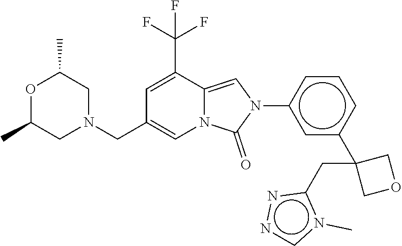 |
| 202 | 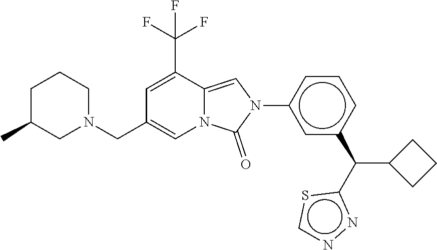 |
| 203 | 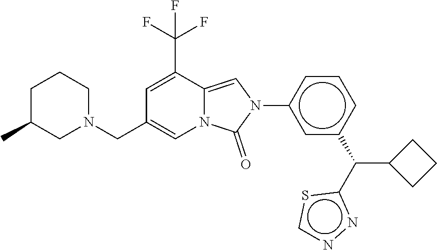 |
| 204 | 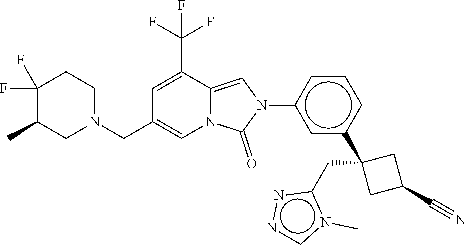 |
| 205 | 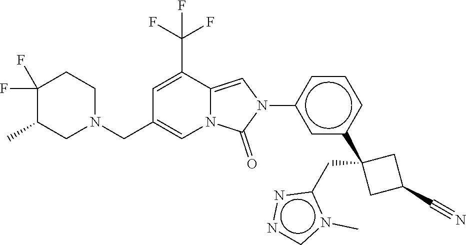 |
| 206 | 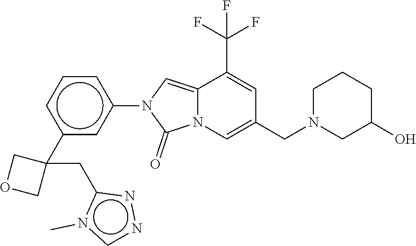 |
| 207 | 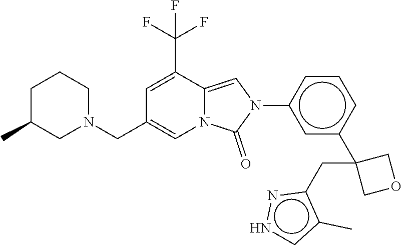 |
| 208 | 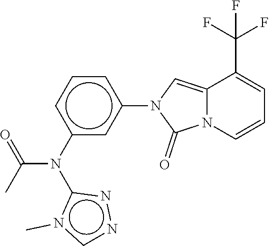 |
| 209 | 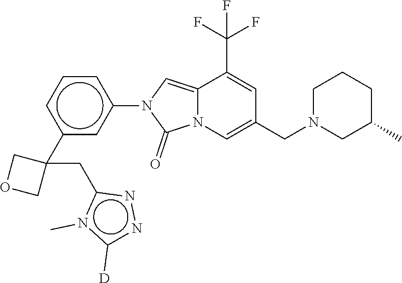 |
| 210 | 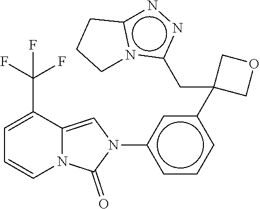 |
| 211 | 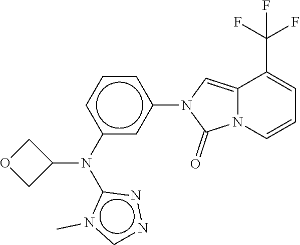 |
| 212 | 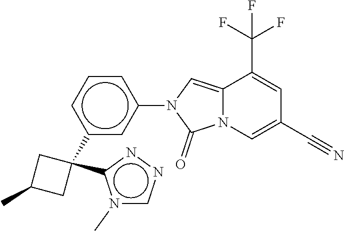 |
| 213 | 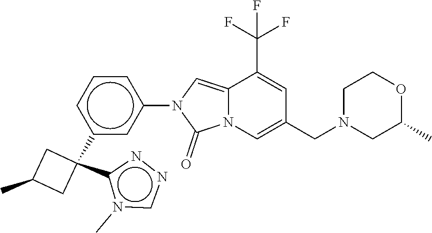 |
| 214 | 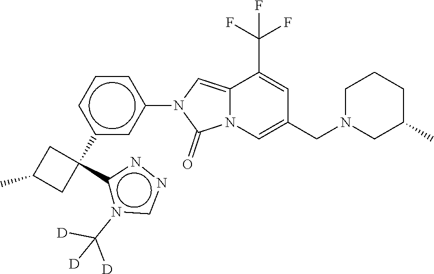 |
| 215 | 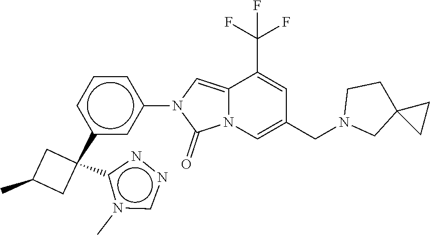 |
| 216 | 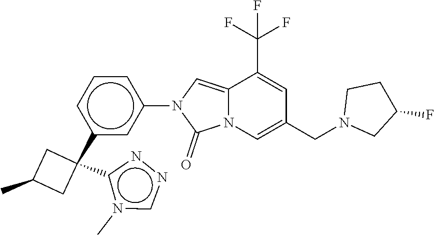 |
| 217 | 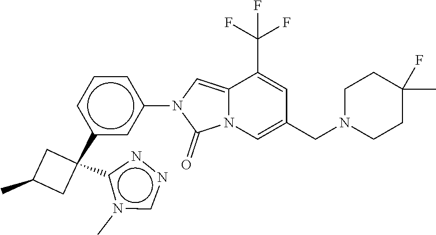 |
| 218 | 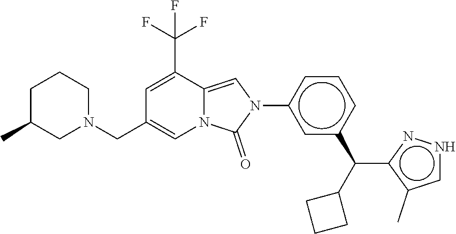 |
| 219 | 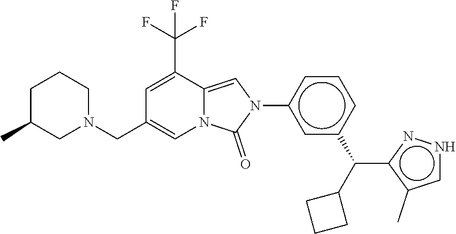 |
| 220 | 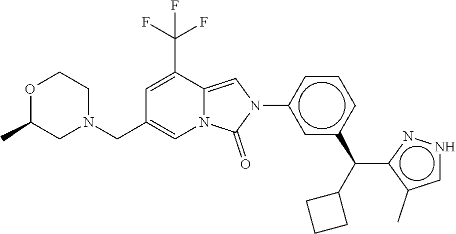 |
| 221 | 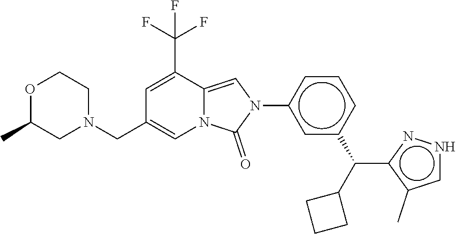 |
| 222 | 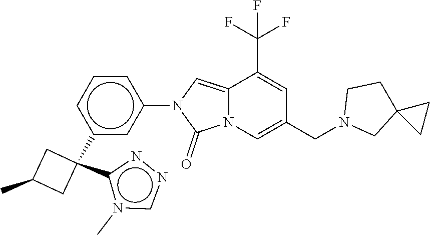 |
| 223 | 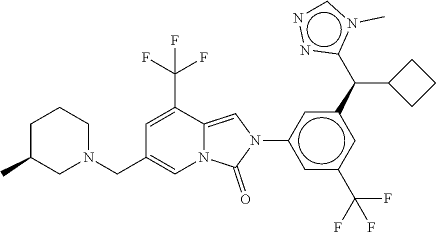 |
| 224 | 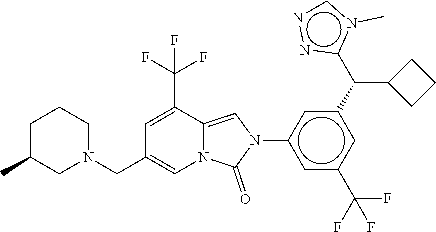 |
| 225 | 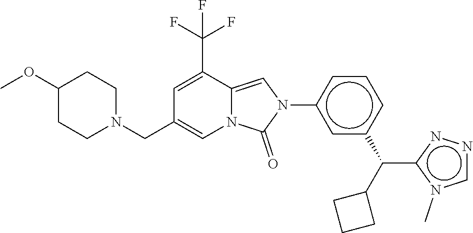 |
| 226 | 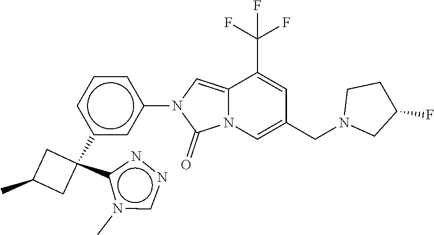 |
| 227 | 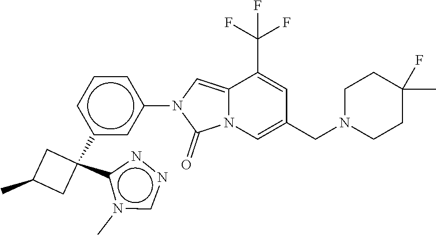 |
| 228 | 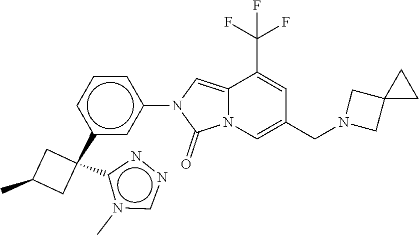 |
| 229 | 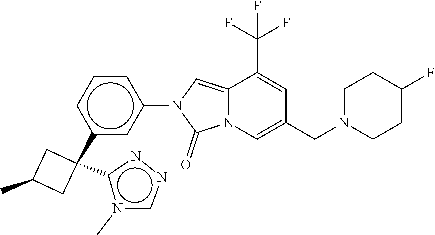 |
| 230 | 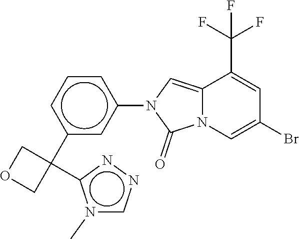 |
| 231 | 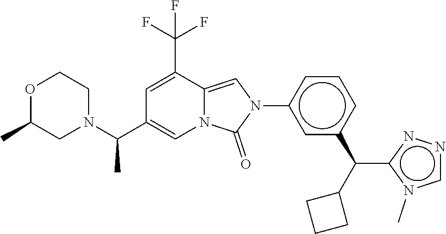 |
| 232 | 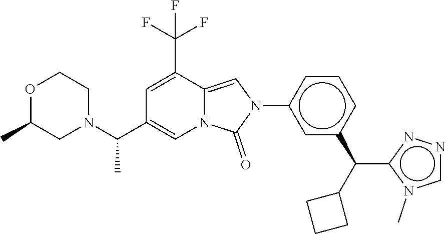 |
| 233 | 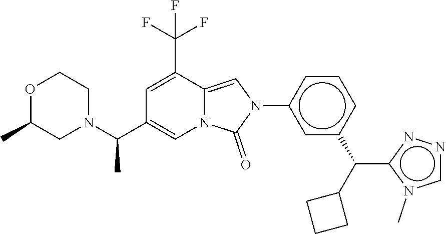 |
| 234 | 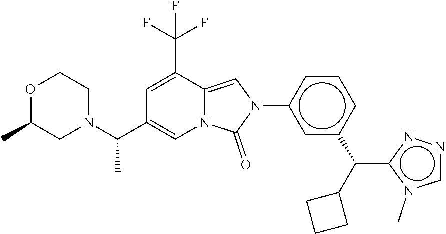 |
| 235 | 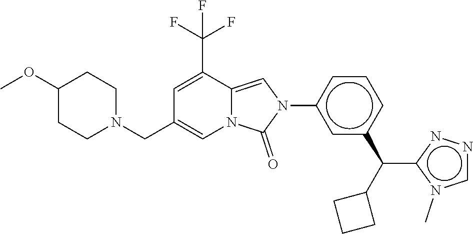 |
| 236 | 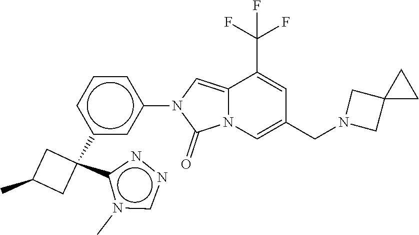 |
| 237 | 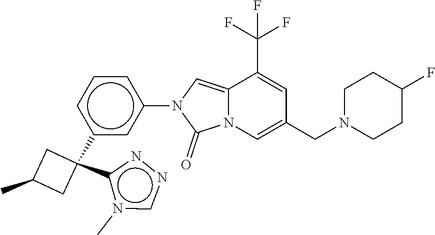 |
| 238 | 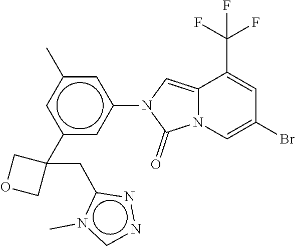 |
| 239 | 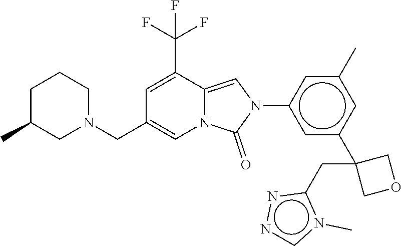 |
| 240 | 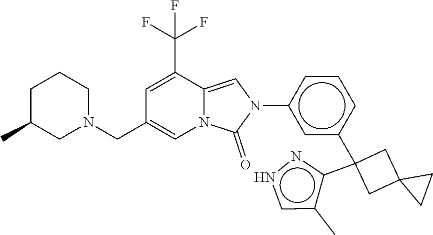 |
| 241 | 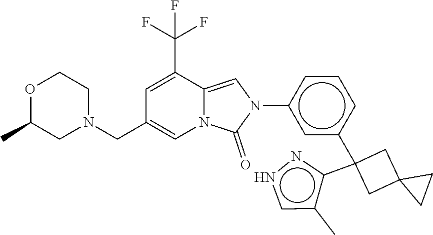 |
| 242 | 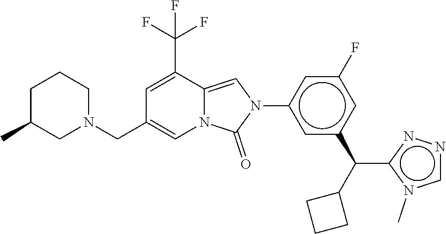 |
| 243 | 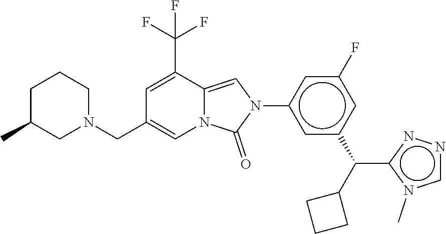 |
| 244 | 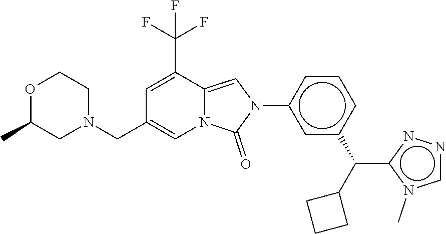 |
| 245 | 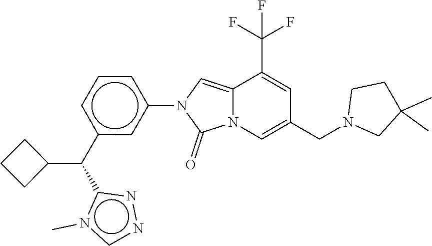 |
| 246 | 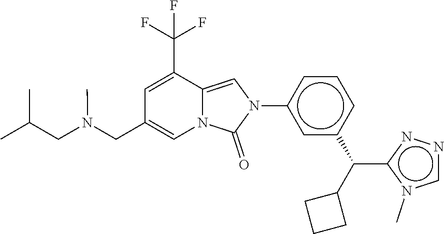 |
| 247 | 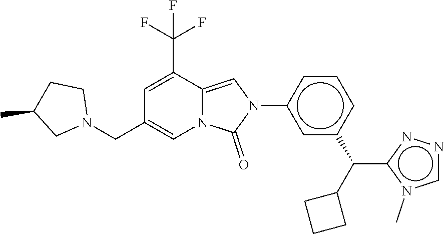 |
| 248 | 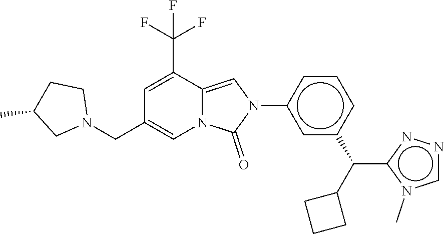 |
| 249 | 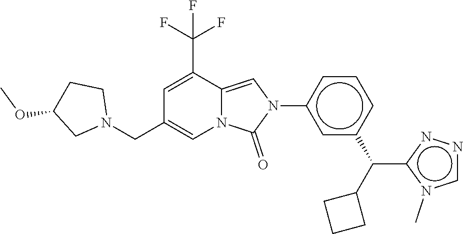 |
| 250 | 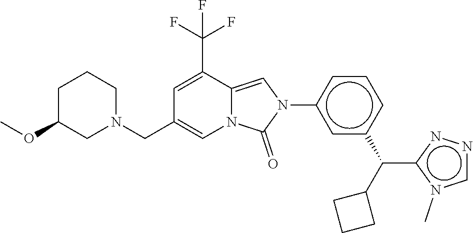 |
| 251 | 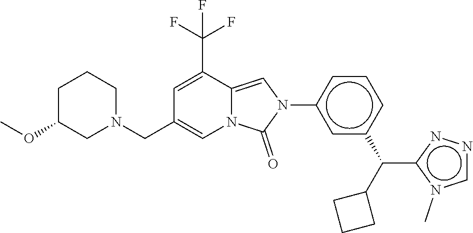 |
| 252 | 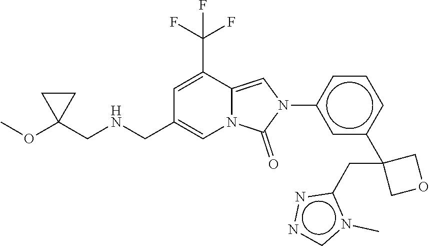 |
| 253 | 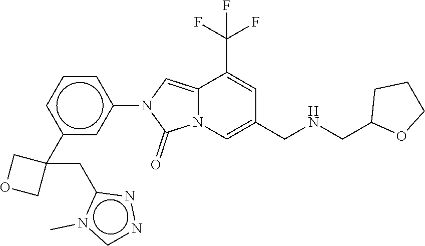 |
| 254 | 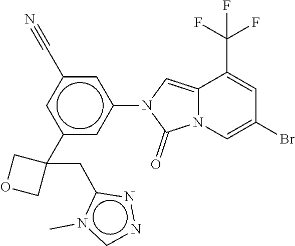 |
| 255 | 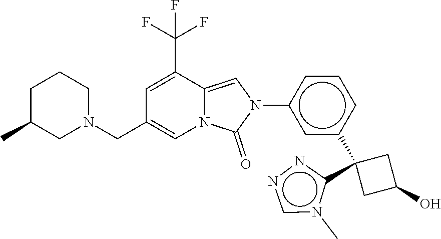 |
| 256 | 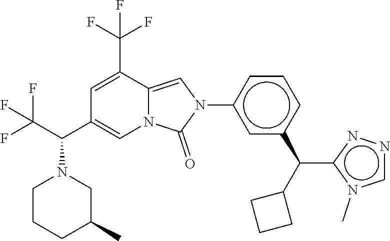 |
| 257 | 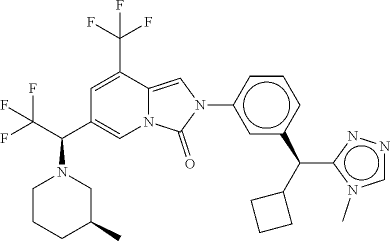 |
| 258 | 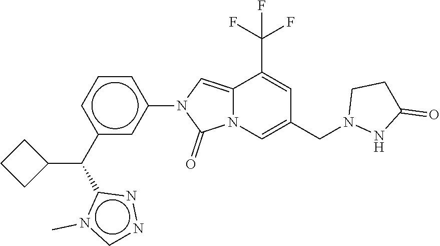 |
| 259 | 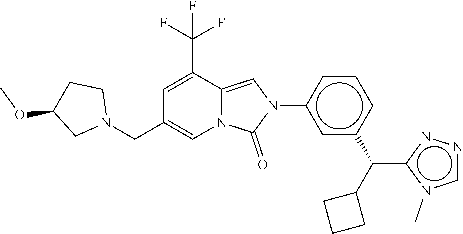 |
| 260 | 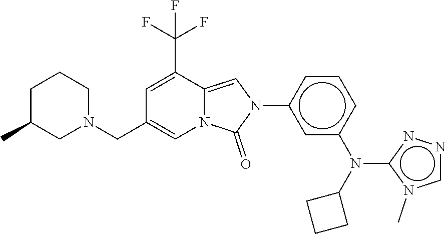 |
| 261 | 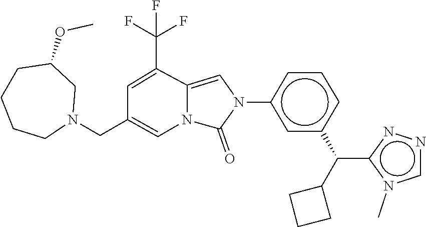 |
| 262 | 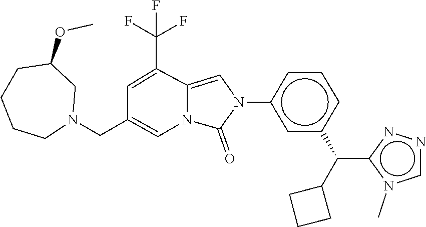 |
| 263 | 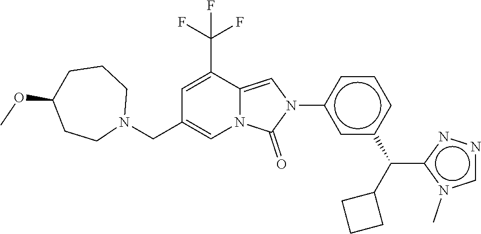 |
| 264 | 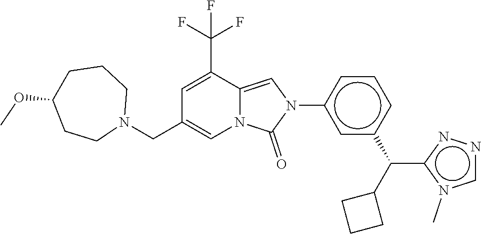 |
| 265 | 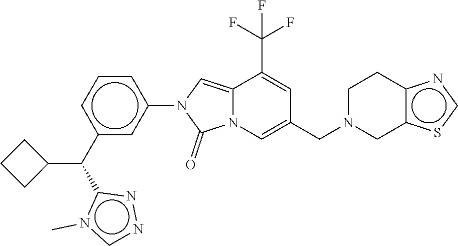 |
| 266 | 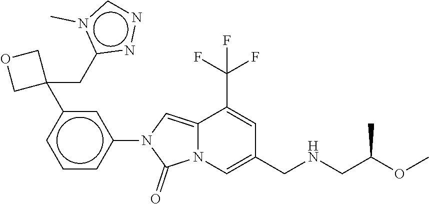 |
| 267 | 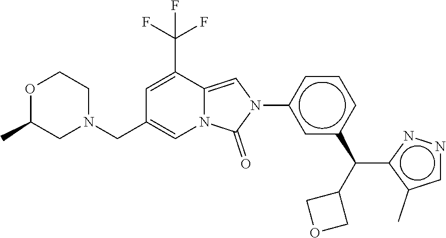 |
| 268 | 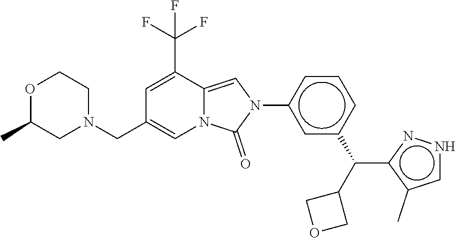 |
| 269 | 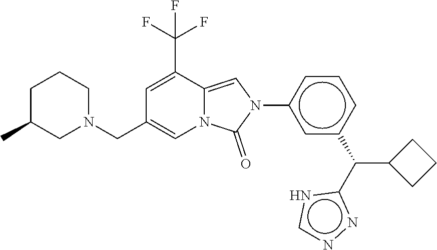 |
| 270 | 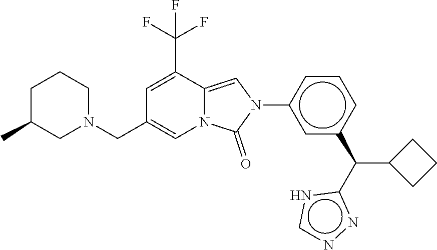 |
| 271 | 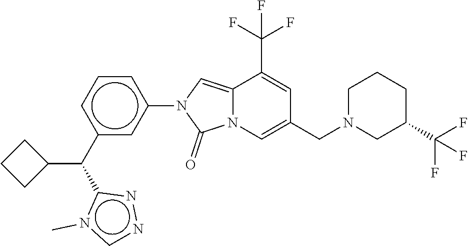 |
| 272 | 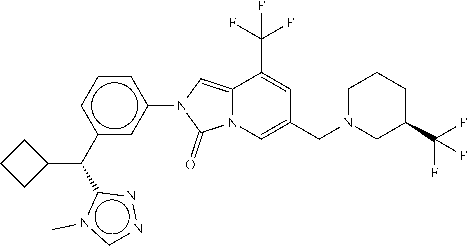 |
| 273 | 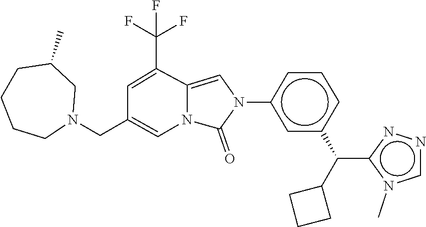 |
| 274 | 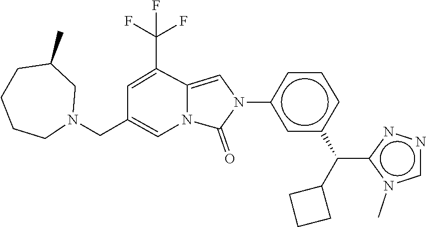 |
| 275 | 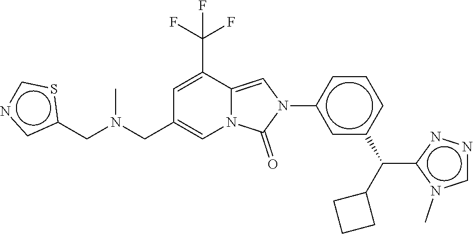 |
| 276 | 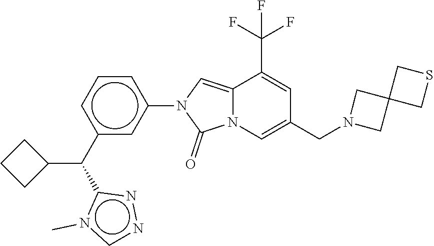 |
| 277 | 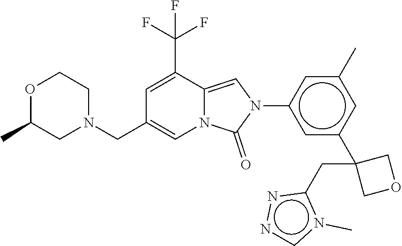 |
| 278_p1 | 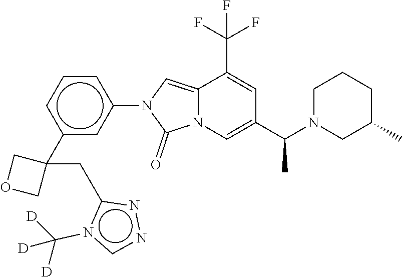 |
| 278_p2 | 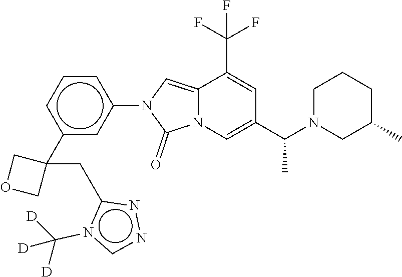 |
| 279 | 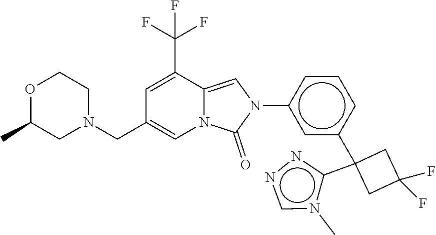 |
| 280 | 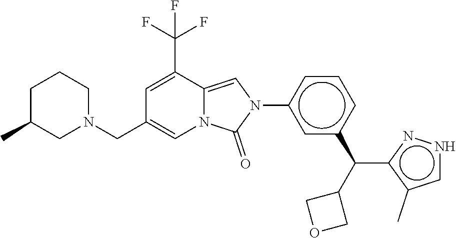 |
| 281 | 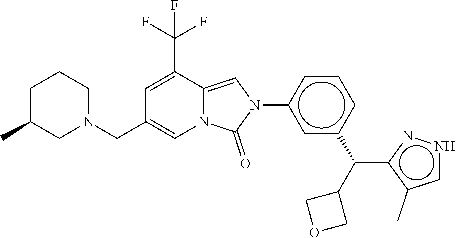 |
| 282 | 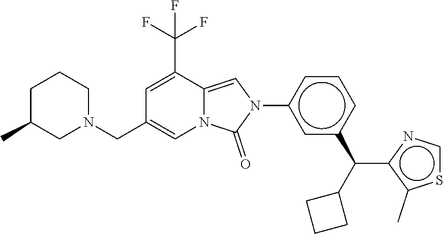 |
| 283 | 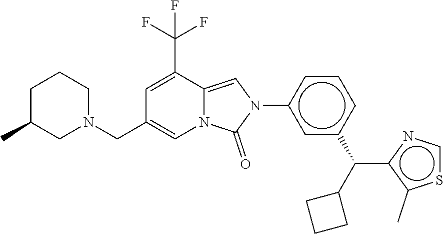 |
| 284 | 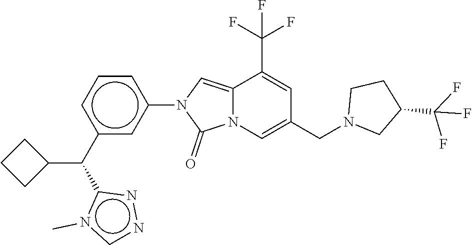 |
| 285 | 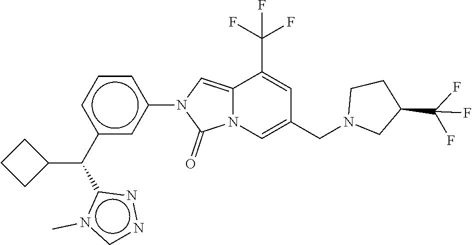 |
| 286 | 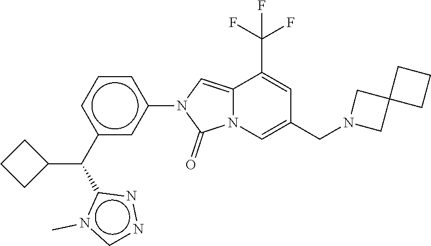 |
| 287_P1 | 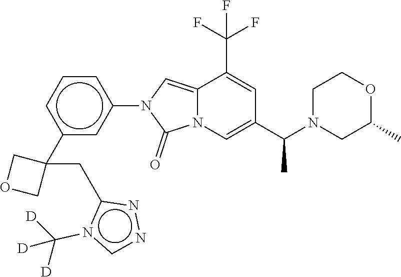 |
| 287_P2 | 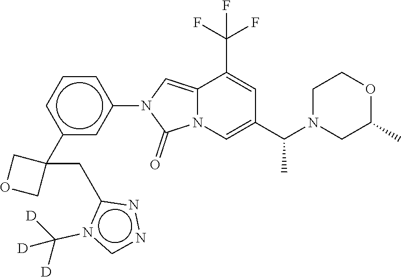 |
| 288 | 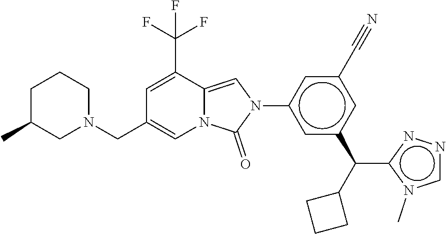 |
| 289 | 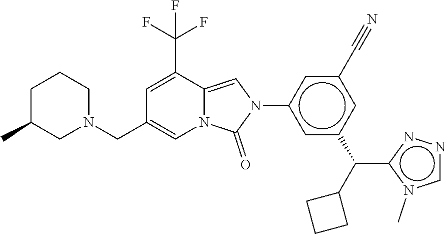 |
| 290 | 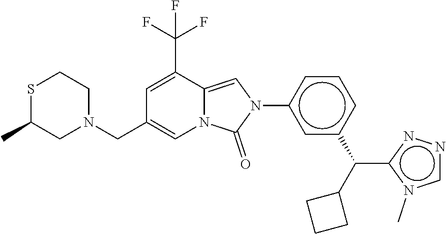 |
| 291 | 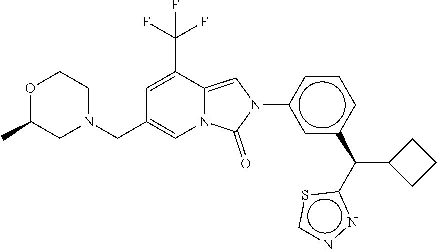 |
| 292 | 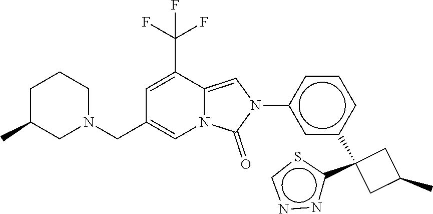 |
| 293 | 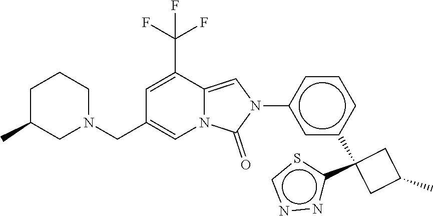 |
| 294 | 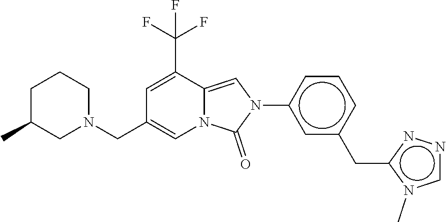 |
| 295 | 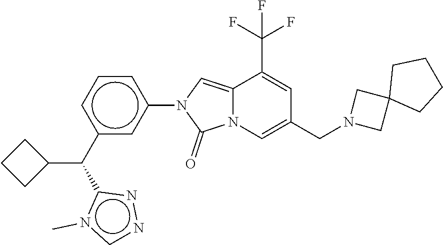 |
| 296 | 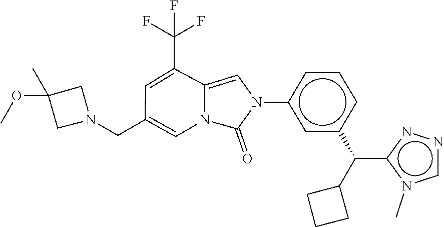 |
| 297 | 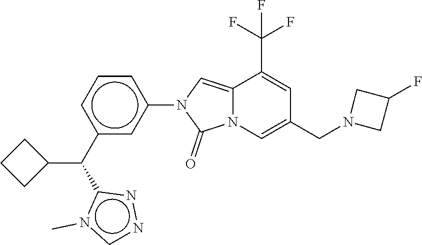 |
| 298 | 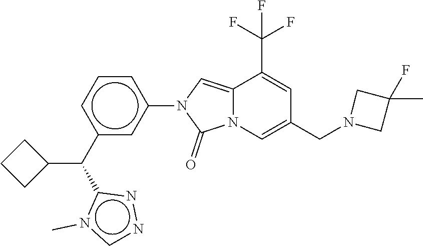 |
| 299 | 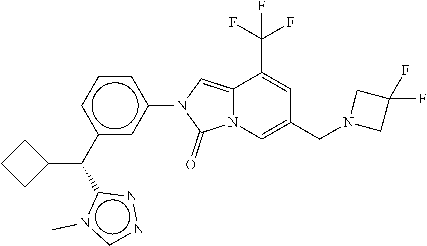 |
| 300 | 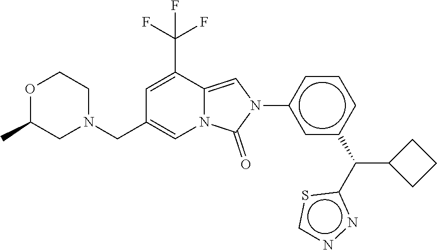 |
| 301 | 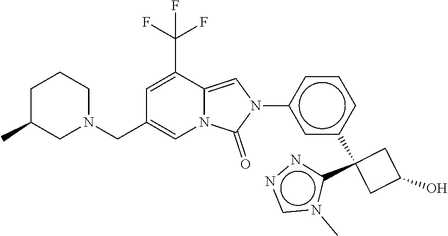 |
| 302 | 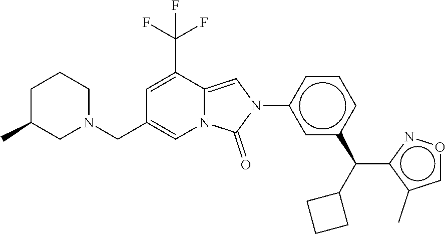 |
| 303 | 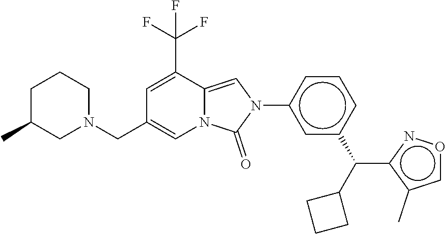 |
| 304 | 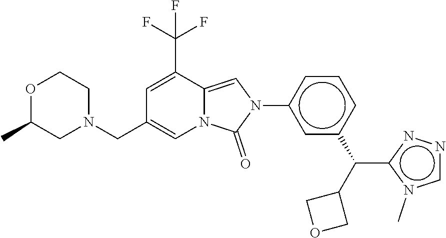 |
| 305 | 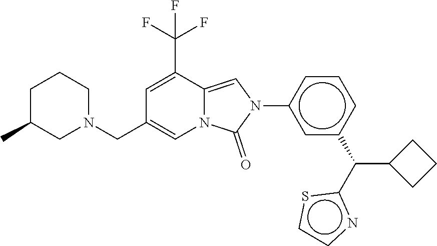 |
| 306 | 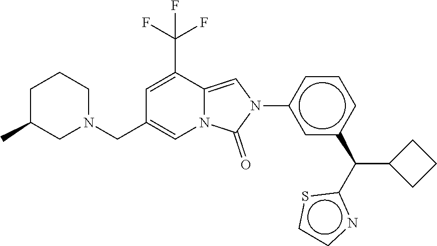 |
| 307 | 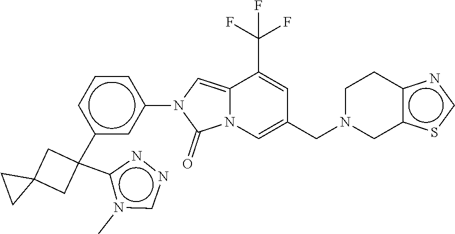 |
| 308 | 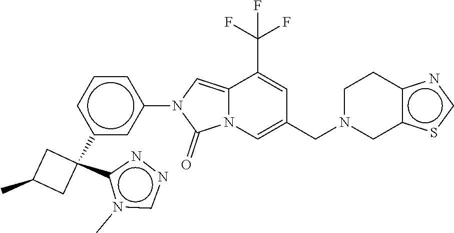 |
| 309 | 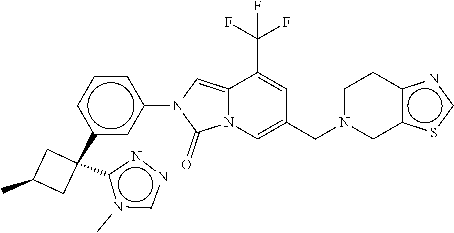 |
| 310 | 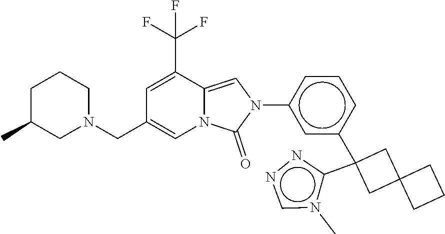 |
| 311 | 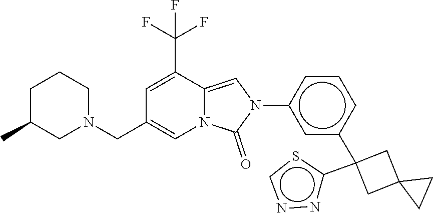 |
| 312 | 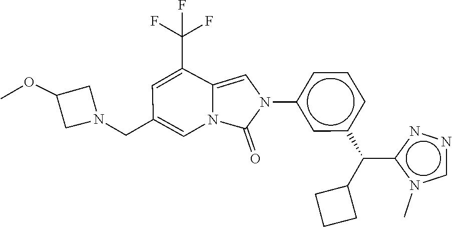 |
| 313 | 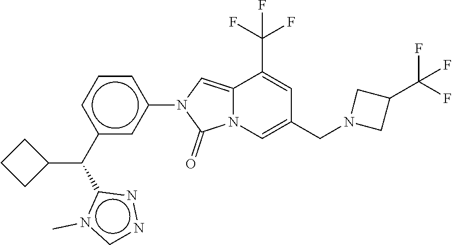 |
| 314 | 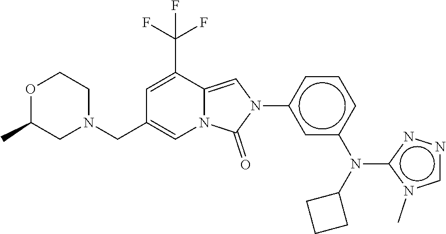 |
| 315 | 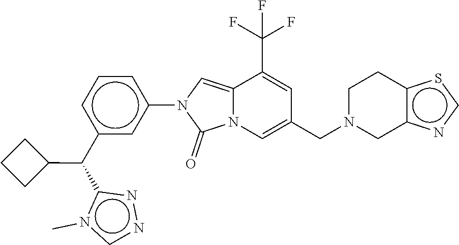 |
| 316 | 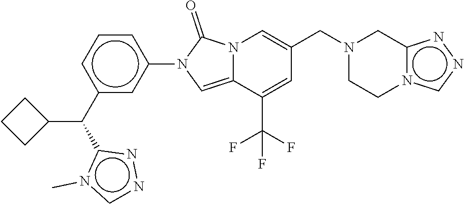 |
| 317 | 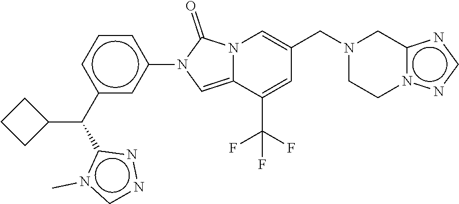 |
| 318 | 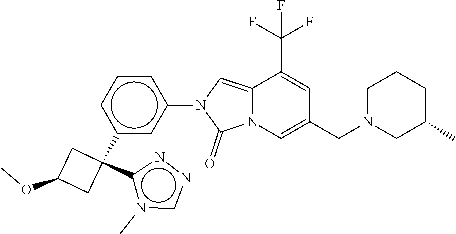 |
| 319 | 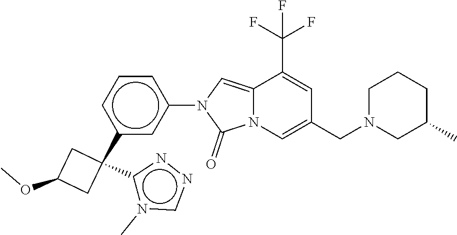 |
| 329 | 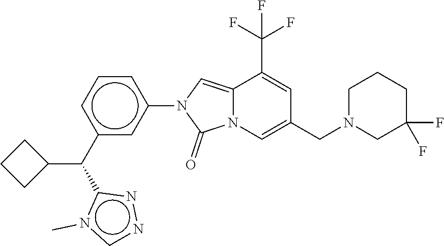 |
| 321 | 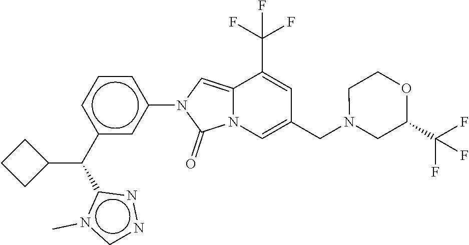 |
| 322 | 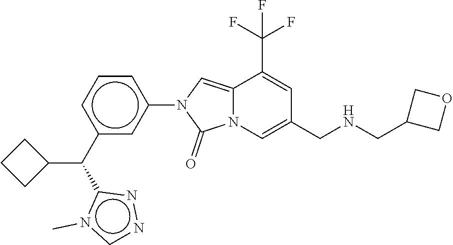 |
| 323 | 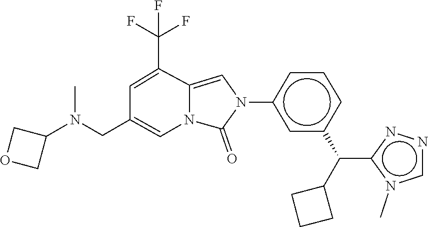 |
| 324 | 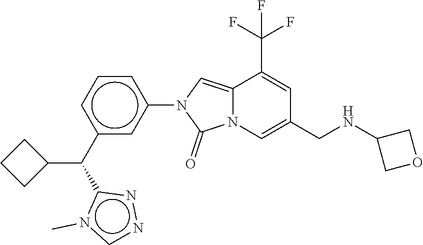 |
| 325 | 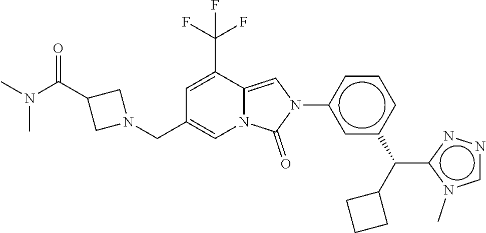 |
| 326 | 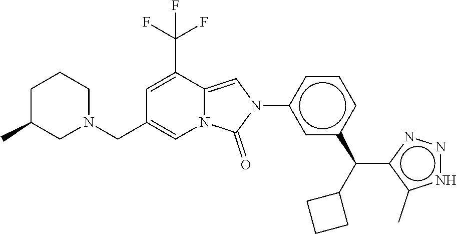 |
| 327 | 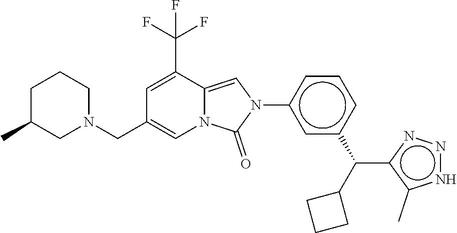 |
| 328 | 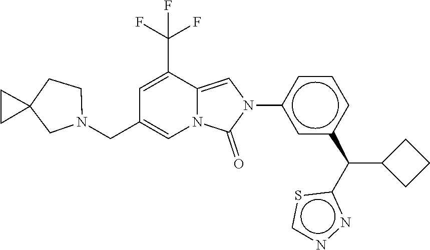 |
| 329 | 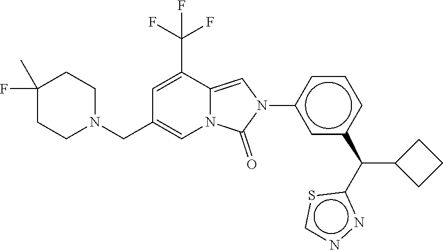 |
| 330 | 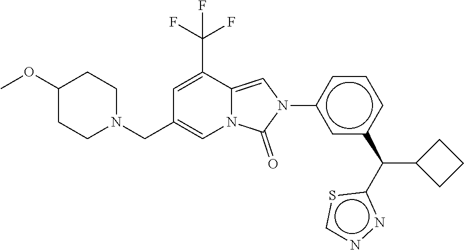 |
| 331 | 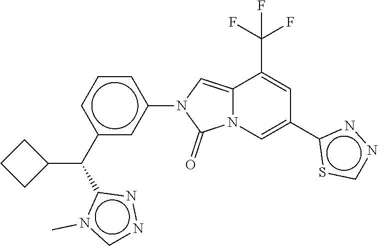 |
| 332_p1 | 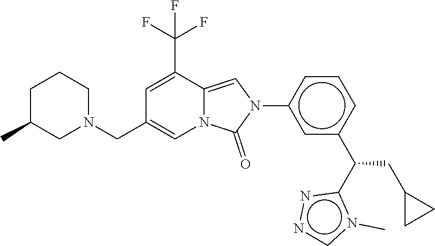 |
| 332_p2 | 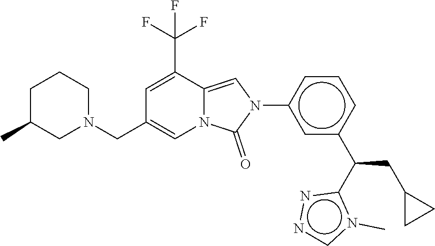 |
| 333 | 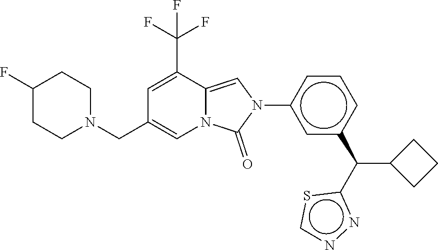 |
| 334 | 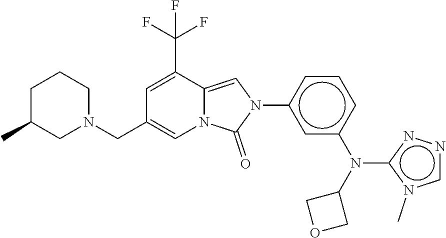 |
| 335 | 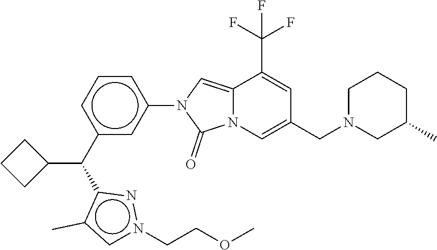 |
| 336 | 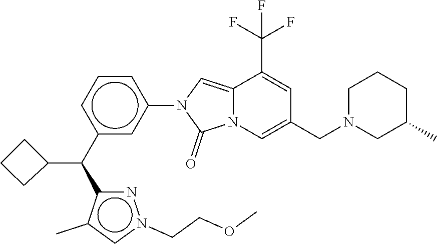 |
| 337 | 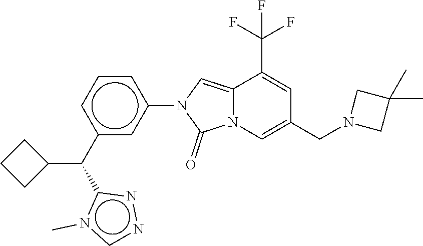 |
| 338 | 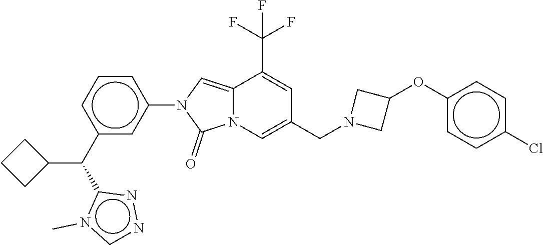 |
| 339_P1 | 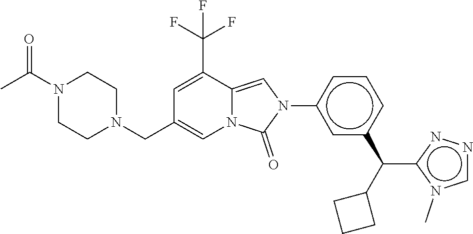 |
| 339_P2 | 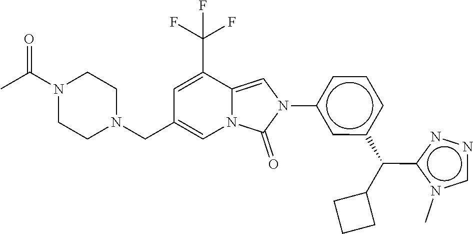 |
| 340_P1 | 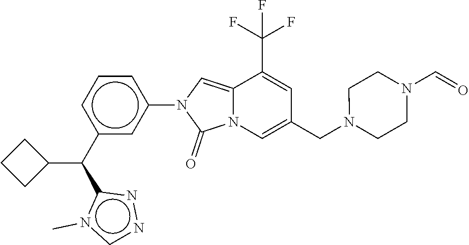 |
| 340_P2 | 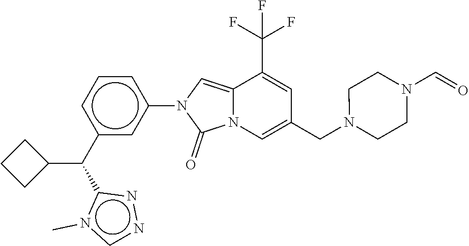 |
| 341 | 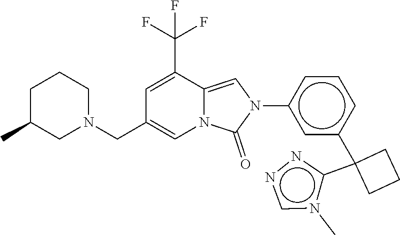 |
| 342 | 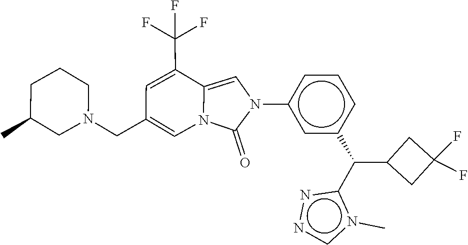 |
| 343 | 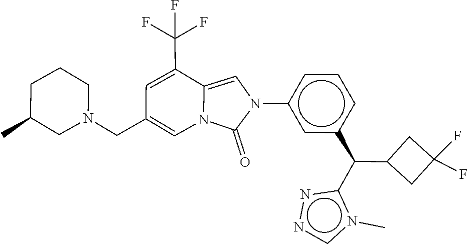 |
| 344 | 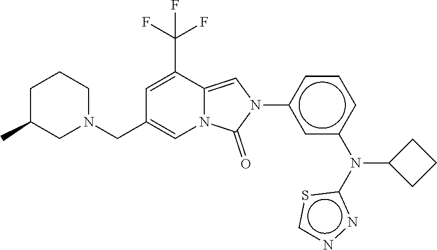 |
| 345 | 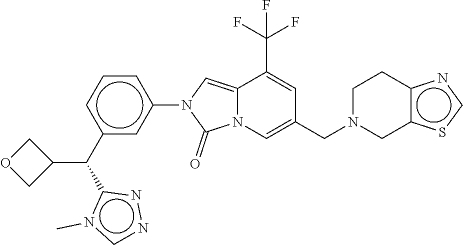 |
| 346 | 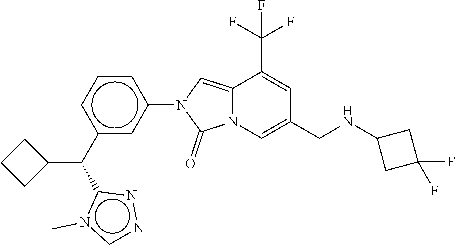 |
| 347 | 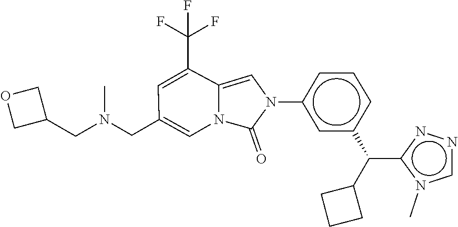 |
| 348 | 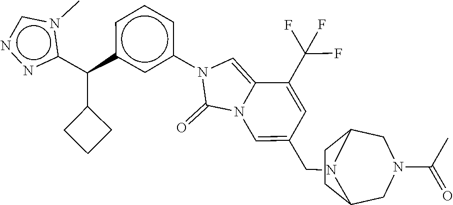 |
| 349 | 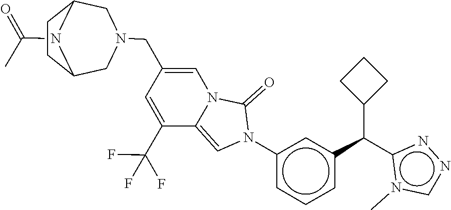 |
| 350 | 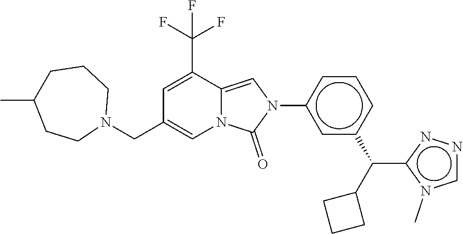 |
| 351_P1 | 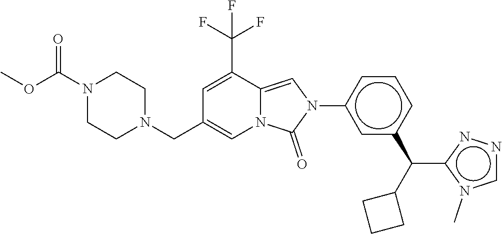 |
| 351_P2 | 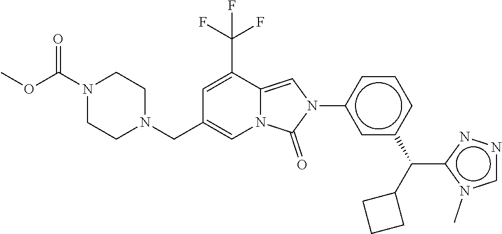 |
| 352_P1 | 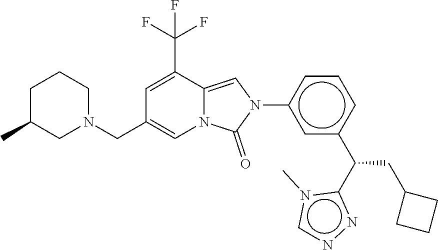 |
| 352_P2 | 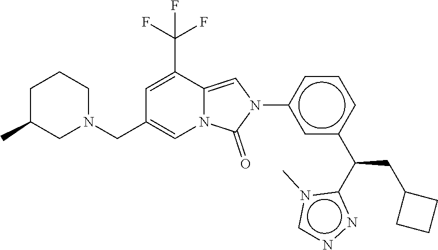 |
| 353 | 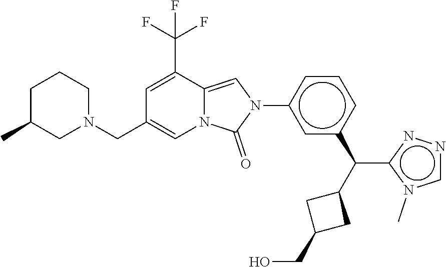 |
| 354 | 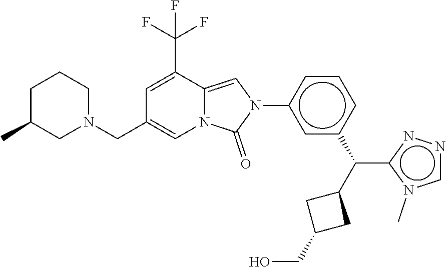 |
| 355 | 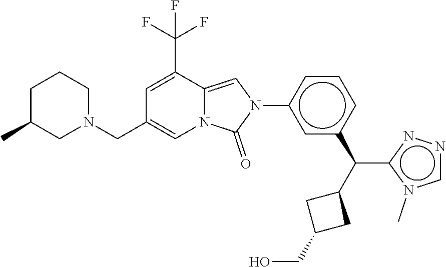 |
| 356 | 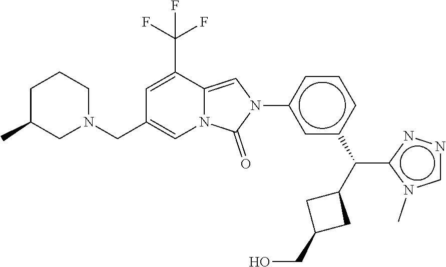 |
| 357 | 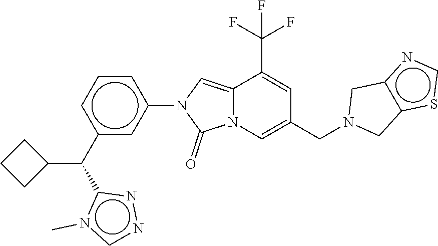 |
| 358 | 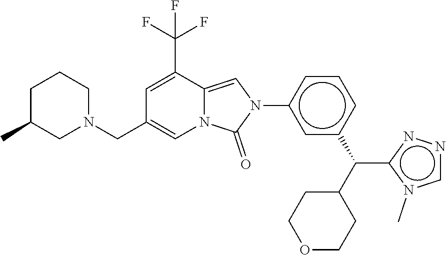 |
| 359 | 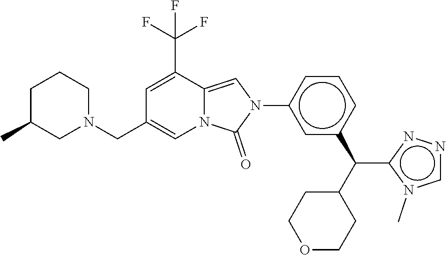 |
| 360 | 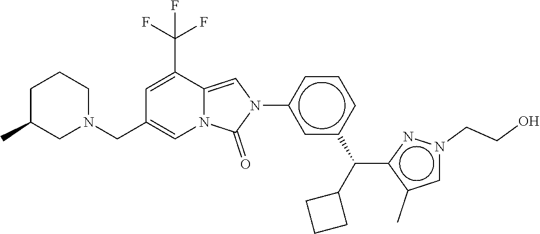 |
| 361 | 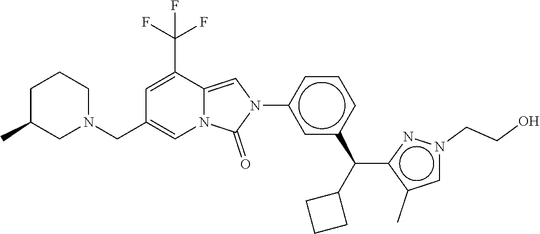 |
| 362_P1 | 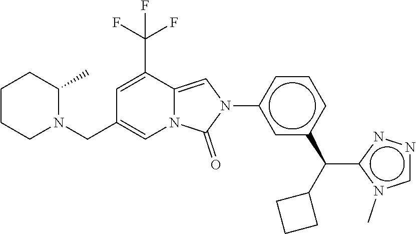 |
| 362_P2 | 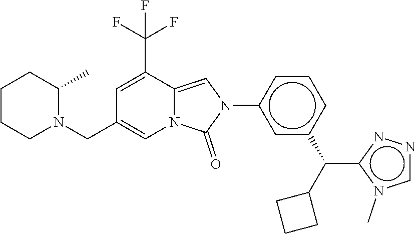 |
| 363_P1 | 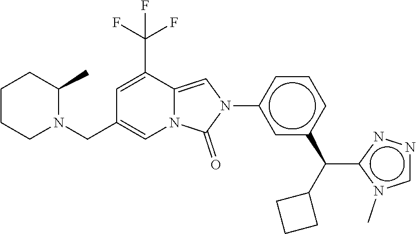 |
| 363_P2 | 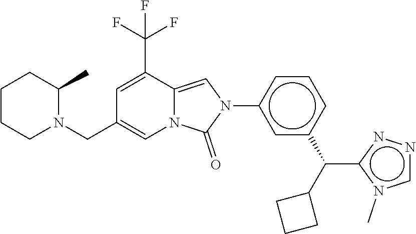 |
| 364_P1 | 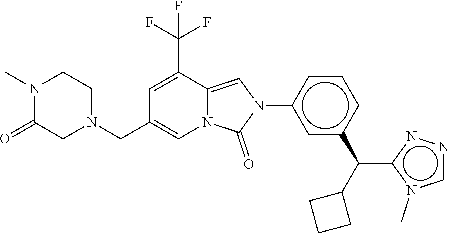 |
| 364_P2 | 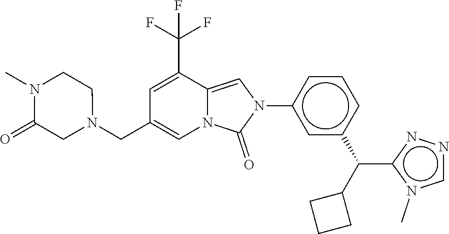 |
| 365 | 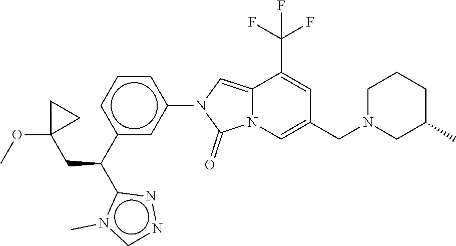 |
| 366 | 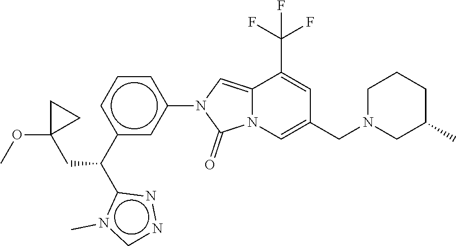 |
| 367 | 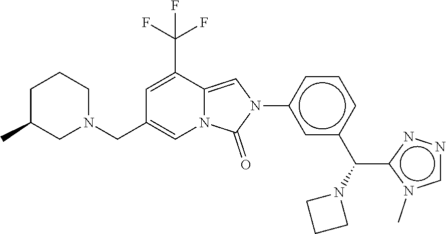 |
| 368 | 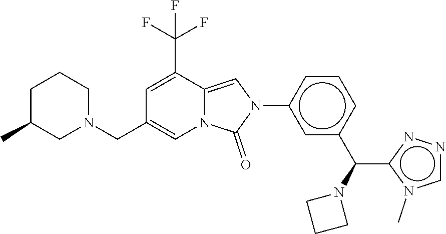 |
| 369 | 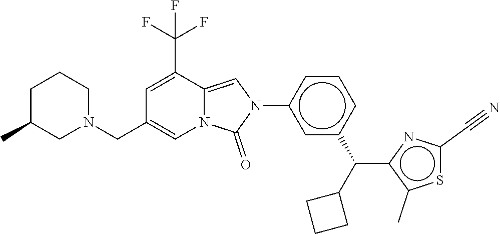 |
| 370 | 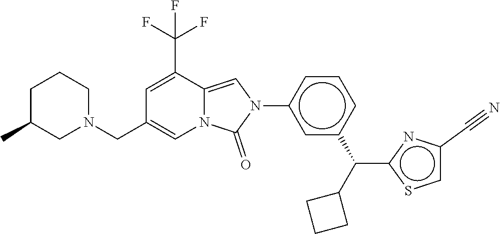 |
| 371 | 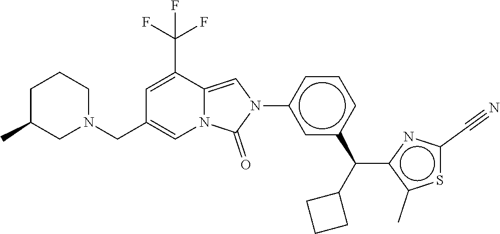 |
| 372 | 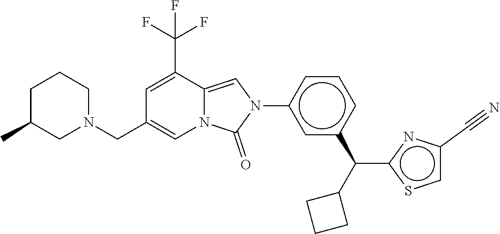 |
| 373 | 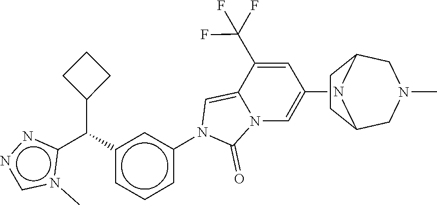 |
| 374 | 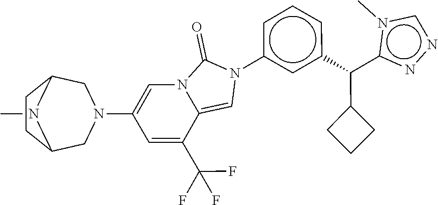 |
| 375 | 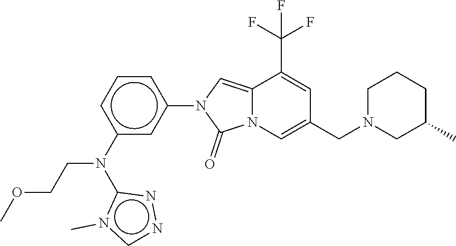 |
| 376 | 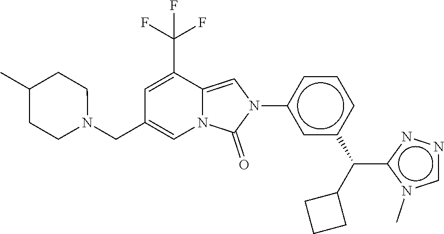 |
| 377 | 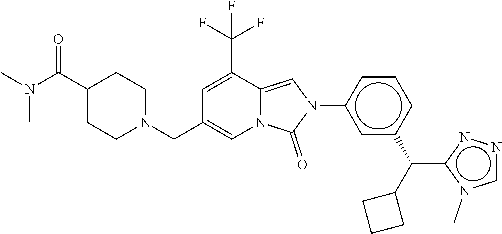 |
| 378 | 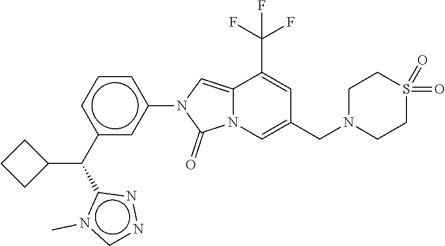 |
| 379 | 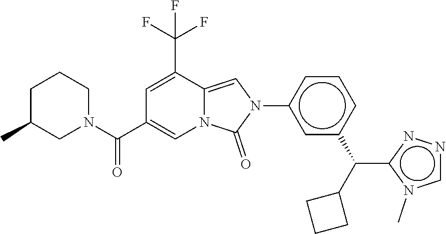 |
| 380_P1 | 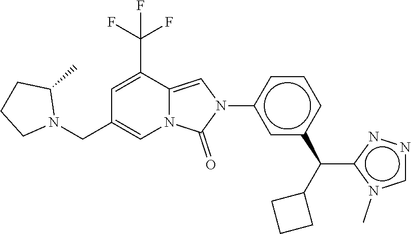 |
| 380_P2 | 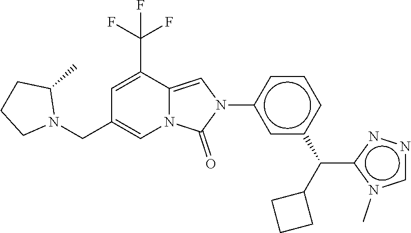 |
| 381_P1 | 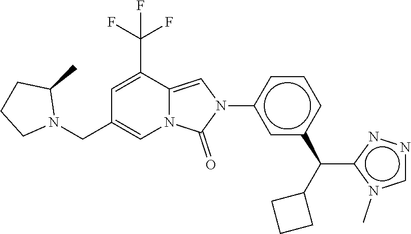 |
| 381_P2 | 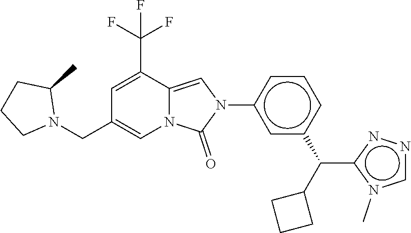 |
| | 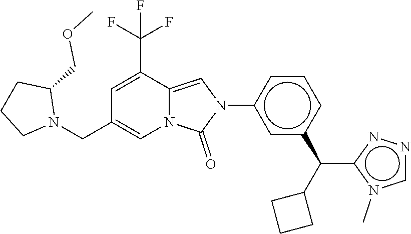 |
| | 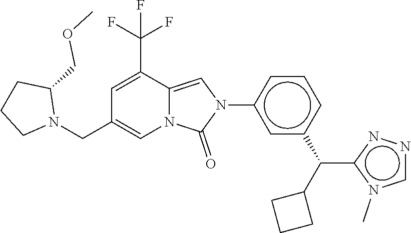 |
| 382_P1 | 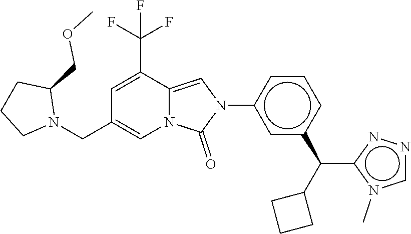 |
| 382_P2 | 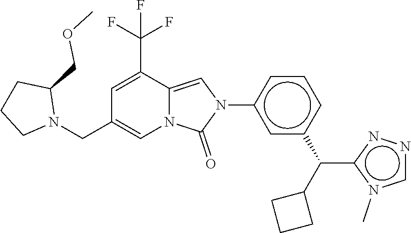 |
| 383 | 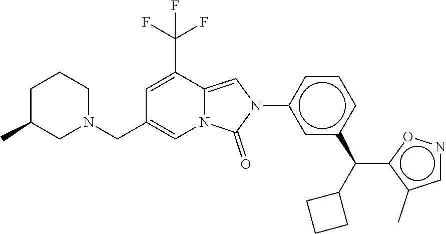 |
| 384 | 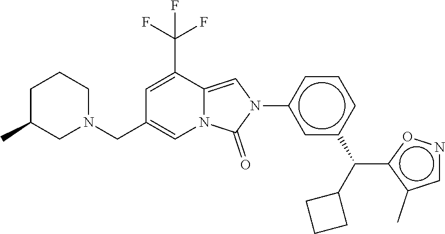 |
| 385 | 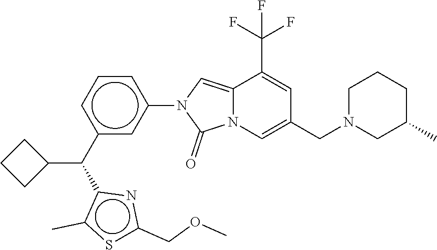 |
| 386 | 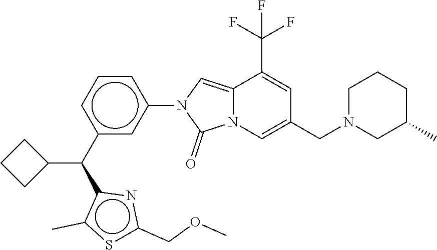 |
| 387 | 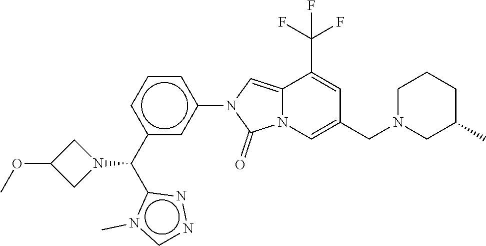 |
| 388 | 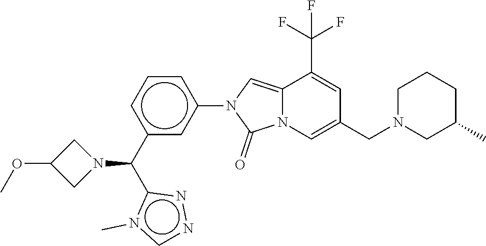 |
| 389 | 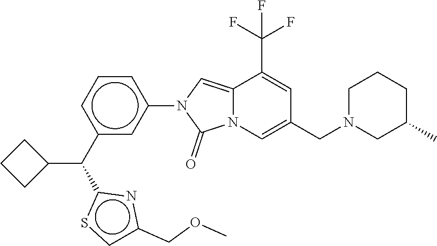 |
| 390 | 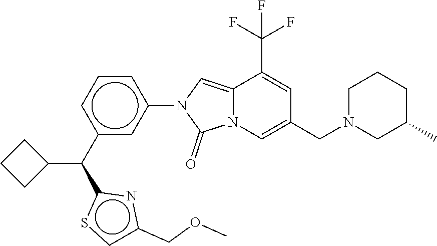 |
| 391 | 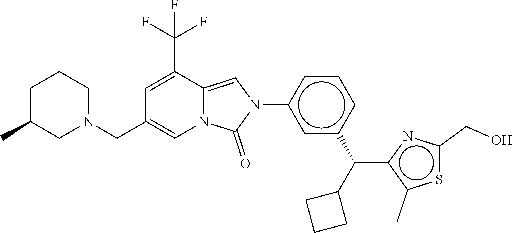 |
| 392 | 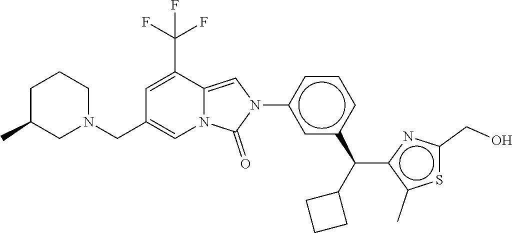 |
| 393 | 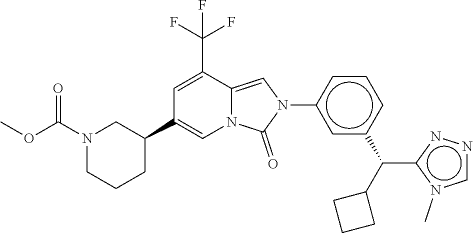 |
| 394 | 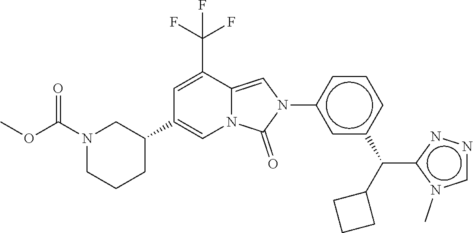 |
| 395 | 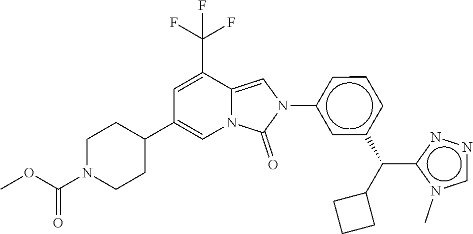 |
| 396 | 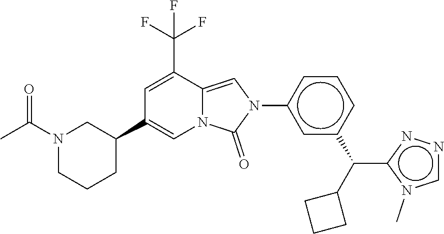 |
| 397 | 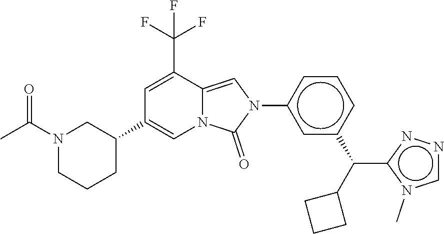 |
| 398 | 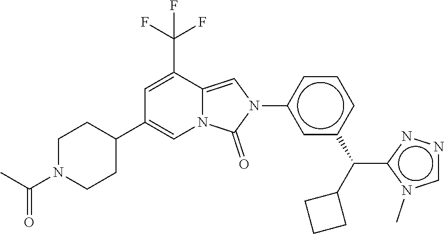 |
| 399 | 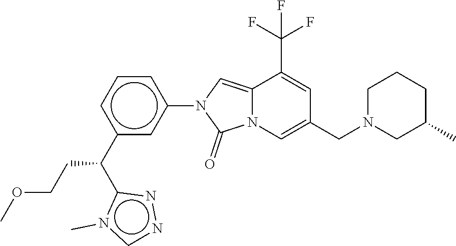 |
| 400 | 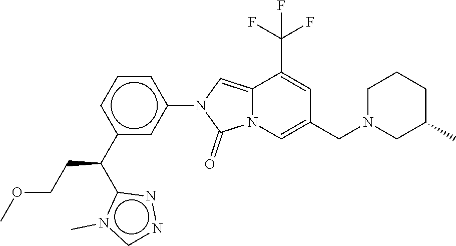 |
| 401_P1 | 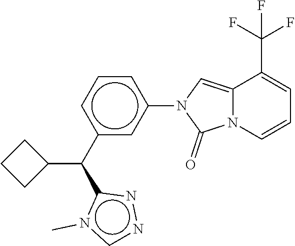 |
| 401_P2 | 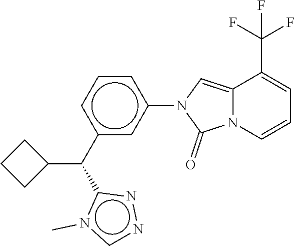 |
| 402 | 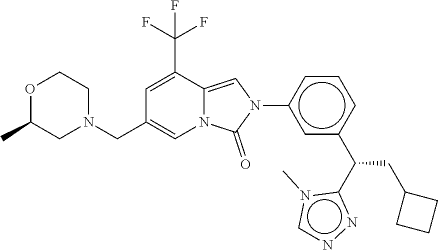 |
| 403_P1 | 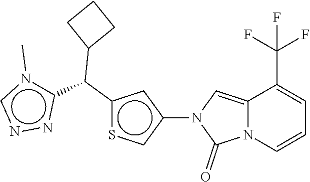 |
| 403_P2 | 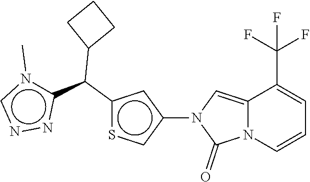 |
| 404 | 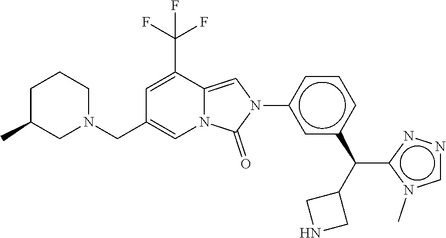 |
| 403 | 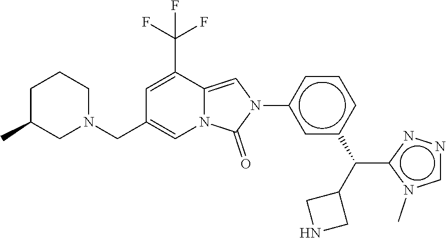 |
| 404 | 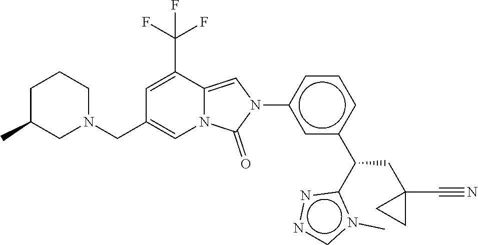 |
| 405 | 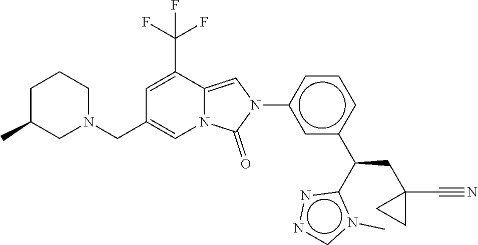 |
| 406 | 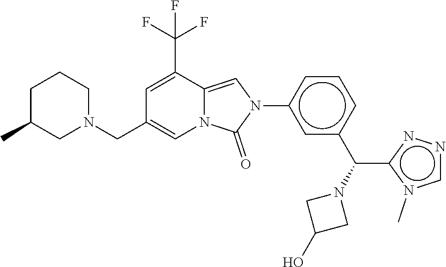 |
| 407 | 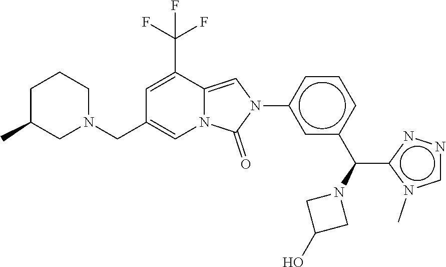 |
| 408 | 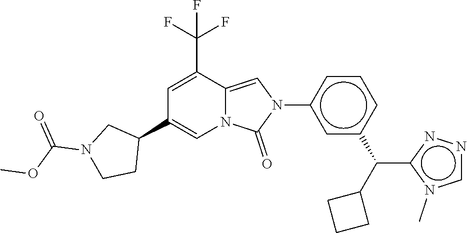 |
| 409 | 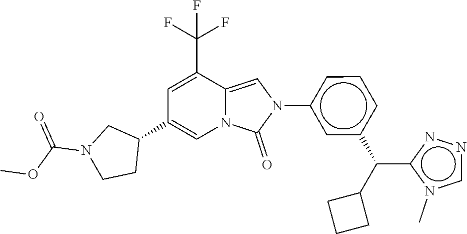 |
| 410 | 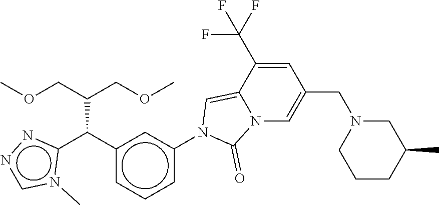 |
| 411 | 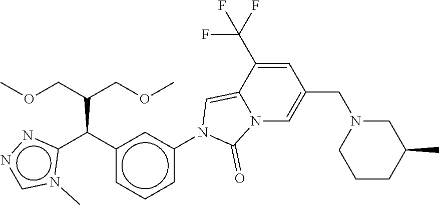 |
| 412 | 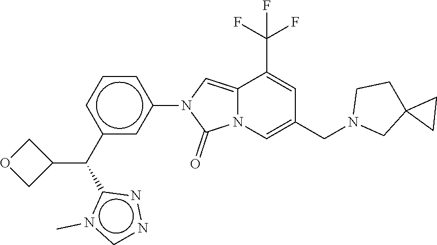 |
| 413 | 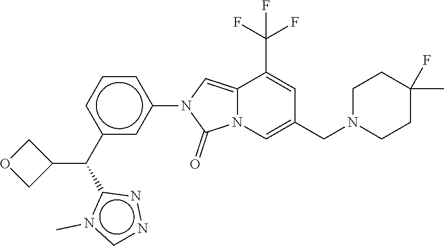 |
| 414 | 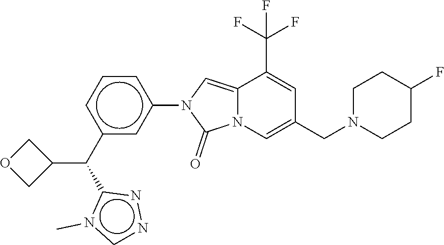 |
| 415 | 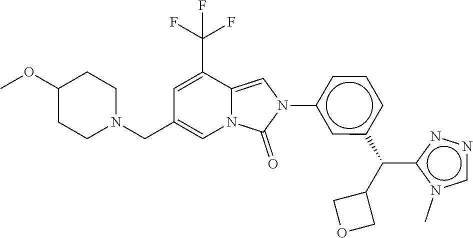 |
| 416 | 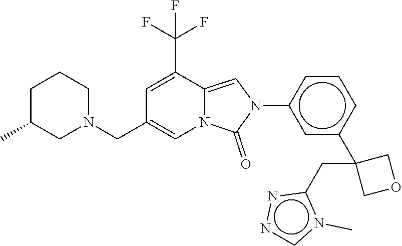 |
| 417 | 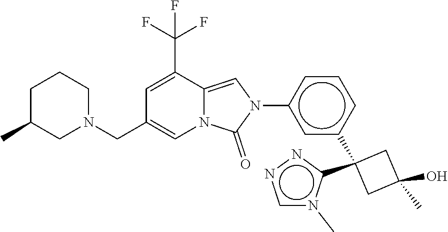 |
| 418 | 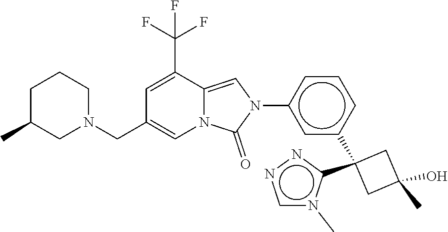 |
| 419 | 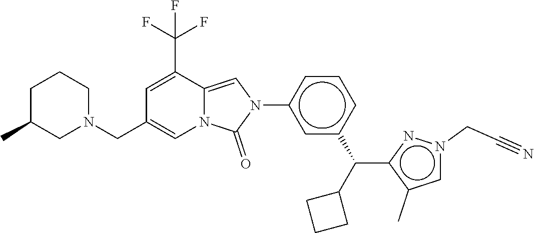 |
| 420 | 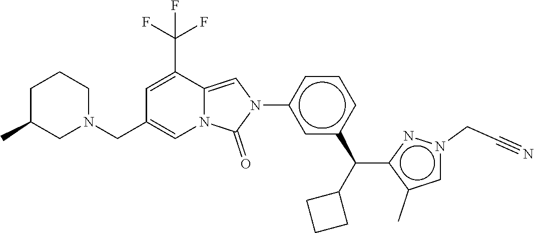 |
| 421 | 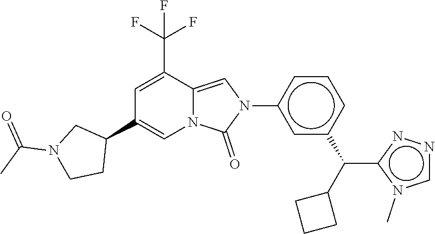 |
| 422 | 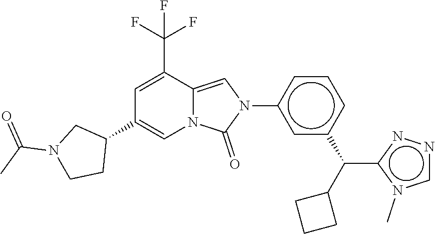 |
| 423 | 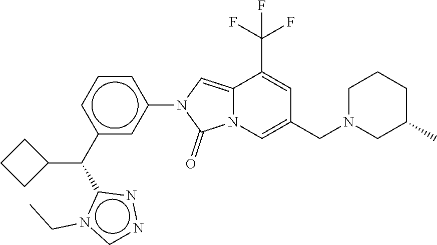 |
| 424 | 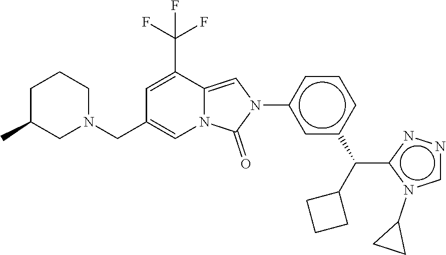 |
| 425 | 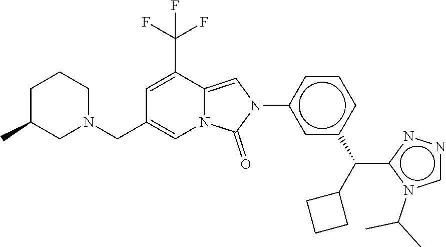 |
| 426 | 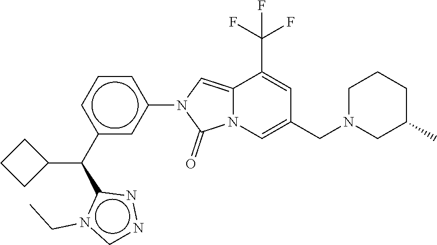 |
| 427 | 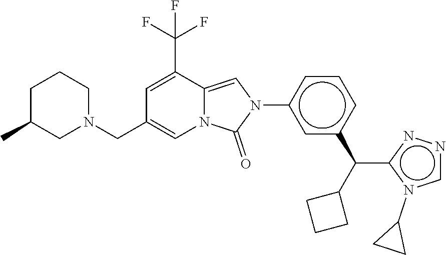 |
| 428 | 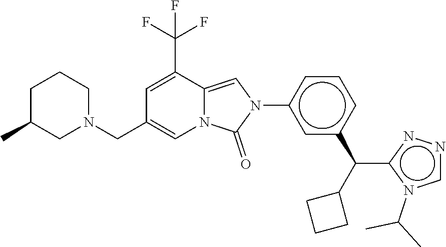 |
| 429 | 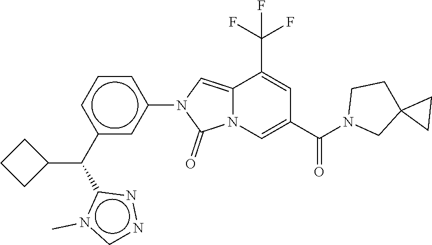 |
| 430 | 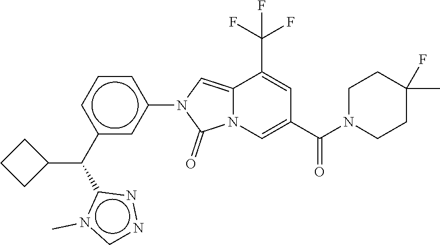 |
| 431 | 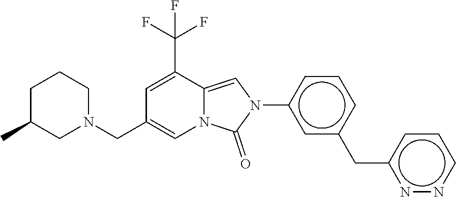 |
| 432 | 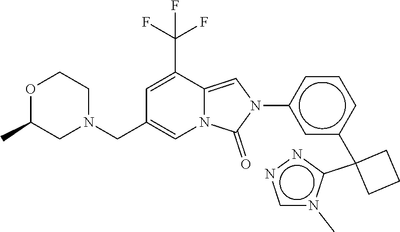 |
| 433 | 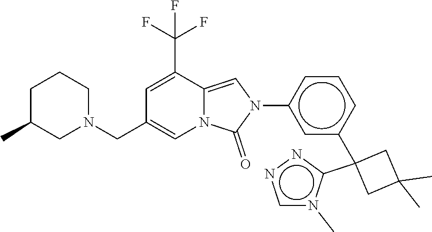 |
| 434_P1 | 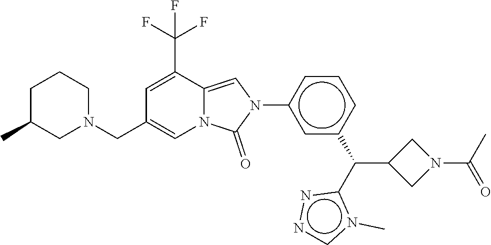 |
| 434_P2 | 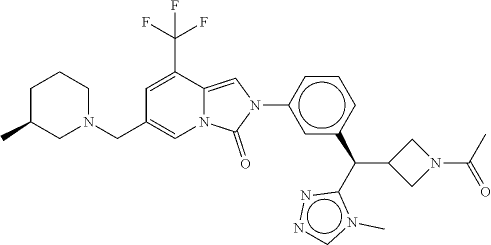 |
| 435 | 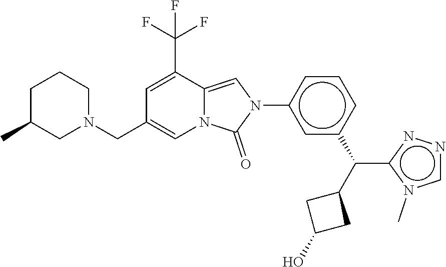 |
| 436 | 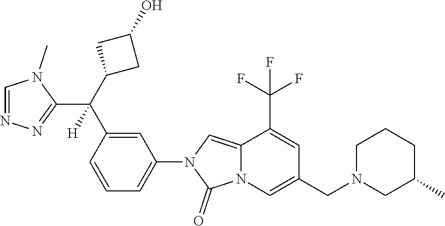 [SMILES: C[C@H]1CCCN(Cc2cc(C(F)(F)F)c3cn(-c4cccc([C@@H](c5nncn5C)[C@H]5C[C@@H](O)C5)c4)c(=O)n3c2)C1] |
| 437 | 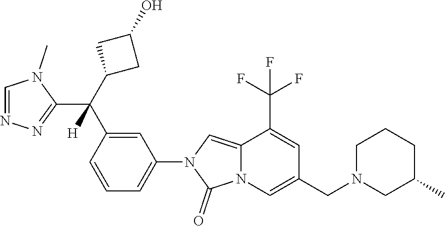 [SMILES: C[C@H]1CCCN(Cc2cc(C(F)(F)F)c3cn(-c4cccc([C@H](c5nncn5C)[C@H]5C[C@@H](O)C5)c4)c(=O)n3c2)C1] |
| 438 | 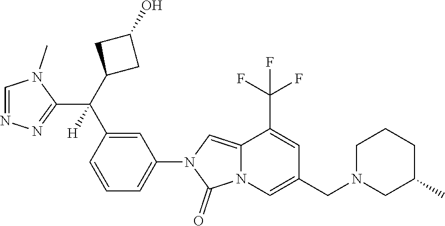 [SMILES: C[C@H]1CCCN(Cc2cc(C(F)(F)F)c3cn(-c4cccc([C@@H](c5nncn5C)[C@H]5C[C@H](O)C5)c4)c(=O)n3c2)C1] |
| 439 | 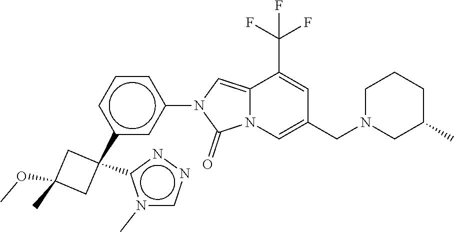 |
| 440 | 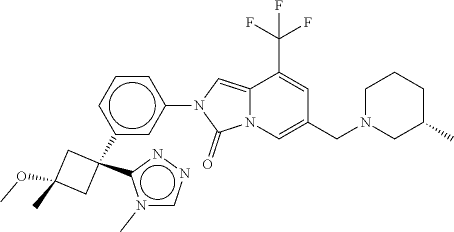 |
| 441 | 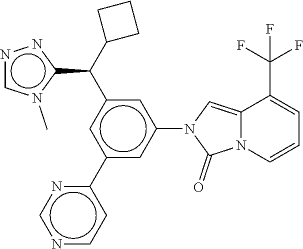 |
| 442 | 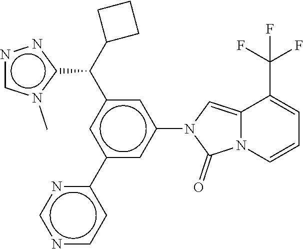 |
| 443 | 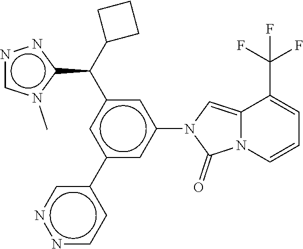 |
| 444 | 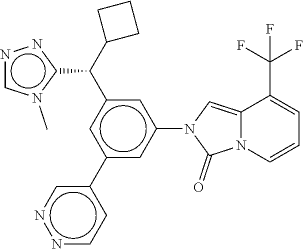 |
| 445 | 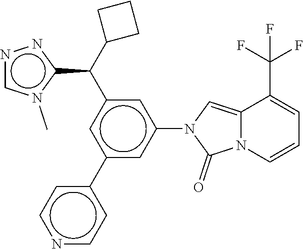 |
| 446 | 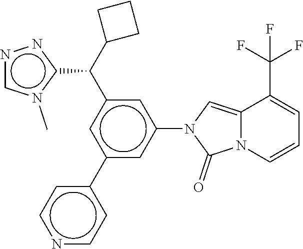 |
| 447 | 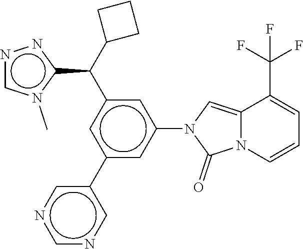 |
| 448 | 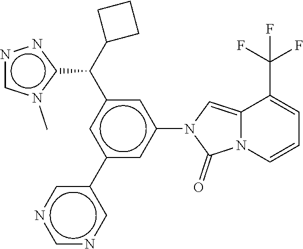 |
| 449 | 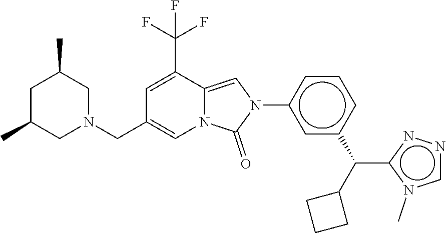 |
| 450 | 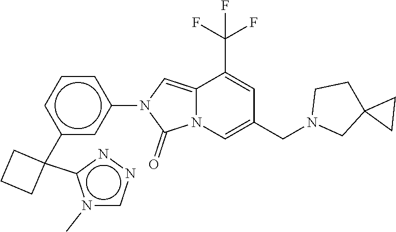 |
| 451 | 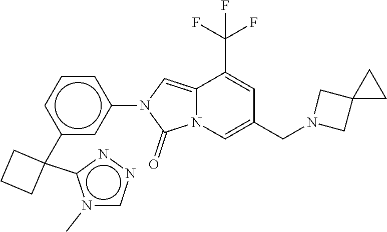 |
| 452 | 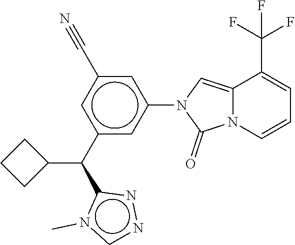 |
| 453 | 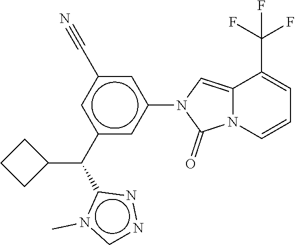 |
| 454 | 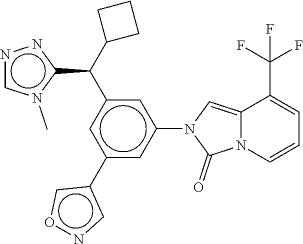 |
| 455 | 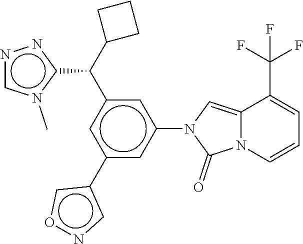 |
| 456 | 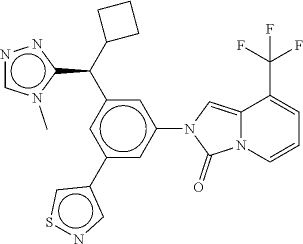 |
| 457 | 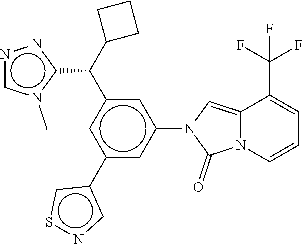 |
| 458 | 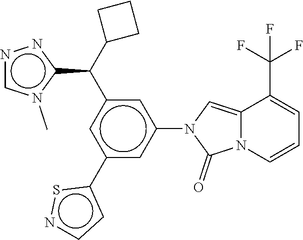 |
| 459 | 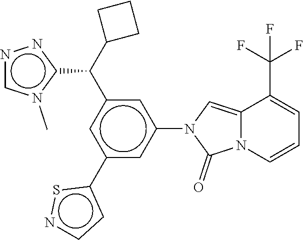 |
| 460 | 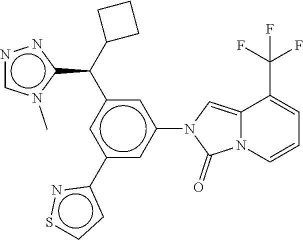 |
| 461 | 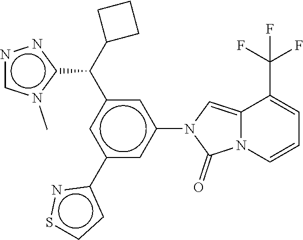 |
| 462 | 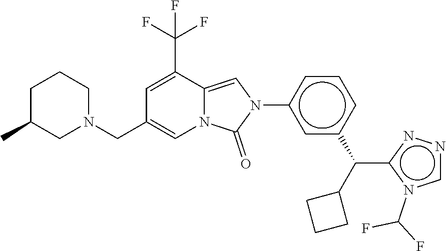 |
| 463 | 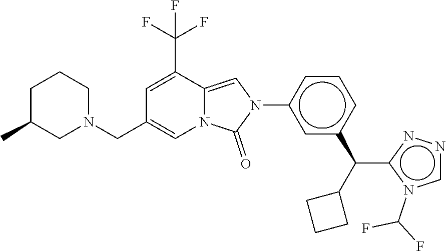 |
| 464 | 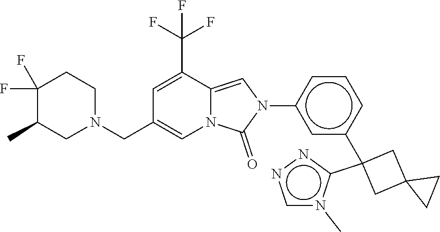 |
| 465 | 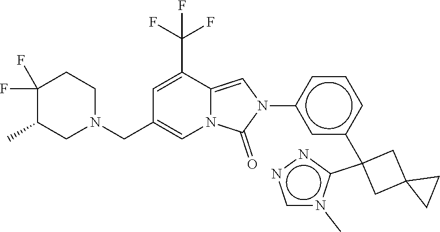 |
| 466 | 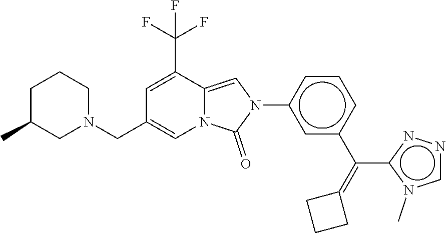 |
| 467 | 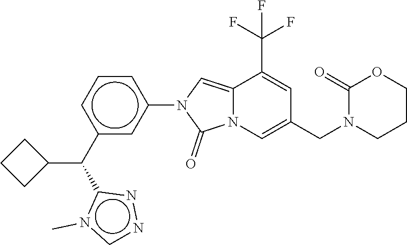 |
| 468 | 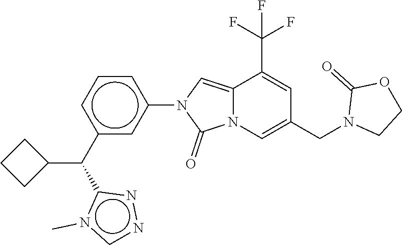 |
| 469 | 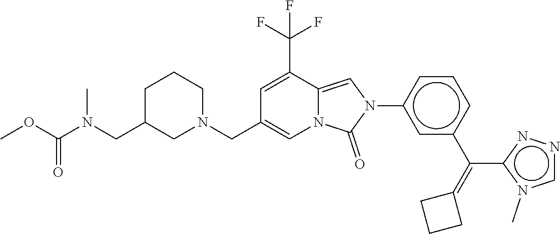 |
| 470_P1 | 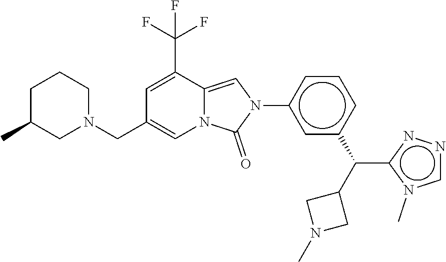 |
| 470_P2 | 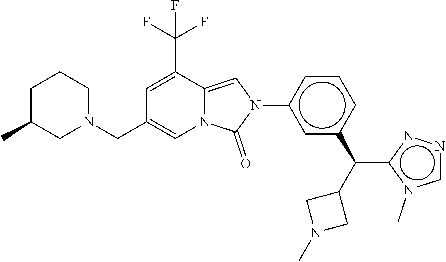 |
| 471 | 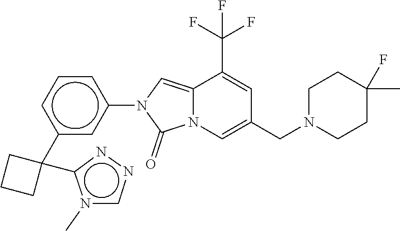 |
| 472_P1 | 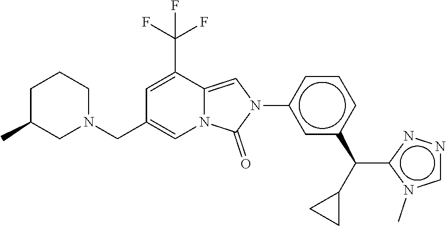 |
| 472_P2 | 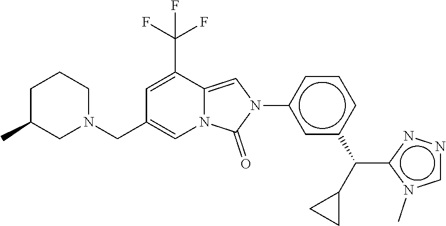 |
| 473 | 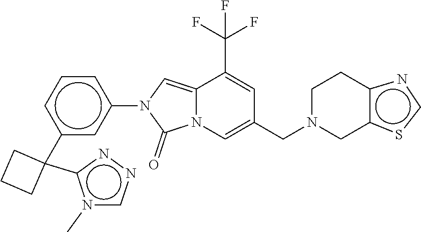 |
| 474 | 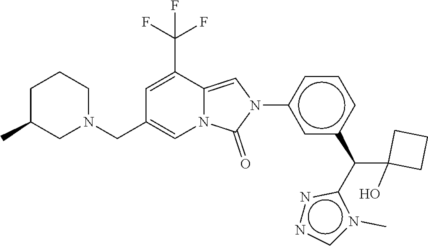 |
| 475 | 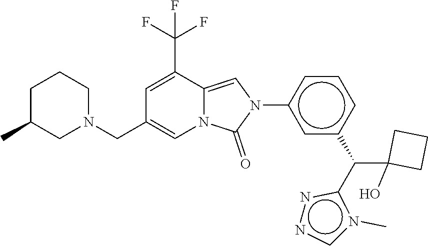 |
| 476 | 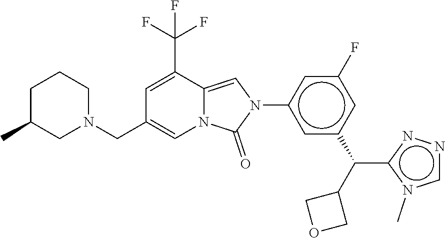 |
| 477 | 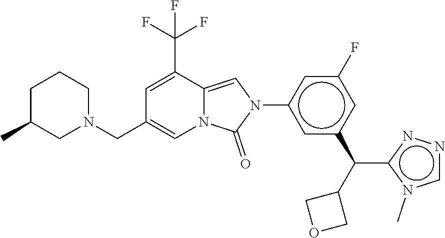 |
| 478 | 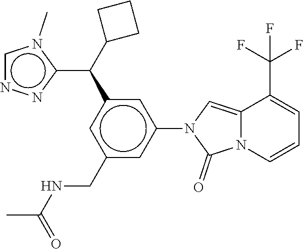 |
| 479 | 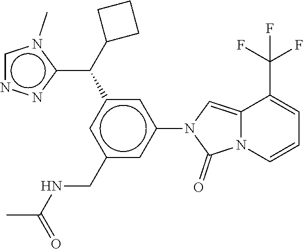 |
| 480 | 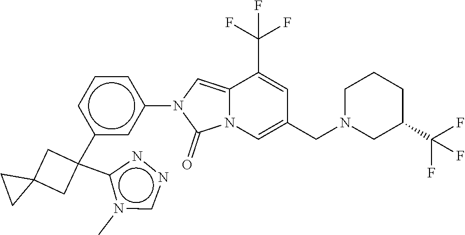 |
| 481 | 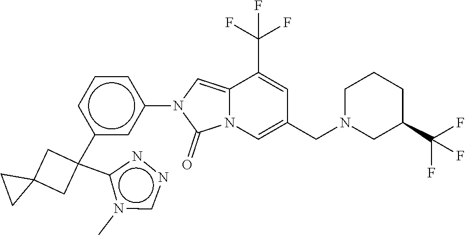 |
| 482 | 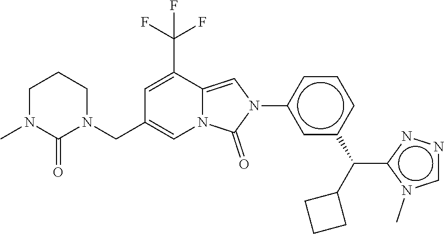 |
| 483 | 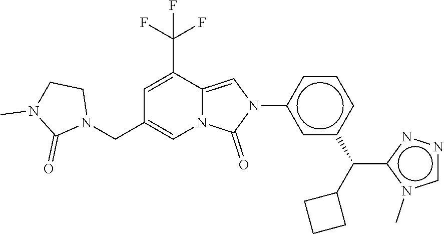 |
| 484 | 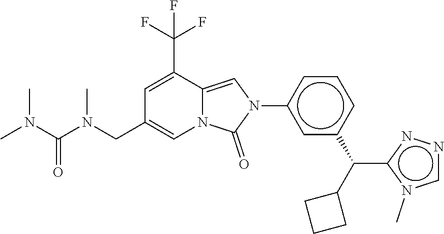 |
| 485 | 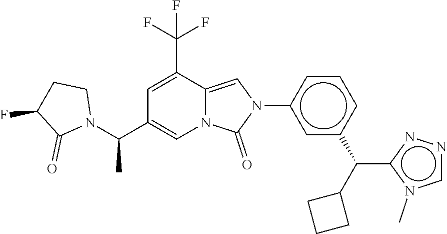 |
| 486 | 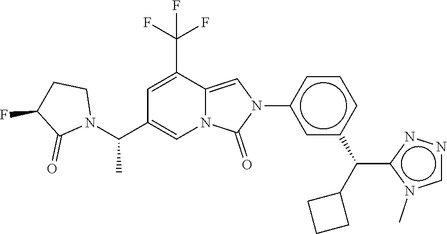 |
| 487 | 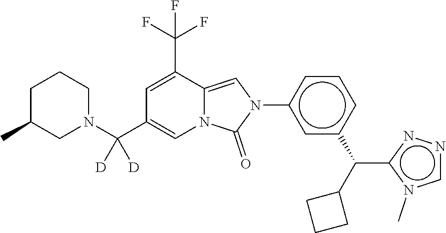 |
| 488 | 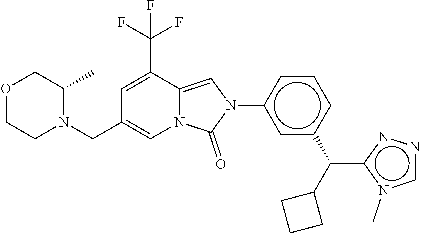 |
| 489 | 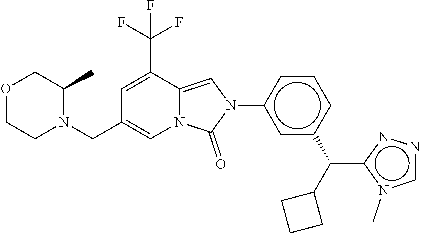 |
| 490 | 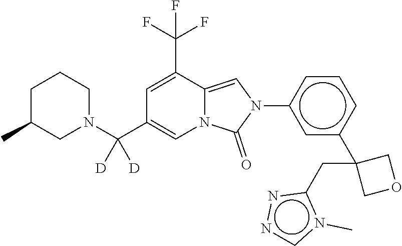 |
| 491 | 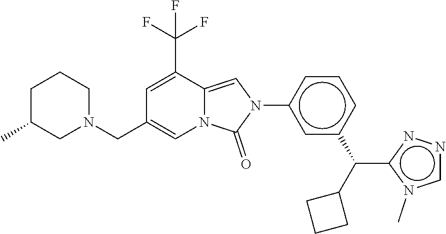 |
| 492 | 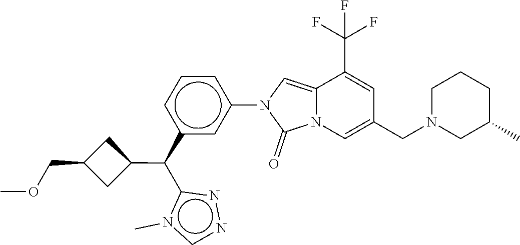 |
| 493 | 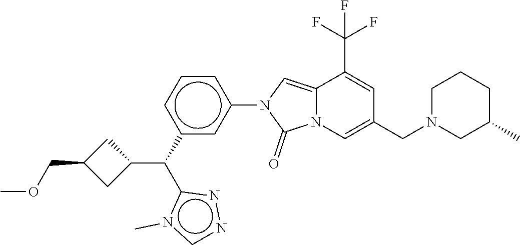 |
| 494 | 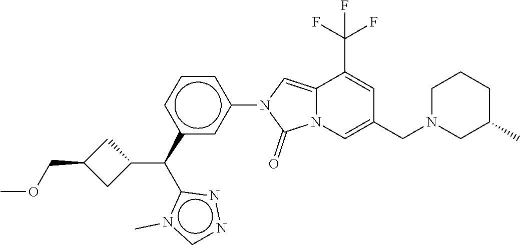 |
| 495 | 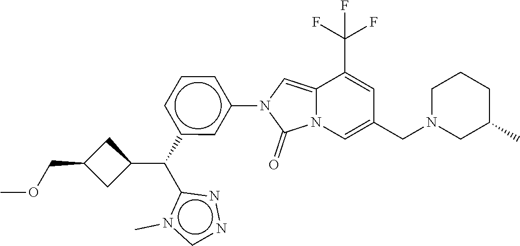 |
| 496 | 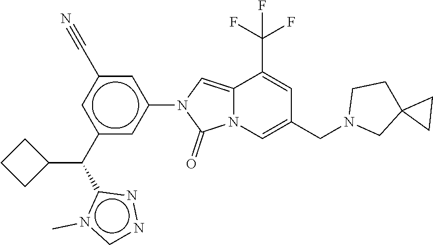 |
| 497 | 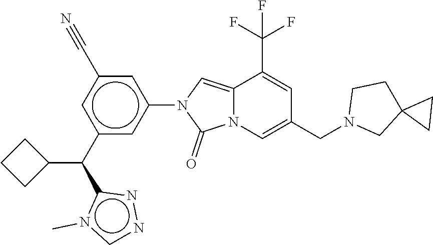 |
| 498 | 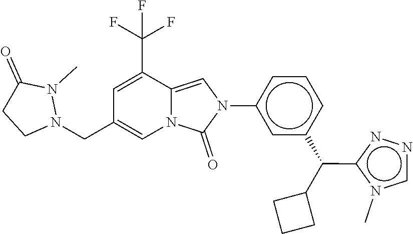 |
| 499 | 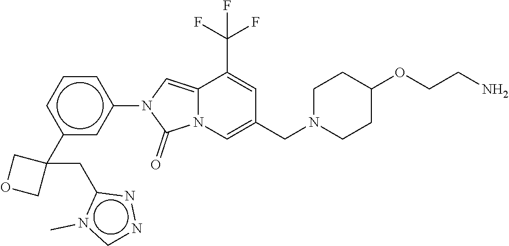 |
| 500 | 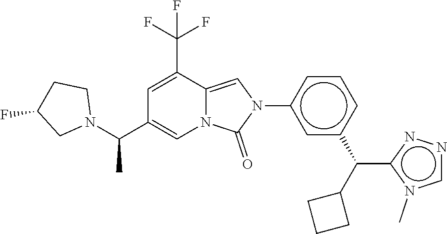 |
| 501 | 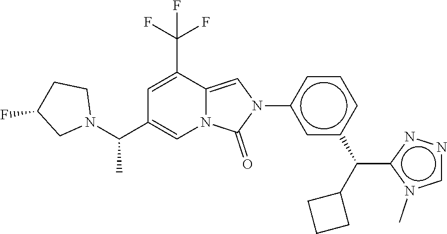 |
| 502 | 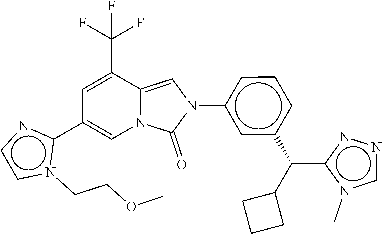 |
| 503 | 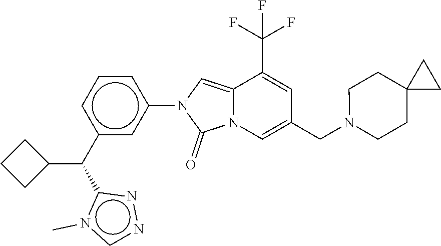 |
| 504 | 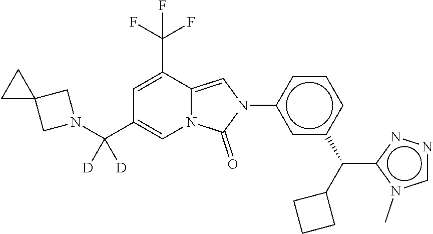 |
| 505 | 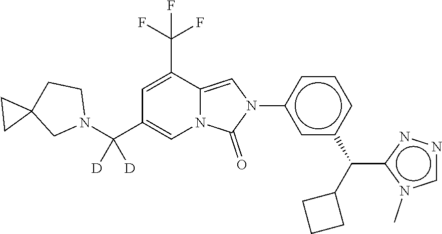 |
| 506_P1 | 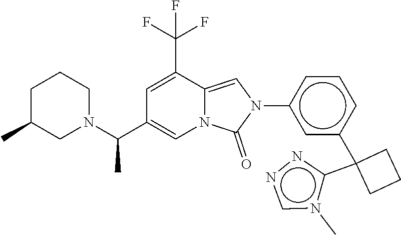 |
| 506_P2 | 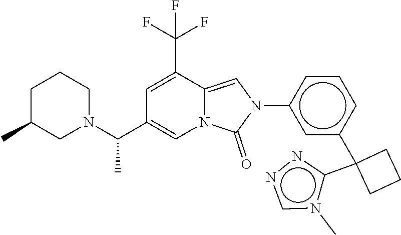 |
| 507 | 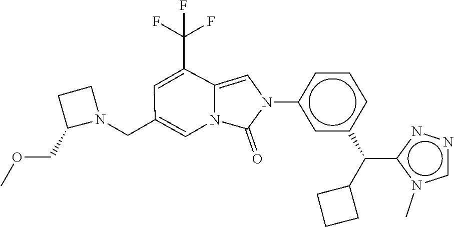 |
| 508 | 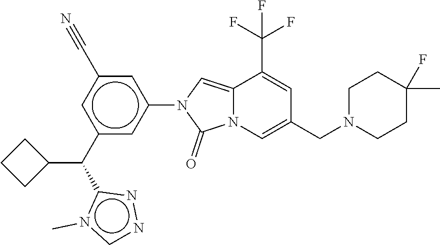 |
| 509 | 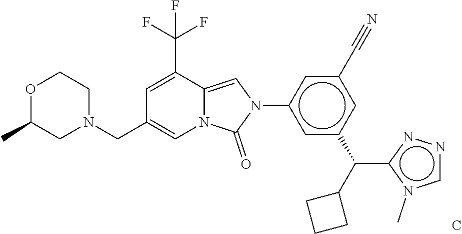 |
| 510 | 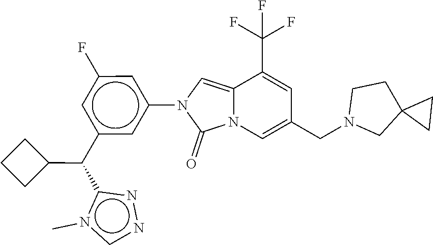 |
| 511 | 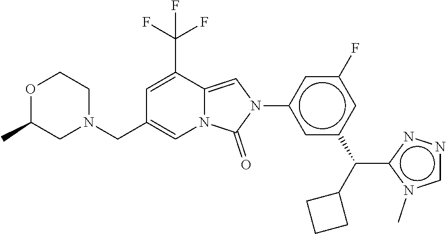 |
| 512 | 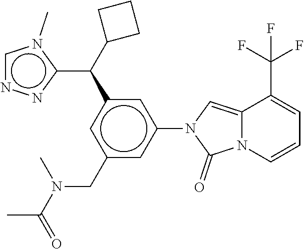 |
| 513 | 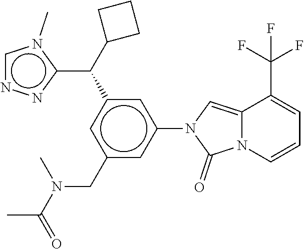 |
| 514 | 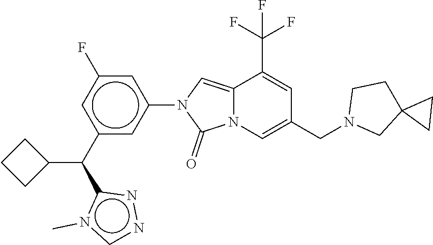 |
| 515 | 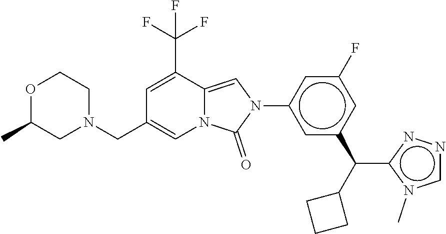 |
| 516 | 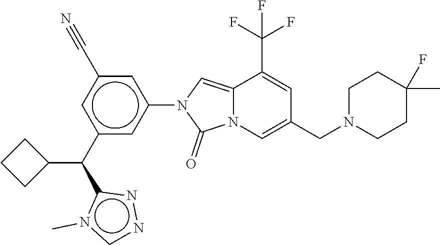 |
| 517 | 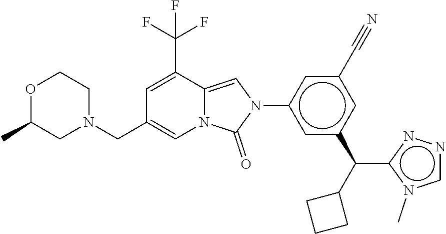 |
| 518 | 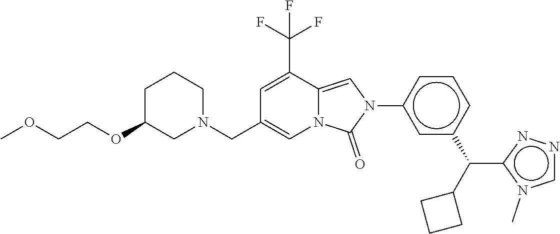 |
| 519 | 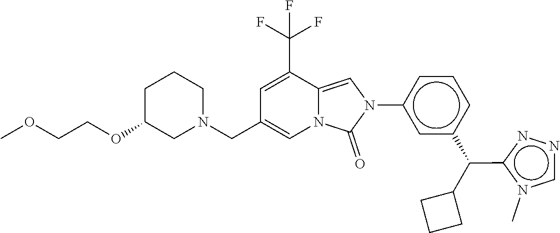 |
| 520 | 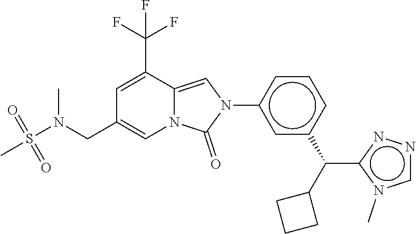 |
| 521 | 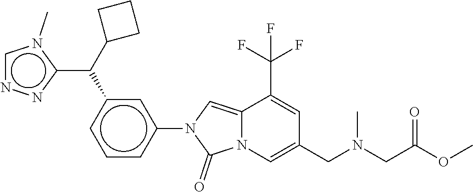 |
| 522 | 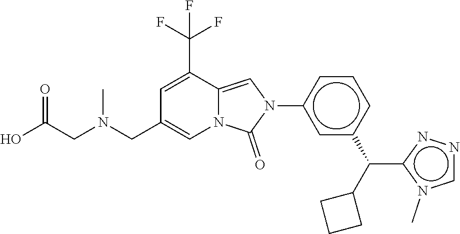 |
| 523 | 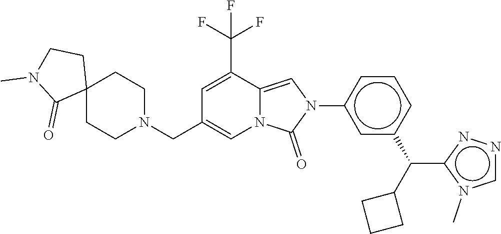 |
| 524 | 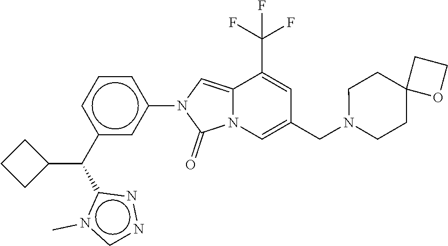 |
| 525 | 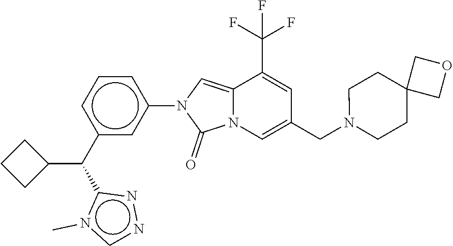 |
| 526 | 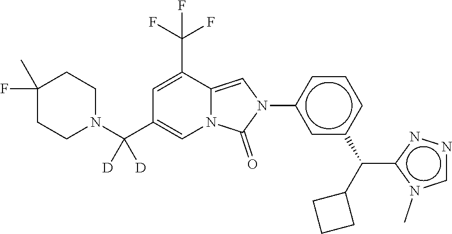 |
| 527 | 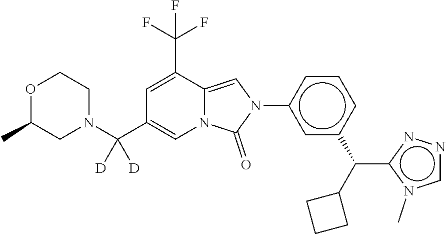 |
| 528 | 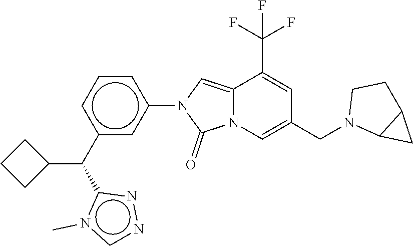 |
| 529 | 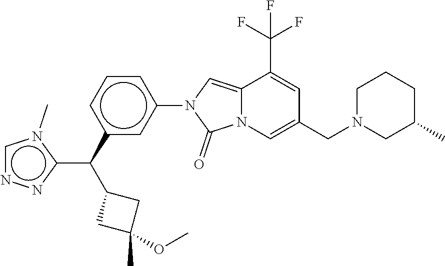 |
| 530 | 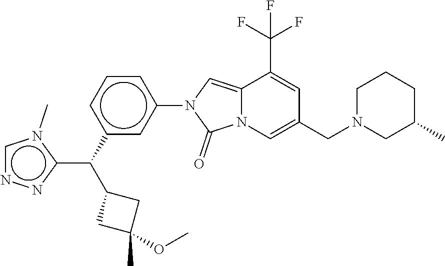 |
| 531 | 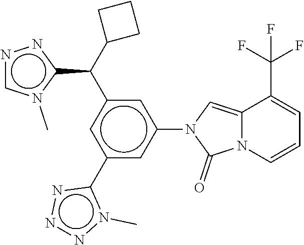 |
| 532 | 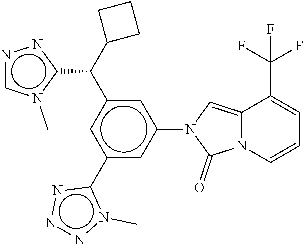 |
| 533 | 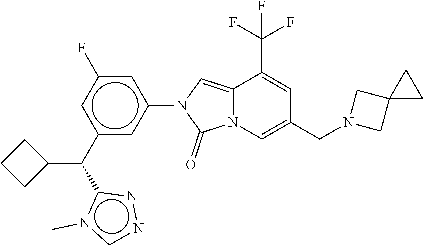 |
| 534 | 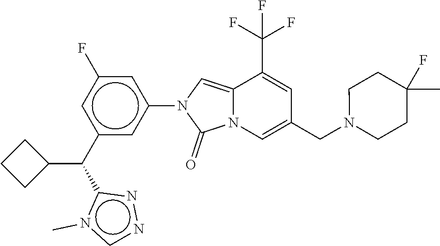 |
| 535 | 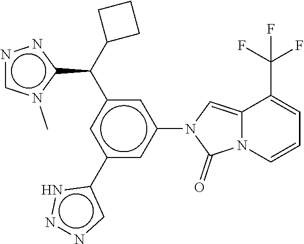 |
| 536 | 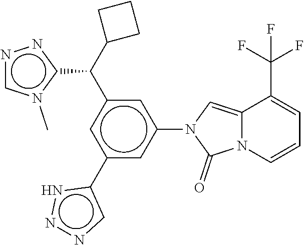 |
| 537 | 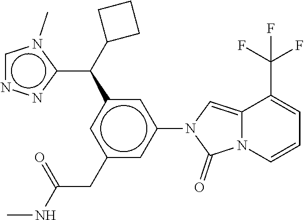 |
| 538 | 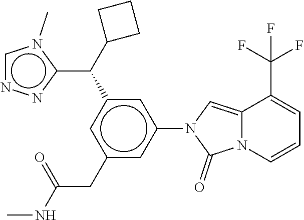 |
| 539 | 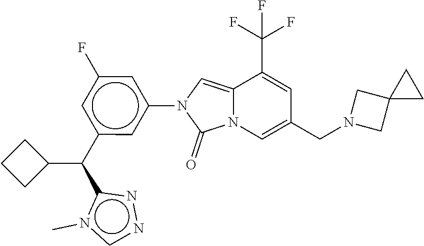 |
| 540 | 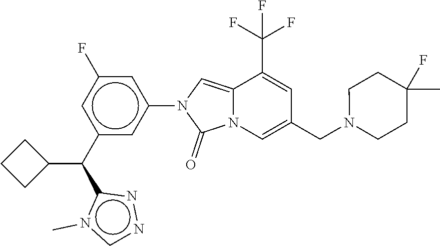 |
| 541 | 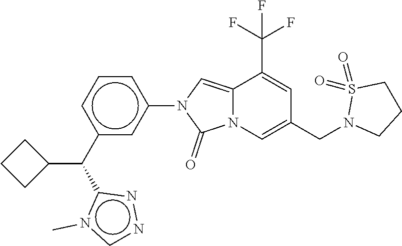 |
| 542 | 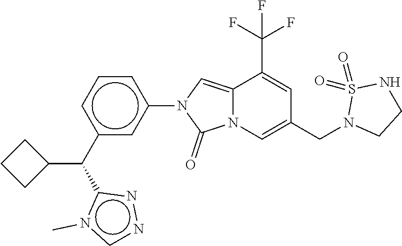 |
| 543 | 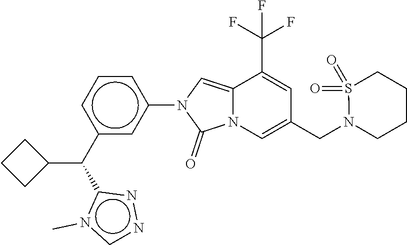 |
| 544 | 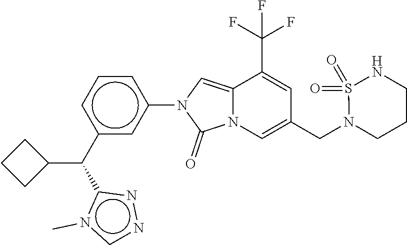 |
| 545 | 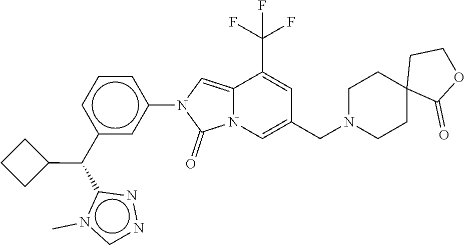 |
| 546 | 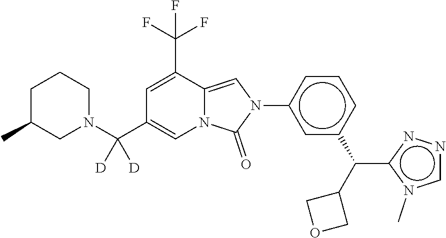 |
| 547 | 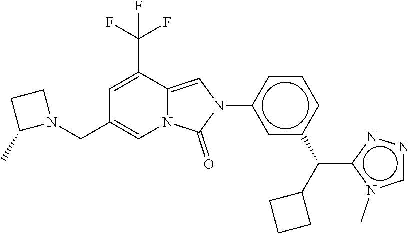 |
| 548 | 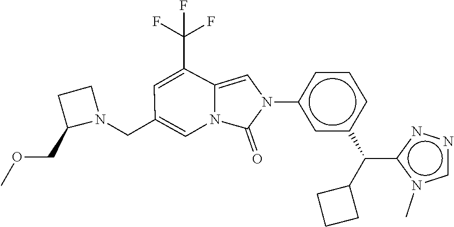 |
| 549 | 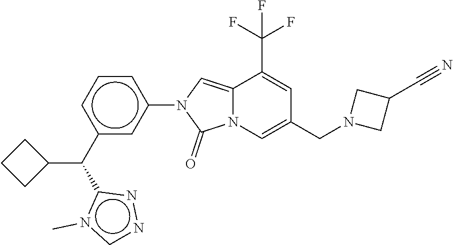 |
| 550_P1 | 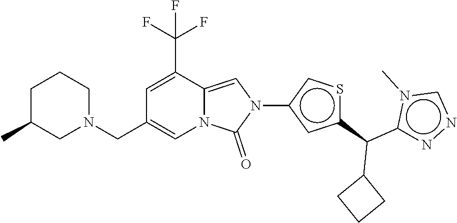 |
| 550_P2 | 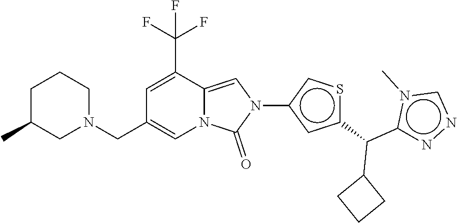 |
| 551 | 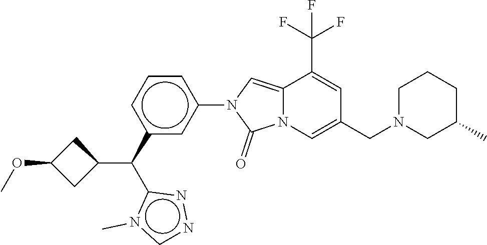 |
| 552 | 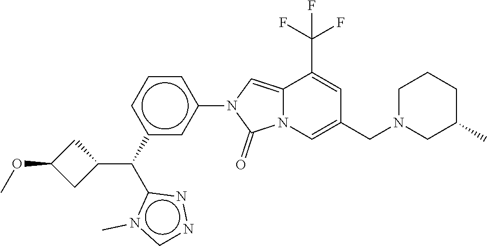 |
| 553 | 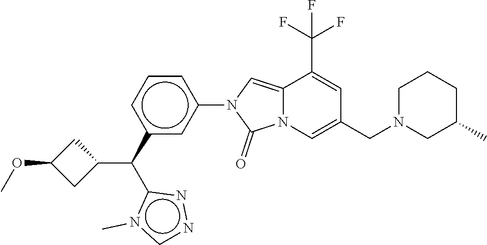 |
| 554 | 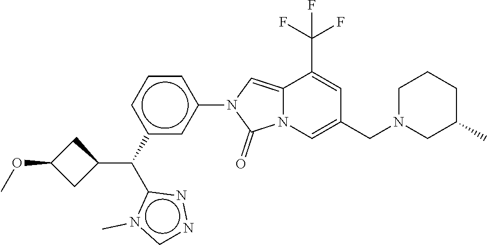 |
| 555 | 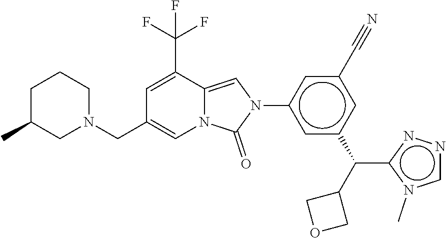 |
| 556 | 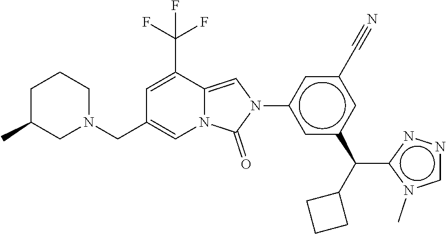 |
| 557 | 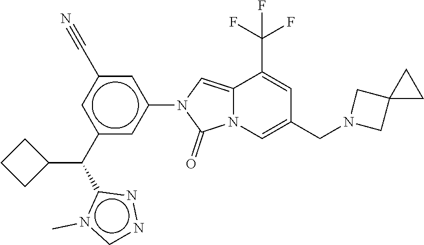 |
| 558 | 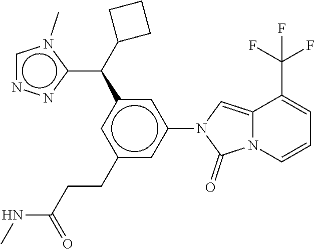 |
| 559 | 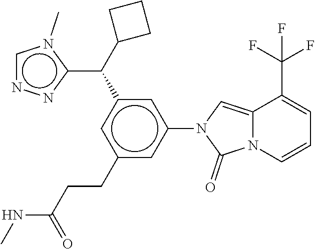 |
| 560 | 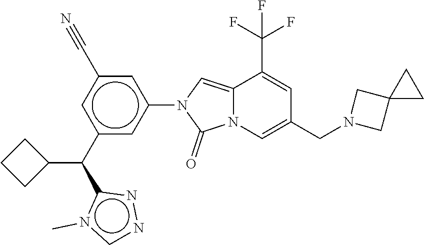 |
| 561 | 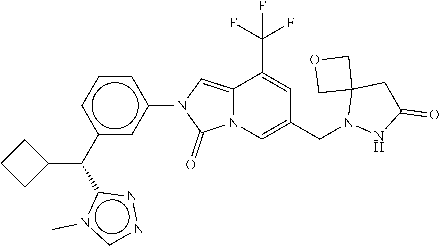 |
| 562 | 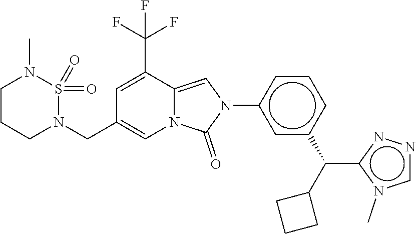 |
| 563 | 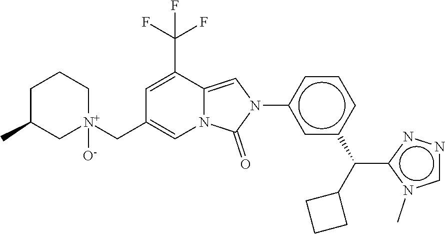 |
| 564 | 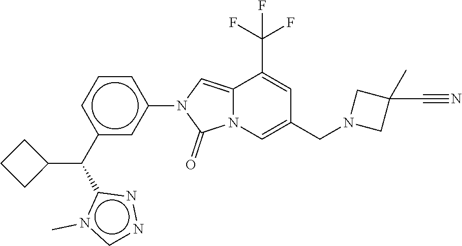 |
| 565_P1 | 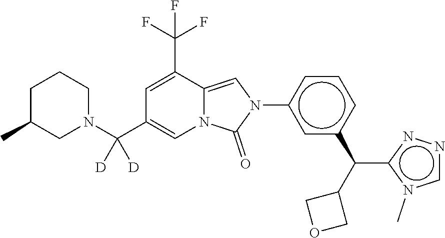 |
| 565_P2 | 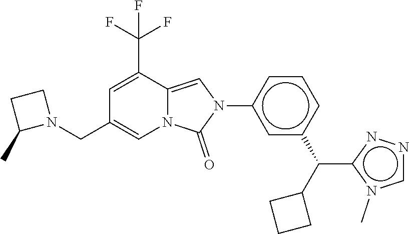 |
| 566 | 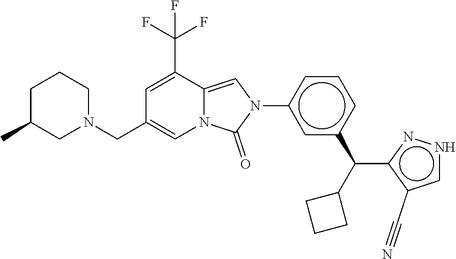 |
| 567 | 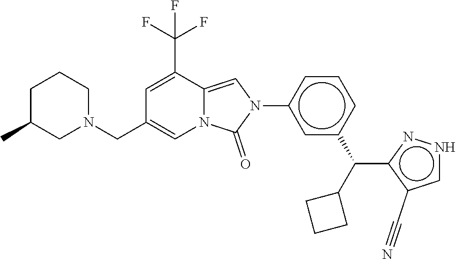 |
| 568 | 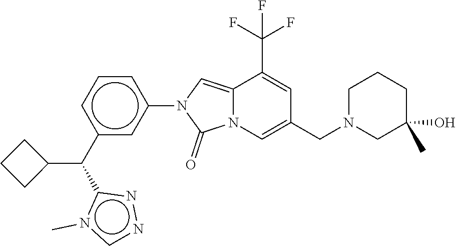 |
| 569 | 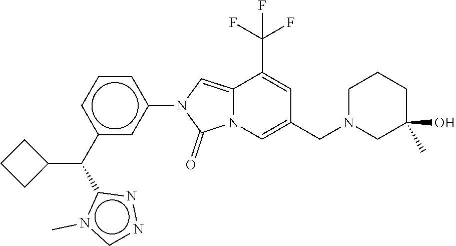 |
| 570 | 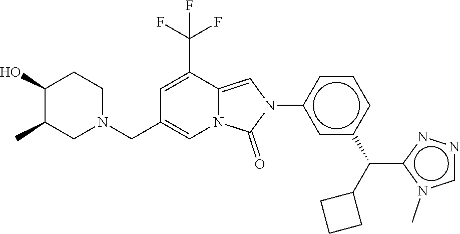 |
| 571 | 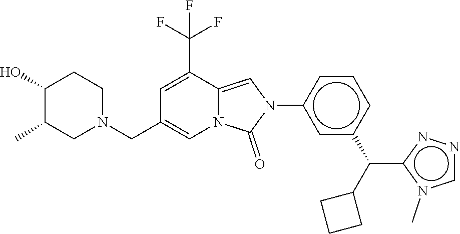 |
| 572 | 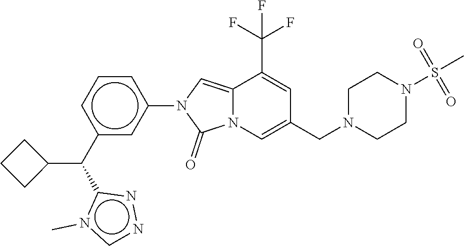 |
| 573_P1 | 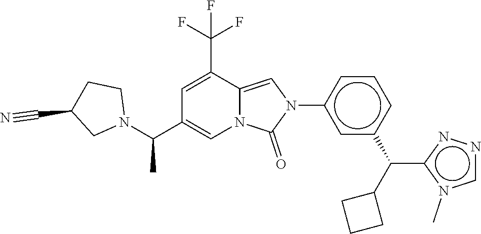 |
| 573_P2 | 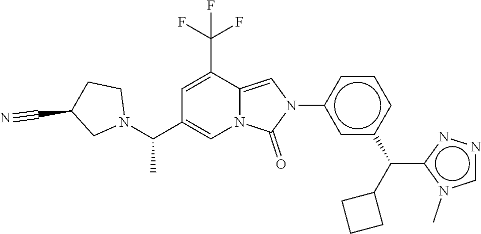 |
| 574 | 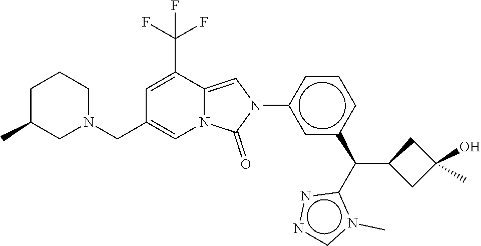 |
| 575 | 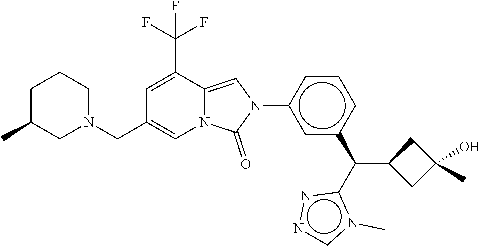 |
| 576 | 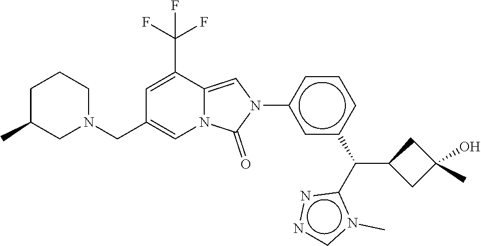 |
| 577 | 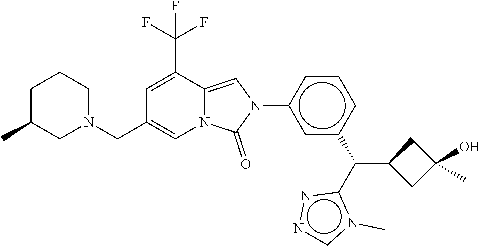 |
| 578 | 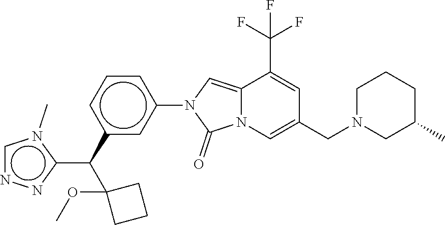 |
| 579 | 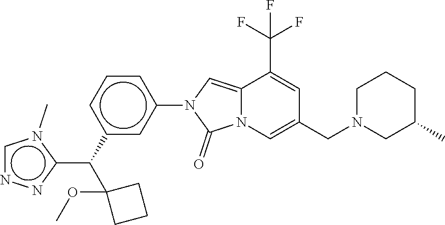 |
| 580 | 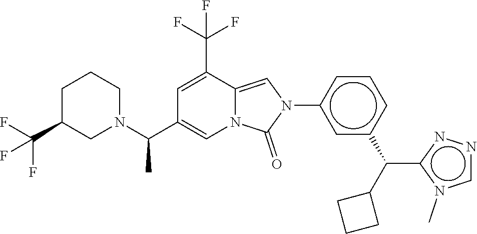 |
| 581 | 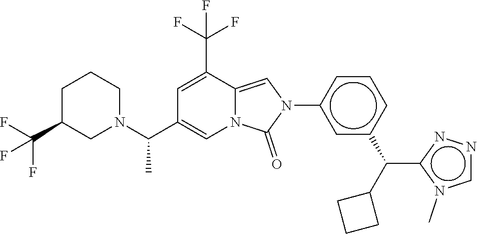 |
| 582 | 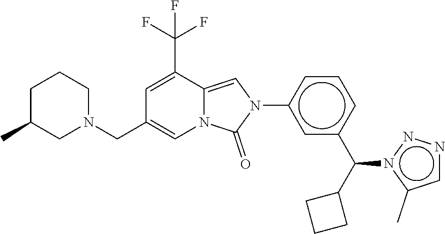 |
| 583 | 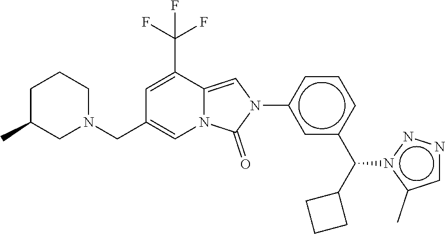 |
| 584 | 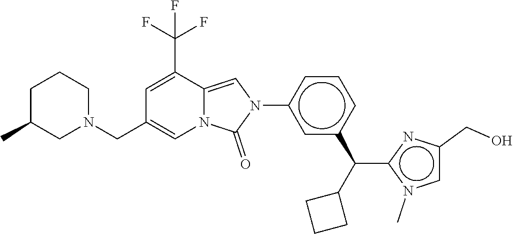 |
| 585 | 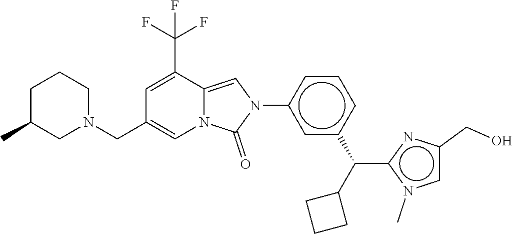 |
| 586 | 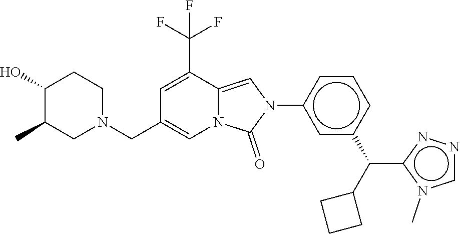 |
| 587 | 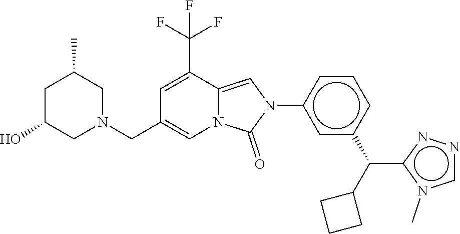 |
| 588 | 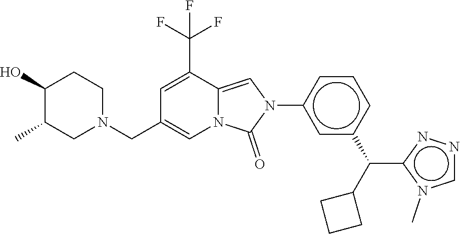 |
| 589 | 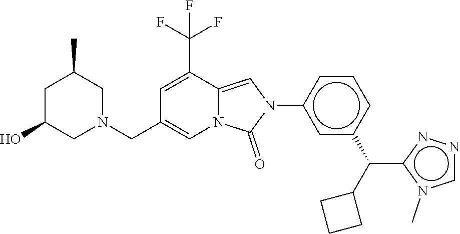 |
| 590 | 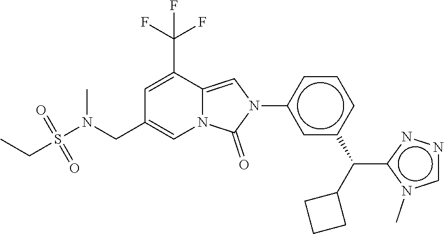 |
| 591 | 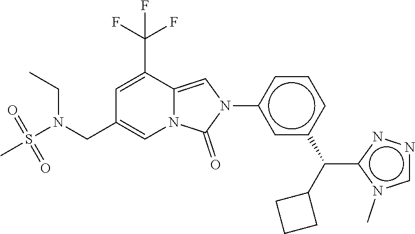 |
| 592 | 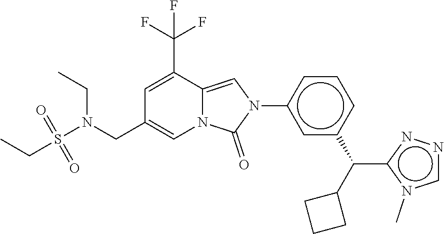 |
| 593 | 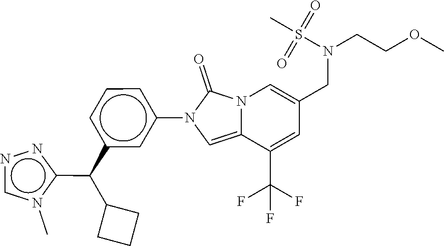 |
| 594 | 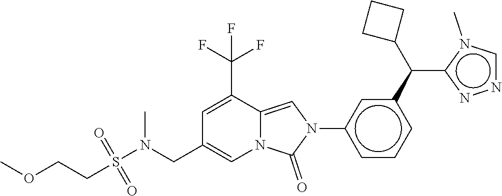 |
| 595 | 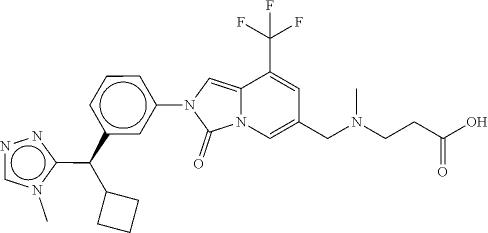 |
| 596 | 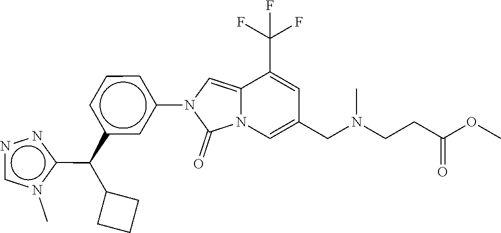 |
| 597_P1 | 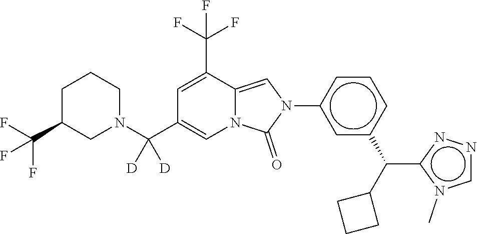 |
| 597_P2 | 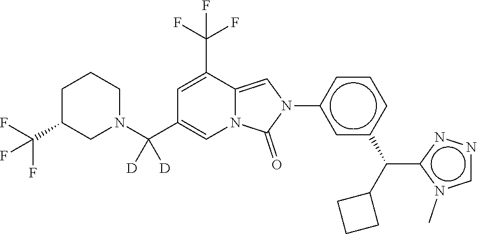 |
| 598 | 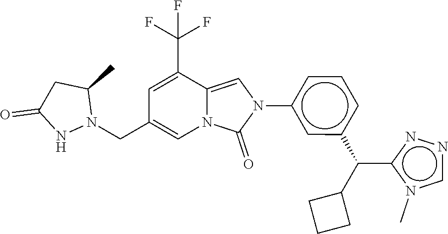 |
| 599 | 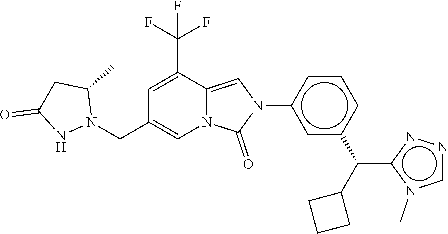 |
| 600 | 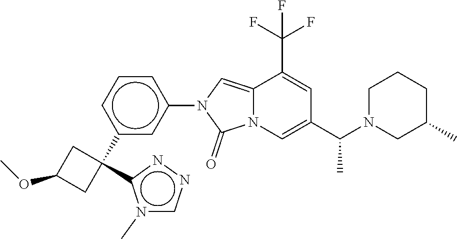 |
| 601 | 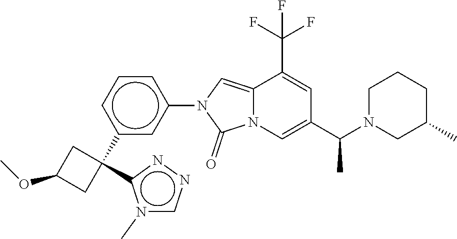 |
| 602 | 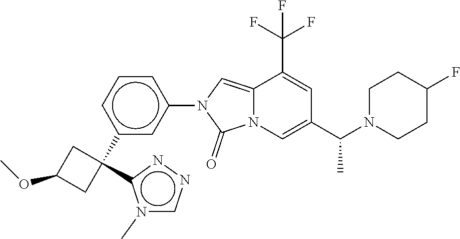 |
| 603 | 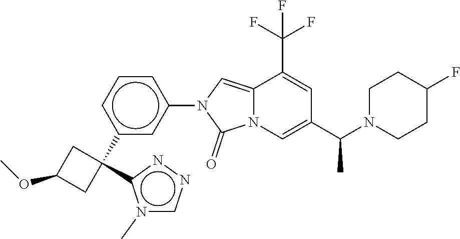 |
| 604 | 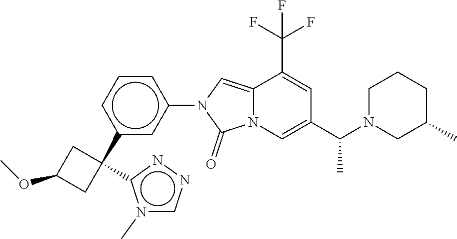 |
| 605 | 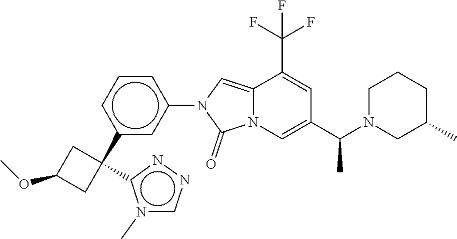 |
| 606 | 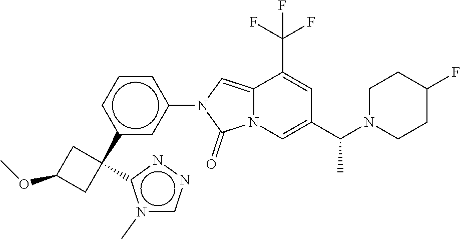 |
| 607 | 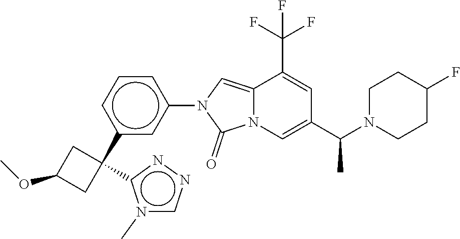 |
| 608 | 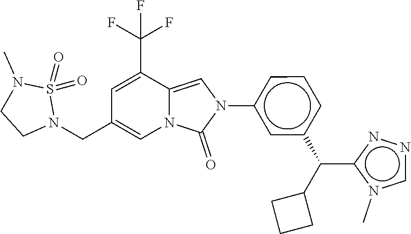 |
| 609 | 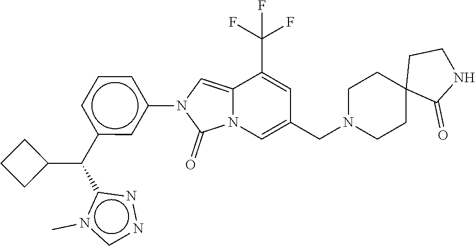 |
| 610_P1 | 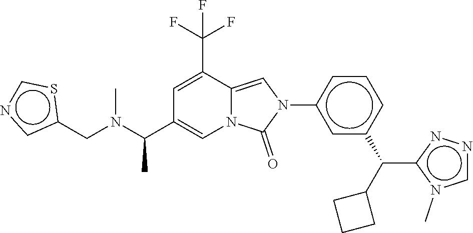 |
| 610_P2 | 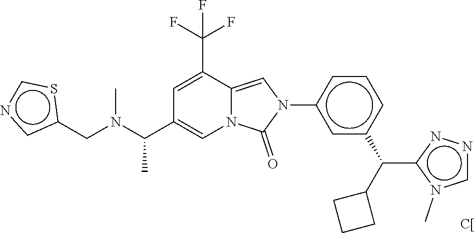 |
| 611 | 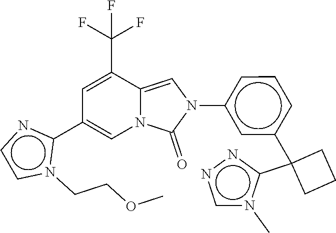 |
| 612_P1 | 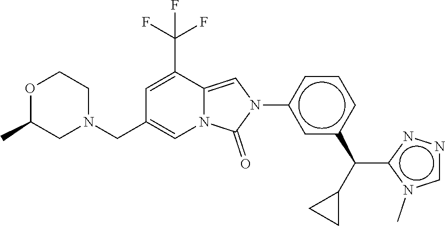 |
| 612_P2 | 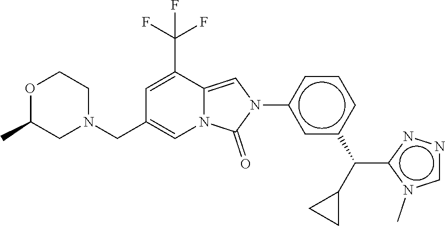 |
| 613 | 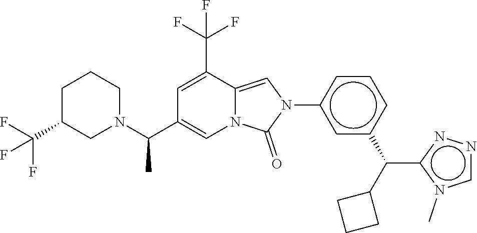 |
| 614 | 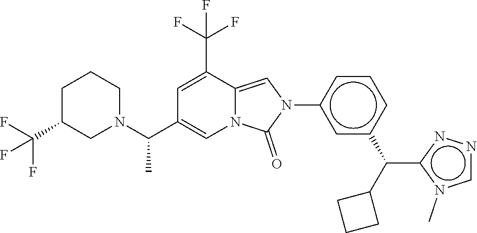 |
| 615 | 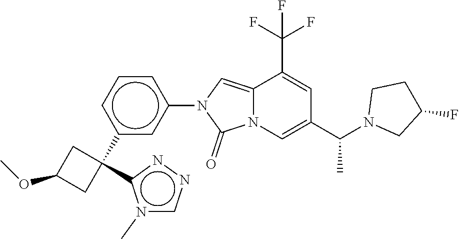 |
| 616 | 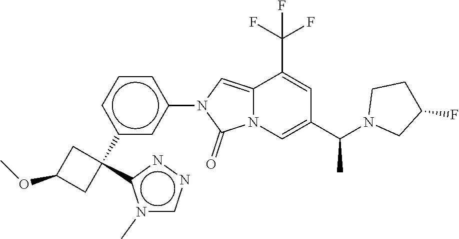 |
| 617 | 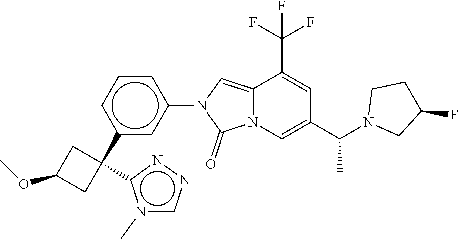 |
| 618 | 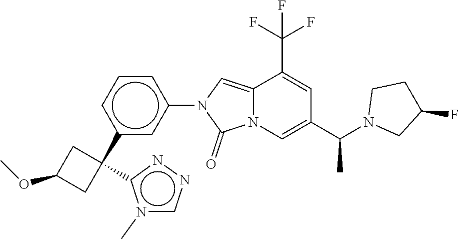 |
| 619 | 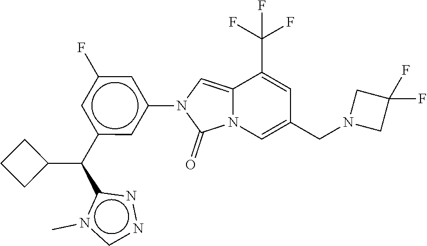 |
| 620 | 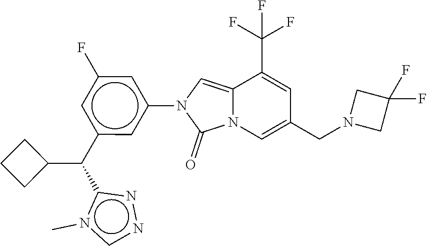 |
| 621 | 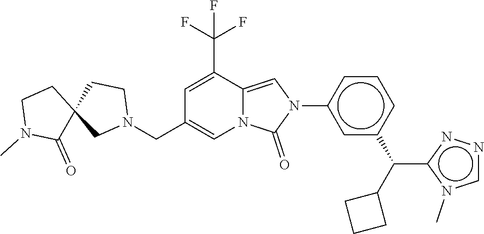 |
| 622 | 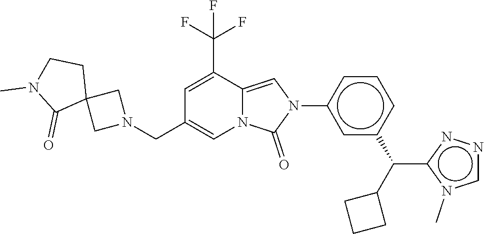 |
| 623 | 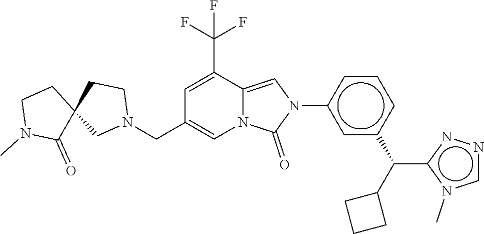 |
| 624 | 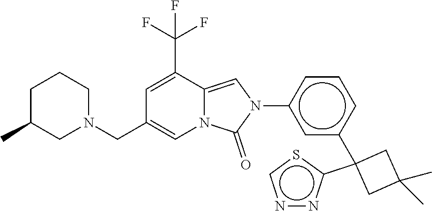 |
| 625 | 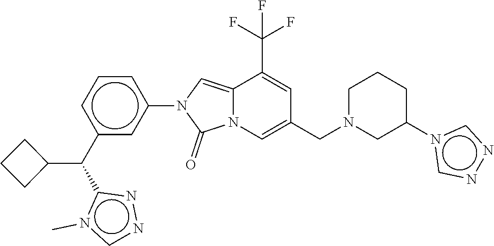 |
| 626 | 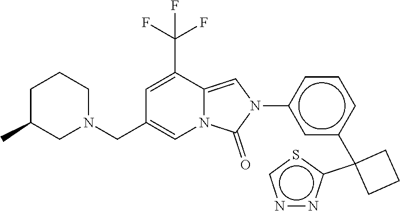 |
| 627_P1 | 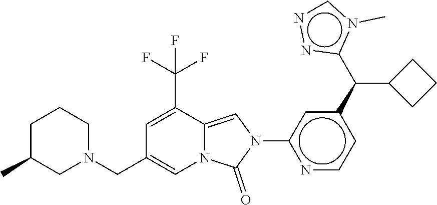 |
| 627_P2 | 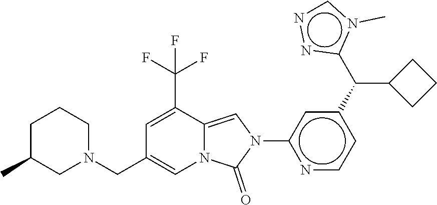 |
| 628_P1 | 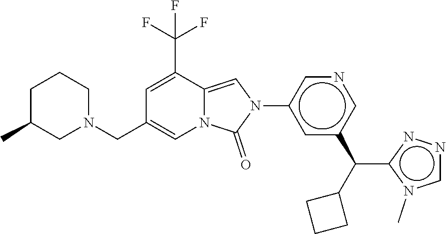 |
| 628_P2 | 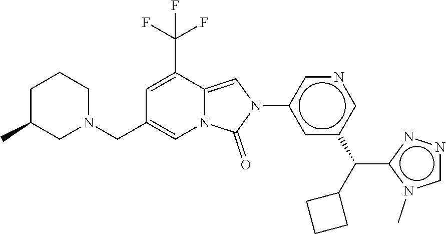 |

or a pharmaceutically acceptable salt thereof.

25. The method of any of the previous claims, wherein the compound is 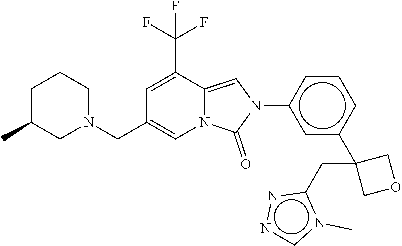
or a pharmaceutically acceptable salt thereof.

26. The method of any of the previous claims, wherein the compound is 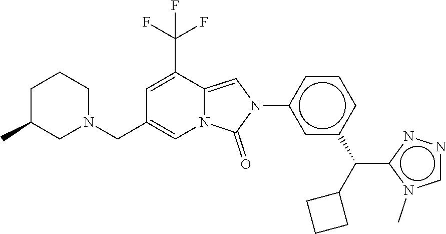
or a pharmaceutically acceptable salt thereof.

27. The method of any of the previous claims, wherein the CBL-B inhibitor is administered at a dose of 60-600 mg.

28. The method of any of the previous claims, wherein the CBL-B inhibitor is administered at a dose of 60-600 mg.

29. The method of any of the previous claims, wherein the CBL-B inhibitor is administered at a dose of 60-100 mg.

30. The method of any of the previous claims, wherein the CBL-B inhibitor is administered at a dose of 100-200 mg.

31. The method of any of the previous claims, wherein the CBL-B inhibitor is administered at a dose of 200-300 mg.

32. The method of any of the previous claims, wherein the CBL-B inhibitor is administered at a dose of 300-400 mg.

33. The method of any of the previous claims, wherein the CBL-B inhibitor is administered at a dose of 400-500 mg.

34. The method of any of the previous claims, wherein the CBL-B inhibitor is administered at a dose of 500-600 mg.

35. The method of any of the previous claims, wherein the CBL-B inhibitor is administered at a dose of about 60 mg.

36. The method of any of the previous claims, wherein the CBL-B inhibitor is administered at a dose of about 100 mg.

37. The method of any of the previous claims, wherein the CBL-B inhibitor is administered at a dose of about 150 mg.

38. The method of any of the previous claims, wherein the CBL-B inhibitor is administered at a dose of about 200 mg.

39. The method of any of the previous claims, wherein the CBL-B inhibitor is administered at a dose of about 250 mg.

40. The method of any of the previous claims, wherein the CBL-B inhibitor is administered at a dose of about 300 mg.

41. The method of any of the previous claims, wherein the CBL-B inhibitor is administered at a dose of about 350 mg.

42. The method of any of the previous claims, wherein the CBL-B inhibitor is administered at a dose of about 400 mg.

43. The method of any of the previous claims, wherein the CBL-B inhibitor is administered at a dose of about 450 mg.

44. The method of any of the previous claims, wherein the CBL-B inhibitor is administered at a dose of about 500 mg.

45. The method of any of the previous claims, wherein the CBL-B inhibitor is administered at a dose of about 550 mg.

46. The method of any of the previous claims, wherein the CBL-B inhibitor is administered at a dose of about 600 mg.

47. The method of any of the previous claims, wherein the additional therapeutic agent is selected from the group consisting of pemetrexed, carboplatin, paclitaxel/nab-paclitaxel, cisplatin, 5-fluorouracil, trastuzumab, capecitabine, oxaliplatin, leucovorin, platinum, bevacizumab, and etoposide.

48. The method of any of the previous claims, wherein the additional therapeutic agent is an additional therapeutic agent is SBRT or Concurrent chemoradiation.

49. The method of any one of claims 1-46, wherein the additional therapeutic agent is selected from the group consisting of axitinib, Lenvatinib, bevacizumab, cabozantinib, anlotinib, IBI305, and apatinib.

50. The method of any one of claims 1-46, wherein the additional therapeutic agent is selected from the group consisting of Ipilimumab, Tremelimumab, LY3321367, Sabatolimab, Relatlimab, COM701, Tiragolumab, PF-05082566, APX005M, KY1044, and GWN323.

51. The method of any one of claims 1-46, wherein the additional therapeutic agent is selected from the group consisting of erlotinib, osimertinib, crizotinib, alectinib, crizotinib, pamiparib, niraparib, olaparib, cobimetinib, AMG 510, MK-8353, dabrafenib, trametinib, encorafenib, cetuximab, copanlisib, ipatasertib, pemigatinib, B-701, savolitinib, abemaciclib, and TNO155.

52. The method of any one of claims 1-46, wherein the additional therapeutic agent is selected from the group consisting of trastuzumab emtansine (T-DM1), trastuzumab deruxtecan (T-DXd), enfortumab veotin (EV), and sacituzumab govitecan.

53. The method of any one of claims 1-46, wherein the additional therapeutic agent is selected from the group consisting of Antibody Product Antibody Product

| Antibody Product | Antibody Product |
|---|---|
| TGF-β × PD-L1 | M7824 |
| | YM101 |
| | SHR-1701 |
| CTLA-4 × PD-L1 | KN046 |
| CTLA-4 × PD-1 | MGD019 |
| | MEDI5752 |
| LAG-3 × PD-L1 | IBI323 |
| LAG-3 × PD-1 | Tebotelimab |
| TIM-3 × PD-L1 | LY3415244 |
| TIGIT × PD-L1 | |
| 4-1BB × PD-L1 | MCLA-145 |
| | ABL503 |
| | PM1003 |
| CD27 × PD-L1 | CDX-527 |
| c-Met × PD-1 | |
| EGFR × PD-L1 | |
| PD-1 × PD-L1 | LY3434172 |
| CD47 × PD-L1 | IBI322 |

54. The method of any one of claims 1-46, wherein the additional therapeutic agent is selected from the group consisting of CAR-T cells are selected from the group consisting of CD19/C19CAR-28-zeta T cells, GD2/iC9-GD2-CD28-OX40 (iC9-GD2) T cells, anti-CD19 CAR T cells, autologous anti-C19CAR-4-1BB-CD3ζ-EGFRt-expressing CD4⁺/CD8⁺ central memory T lymphocytes CAR014 T cells, CD19/KTE-C19 T cells, JCAR017 T cells, and CART-EGFRvII T cells.

55. The method of any one of claims 1-46, wherein the additional therapeutic agent is selected from the group consisting of herpes virus (e.g., HSV1716, G47Δ-mIL-12), adenovirus (e.g., hTertAd, ISF35, D24-RGDOX, ADV-TK, rHu-hDCT, ChAdOx I-STEAP I, ChAdOx I-h5T4), vaccinia virus (e.g., WR-mAb I), and myxoma virus (e.g., vPD1).

56. The method of any one of claims 1-46, wherein the additional therapeutic agent is selected from the group consisting of
Pemetrexed and carboplatin
Paclitaxel/nab-paclitaxel and carboplatin
Paclitaxel; nab-paclitaxel;
gemcitabine and carboplatin
Cisplatin and 5-fluorouracil
Trastuzumab plus either 5-fluorouracil plus cisplatin
or capecitabine plus oxaliplatin
5-fluorouracil and leucovorin plus oxaliplatin;
capecitabine and oxaliplatin
Pemetrexed and platinum
Gemcitabine and platinum
Carboplatin and pemetrexed
Gemcitabine and cisplatin
Platinum and pemetrexed
Bevacizumab plus paclitaxel and carboplatin
Carboplatin and etoposide
Nab-paclitaxel
Carboplatin and nab-paclitaxel
Etoposide and carboplatin/cisplatin

57. The method of any one of claims 1-46, wherein the additional therapeutic agent is doxrubicin.

58. The method of any one of claims 1-46, wherein the additional therapeutic agent is gemcitabine.

59. The method of any one of claims 1-46, wherein the additional therapeutic agent is paclitaxel.

60. The method of any one of claims 1-46, wherein the additional therapeutic agent is ALT-803.

61. The method of any of the previous claims, wherein the disease or condition associated with cell proliferation is hyperplasia or cancer.

62. The method of claim 61, wherein cancer is colorectal cancer.

63. The method of claim 61, wherein cancer is a hematologic cancer.

64. The method of claim 63, wherein the hematologic cancer is selected from a group consisting of lymphoma, leukemia, and myeloma.

65. The method of claim 64, wherein cancer is a non-hematologic cancer.

66. The method of claim 65, wherein the non-hematologic cancer is a sarcoma or a carcinoma.

67. The method of any one of claims 1-66, wherein the subject has one or more of increased T-cell activation, increased T-cell proliferation, decreased T-cell exhaustion, decreased T-cell anergy and decreased T-cell tolerance after administration of compound of any of claims 1-15 or a pharmaceutical composition of claim 16.

68. The method of claim 67, wherein increased T-cell activation comprises increased production of a cytokines.

69. The method of claims 1-66, wherein the subject has increased NK-cell activation.

70. The method of 69, the increased NK-cell activation comprises increased production of cytokines.
